SEQUENCE LISTING (172 sequences)

SEQ ID NO: 1 - protein, 7 aa, synthetic construct
SSHYYWG

SEQ ID NO: 2 - protein, 16 aa, synthetic construct
SIYFNGITYYSPSLKS

SEQ ID NO: 3 - protein, 16 aa, synthetic construct
HNNYYDSSGYWYYFDY

SEQ ID NO: 4 - protein, 7 aa, synthetic construct
SSSYYWG

SEQ ID NO: 5 - protein, 16 aa, synthetic construct
NIYYTGITYYNPSLKS

SEQ ID NO: 6 - protein, 12 aa, synthetic construct
VGGYSYGYLFDY

SEQ ID NO: 7 - protein, 7 aa, synthetic construct
SSSYYWG

SEQ ID NO: 8 - protein, 16 aa, synthetic construct
SIYYTGNTYYNPSLKS

SEQ ID NO: 9 - protein, 11 aa, synthetic construct
VGFSYGHPFDY

SEQ ID NO: 10 - protein, 7 aa, synthetic construct
SSSSYWG

SEQ ID NO: 11 - protein, 16 aa, synthetic construct
SIYYSGITYYNPSLKS

SEQ ID NO: 12 - protein, 9 aa, synthetic construct
LNWNQVFDY

SEQ ID NO: 13 - protein, 9 aa, synthetic construct
GDSIRSSHY

SEQ ID NO: 14 - protein, 5 aa, synthetic construct
YFNGI

SEQ ID NO: 15 - protein, 16 aa, synthetic construct
HNNYYDSSGYWYYFDY

SEQ ID NO: 16 - protein, 9 aa, synthetic construct
GGSISSSSY

SEQ ID NO: 17 - protein, 5 aa, synthetic construct
YYTGI

SEQ ID NO: 18 - protein, 12 aa, synthetic construct
VGGYSYGYLFDY

SEQ ID NO: 19 - protein, 9 aa, synthetic construct
GGSISSSSY

SEQ ID NO: 20 - protein, 5 aa, synthetic construct
YYTGN

SEQ ID NO: 21 - protein, 11 aa, synthetic construct
VGFSYGHPFDY

SEQ ID NO: 22 - protein, 9 aa, synthetic construct
GGSISSSSS

SEQ ID NO: 23 - protein, 5 aa, synthetic construct
YYSGI

SEQ ID NO: 24 - protein, 9 aa, synthetic construct
LNWNQVFDY

SEQ ID NO: 25 - protein, 126 aa, synthetic construct
QVQLVESGPGLVKPSETLSLTCTVSGDSIRSSHYYWGWIRQPPGKGLEWIGSIYFNGITYYSPSLKSRVTISVDTSKNQFSLKLTSVTAADTAVFYCARHNNYYDSSGYWYYFDYWGQGTLVTVSS

SEQ ID NO: 26 - protein, 122 aa, synthetic construct
QVQLVESGPGLVKPSETLSLTCAVSGGSISSSSYYWGWIRQPPGKGLEWIGNIYYTGITYYNPSLKSRITISVDTSKNQFSLKLSSVTAADTAVYYCARVGGYSYGYLFDYWGQGTLVTVSS

SEQ ID NO: 27 - protein, 121 aa, synthetic construct
QVQLVESGPGLVKPSETLSLTCTVSGGSISSSSYYWGWIRQPPGKGLEWIGSIYYTGNTYYNPSLKSRVTISVDTSKNQFSLKLSSVTAADTAVYYCARVGFSYGHPFDYWGQGTLVTVSS

SEQ ID NO: 28 - protein, 119 aa, synthetic construct
QVQLVESGPGLLKPSETLSLTCTVSGGSISSSSSYWGWIRQPPGKGLEWIGSIYYSGITYYNPSLKSRVTISVDTSKNQFSLKVSSVTAADTAVYYCARLNWNQVFDYWGQGTLVTVSS

SEQ ID NO: 29 - protein, 11 aa, synthetic construct
RASQSVSSYLA

SEQ ID NO: 30 - protein, 7 aa, synthetic construct
DASNRAT

SEQ ID NO: 31 - protein, 9 aa, synthetic construct
QQRSNWPPT

SEQ ID NO: 32 - protein, 11 aa, synthetic construct
RASQSVSSYLA

SEQ ID NO: 33 - protein, 7 aa, synthetic construct
DASNRAT

SEQ ID NO: 34 - protein, 9 aa, synthetic construct
QQRSNWPPT

SEQ ID NO: 35 - protein, 107 aa, synthetic construct [sequence omitted]

SEQ ID NO: 36 - protein, 5 aa, synthetic construct
NYEMN

SEQ ID NO: 37 - protein, 17 aa, synthetic construct
YIGSSGSTIYYADSVKG

SEQ ID NO: 38 - protein, 6 aa, synthetic construct
DYIFDY

SEQ ID NO: 39 - protein, 5 aa, synthetic construct
DSYMS

SEQ ID NO: 40 - protein, 17 aa, synthetic construct
YISSSISYTKYADSVKG

SEQ ID NO: 41 - protein, 14 aa, synthetic construct
DYYDSGNYYYYFDY

SEQ ID NO: 42 - protein, 5 aa, synthetic construct
NNYMS

SEQ ID NO: 43 - protein, 16 aa, synthetic construct
VIYGGGDTYYADSVKG

SEQ ID NO: 44 - protein, 12 aa, synthetic construct
GLTIFGVVPFDP

SEQ ID NO: 45 - protein, 7 aa, synthetic construct
TSGVGVG

SEQ ID NO: 46 - protein, 16 aa, synthetic construct
LIYWDDDKRYNPSLKS

SEQ ID NO: 47 - protein, 10 aa, synthetic construct
RAHSSSSSDY

SEQ ID NO: 48 - protein, 5 aa, synthetic construct
DYYWS

SEQ ID NO: 49 - protein, 16 aa, synthetic construct
EINHSGITNYNPSLKS

SEQ ID NO: 50 - protein, 8 aa, synthetic construct
GGDWNYGG

SEQ ID NO: 51 - protein, 7 aa, synthetic construct
TTRVGVG

SEQ ID NO: 52 - protein, 16 aa, synthetic construct
LIYWDDDKRYSPSLKN

SEQ ID NO: 53 - protein, 11 aa, synthetic construct
FGSGTYYNVDY

SEQ ID NO: 54 - protein, 7 aa, synthetic construct
TSGVGVG

SEQ ID NO: 55 - protein, 16 aa, synthetic construct
LIYWDDDKRYSPSLKS

SEQ ID NO: 56 - protein, 8 aa, synthetic construct
SQLGRDDY

SEQ ID NO: 57 - protein, 7 aa, synthetic construct
SRGVGVG

SEQ ID NO: 58 - protein, 16 aa, synthetic construct
LIYWDDDKRYSPSLKS

SEQ ID NO: 59 - protein, 8 aa, synthetic construct
SHRGYENF

SEQ ID NO: 60 - protein, 7 aa, synthetic construct
GFTFSNY

SEQ ID NO: 61 - protein, 6 aa, synthetic construct
GSSGST

SEQ ID NO: 62 - protein, 6 aa, synthetic construct
DYIFDY

SEQ ID NO: 63 - protein, 7 aa, synthetic construct
GFTFSDS

SEQ ID NO: 64 - protein, 6 aa, synthetic construct
SSSISY

SEQ ID NO: 65 - protein, 14 aa, synthetic construct
DYYDSGNYYYYFDY

SEQ ID NO: 66 - protein, 7 aa, synthetic construct
GFTVSNN

SEQ ID NO: 67 - protein, 5 aa, synthetic construct
YGGGD

SEQ ID NO: 68 - protein, 12 aa, synthetic construct
GLTIFGVVPFDP

SEQ ID NO: 69 - protein, 9 aa, synthetic construct
GFSLSTSGV

SEQ ID NO: 70 - protein, 5 aa, synthetic construct
YWDDD

SEQ ID NO: 71 - protein, 10 aa, synthetic construct
RAHSSSSSDY

SEQ ID NO: 72 - protein, 7 aa, synthetic construct
GGSFSDY

SEQ ID NO: 73 - protein, 11 aa, synthetic construct
NHSGEINHSGI

SEQ ID NO: 74 - protein, 8 aa, synthetic construct
GGDWNYGG

SEQ ID NO: 75 - protein, 9 aa, synthetic construct
GFSLSTTRV

SEQ ID NO: 76 - protein, 5 aa, synthetic construct
YWDDD

SEQ ID NO: 77 - protein, 11 aa, synthetic construct
FGSGTYYNVDY

SEQ ID NO: 78 - protein, 9 aa, synthetic construct
GFSLSTSGV

SEQ ID NO: 79 - protein, 5 aa, synthetic construct
YWDDD

SEQ ID NO: 80 - protein, 8 aa, synthetic construct
SQLGRDDY

SEQ ID NO: 81 - protein, 9 aa, synthetic construct
GFSLSSRGV

SEQ ID NO: 82 - protein, 5 aa, synthetic construct
YWDDD

SEQ ID NO: 83 - protein, 8 aa, synthetic construct
SHRGYENF

SEQ ID NO: 84 - protein, 115 aa, synthetic construct
EVQLVESGGGLVQPGGSLRLSCAASGFTFSNYEMNWVRQAPGKGLEWISYIGSSGSTIYYADSVKGRFTISRDNAKNSLYLQMNSLRAEDTAVYYCAVDYIFDYWGQGTLVTVSS

SEQ ID NO: 85 - protein, 123 aa, synthetic construct
EVQLVQSGGGLVKPGGSLRLSCAASGFTFSDSYMSWIRQAPGKGLEWISYISSSISYTKYADSVKGRFTISRDNAKNSLYLEMNSLRAEDTAVYYCARDYYDSGNYYYYFDYWGQGTLVTVSS

SEQ ID NO: 86 - protein, 120 aa, synthetic construct
QVQLVESGGGLVQPGGSLRLSCAASGFTVSNNYMSWVRQAPGKGLEWVSVIYGGGDTYYADSVKGRFTISRDNSQNTLYLQMTSLRADDTAVYYCARGLTIFGVVPFDPWGQGTLVTVSS

SEQ ID NO: 87 - protein, 120 aa, synthetic construct
QVQLVESGPTLVKPTQTLTLTCTFSGFSLSTSGVGVGWIRQSPGKALEWLSLIYWDDDKRYNPSLKSRLTVTKDTSKNQVVLAMTNMDPVDTATYFCAHRAHSSSSSDYWGQGTLVTVSS

SEQ ID NO: 88 - protein, 126 aa, synthetic construct
QVQLQQWGAGLLKPSETLSLTCAVYGGSFSDYYWSWIRQPPGKGLEWIGEINHSGEINHSGITNYNPSLKSRVTISVDTSKNQFSLKLSSVTAADTAVYYCARGGDWNYGGWFDPWGQGTLVTVSS

SEQ ID NO: 89 - protein, 123 aa, synthetic construct
QVQLVESGPTLVKPTQTLTLTCTFSGFSLSTTRVGVGWIRQPPGKALEWLALIYWDDDKRYSPSLKNRLTLTKDTSKNQVVLTMTNMDPVDTATYYCAHFGSGTYYNVDYWGQGTLVTVSSAS

SEQ ID NO: 90 - protein, 118 aa, synthetic construct
QITLKESGPTLVKPAQTLTLTCTFSGFSLSTSGVGVGWIRQPPGKALEWLALIYWDDDKLYSPSLKSRLTITKDTSKNQVVLAMTNMDPVDTATYYCAHSQLGRDDYWGQGTLVTVSS

SEQ ID NO: 91 - protein, 118 aa, synthetic construct
QITLKESGPTLVKPTQTLTLTCTFSGFSLSSRGVGVGWIRQPPGKALEWLALIYWDDDKRYSPSLKSRLTITKDTSKNQVVLTMANMDPVDTATYYCAHSHRGYENFWGQGTLVTVSS

SEQ ID NO: 92 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 93 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 94 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 95 - protein, 107 aa, synthetic construct [sequence omitted]

SEQ ID NO: 96 - protein, 326 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 97 - protein, 107 aa, synthetic construct [sequence omitted]

SEQ ID NO: 98 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 99 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 100 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 101 - protein, 107 aa, synthetic construct [sequence omitted]

SEQ ID NO: 102 - protein, 223 aa, synthetic construct
MACLGLRRYKAQLQLPSRTWPFVALLTLLFIPVFSEAIQVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILVAVSLGLFFYSFLVTAVSLSKMLKKRSPLTTGVYVKMPPTEPECEKQFQPYFIPIN

SEQ ID NO: 103 - protein, 334 aa, synthetic construct
KTTAPSVYPLAPVCGGTTGSSVTLGCLVKGYFPEPVTLTWNSGSLSSGVHTFPALLQSGLYTLSSSVTVTSNTWPSQTITCNVAHPASSTKVDKKIEPRVPITQNPCPPLKECPPCAAPDLLGGPSVFIFPPKIKDVLMISLSPMVTCVVVDVSEDDPDVQISWFVNNVEVHTAQTQTHREDYNSTLRVVSALPIQHQDWMSGKEFKCKVNNRALPSPIEKTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEVLHNHLTTKTISRSLGK

SEQ ID NO: 104 - protein, 270 aa, synthetic construct
MYVWVQQPTALLLLGLTLGVTARRLNCVKHTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSELPSPPTLVTPEGPAFAVLLGLGLGLLAPLTVLLALYLLRKAWRLPNTPKPCWGNSFRTPIQEEHTDAHFTLAKI

SEQ ID NO: 105 - protein, 118 aa, synthetic construct
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSS

SEQ ID NO: 106 - protein, 107 aa, synthetic construct
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEI

SEQ ID NO: 107 - protein, 120 aa, synthetic construct
QVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSS

SEQ ID NO: 108 - protein, 106 aa, synthetic construct
DIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEI

SEQ ID NO: 109 - protein, 161 aa, Homo sapiens
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSD

SEQ ID NO: 110 - protein, 214 aa, Homo sapiens
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRA

SEQ ID NO: 111 - protein, 453 aa, synthetic construct
EVQLVQSGGGLVKPGGSLRLSCAASGFTFSDSYMSWIRQAPGKGLEWISYISSSISYTKYADSVKGRFTISRDNAKNSLYLEMNSLRAEDTAVYYCARDYYDSGNYYYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 112 - protein, 456 aa, synthetic construct
QVQLVESGPGLVKPSETLSLTCTVSGDSIRSSHYYWGWIRQPPGKGLEWIGSIYFNGITYYSPSLKSRVTISVDTSKNQFSLKLTSVTAADTAVFYCARHNNYYDSSGYWYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 113 - protein, 450 aa, synthetic construct
QVQLVESGGGLVQPGGSLRLSCAASGFTVSNNYMSWVRQAPGKGLEWVSVIYGGGDTYYADSVKGRFTISRDNSQNTLYLQMTSLRADDTAVYYCARGLTIFGVVPFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 114 - protein, 456 aa, synthetic construct; identical to SEQ ID NO: 112

SEQ ID NO: 115 - protein, 449 aa, synthetic construct
QVQLVESGPGLLKPSETLSLTCTVSGGSISSSSSYWGWIRQPPGKGLEWIGSIYYSGITYYNPSLKSRVTISVDTSKNQFSLKVSSVTAADTAVYYCARLNWNQVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 116 - protein, 450 aa, synthetic construct
QVQLVESGGGLVQPGGSLRLSCAASGFTVSNNYMSWVRQAPGKGLEWVSVIYGGGDTYYADSVKGRFTISRDNSQNTLYLQMTSLRADDTAVYYCARGLTIFGVVPFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 117 - protein, 449 aa, synthetic construct; identical to SEQ ID NO: 115

SEQ ID NO: 118 - protein, 456 aa, synthetic construct
QVQLQQWGAGLLKPSETLSLTCAVYGGSFSDYYWSWIRQPPGKGLEWIGEINHSGEINHSGITNYNPSLKSRVTISVDTSKNQFSLKLSSVTAADTAVYYCARGGDWNYGGWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 119 - protein, 456 aa, synthetic construct
QVQLQQWGAGLLKPSETLSLTCAVYGGSFSDYYWSWIRQPPGKGLEWIGEINHSGEINHSGITNYNPSLKSRVTISVDTSKNQFSLKLSSVTAADTAVYYCARGGDWNYGGWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 120 - protein, 452 aa, synthetic construct
QVQLVESGPGLVKPSETLSLTCAVSGGSISSSSYYWGWIRQPPGKGLEWIGNIYYTGITYYNPSLKSRITISVDTSKNQFSLKLSSVTAADTAVYYCARVGGYSYGYLFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 121 - protein, 451 aa, synthetic construct
QVQLVESGPGLVKPSETLSLTCTVSGGSISSSSYYWGWIRQPPGKGLEWIGSIYYTGNTYYNPSLKSRVTISVDTSKNQFSLKLSSVTAADTAVYYCARVGFSYGHPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 122 - protein, 450 aa, synthetic construct; identical to SEQ ID NO: 116

SEQ ID NO: 123 - protein, 451 aa, synthetic construct; identical to SEQ ID NO: 121

SEQ ID NO: 124 - protein, 456 aa, synthetic construct; identical to SEQ ID NO: 118

SEQ ID NO: 125 - protein, 453 aa, synthetic construct; identical to SEQ ID NO: 111

SEQ ID NO: 126 - protein, 452 aa, synthetic construct; identical to SEQ ID NO: 120

SEQ ID NO: 127 - protein, 450 aa, synthetic construct; identical to SEQ ID NO: 113

SEQ ID NO: 128 - protein, 452 aa, synthetic construct; identical to SEQ ID NO: 120

SEQ ID NO: 129 - protein, 456 aa, synthetic construct; identical to SEQ ID NO: 119

SEQ ID NO: 130 - protein, 456 aa, synthetic construct; identical to SEQ ID NO: 112

SEQ ID NO: 131 - protein, 449 aa, synthetic construct; identical to SEQ ID NO: 115

SEQ ID NO: 132 - protein, 453 aa, synthetic construct
EVQLVQSGGGLVKPGGSLRLSCAASGFTFSDSYMSWIRQAPGKGLEWISYISSSISYTKYADSVKGRFTISRDNAKNSLYLEMNSLRAEDTAVYYCARDYYDSGNYYYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 133 - protein, 451 aa, synthetic construct; identical to SEQ ID NO: 121

SEQ ID NO: 134 - protein, 453 aa, synthetic construct; identical to SEQ ID NO: 132

SEQ ID NO: 135 - protein, 445 aa, synthetic construct
EVQLVESGGGLVQPGGSLRLSCAASGFTFSNYEMNWVRQAPGKGLEWISYIGSSGSTIYYADSVKGRFTISRDNAKNSLYLQMNSLRAEDTAVYYCAVDYIFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 136 - protein, 456 aa, synthetic construct; identical to SEQ ID NO: 112

SEQ ID NO: 137 - protein, 450 aa, synthetic construct
QVQLVESGPTLVKPTQTLTLTCTFSGFSLSTSGVGVGWIRQSPGKALEWLSLIYWDDDKRYNPSLKSRLTVTKDTSKNQVVLAMTNMDPVDTATYFCAHRAHSSSSSDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 138 - protein, 456 aa, synthetic construct; identical to SEQ ID NO: 112

SEQ ID NO: 139 - protein, 448 aa, synthetic construct
QITLKESGPTLVKPAQTLTLTCTFSGFSLSTSGVGVGWIRQPPGKALEWLALIYWDDDKLYSPSLKSRLTITKDTSKNQVVLAMTNMDPVDTATYYCAHSQLGRDDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 140 - protein, 456 aa, synthetic construct; identical to SEQ ID NO: 112

SEQ ID NO: 141 - protein, 448 aa, synthetic construct
QITLKESGPTLVKPTQTLTLTCTFSGFSLSSRGVGVGWIRQPPGKALEWLALIYWDDDKRYSPSLKSRLTITKDTSKNQVVLTMANMDPVDTATYYCAHSHRGYENFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 142 - protein, 456 aa, synthetic construct; identical to SEQ ID NO: 112

SEQ ID NO: 143 - protein, 453 aa, synthetic construct
QVQLVESGPTLVKPTQTLTLTCTFSGFSLSTTRVGVGWIRQPPGKALEWLALIYWDDDKRYSPSLKNRLTLTKDTSKNQVVLTMTNMDPVDTATYYCAHFGSGTYYNVDYWGQGTLVTVSSASASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 144 - protein, 456 aa, synthetic construct; identical to SEQ ID NO: 112

SEQ ID NO: 145 - protein, 450 aa, synthetic construct; identical to SEQ ID NO: 113

SEQ ID NO: 146 - protein, 456 aa, synthetic construct; identical to SEQ ID NO: 112

SEQ ID NO: 147 - protein, 214 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 148 - protein, 254 aa, Homo sapiens
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQVSFCLVMVLLFAVDTGLYFSVKTNIRSSTRDWKDHKFKWRKDPQDK

SEQ ID NO: 149 - protein, 11 aa, synthetic construct
RASQSVSNYLA

SEQ ID NO: 150 - protein, 7 aa, synthetic construct
AASSRAT

SEQ ID NO: 151 - protein, 9 aa, synthetic construct
QQRRDWPPT

SEQ ID NO: 152 - protein, 11 aa, synthetic construct
RASQSINSYLA

SEQ ID NO: 153 - protein, 7 aa, synthetic construct
LASNRAT

SEQ ID NO: 154 - protein, 9 aa, synthetic construct
QQRSDWPPT

SEQ ID NO: 155 - protein, 11 aa, synthetic construct
RASQSVSNYLA

SEQ ID NO: 156 - protein, 7 aa, synthetic construct
AASSRAT

SEQ ID NO: 157 - protein, 9 aa, synthetic construct
QQRRDWPPT

SEQ ID NO: 158 - protein, 11 aa, synthetic construct
RASQSINSYLA

SEQ ID NO: 159 - protein, 7 aa, synthetic construct
LASNRAT

SEQ ID NO: 160 - protein, 9 aa, synthetic construct
QQRSDWPPT

SEQ ID NO: 161 - protein, 107 aa, synthetic construct
EIVLTQSPATLCLSPGERATLSCRASQSVSNYLAWYQQKPGQAPRLLIYAASSRATGIPARFSGSGSGTDFTLTISTLEPEDFAVYYCQQRRDWPPTFGQGTKVEIK

SEQ ID NO: 162 - protein, 107 aa, synthetic construct
EIVLTQSPATLSLSPGERATLSCRASQSINSYLAWYQQNPGQAPRLLIYLASNRATGIPARFSGSGSGTDFTLTITSLEPEDFAVYYCQQRSDWPPTFGQGTKVEIK

SEQ ID NO: 163 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 164 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 165 - protein, 120 aa, synthetic construct
EVQLQESGPGLVKPSQSLSLTCSVTGYSITSSYRWNWIRKFPGNRLEWMGYINSAGISNYNPSLKRRISITRDTSKNQFFLQVNSVTTEDAATYYCARSDNMGTTPFTYWGQGTLVTVSS

SEQ ID NO: 166 - protein, 111 aa, synthetic construct
DIVMTQGTLPNPVPSGESVSITCRSSKSLLYSDGKTYLNWYLQRPGQSPQLLIYWMSTRASGVSDRFSGSGSGTDFTLKISGVEAEDVGIYYCQQGLEFPTFGGGTKLELK

SEQ ID NO: 167 - protein, 118 aa, synthetic construct [sequence omitted]

SEQ ID NO: 168 - protein, 107 aa, synthetic construct [sequence omitted]

SEQ ID NO: 169 - protein, 288 aa, synthetic construct
MWVRQVPWSFTWAVLQLSWQSGWLLEVPNGPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELVVTERILETSTRYPSPSPKPEGRFQGMVIGIMSALVGIPVLLLLAWALAVFCSTSMSEARGAGSKDDTLKEEPSAAPVPSVAYEELDFQGREKTPELPTACVHTEYATIVFTEGLGASAMGRRGSADGLQGPRPPRHEDGHCSWPL

SEQ ID NO: 170 - protein, 289 aa, synthetic construct
MVSLPRLCALWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALLVIPVVMGILITIFGVFLYIKKVVKKPKDNEILPPAARRQDPQEMEDYPGHNTAAPVQETLHGCQPVTQEDGKESRISVQERQVTDSIALRPLV

SEQ ID NO: 171 - protein, 713 aa, synthetic construct
EVQLVESGGGLVQPGGSLRLSCAASGFTFSSYSMSWVRQAPGKRLEWVAHISSGGSSTYYPDTVKGRFTISRDNSKNTLYLQMNSLRAEDTAMYYCTRQFYYGSSFWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSDVVMTQSPLSLPVTLGQPASISCRSSQSLLHSNGNTYLHWYQQRPGQSPNHLIYQVSNRFSGVPDRFSGSGSGTDFTLKISRVEAEDVGVYFCSQTTHVPWTFGCGTKVEIKGGGGSGGGGSGGGGSGGGGSQVQLVQSGAEVKKPGASVKLSCKASGYTFISYYIYWVKQAPGQCLEWIGGINPRNGGTNFNEKFKSRATLTVDTSISTAYMELSRLRSEDTAVYYCTRHGNGVYWGQGTTLTVSS

SEQ ID NO: 172 - protein, 218 aa, synthetic construct
DNVLTQSPATLSVSPGERATLSCKASQSVDFDGDSYMNWYQQKPGQPPRLLIYAASNLESGIPARFSGSGSGTDFTLTISSVEPEDFATYYCQQSNEDPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2025/0115668 A1
(43) Pub. Date: Apr. 10, 2025

(54) ANTI-CTLA4/OX40 BISPECIFIC ANTIBODIES AND USES THEREOF

(71) Applicant: Biocytogen Pharmaceuticals (Beijing) Co., Ltd., Beijing (CN)

(72) Inventors: Baihong Liu, Beijing (CN); Yanfei Han, Beijing (CN); Yan Huang, Beijing (CN); Xin Ji, Beijing (CN); Yi Yang, Beijing (CN); Yuelei Shen, Beijing (CN)

(21) Appl. No.: 18/729,205

(22) PCT Filed: Jan. 30, 2023
(86) PCT No.: PCT/CN2023/073772

§ 371 Date: Jul. 16, 2024

(30) Foreign Application Priority Data
Jan. 30, 2022 (WO) PCT/CN2022/075189
Dec. 21, 2022 (WO) PCT/CN2022/140544

(51) Int. Cl. C07K 16/28; A61K 39/00; A61P 35/00

(52) U.S. Cl. CPC C07K 16/2818; A61P 35/00; C07K 16/2875; A61K 2039/505; C07K 2317/14; C07K 2317/24; C07K 2317/31; C07K 2317/33; C07K 2317/565; C07K 2317/622; C07K 2317/732

(57) ABSTRACT

An antigen-binding protein constructs (e.g., bispecific antibodies or antigen-binding fragments thereof), wherein the antigen-binding protein constructs specifically bind to two different antigens (e.g., CTLA4 and OX40).

### CLAIM OF PRIORITY

This application claims the benefit of PCT Application No. PCT/CN2022/075189, filed on Jan. 30, 2022, and PCT Application No. PCT/CN2022/140544, filed on Dec. 21, 2022. The entire contents of the foregoing are incorporated herein by reference.

### TECHNICAL FIELD

This disclosure relates to antigen-binding protein constructs (e.g., bispecific antibodies or antigen-binding fragments thereof).

### BACKGROUND

A bispecific antibody is an artificial protein that can simultaneously bind to two different types of antigens or two different epitopes. This dual specificity opens up a wide range of applications, including redirecting T cells to tumor cells, dual targeting of different disease mediators, and delivering payloads to targeted sites. The approval of catumaxomab (anti-EpCAM and anti-CD3) and blinatumomab (anti-CD19 and anti-CD3) has become a major milestone in the development of bispecific antibodies.

As bispecific antibodies have various applications, there is a need to continue to develop various therapeutics based on bispecific antibodies.

### SUMMARY

This disclosure relates to antigen-binding protein constructs, wherein the antigen-binding protein construct specifically bind to two different antigens (e.g., CTLA4 and OX40). In some embodiments, the multispecific antibody (e.g., bispecific antibody) has identical light chain variable regions. In some embodiments, the multispecific antibody (e.g., bispecific antibody) has a common light chain.

In one aspect, the disclosure is related to an antibody or antigen-binding fragment thereof that binds to CTLA4 (Cytotoxic T-lymphocyte antigen-4) comprising: a heavy chain variable region (VH) comprising complementarity determining regions (CDRs) 1, 2, and 3, in some embodiments, the VH CDR1 region comprises an amino acid sequence that is at least 80% identical to a selected VH CDR1 amino acid sequence, the VH CDR2 region comprises an amino acid sequence that is at least 80% identical to a selected VH CDR2 amino acid sequence, and the VH CDR3 region comprises an amino acid sequence that is at least 80% identical to a selected VH CDR3 amino acid sequence; and a light chain variable region (VL) comprising CDRs 1, 2, and 3, in some embodiments, the VL CDR1 region comprises an amino acid sequence that is at least 80% identical to a selected VL CDR1 amino acid sequence, the VL CDR2 region comprises an amino acid sequence that is at least 80% identical to a selected VL CDR2 amino acid sequence, and the VL CDR3 region comprises an amino acid sequence that is at least 80% identical to a selected VL CDR3 amino acid sequence, in some embodiments, the selected VH CDRs 1, 2, and 3 amino acid sequences and the selected VL CDRs, 1, 2, and 3 amino acid sequences are one of the following:
(1) the selected VH CDRs 1, 2, 3 amino acid sequences are set forth in SEQ ID NOs: 1-3, respectively, and the selected VL CDRs 1, 2, 3 amino acid sequences are set forth in SEQ ID NOs: 29-31, respectively;
(2) the selected VH CDRs 1, 2, 3 amino acid sequences are set forth in SEQ ID NOs: 4-6, respectively, and the selected VL CDRs 1, 2, 3 amino acid sequences are set forth in SEQ ID NOs: 29-31, respectively;
(3) the selected VH CDRs 1, 2, 3 amino acid sequences are set forth in SEQ ID NOs: 7-9, respectively, and the selected VL CDRs 1, 2, 3 amino acid sequences are set forth in SEQ ID NOs: 29-31, respectively;
(4) the selected VH CDRs 1, 2, 3 amino acid sequences are set forth in SEQ ID NOs: 10-12, respectively, and the selected VL CDRs 1, 2, 3 amino acid sequences are set forth in SEQ ID NOs: 29-31, respectively;
(5) the selected VH CDRs 1, 2, 3 amino acid sequences are set forth in SEQ ID NOs: 13-15, respectively, and the selected VL CDRs 1, 2, 3 amino acid sequences are set forth in SEQ ID NOs: 32-34, respectively;
(6) the selected VH CDRs 1, 2, 3 amino acid sequences are set forth in SEQ ID NOs: 16-18, respectively, and the selected VL CDRs 1, 2, 3 amino acid sequences are set forth in SEQ ID NOs: 32-34, respectively;
(7) the selected VH CDRs 1, 2, 3 amino acid sequences are set forth in SEQ ID NOs: 19-21, respectively, and the selected VL CDRs 1, 2, 3 amino acid sequences are set forth in SEQ ID NOs: 32-34, respectively; and
(8) the selected VH CDRs 1, 2, 3 amino acid sequences are set forth in SEQ ID NOs: 22-24, respectively, and the selected VL CDRs 1, 2, 3 amino acid sequences are set forth in SEQ ID NOs: 32-34, respectively.

In some embodiments, the VH comprises CDRs 1, 2, 3 with the amino acid sequences set forth in SEQ ID NOs: 1-3, respectively, and the VL comprises CDRs 1, 2, 3 with the amino acid sequences set forth in SEQ ID NOs: 29-31, respectively, according to the Kabat numbering scheme. In some embodiments, the VH comprises CDRs 1, 2, 3 with the amino acid sequences set forth in SEQ ID NOs: 4-6, respectively, and the VL comprises CDRs 1, 2, 3 with the amino acid sequences set forth in SEQ ID NOs: 29-31, respectively, according to the Kabat numbering scheme. In some embodiments, the VH comprises CDRs 1, 2, 3 with the amino acid sequences set forth in SEQ ID NOs: 7-9, respectively, and the VL comprises CDRs 1, 2, 3 with the amino acid sequences set forth in SEQ ID NOs: 29-31, respectively, according to the Kabat numbering scheme. In some embodiments, the VH comprises CDRs 1, 2, 3 with the amino acid sequences set forth in SEQ ID NOs: 10-12, respectively, and the VL comprises CDRs 1, 2, 3 with the amino acid sequences set forth in SEQ ID NOs: 29-31, respectively, according to the Kabat numbering scheme. In some embodiments, the VH comprises CDRs 1, 2, 3 with the amino acid sequences set forth in SEQ ID NOs: 13-15, respectively, and the VL comprises CDRs 1, 2, 3 with the amino acid sequences set forth in SEQ ID NOs: 32-34, respectively, according to the Chothia numbering scheme. In some embodiments, the VH comprises CDRs 1, 2, 3 with the amino acid sequences set forth in SEQ ID NOs: 16-18, respectively, and the VL comprises CDRs 1, 2, 3 with the amino acid sequences set forth in SEQ ID NOs: 32-34, respectively, according to the Chothia numbering scheme. In some embodiments, the VH comprises CDRs 1, 2, 3 with the amino acid sequences set forth in SEQ ID NOs: 19-21, respectively, and the VL comprises CDRs 1, 2, 3 with the amino acid sequences set forth in SEQ ID NOs: 32-34, respectively, according to the Chothia numbering scheme. In some embodiments, the VH comprises CDRs 1, 2, 3 with the amino acid sequences set forth in SEQ ID NOs: 22-24, respectively, and the VL comprises CDRs 1, 2, 3 with the amino acid sequences set forth in SEQ ID NOs: 32-34, respectively, according to the Chothia numbering scheme.

In one aspect, the disclosure is related to an antibody or antigen-binding fragment thereof that binds to CTLA4 comprising a heavy chain variable region (VH) comprising an amino acid sequence that is at least 90% identical to a selected VH sequence, and a light chain variable region (VL) comprising an amino acid sequence that is at least 90% identical to a selected VL sequence, in some embodiments, the selected VH sequence and the selected VL sequence are one of the following: (1) the selected VH sequence is SEQ ID NO: 25, and the selected VL sequence is SEQ ID NO: 35; (2) the selected VH sequence is SEQ ID NO: 26, and the selected VL sequence is SEQ ID NO: 35; (3) the selected VH sequence is SEQ ID NO: 27, and the selected VL sequence is SEQ ID NO: 35; and (4) the selected VH sequence is SEQ ID NO: 28, and the selected VL sequence is SEQ ID NO: 35. In some embodiments, the VH comprises the sequence of SEQ ID NO: 25 and the VL comprises the sequence of SEQ ID NO: 35. In some embodiments, the VH comprises the sequence of SEQ ID NO: 26 and the VL comprises the sequence of SEQ ID NO: 35. In some embodiments, the VH comprises the sequence of SEQ ID NO: 27 and the VL comprises the sequence of SEQ ID NO: 35. In some embodiments, the VH comprises the sequence of SEQ ID NO: 28 and the VL comprises the sequence of SEQ ID NO: 35.

In one aspect, the disclosure is related to an antibody or antigen-binding fragment thereof that binds to CTLA4 comprising a heavy chain variable region (VH) comprising VH CDR1, VH CDR2, and VH CDR3 that are identical to VH CDR1, VH CDR2, and VH CDR3 of a selected VH sequence; and a light chain variable region (VL) comprising VL CDR1, VL CDR2, and VL CDR3 that are identical to VL CDR1, VL CDR2, and VL CDR3 of a selected VL sequence, in some embodiments, the selected VH sequence and the selected VL sequence are one of the following: (1) the selected VH sequence is SEQ ID NO: 25, and the selected VL sequence is SEQ ID NO: 35; (2) the selected VH sequence is SEQ ID NO: 26, and the selected VL sequence is SEQ ID NO: 35; (3) the selected VH sequence is SEQ ID NO: 27, and the selected VL sequence is SEQ ID NO: 35; and (4) the selected VH sequence is SEQ ID NO: 28, and the selected VL sequence is SEQ ID NO: 35.

In some embodiments, the antibody or antigen-binding fragment thereof specifically binds to human or monkey CTLA4. In some embodiments, the antibody or antigen-binding fragment is a human or humanized antibody or antigen-binding fragment thereof. In some embodiments, the antibody or antigen-binding fragment is a single-chain variable fragment (scFV). In some embodiments, the antibody or antigen-binding fragment thereof is a bispecific or a multispecific antibody or an antigen-binding fragment thereof. In some embodiments, the antibody or antigen-binding fragment thereof further specifically binds to OX40.

In one aspect, the disclosure is related to an antibody or antigen-binding fragment thereof that cross-competes with the antibody or antigen-binding fragment thereof as described herein.

In one aspect, the disclosure is related to a nucleic acid comprising a polynucleotide encoding a polypeptide comprising:
(1) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 1-3, respectively, and in some embodiments, the VH, when paired with a light chain variable region (VL) comprising the amino acid sequence set forth in SEQ ID NO: 35, binds to CTLA4;
(2) an immunoglobulin light chain or a fragment thereof comprising a light chain variable region (VL) comprising complementarity determining regions (CDRs) 1, 2, and 3 comprising the amino acid sequences set forth in SEQ ID NOs: 29-31, respectively, and in some embodiments, the VL, when paired with a heavy chain variable region (VH) comprising the amino acid sequence set forth in SEQ ID NO: 25, binds to CTLA4;
(3) an immunoglobulin heavy chain or a fragment thereof comprising a VH comprising CDRs 1, 2, and 3 comprising the amino acid sequences set forth in SEQ ID NOs: 4-6, respectively, and in some embodiments, the VH, when paired with a VL comprising the amino acid sequence set forth in SEQ ID NO: 35, binds to CTLA4;
(4) an immunoglobulin light chain or a fragment thereof comprising a VL comprising CDRs 1, 2, and 3 comprising the amino acid sequences set forth in SEQ ID NOs: 29-31, respectively, and in some embodiments, the VL, when paired with a VH comprising the amino acid sequence set forth in SEQ ID NO: 26, binds to CTLA4;
(5) an immunoglobulin heavy chain or a fragment thereof comprising a VH comprising CDRs 1, 2, and 3 comprising the amino acid sequences set forth in SEQ ID NOs: 7-9, respectively, and in some embodiments, the VH, when paired with a VL comprising the amino acid sequence set forth in SEQ ID NO: 35, binds to CTLA4;
(6) an immunoglobulin light chain or a fragment thereof comprising a VL comprising CDRs 1, 2, and 3 comprising the amino acid sequences set forth in SEQ ID NOs: 29-31, respectively, and in some embodiments, the VL, when paired with a VH comprising the amino acid sequence set forth in SEQ ID NO: 27, binds to CTLA4;
(7) an immunoglobulin heavy chain or a fragment thereof comprising a VH comprising CDRs 1, 2, and 3 comprising the amino acid sequences set forth in SEQ ID NOs: 10-12, respectively, and in some embodiments, the VH, when paired with a VL comprising the amino acid sequence set forth in SEQ ID NO: 35, binds to CTLA4;
(8) an immunoglobulin light chain or a fragment thereof comprising a VL comprising CDRs 1, 2, and 3 comprising the amino acid sequences set forth in SEQ ID NOs: 29-31, respectively, and in some embodiments, the VL, when paired with a VH comprising the amino acid sequence set forth in SEQ ID NO: 28, binds to CTLA4;
(9) an immunoglobulin heavy chain or a fragment thereof comprising a VH comprising CDRs 1, 2, and 3 comprising the amino acid sequences set forth in SEQ ID NOs: 13-15, respectively, and in some embodiments, the VH, when paired with a VL comprising the amino acid sequence set forth in SEQ ID NO: 35, binds to CTLA4;
(10) an immunoglobulin light chain or a fragment thereof comprising a VL comprising CDRs 1, 2, and 3 comprising the amino acid sequences set forth in SEQ ID NOs: 32-34, respectively, and in some embodiments, the VL, when paired with a VH comprising the amino acid sequence set forth in SEQ ID NO: 25, binds to CTLA4;
(11) an immunoglobulin heavy chain or a fragment thereof comprising a VH comprising CDRs 1, 2, and 3 comprising the amino acid sequences set forth in SEQ ID NOs: 16-18, respectively, and in some embodiments, the VH, when paired with a VL comprising the amino acid sequence set forth in SEQ ID NO: 35, binds to CTLA4;
(12) an immunoglobulin light chain or a fragment thereof comprising a VL comprising CDRs 1, 2, and 3 comprising the amino acid sequences set forth in SEQ ID NOs: 32-34, respectively, and in some embodiments, the VL, when paired with a VH comprising the amino acid sequence set forth in SEQ ID NO: 26, binds to CTLA4;
(13) an immunoglobulin heavy chain or a fragment thereof comprising a VH comprising CDRs 1, 2, and 3 comprising the amino acid sequences set forth in SEQ ID NOs: 19-21, respectively, and in some embodiments, the VH, when paired with a VL comprising the amino acid sequence set forth in SEQ ID NO: 35, binds to CTLA4;
(14) an immunoglobulin light chain or a fragment thereof comprising a VL comprising CDRs 1, 2, and 3 comprising the amino acid sequences set forth in SEQ ID NOs: 32-34, respectively, and in some embodiments, the VL, when paired with a VH comprising the amino acid sequence set forth in SEQ ID NO: 27, binds to CTLA4;
(15) an immunoglobulin heavy chain or a fragment thereof comprising a VH comprising CDRs 1, 2, and 3 comprising the amino acid sequences set forth in SEQ ID NOs: 22-24, respectively, and in some embodiments, the VH, when paired with a VL comprising the amino acid sequence set forth in SEQ ID NO: 35, binds to CTLA4; and
(16) an immunoglobulin light chain or a fragment thereof comprising a VL comprising CDRs 1, 2, and 3 comprising the amino acid sequences set forth in SEQ ID NOs: 32-34, respectively, and in some embodiments, the VL, when paired with a VH comprising the amino acid sequence set forth in SEQ ID NO: 28, binds to CTLA4.

In some embodiments, the VH when paired with a VL specifically binds to human or monkey CTLA4. In some embodiments, the immunoglobulin heavy chain or the fragment thereof is a human or humanized immunoglobulin heavy chain or a fragment thereof. In some embodiments, the nucleic acid encodes a single-chain variable fragment (scFv). In some embodiments, the nucleic acid is cDNA.

In one aspect, the disclosure is related to an antibody or antigen-binding fragment thereof that binds to OX40 (Tumor necrosis factor receptor superfamily, member 4) comprising: a heavy chain variable region (VH) comprising complementarity determining regions (CDRs) 1, 2, and 3, in some embodiments, the VH CDR1 region comprises an amino acid sequence that is at least 80% identical to a selected VH CDR1 amino acid sequence, the VH CDR2 region comprises an amino acid sequence that is at least 80% identical to a selected VH CDR2 amino acid sequence, and the VH CDR3 region comprises an amino acid sequence that is at least 80% identical to a selected VH CDR3 amino acid sequence; and a light chain variable region (VL) comprising CDRs 1, 2, and 3, in some embodiments, the VL CDR1 region comprises an amino acid sequence that is at least 80% identical to a selected VL CDR1 amino acid sequence, the VL CDR2 region comprises an amino acid sequence that is at least 80% identical to a selected VL CDR2 amino acid sequence, and the VL CDR3 region comprises an amino acid sequence that is at least 80% identical to a selected VL CDR3 amino acid sequence, in some embodiments, the selected VH CDRs 1, 2, and 3 amino acid sequences and the selected VL CDRs, 1, 2, and 3 amino acid sequences are one of the following:
(1) the selected VH CDRs 1, 2, 3 amino acid sequences are set forth in SEQ ID NOs: 36-38, respectively, and the selected VL CDRs 1, 2, 3 amino acid sequences are set forth in SEQ ID NOs: 29-31, respectively;
(2) the selected VH CDRs 1, 2, 3 amino acid sequences are set forth in SEQ ID NOs: 39-41, respectively, and the selected VL CDRs 1, 2, 3 amino acid sequences are set forth in SEQ ID NOs: 29-31, respectively;
(3) the selected VH CDRs 1, 2, 3 amino acid sequences are set forth in SEQ ID NOs: 42-44, respectively, and the selected VL CDRs 1, 2, 3 amino acid sequences are set forth in SEQ ID NOs: 29-31, respectively;
(4) the selected VH CDRs 1, 2, 3 amino acid sequences are set forth in SEQ ID NOs: 45-47, respectively, and the selected VL CDRs 1, 2, 3 amino acid sequences are set forth in SEQ ID NOs: 29-31, respectively;
(5) the selected VH CDRs 1, 2, 3 amino acid sequences are set forth in SEQ ID NOs: 48-50, respectively, and the selected VL CDRs 1, 2, 3 amino acid sequences are set forth in SEQ ID NOs: 29-31, respectively;
(6) the selected VH CDRs 1, 2, 3 amino acid sequences are set forth in SEQ ID NOs: 51-53, respectively, and the selected VL CDRs 1, 2, 3 amino acid sequences are set forth in SEQ ID NOs: 29-31, respectively;
(7) the selected VH CDRs 1, 2, 3 amino acid sequences are set forth in SEQ ID NOs: 54-56, respectively, and the selected VL CDRs 1, 2, 3 amino acid sequences are set forth in SEQ ID NOs: 29-31, respectively; alternatively, the selected VH CDRs 1, 2, 3 amino acid sequences are set forth in SEQ ID NOs: 54-56, respectively, and the selected VL CDRs 1, 2, 3 amino acid sequences are set forth in SEQ ID NOs: 149-151, respectively;
(8) the selected VH CDRs 1, 2, 3 amino acid sequences are set forth in SEQ ID NOs: 57-59, respectively, and the selected VL CDRs 1, 2, 3 amino acid sequences are set forth in SEQ ID NOs: 29-31, respectively; alternatively, the selected VH CDRs 1, 2, 3 amino acid sequences are set forth in SEQ ID NOs: 57-59, respectively, and the selected VL CDRs 1, 2, 3 amino acid sequences are set forth in SEQ ID NOs: 152-154, respectively;
(9) the selected VH CDRs 1, 2, 3 amino acid sequences are set forth in SEQ ID NOs: 60-62, respectively, and the selected VL CDRs 1, 2, 3 amino acid sequences are set forth in SEQ ID NOs: 32-34, respectively;
(10) the selected VH CDRs 1, 2, 3 amino acid sequences are set forth in SEQ ID NOs: 63-65, respectively, and the selected VL CDRs 1, 2, 3 amino acid sequences are set forth in SEQ ID NOs: 32-34, respectively;
(11) the selected VH CDRs 1, 2, 3 amino acid sequences are set forth in SEQ ID NOs: 66-68, respectively, and the selected VL CDRs 1, 2, 3 amino acid sequences are set forth in SEQ ID NOs: 32-34, respectively;
(12) the selected VH CDRs 1, 2, 3 amino acid sequences are set forth in SEQ ID NOs: 69-71, respectively, and the selected VL CDRs 1, 2, 3 amino acid sequences are set forth in SEQ ID NOs: 32-34, respectively;
(13) the selected VH CDRs 1, 2, 3 amino acid sequences are set forth in SEQ ID NOs: 72-74, respectively, and the selected VL CDRs 1, 2, 3 amino acid sequences are set forth in SEQ ID NOs: 32-34, respectively;
(14) the selected VH CDRs 1, 2, 3 amino acid sequences are set forth in SEQ ID NOs: 75-77, respectively, and the selected VL CDRs 1, 2, 3 amino acid sequences are set forth in SEQ ID NOs: 32-34, respectively;
(15) the selected VH CDRs 1, 2, 3 amino acid sequences are set forth in SEQ ID NOs: 78-80, respectively, and the selected VL CDRs 1, 2, 3 amino acid sequences are set forth in SEQ ID NOs: 32-34, respectively; alternatively, the selected VH CDRs 1, 2, 3 amino acid sequences are set forth in SEQ ID NOs: 78-80, respectively, and the selected VL CDRs 1, 2, 3 amino acid sequences are set forth in SEQ ID NOs: 155-157, respectively; and
(16) the selected VH CDRs 1, 2, 3 amino acid sequences are set forth in SEQ ID NOs: 81-83, respectively, and the selected VL CDRs 1, 2, 3 amino acid sequences are set forth in SEQ ID NOs: 32-34, respectively; alternatively, the selected VH CDRs 1, 2, 3 amino acid sequences are set forth in SEQ ID NOs: 81-83, respectively, and the selected VL CDRs 1, 2, 3 amino acid sequences are set forth in SEQ ID NOs: 158-160, respectively.

In some embodiments, the VH comprises CDRs 1, 2, 3 with the amino acid sequences set forth in SEQ ID NOs: 36-38, respectively, and the VL comprises CDRs 1, 2, 3 with the amino acid sequences set forth in SEQ ID NOs: 29-31, respectively, according to the Kabat numbering scheme. In some embodiments, the VH comprises CDRs 1, 2, 3 with the amino acid sequences set forth in SEQ ID NOs: 39-41, respectively, and the VL comprises CDRs 1, 2, 3 with the amino acid sequences set forth in SEQ ID NOs: 29-31, respectively, according to the Kabat numbering scheme. In some embodiments, the VH comprises CDRs 1, 2, 3 with the amino acid sequences set forth in SEQ ID NOs: 42-44, respectively, and the VL comprises CDRs 1, 2, 3 with the amino acid sequences set forth in SEQ ID NOs: 29-31, respectively, according to the Kabat numbering scheme. In some embodiments, the VH comprises CDRs 1, 2, 3 with the amino acid sequences set forth in SEQ ID NOs: 45-47, respectively, and the VL comprises CDRs 1, 2, 3 with the amino acid sequences set forth in SEQ ID NOs: 29-31, respectively, according to the Kabat numbering scheme. In some embodiments, the VH comprises CDRs 1, 2, 3 with the amino acid sequences set forth in SEQ ID NOs: 48-50, respectively, and the VL comprises CDRs 1, 2, 3 with the amino acid sequences set forth in SEQ ID NOs: 29-31, respectively, according to the Kabat numbering scheme. In some embodiments, the VH comprises CDRs 1, 2, 3 with the amino acid sequences set forth in SEQ ID NOs: 51-53, respectively, and the VL comprises CDRs 1, 2, 3 with the amino acid sequences set forth in SEQ ID NOs: 29-31, respectively, according to the Kabat numbering scheme. In some embodiments, (1) the VH comprises CDRs 1, 2, 3 with the amino acid sequences set forth in SEQ ID NOs: 54-56, respectively, and the VL comprises CDRs 1, 2, 3 with the amino acid sequences set forth in SEQ ID NOs: 29-31, respectively, according to the Kabat numbering scheme; or (2) the VH comprises CDRs 1, 2, 3 with the amino acid sequences set forth in SEQ ID NOs: 54-56, respectively, and the VL comprises CDRs 1, 2, 3 with the amino acid sequences set forth in SEQ ID NOs: 149-151, respectively, according to the Kabat numbering scheme. In some embodiments, (1) the VH comprises CDRs 1, 2, 3 with the amino acid sequences set forth in SEQ ID NOs: 57-59, respectively, and the VL comprises CDRs 1, 2, 3 with the amino acid sequences set forth in SEQ ID NOs: 29-31, respectively, according to the Kabat numbering scheme; or (2) the VH comprises CDRs 1, 2, 3 with the amino acid sequences set forth in SEQ ID NOs: 57-59, respectively, and the VL comprises CDRs 1, 2, 3 with the amino acid sequences set forth in SEQ ID NOs: 152-154, respectively, according to the Kabat numbering scheme. In some embodiments, the VH comprises CDRs 1, 2, 3 with the amino acid sequences set forth in SEQ ID NOs: 60-62, respectively, and the VL comprises CDRs 1, 2, 3 with the amino acid sequences set forth in SEQ ID NOs: 32-34, respectively, according to the Chothia numbering scheme. In some embodiments, the VH comprises CDRs 1, 2, 3 with the amino acid sequences set forth in SEQ ID NOs: 63-65, respectively, and the VL comprises CDRs 1, 2, 3 with the amino acid sequences set forth in SEQ ID NOs: 32-34, respectively, according to the Chothia numbering scheme. In some embodiments, the VH comprises CDRs 1, 2, 3 with the amino acid sequences set forth in SEQ ID NOs: 66-68, respectively, and the VL comprises CDRs 1, 2, 3 with the amino acid sequences set forth in SEQ ID NOs: 32-34, respectively, according to the Chothia numbering scheme. In some embodiments, the VH comprises CDRs 1, 2, 3 with the amino acid sequences set forth in SEQ ID NOs: 69-71, respectively, and the VL comprises CDRs 1, 2, 3 with the amino acid sequences set forth in SEQ ID NOs: 32-34, respectively, according to the Chothia numbering scheme. In some embodiments, the VH comprises CDRs 1, 2, 3 with the amino acid sequences set forth in SEQ ID NOs: 72-74, respectively, and the VL comprises CDRs 1, 2, 3 with the amino acid sequences set forth in SEQ ID NOs: 32-34, respectively, according to the Chothia numbering scheme. In some embodiments, the VH comprises CDRs 1, 2, 3 with the amino acid sequences set forth in SEQ ID NOs: 75-77, respectively, and the VL comprises CDRs 1, 2, 3 with the amino acid sequences set forth in SEQ ID NOs: 32-34, respectively, according to the Chothia numbering scheme. In some embodiments, (1) the VH comprises CDRs 1, 2, 3 with the amino acid sequences set forth in SEQ ID NOs: 78-80, respectively, and the VL comprises CDRs 1, 2, 3 with the amino acid sequences set forth in SEQ ID NOs: 32-34, respectively, according to the Chothia numbering scheme; or (2) the VH comprises CDRs 1, 2, 3 with the amino acid sequences set forth in SEQ ID NOs: 78-80, respectively, and the VL comprises CDRs 1, 2, 3 with the amino acid sequences set forth in SEQ ID NOs: 155-157, respectively, according to the Chothia numbering scheme. In some embodiments, (1) the VH comprises CDRs 1, 2, 3 with the amino acid sequences set forth in SEQ ID NOs: 81-83, respectively, and the VL comprises CDRs 1, 2, 3 with the amino acid sequences set forth in SEQ ID NOs: 32-34, respectively, according to the Chothia numbering scheme; or (2) the VH comprises CDRs 1, 2, 3 with the amino acid sequences set forth in SEQ ID NOs: 81-83, respectively, and the VL comprises CDRs 1, 2, 3 with the amino acid sequences set forth in SEQ ID NOs: 158-160, respectively, according to the Chothia numbering scheme.

In one aspect, the disclosure is related to an antibody or antigen-binding fragment thereof that binds to OX40 comprising a heavy chain variable region (VH) comprising an amino acid sequence that is at least 90% identical to a selected VH sequence, and a light chain variable region (VL) comprising an amino acid sequence that is at least 90% identical to a selected VL sequence, in some embodiments, the selected VH sequence and the selected VL sequence are one of the following: (1) the selected VH sequence is SEQ ID NO: 84, and the selected VL sequence is SEQ ID NO: 35; (2) the selected VH sequence is SEQ ID NO: 85, and the selected VL sequence is SEQ ID NO: 35; (3) the selected VH sequence is SEQ ID NO: 86, and the selected VL sequence is SEQ ID NO: 35; (4) the selected VH sequence is SEQ ID NO: 87, and the selected VL sequence is SEQ ID NO: 35; (5) the selected VH sequence is SEQ ID NO: 88, and the selected VL sequence is SEQ ID NO: 35; (6) the selected VH sequence is SEQ ID NO: 89, and the selected VL sequence is SEQ ID NO: 35; (7) the selected VH sequence is SEQ ID NO: 90, and the selected VL sequence is SEQ ID NO: 35 or 161; and (8) the selected VH sequence is SEQ ID NO: 91, and the selected VL sequence is SEQ ID NO: 35 or 162. In some embodiments, the VH comprises the sequence of SEQ ID NO: 84 and the VL comprises the sequence of SEQ ID NO: 35. In some embodiments, the VH comprises the sequence of SEQ ID NO: 85 and the VL comprises the sequence of SEQ ID NO: 35. In some embodiments, the VH comprises the sequence of SEQ ID NO: 86 and the VL comprises the sequence of SEQ ID NO: 35. In some embodiments, the VH comprises the sequence of SEQ ID NO: 87 and the VL comprises the sequence of SEQ ID NO: 35. In some embodiments, the VH comprises the sequence of SEQ ID NO: 88 and the VL comprises the sequence of SEQ ID NO: 35. In some embodiments, the VH comprises the sequence of SEQ ID NO: 89 and the VL comprises the sequence of SEQ ID NO: 35. In some embodiments, the VH comprises the sequence of SEQ ID NO: 90 and the VL comprises the sequence of SEQ ID NO: 35 or 161. In some embodiments, the VH comprises the sequence of SEQ ID NO: 91 and the VL comprises the sequence of SEQ ID NO: 35 or 162.

In one aspect, the disclosure is related to an antibody or antigen-binding fragment thereof that binds to OX40 comprising a heavy chain variable region (VH) comprising VH CDR1, VH CDR2, and VH CDR3 that are identical to VH CDR1, VH CDR2, and VH CDR3 of a selected VH sequence; and a light chain variable region (VL) comprising VL CDR1, VL CDR2, and VL CDR3 that are identical to VL CDR1, VL CDR2, and VL CDR3 of a selected VL sequence, in some embodiments, the selected VH sequence and the selected VL sequence are one of the following: (1) the selected VH sequence is SEQ ID NO: 84, and the selected VL sequence is SEQ ID NO: 35; (2) the selected VH sequence is SEQ ID NO: 85, and the selected VL sequence is SEQ ID NO: 35; (3) the selected VH sequence is SEQ ID NO: 86, and the selected VL sequence is SEQ ID NO: 35; (4) the selected VH sequence is SEQ ID NO: 87, and the selected VL sequence is SEQ ID NO: 35; (5) the selected VH sequence is SEQ ID NO: 88, and the selected VL sequence is SEQ ID NO: 35; (6) the selected VH sequence is SEQ ID NO: 89, and the selected VL sequence is SEQ ID NO: 35; (7) the selected VH sequence is SEQ ID NO: 90, and the selected VL sequence is SEQ ID NO: 35 or 161; and (8) the selected VH sequence is SEQ ID NO: 91, and the selected VL sequence is SEQ ID NO: 35 or 162.

In some embodiments, the antibody or antigen-binding fragment thereof specifically binds to human or monkey OX40. In some embodiments, the antibody or antigen-binding fragment is a human or humanized antibody or antigen-binding fragment thereof. In some embodiments, the antibody or antigen-binding fragment is a single-chain variable fragment (scFV). In some embodiments, the antibody or antigen-binding fragment thereof is a bispecific or a multispecific antibody or an antigen-binding fragment thereof. In some embodiments, the antibody or antigen-binding fragment thereof further specifically binds to CTLA4.

In one aspect, the disclosure is related to an antibody or antigen-binding fragment thereof that cross-competes with the antibody or antigen-binding fragment thereof as described herein.

In one aspect, the disclosure is related to a nucleic acid comprising a polynucleotide encoding a polypeptide comprising:
(1) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 36-38, respectively, and in some embodiments, the VH, when paired with a light chain variable region (VL) comprising the amino acid sequence set forth in SEQ ID NO: 35, binds to OX40;
(2) an immunoglobulin light chain or a fragment thereof comprising a light chain variable region (VL) comprising complementarity determining regions (CDRs) 1, 2, and 3 comprising the amino acid sequences set forth in SEQ ID NOs: 29-31, respectively, and in some embodiments, the VL, when paired with a heavy chain variable region (VH) comprising the amino acid sequence set forth in SEQ ID NO: 84, binds to OX40;
(3) an immunoglobulin heavy chain or a fragment thereof comprising a VH comprising CDRs 1, 2, and 3 comprising the amino acid sequences set forth in SEQ ID NOs: 39-41, respectively, and in some embodiments, the VH, when paired with a VL comprising the amino acid sequence set forth in SEQ ID NO: 35, binds to OX40;
(4) an immunoglobulin light chain or a fragment thereof comprising a VL comprising CDRs 1, 2, and 3 comprising the amino acid sequences set forth in SEQ ID NOs: 29-31, respectively, and in some embodiments, the VL, when paired with a VH comprising the amino acid sequence set forth in SEQ ID NO: 85, binds to OX40;
(5) an immunoglobulin heavy chain or a fragment thereof comprising a VH comprising CDRs 1, 2, and 3 comprising the amino acid sequences set forth in SEQ ID NOs: 42-44, respectively, and in some embodiments, the VH, when paired with a VL comprising the amino acid sequence set forth in SEQ ID NO: 35, binds to OX40;
(6) an immunoglobulin light chain or a fragment thereof comprising a VL comprising CDRs 1, 2, and 3 comprising the amino acid sequences set forth in SEQ ID NOs: 29-31, respectively, and in some embodiments, the VL, when paired with a VH comprising the amino acid sequence set forth in SEQ ID NO: 86, binds to OX40;
(7) an immunoglobulin heavy chain or a fragment thereof comprising a VH comprising CDRs 1, 2, and 3 comprising the amino acid sequences set forth in SEQ ID NOs: 45-47, respectively, and in some embodiments, the VH, when paired with a VL comprising the amino acid sequence set forth in SEQ ID NO: 35, binds to OX40;
(8) an immunoglobulin light chain or a fragment thereof comprising a VL comprising CDRs 1, 2, and 3 comprising the amino acid sequences set forth in SEQ ID NOs: 29-31, respectively, and in some embodiments, the VL, when paired with a VH comprising the amino acid sequence set forth in SEQ ID NO: 87, binds to OX40;
(9) an immunoglobulin heavy chain or a fragment thereof comprising a VH comprising CDRs 1, 2, and 3 comprising the amino acid sequences set forth in SEQ ID NOs: 48-50, respectively, and in some embodiments, the VH, when paired with a VL comprising the amino acid sequence set forth in SEQ ID NO: 35, binds to OX40;
(10) an immunoglobulin light chain or a fragment thereof comprising a VL comprising CDRs 1, 2, and 3 comprising the amino acid sequences set forth in SEQ ID NOs: 29-31, respectively, and in some embodiments, the VL, when paired with a VH comprising the amino acid sequence set forth in SEQ ID NO: 88, binds to OX40;
(11) an immunoglobulin heavy chain or a fragment thereof comprising a VH comprising CDRs 1, 2, and 3 comprising the amino acid sequences set forth in SEQ ID NOs: 51-53, respectively, and in some embodiments, the VH, when paired with a VL comprising the amino acid sequence set forth in SEQ ID NO: 35, binds to OX40;
(12) an immunoglobulin light chain or a fragment thereof comprising a VL comprising CDRs 1, 2, and 3 comprising the amino acid sequences set forth in SEQ ID NOs: 29-31, respectively, and in some embodiments, the VL, when paired with a VH comprising the amino acid sequence set forth in SEQ ID NO: 89, binds to OX40;
(13) an immunoglobulin heavy chain or a fragment thereof comprising a VH comprising CDRs 1, 2, and 3 comprising the amino acid sequences set forth in SEQ ID NOs: 54-56, respectively, and in some embodiments, the VH, when paired with a VL comprising the amino acid sequence set forth in SEQ ID NO: 35 or 161, binds to OX40;
(14) an immunoglobulin light chain or a fragment thereof comprising a VL comprising CDRs 1, 2, and 3 comprising the amino acid sequences set forth in SEQ ID NOs: 29-31 or SEQ ID NOs: 149-151, respectively, and in some embodiments, the VL, when paired with a VH comprising the amino acid sequence set forth in SEQ ID NO: 90, binds to OX40;
(15) an immunoglobulin heavy chain or a fragment thereof comprising a VH comprising CDRs 1, 2, and 3 comprising the amino acid sequences set forth in SEQ ID NOs: 57-59, respectively, and in some embodiments, the VH, when paired with a VL comprising the amino acid sequence set forth in SEQ ID NO: 35 or 162, binds to OX40;
(16) an immunoglobulin light chain or a fragment thereof comprising a VL comprising CDRs 1, 2, and 3 comprising the amino acid sequences set forth in SEQ ID NOs: 29-31 or SEQ ID NOs: 152-154, respectively, and in some embodiments, the VL, when paired with a VH comprising the amino acid sequence set forth in SEQ ID NO: 91, binds to OX40;
(17) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 60-62, respectively, and in some embodiments, the VH, when paired with a light chain variable region (VL) comprising the amino acid sequence set forth in SEQ ID NO: 35, binds to OX40;
(18) an immunoglobulin light chain or a fragment thereof comprising a light chain variable region (VL) comprising complementarity determining regions (CDRs) 1, 2, and 3 comprising the amino acid sequences set forth in SEQ ID NOs: 32-34, respectively, and in some embodiments, the VL, when paired with a heavy chain variable region (VH) comprising the amino acid sequence set forth in SEQ ID NO: 84, binds to OX40;
(19) an immunoglobulin heavy chain or a fragment thereof comprising a VH comprising CDRs 1, 2, and 3 comprising the amino acid sequences set forth in SEQ ID NOs: 63-65, respectively, and in some embodiments, the VH, when paired with a VL comprising the amino acid sequence set forth in SEQ ID NO: 35, binds to OX40;
(20) an immunoglobulin light chain or a fragment thereof comprising a VL comprising CDRs 1, 2, and 3 comprising the amino acid sequences set forth in SEQ ID NOs: 32-34, respectively, and in some embodiments, the VL, when paired with a VH comprising the amino acid sequence set forth in SEQ ID NO: 85, binds to OX40;
(21) an immunoglobulin heavy chain or a fragment thereof comprising a VH comprising CDRs 1, 2, and 3 comprising the amino acid sequences set forth in SEQ ID NOs: 66-68, respectively, and in some embodiments, the VH, when paired with a VL comprising the amino acid sequence set forth in SEQ ID NO: 35, binds to OX40;
(22) an immunoglobulin light chain or a fragment thereof comprising a VL comprising CDRs 1, 2, and 3 comprising the amino acid sequences set forth in SEQ ID NOs: 32-34, respectively, and in some embodiments, the VL, when paired with a VH comprising the amino acid sequence set forth in SEQ ID NO: 86, binds to OX40;
(23) an immunoglobulin heavy chain or a fragment thereof comprising a VH comprising CDRs 1, 2, and 3 comprising the amino acid sequences set forth in SEQ ID NOs: 69-71, respectively, and in some embodiments, the VH, when paired with a VL comprising the amino acid sequence set forth in SEQ ID NO: 35, binds to OX40;
(24) an immunoglobulin light chain or a fragment thereof comprising a VL comprising CDRs 1, 2, and 3 comprising the amino acid sequences set forth in SEQ ID NOs: 32-34, respectively, and in some embodiments, the VL, when paired with a VH comprising the amino acid sequence set forth in SEQ ID NO: 87, binds to OX40;
(25) an immunoglobulin heavy chain or a fragment thereof comprising a VH comprising CDRs 1, 2, and 3 comprising the amino acid sequences set forth in SEQ ID NOs: 72-74, respectively, and in some embodiments, the VH, when paired with a VL comprising the amino acid sequence set forth in SEQ ID NO: 35, binds to OX40;
(26) an immunoglobulin light chain or a fragment thereof comprising a VL comprising CDRs 1, 2, and 3 comprising the amino acid sequences set forth in SEQ ID NOs: 32-34, respectively, and in some embodiments, the VL, when paired with a VH comprising the amino acid sequence set forth in SEQ ID NO: 88, binds to OX40;
(27) an immunoglobulin heavy chain or a fragment thereof comprising a VH comprising CDRs 1, 2, and 3 comprising the amino acid sequences set forth in SEQ ID NOs: 75-77, respectively, and in some embodiments, the VH, when paired with a VL comprising the amino acid sequence set forth in SEQ ID NO: 35, binds to OX40;
(28) an immunoglobulin light chain or a fragment thereof comprising a VL comprising CDRs 1, 2, and 3 comprising the amino acid sequences set forth in SEQ ID NOs: 32-34, respectively, and in some embodiments, the VL, when paired with a VH comprising the amino acid sequence set forth in SEQ ID NO: 89, binds to OX40;
(29) an immunoglobulin heavy chain or a fragment thereof comprising a VH comprising CDRs 1, 2, and 3 comprising the amino acid sequences set forth in SEQ ID NOs: 78-80, respectively, and in some embodiments, the VH, when paired with a VL comprising the amino acid sequence set forth in SEQ ID NO: 35 or 161, binds to OX40;
(30) an immunoglobulin light chain or a fragment thereof comprising a VL comprising CDRs 1, 2, and 3 comprising the amino acid sequences set forth in SEQ ID NOs: 32-34 or SEQ ID NOs: 155-157, respectively, and in some embodiments, the VL, when paired with a VH comprising the amino acid sequence set forth in SEQ ID NO: 90, binds to OX40;
(31) an immunoglobulin heavy chain or a fragment thereof comprising a VH comprising CDRs 1, 2, and 3 comprising the amino acid sequences set forth in SEQ ID NOs: 81-83, respectively, and in some embodiments, the VH, when paired with a VL comprising the amino acid sequence set forth in SEQ ID NO: 35 or 162, binds to OX40; and
(32) an immunoglobulin light chain or a fragment thereof comprising a VL comprising CDRs 1, 2, and 3 comprising the amino acid sequences set forth in SEQ ID NOs: 32-34 or SEQ ID NOs: 158-160, respectively, and in some embodiments, the VL, when paired with a VH comprising the amino acid sequence set forth in SEQ ID NO: 91, binds to OX40.

In some embodiments, the VH when paired with a VL specifically binds to human or monkey OX40. In some embodiments, the immunoglobulin heavy chain or the fragment thereof is a human or humanized immunoglobulin heavy chain or a fragment thereof. In some embodiments, the nucleic acid encodes a single-chain variable fragment (scFv). In some embodiments, the nucleic acid is cDNA.

In one aspect, the disclosure is related to an antigen-binding protein construct, comprising: a first antigen-binding domain that specifically binds to CTLA4; and a second antigen-binding domain that specially binds to OX40. In some embodiments, the first antigen-binding domain comprises a first heavy chain variable region (VH1) and a first light chain variable region (VL1); and the second antigen-binding domain comprises a second heavy chain variable region (VH2) and a second light chain variable region (VL2).

In some embodiments, the first heavy chain variable region (VH1) comprises complementarity determining regions (CDRs) 1, 2, and 3, in some embodiments, the VH1 CDR1 region comprises an amino acid sequence that is at least 80% identical to a selected VH1 CDR1 amino acid sequence, the VH1 CDR2 region comprises an amino acid sequence that is at least 80% identical to a selected VH1 CDR2 amino acid sequence, and the VH1 CDR3 region comprises an amino acid sequence that is at least 80% identical to a selected VH1 CDR3 amino acid sequence; and the first light chain variable region (VL1) comprises CDRs 1, 2, and 3, in some embodiments, the VL1 CDR1 region comprises an amino acid sequence that is at least 80% identical to a selected VL1 CDR1 amino acid sequence, the VL1 CDR2 region comprises an amino acid sequence that is at least 80% identical to a selected VL1 CDR2 amino acid sequence, and the VL1 CDR3 region comprises an amino acid sequence that is at least 80% identical to a selected VL1 CDR3 amino acid sequence, in some embodiments, the selected VH1 CDRs 1, 2, and 3 amino acid sequences, the selected VL1 CDRs 1, 2, and 3 amino acid sequences are one of the following:
(1) the selected VH1 CDRs 1, 2, 3 amino acid sequences are set forth in SEQ ID NOs: 1-3, respectively, and the selected VL1 CDRs 1, 2, 3 amino acid sequences are set forth in SEQ ID NOs: 29-31, respectively;
(2) the selected VH1 CDRs 1, 2, 3 amino acid sequences are set forth in SEQ ID NOs: 4-6, respectively, and the selected VL1 CDRs 1, 2, 3 amino acid sequences are set forth in SEQ ID NOs: 29-31, respectively;
(3) the selected VH1 CDRs 1, 2, 3 amino acid sequences are set forth in SEQ ID NOs: 7-9, respectively, and the selected VL1 CDRs 1, 2, 3 amino acid sequences are set forth in SEQ ID NOs: 29-31, respectively;
(4) the selected VH1 CDRs 1, 2, 3 amino acid sequences are set forth in SEQ ID NOs: 10-12, respectively, and the selected VL1 CDRs 1, 2, 3 amino acid sequences are set forth in SEQ ID NOs: 29-31, respectively;
(5) the selected VH1 CDRs 1, 2, 3 amino acid sequences are set forth in SEQ ID NOs: 13-15, respectively, and the selected VL1 CDRs 1, 2, 3 amino acid sequences are set forth in SEQ ID NOs: 32-34, respectively;
(6) the selected VH1 CDRs 1, 2, 3 amino acid sequences are set forth in SEQ ID NOs: 16-18, respectively, and the selected VL1 CDRs 1, 2, 3 amino acid sequences are set forth in SEQ ID NOs: 32-34, respectively;
(7) the selected VH1 CDRs 1, 2, 3 amino acid sequences are set forth in SEQ ID NOs: 19-21, respectively, and the selected VL1 CDRs 1, 2, 3 amino acid sequences are set forth in SEQ ID NOs: 32-34, respectively; and
(8) the selected VH1 CDRs 1, 2, 3 amino acid sequences are set forth in SEQ ID NOs: 22-24, respectively, and the selected VL1 CDRs 1, 2, 3 amino acid sequences are set forth in SEQ ID NOs: 32-34, respectively.

In some embodiments, the second heavy chain variable region (VH2) comprises CDRs 1, 2, and 3, in some embodiments, the VH2 CDR1 region comprises an amino acid sequence that is at least 80% identical to a selected VH2 CDR1 amino acid sequence, the VH2 CDR2 region comprises an amino acid sequence that is at least 80% identical to a selected VH2 CDR2 amino acid sequence, and the VH2 CDR3 region comprises an amino acid sequence that is at least 80% identical to a selected VH2 CDR3 amino acid sequence; and the second light chain variable region (VL2) comprises CDRs 1, 2, and 3, in some embodiments, the VL2 CDR1 region comprises an amino acid sequence that is at least 80% identical to a selected VL2 CDR1 amino acid sequence, the VL2 CDR2 region comprises an amino acid sequence that is at least 80% identical to a selected VL2 CDR2 amino acid sequence, and the VL2 CDR3 region comprises an amino acid sequence that is at least 80% identical to a selected VL2 CDR3 amino acid sequence, in some embodiments, the selected VH2 CDRs 1, 2, and 3 amino acid sequences, and the selected VL2 CDRs 1, 2, and 3 amino acid sequences are one of the following:
(1) the selected VH2 CDRs 1, 2, 3 amino acid sequences are set forth in SEQ ID NOs: 36-38, respectively, and the selected VL2 CDRs 1, 2, 3 amino acid sequences are set forth in SEQ ID NOs: 29-31, respectively;
(2) the selected VH2 CDRs 1, 2, 3 amino acid sequences are set forth in SEQ ID NOs: 39-41, respectively, and the selected VL2 CDRs 1, 2, 3 amino acid sequences are set forth in SEQ ID NOs: 29-31, respectively;
(3) the selected VH2 CDRs 1, 2, 3 amino acid sequences are set forth in SEQ ID NOs: 42-44, respectively, and the selected VL2 CDRs 1, 2, 3 amino acid sequences are set forth in SEQ ID NOs: 29-31, respectively;
(4) the selected VH2 CDRs 1, 2, 3 amino acid sequences are set forth in SEQ ID NOs: 45-47, respectively, and the selected VL2 CDRs 1, 2, 3 amino acid sequences are set forth in SEQ ID NOs: 29-31, respectively;
(5) the selected VH2 CDRs 1, 2, 3 amino acid sequences are set forth in SEQ ID NOs: 48-50, respectively, and the selected VL2 CDRs 1, 2, 3 amino acid sequences are set forth in SEQ ID NOs: 29-31, respectively;
(6) the selected VH2 CDRs 1, 2, 3 amino acid sequences are set forth in SEQ ID NOs: 51-53, respectively, and the selected VL2 CDRs 1, 2, 3 amino acid sequences are set forth in SEQ ID NOs: 29-31, respectively;
(7) the selected VH2 CDRs 1, 2, 3 amino acid sequences are set forth in SEQ ID NOs: 54-56, respectively, and the selected VL2 CDRs 1, 2, 3 amino acid sequences are set forth in SEQ ID NOs: 29-31, respectively; alternatively, the selected VH2 CDRs 1, 2, 3 amino acid sequences are set forth in SEQ ID NOs: 54-56, respectively, and the selected VL2 CDRs 1, 2, 3 amino acid sequences are set forth in SEQ ID NOs: 149-151, respectively;
(8) the selected VH2 CDRs 1, 2, 3 amino acid sequences are set forth in SEQ ID NOs: 57-59, respectively, and the selected VL2 CDRs 1, 2, 3 amino acid sequences are set forth in SEQ ID NOs: 29-31, respectively; alternatively, the selected VH2 CDRs 1, 2, 3 amino acid sequences are set forth in SEQ ID NOs: 57-59, respectively, and the selected VL2 CDRs 1, 2, 3 amino acid sequences are set forth in SEQ ID NOs: 152-154, respectively;
(9) the selected VH2 CDRs 1, 2, 3 amino acid sequences are set forth in SEQ ID NOs: 60-62, respectively, and the selected VL2 CDRs 1, 2, 3 amino acid sequences are set forth in SEQ ID NOs: 32-34, respectively;
(10) the selected VH2 CDRs 1, 2, 3 amino acid sequences are set forth in SEQ ID NOs: 63-65, respectively, and the selected VL2 CDRs 1, 2, 3 amino acid sequences are set forth in SEQ ID NOs: 32-34, respectively;
(11) the selected VH2 CDRs 1, 2, 3 amino acid sequences are set forth in SEQ ID NOs: 66-68, respectively, and the selected VL2 CDRs 1, 2, 3 amino acid sequences are set forth in SEQ ID NOs: 32-34, respectively;
(12) the selected VH2 CDRs 1, 2, 3 amino acid sequences are set forth in SEQ ID NOs: 69-71, respectively, and the selected VL2 CDRs 1, 2, 3 amino acid sequences are set forth in SEQ ID NOs: 32-34, respectively;
(13) the selected VH2 CDRs 1, 2, 3 amino acid sequences are set forth in SEQ ID NOs: 72-74, respectively, and the selected VL2 CDRs 1, 2, 3 amino acid sequences are set forth in SEQ ID NOs: 32-34, respectively;
(14) the selected VH2 CDRs 1, 2, 3 amino acid sequences are set forth in SEQ ID NOs: 75-77, respectively, and the selected VL2 CDRs 1, 2, 3 amino acid sequences are set forth in SEQ ID NOs: 32-34, respectively;
(15) the selected VH2 CDRs 1, 2, 3 amino acid sequences are set forth in SEQ ID NOs: 78-80, respectively, and the selected VL2 CDRs 1, 2, 3 amino acid sequences are set forth in SEQ ID NOs: 32-34, respectively; alternatively, the selected VH2 CDRs 1, 2, 3 amino acid sequences are set forth in SEQ ID NOs: 78-80, respectively, and the selected VL2 CDRs 1, 2, 3 amino acid sequences are set forth in SEQ ID NOs: 155-157, respectively; and
(16) the selected VH2 CDRs 1, 2, 3 amino acid sequences are set forth in SEQ ID NOs: 81-83, respectively, and the selected VL2 CDRs 1, 2, 3 amino acid sequences are set forth in SEQ ID NOs: 32-34, respectively; alternatively, the selected VH2 CDRs 1, 2, 3 amino acid sequences are set forth in SEQ ID NOs: 81-83, respectively, and the selected VL2 CDRs 1, 2, 3 amino acid sequences are set forth in SEQ ID NOs: 158-160, respectively.

In some embodiments, one or more of the following can be true:
(1) the selected VH1 CDRs 1, 2, 3 amino acid sequences are set forth in SEQ ID NOs: 1-3, respectively, and the selected VL1 CDRs 1, 2, 3 amino acid sequences are set forth in SEQ ID NOs: 29-31, respectively, and the selected VH2 CDRs 1, 2, 3 amino acid sequences are set forth in SEQ ID NOs: 36-38, respectively, and the selected VL2 CDRs 1, 2, 3 amino acid sequences are set forth in SEQ ID NOs: 29-31, respectively;
(2) the selected VH1 CDRs 1, 2, 3 amino acid sequences are set forth in SEQ ID NOs: 1-3, respectively, and the selected VL1 CDRs 1, 2, 3 amino acid sequences are set forth in SEQ ID NOs: 29-31, respectively, and the selected VH2 CDRs 1, 2, 3 amino acid sequences are set forth in SEQ ID NOs: 39-41, respectively, and the selected VL2 CDRs 1, 2, 3 amino acid sequences are set forth in SEQ ID NOs: 29-31, respectively;
(3) the selected VH1 CDRs 1, 2, 3 amino acid sequences are set forth in SEQ ID NOs: 1-3, respectively, and the selected VL1 CDRs 1, 2, 3 amino acid sequences are set forth in SEQ ID NOs: 29-31, respectively, and the selected VH2 CDRs 1, 2, 3 amino acid sequences are set forth in SEQ ID NOs: 42-44, respectively, and the selected VL2 CDRs 1, 2, 3 amino acid sequences are set forth in SEQ ID NOs: 29-31, respectively;
(4) the selected VH1 CDRs 1, 2, 3 amino acid sequences are set forth in SEQ ID NOs: 1-3, respectively, and the selected VL1 CDRs 1, 2, 3 amino acid sequences are set forth in SEQ ID NOs: 29-31, respectively, and the selected VH2 CDRs 1, 2, 3 amino acid sequences are set forth in SEQ ID NOs: 45-47, respectively, and the selected VL2 CDRs 1, 2, 3 amino acid sequences are set forth in SEQ ID NOs: 29-31, respectively;
(5) the selected VH1 CDRs 1, 2, 3 amino acid sequences are set forth in SEQ ID NOs: 1-3, respectively, and the selected VL1 CDRs 1, 2, 3 amino acid sequences are set forth in SEQ ID NOs: 29-31, respectively, and the selected VH2 CDRs 1, 2, 3 amino acid sequences are set forth in SEQ ID NOs: 48-50, respectively, and the selected VL2 CDRs 1, 2, 3 amino acid sequences are set forth in SEQ ID NOs: 29-31, respectively;
(6) the selected VH1 CDRs 1, 2, 3 amino acid sequences are set forth in SEQ ID NOs: 1-3, respectively, and the selected VL1 CDRs 1, 2, 3 amino acid sequences are set forth in SEQ ID NOs: 29-31, respectively, and the selected VH2 CDRs 1, 2, 3 amino acid sequences are set forth in SEQ ID NOs: 51-53, respectively, and the selected VL2 CDRs 1, 2, 3 amino acid sequences are set forth in SEQ ID NOs: 29-31, respectively;
(7) the selected VH1 CDRs 1, 2, 3 amino acid sequences are set forth in SEQ ID NOs: 1-3, respectively, and the selected VL1 CDRs 1, 2, 3 amino acid sequences are set forth in SEQ ID NOs: 29-31, respectively, and the selected VH2 CDRs 1, 2, 3 amino acid sequences are set forth in SEQ ID NOs: 54-56, respectively, and the selected VL2 CDRs 1, 2, 3 amino acid sequences are set forth in SEQ ID NOs: 29-31, respectively;
(8) the selected VH1 CDRs 1, 2, 3 amino acid sequences are set forth in SEQ ID NOs: 1-3, respectively, and the selected VL1 CDRs 1, 2, 3 amino acid sequences are set forth in SEQ ID NOs: 29-31, respectively, and the selected VH2 CDRs 1, 2, 3 amino acid sequences are set forth in SEQ ID NOs: 57-59, respectively, and the selected VL2 CDRs 1, 2, 3 amino acid sequences are set forth in SEQ ID NOs: 29-31, respectively;
(9) the selected VH1 CDRs 1, 2, 3 amino acid sequences are set forth in SEQ ID NOs: 4-6, respectively, and the selected VL1 CDRs 1, 2, 3 amino acid sequences are set forth in SEQ ID NOs: 29-31, respectively, and the selected VH2 CDRs 1, 2, 3 amino acid sequences are set forth in SEQ ID NOs: 36-38, respectively, and the selected VL2 CDRs 1, 2, 3 amino acid sequences are set forth in SEQ ID NOs: 29-31, respectively;
(10) the selected VH1 CDRs 1, 2, 3 amino acid sequences are set forth in SEQ ID NOs: 4-6, respectively, and the selected VL1 CDRs 1, 2, 3 amino acid sequences are set forth in SEQ ID NOs: 29-31, respectively, and the selected VH2 CDRs 1, 2, 3 amino acid sequences are set forth in SEQ ID NOs: 39-41, respectively, and the selected VL2 CDRs 1, 2, 3 amino acid sequences are set forth in SEQ ID NOs: 29-31, respectively;
(11) the selected VH1 CDRs 1, 2, 3 amino acid sequences are set forth in SEQ ID NOs: 4-6, respectively, and the selected VL1 CDRs 1, 2, 3 amino acid sequences are set forth in SEQ ID NOs: 29-31, respectively, and the selected VH2 CDRs 1, 2, 3 amino acid sequences are set forth in SEQ ID NOs: 42-44, respectively, and the selected VL2 CDRs 1, 2, 3 amino acid sequences are set forth in SEQ ID NOs: 29-31, respectively;
(12) the selected VH1 CDRs 1, 2, 3 amino acid sequences are set forth in SEQ ID NOs: 4-6, respectively, and the selected VL1 CDRs 1, 2, 3 amino acid sequences are set forth in SEQ ID NOs: 29-31, respectively, and the selected VH2 CDRs 1, 2, 3 amino acid sequences are set forth in SEQ ID NOs: 45-47, respectively, and the selected VL2 CDRs 1, 2, 3 amino acid sequences are set forth in SEQ ID NOs: 29-31, respectively;
(13) the selected VH1 CDRs 1, 2, 3 amino acid sequences are set forth in SEQ ID NOs: 4-6, respectively, and the selected VL1 CDRs 1, 2, 3 amino acid sequences are set forth in SEQ ID NOs: 29-31, respectively, and the selected VH2 CDRs 1, 2, 3 amino acid sequences are set forth in SEQ ID NOs: 48-50, respectively, and the selected VL2 CDRs 1, 2, 3 amino acid sequences are set forth in SEQ ID NOs: 29-31, respectively;
(14) the selected VH1 CDRs 1, 2, 3 amino acid sequences are set forth in SEQ ID NOs: 4-6, respectively, and the selected VL1 CDRs 1, 2, 3 amino acid sequences are set forth in SEQ ID NOs: 29-31, respectively, and the selected VH2 CDRs 1, 2, 3 amino acid sequences are set forth in SEQ ID NOs: 51-53, respectively, and the selected VL2 CDRs 1, 2, 3 amino acid sequences are set forth in SEQ ID NOs: 29-31, respectively;
(15) the selected VH1 CDRs 1, 2, 3 amino acid sequences are set forth in SEQ ID NOs: 4-6, respectively, and the selected VL1 CDRs 1, 2, 3 amino acid sequences are set forth in SEQ ID NOs: 29-31, respectively, and the selected VH2 CDRs 1, 2, 3 amino acid sequences are set forth in SEQ ID NOs: 54-56, respectively, and the selected VL2 CDRs 1, 2, 3 amino acid sequences are set forth in SEQ ID NOs: 29-31, respectively;
(16) the selected VH1 CDRs 1, 2, 3 amino acid sequences are set forth in SEQ ID NOs: 4-6, respectively, and the selected VL1 CDRs 1, 2, 3 amino acid sequences are set forth in SEQ ID NOs: 29-31, respectively, and the selected VH2 CDRs 1, 2, 3 amino acid sequences are set forth in SEQ ID NOs: 57-59, respectively, and the selected VL2 CDRs 1, 2, 3 amino acid sequences are set forth in SEQ ID NOs: 29-31, respectively;
(17) the selected VH1 CDRs 1, 2, 3 amino acid sequences are set forth in SEQ ID NOs: 7-9, respectively, and the selected VL1 CDRs 1, 2, 3 amino acid sequences are set forth in SEQ ID NOs: 29-31, respectively, and the selected VH2 CDRs 1, 2, 3 amino acid sequences are set forth in SEQ ID NOs: 36-38, respectively, and the selected VL2 CDRs 1, 2, 3 amino acid sequences are set forth in SEQ ID NOs: 29-31, respectively;
(18) the selected VH1 CDRs 1, 2, 3 amino acid sequences are set forth in SEQ ID NOs: 7-9, respectively, and the selected VL1 CDRs 1, 2, 3 amino acid sequences are set forth in SEQ ID NOs: 29-31, respectively, and the selected VH2 CDRs 1, 2, 3 amino acid sequences are set forth in SEQ ID NOs: 39-41, respectively, and the selected VL2 CDRs 1, 2, 3 amino acid sequences are set forth in SEQ ID NOs: 29-31, respectively;
(19) the selected VH1 CDRs 1, 2, 3 amino acid sequences are set forth in SEQ ID NOs: 7-9, respectively, and the selected VL1 CDRs 1, 2, 3 amino acid sequences are set forth in SEQ ID NOs: 29-31, respectively, and the selected VH2 CDRs 1, 2, 3 amino acid sequences are set forth in SEQ ID NOs: 42-44, respectively, and the selected VL2 CDRs 1, 2, 3 amino acid sequences are set forth in SEQ ID NOs: 29-31, respectively;
(20) the selected VH1 CDRs 1, 2, 3 amino acid sequences are set forth in SEQ ID NOs: 7-9, respectively, and the selected VL1 CDRs 1, 2, 3 amino acid sequences are set forth in SEQ ID NOs: 29-31, respectively, and the selected VH2 CDRs 1, 2, 3 amino acid sequences are set forth in SEQ ID NOs: 45-47, respectively, and the selected VL2 CDRs 1, 2, 3 amino acid sequences are set forth in SEQ ID NOs: 29-31, respectively;
(21) the selected VH1 CDRs 1, 2, 3 amino acid sequences are set forth in SEQ ID NOs: 7-9, respectively, and the selected VL1 CDRs 1, 2, 3 amino acid sequences are set forth in SEQ ID NOs: 29-31, respectively, and the selected VH2 CDRs 1, 2, 3 amino acid sequences are set forth in SEQ ID NOs: 48-50, respectively, and the selected VL2 CDRs 1, 2, 3 amino acid sequences are set forth in SEQ ID NOs: 29-31, respectively;
(22) the selected VH1 CDRs 1, 2, 3 amino acid sequences are set forth in SEQ ID NOs: 7-9, respectively, and the selected VL1 CDRs 1, 2, 3 amino acid sequences are set forth in SEQ ID NOs: 29-31, respectively, and the selected VH2 CDRs 1, 2, 3 amino acid sequences are set forth in SEQ ID NOs: 51-53, respectively, and the selected VL2 CDRs 1, 2, 3 amino acid sequences are set forth in SEQ ID NOs: 29-31, respectively;
(23) the selected VH1 CDRs 1, 2, 3 amino acid sequences are set forth in SEQ ID NOs: 7-9, respectively, and the selected VL1 CDRs 1, 2, 3 amino acid sequences are set forth in SEQ ID NOs: 29-31, respectively, and the selected VH2 CDRs 1, 2, 3 amino acid sequences are set forth in SEQ ID NOs: 54-56, respectively, and the selected VL2 CDRs 1, 2, 3 amino acid sequences are set forth in SEQ ID NOs: 29-31, respectively;
(24) the selected VH1 CDRs 1, 2, 3 amino acid sequences are set forth in SEQ ID NOs: 7-9, respectively, and the selected VL1 CDRs 1, 2, 3 amino acid sequences are set forth in SEQ ID NOs: 29-31, respectively, and the selected VH2 CDRs 1, 2, 3 amino acid sequences are set forth in SEQ ID NOs: 57-59, respectively, and the selected VL2 CDRs 1, 2, 3 amino acid sequences are set forth in SEQ ID NOs: 29-31, respectively;
(25) the selected VH1 CDRs 1, 2, 3 amino acid sequences are set forth in SEQ ID NOs: 10-12, respectively, and the selected VL1 CDRs 1, 2, 3 amino acid sequences are set forth in SEQ ID NOs: 29-31, respectively, and the selected VH2 CDRs 1, 2, 3 amino acid sequences are set forth in SEQ ID NOs: 36-38, respectively, and the selected VL2 CDRs 1, 2, 3 amino acid sequences are set forth in SEQ ID NOs: 29-31, respectively;
(26) the selected VH1 CDRs 1, 2, 3 amino acid sequences are set forth in SEQ ID NOs: 10-12, respectively, and the selected VL1 CDRs 1, 2, 3 amino acid sequences are set forth in SEQ ID NOs: 29-31, respectively, and the selected VH2 CDRs 1, 2, 3 amino acid sequences are set forth in SEQ ID NOs: 39-41, respectively, and the selected VL2 CDRs 1, 2, 3 amino acid sequences are set forth in SEQ ID NOs: 29-31, respectively;
(27) the selected VH1 CDRs 1, 2, 3 amino acid sequences are set forth in SEQ ID NOs: 10-12, respectively, and the selected VL1 CDRs 1, 2, 3 amino acid sequences are set forth in SEQ ID NOs: 29-31, respectively, and the selected VH2 CDRs 1, 2, 3 amino acid sequences are set forth in SEQ ID NOs: 42-44, respectively, and the selected VL2 CDRs 1, 2, 3 amino acid sequences are set forth in SEQ ID NOs: 29-31, respectively;
(28) the selected VH1 CDRs 1, 2, 3 amino acid sequences are set forth in SEQ ID NOs: 10-12, respectively, and the selected VL1 CDRs 1, 2, 3 amino acid sequences are set forth in SEQ ID NOs: 29-31, respectively, and the selected VH2 CDRs 1, 2, 3 amino acid sequences are set forth in SEQ ID NOs: 45-47, respectively, and the selected VL2 CDRs 1, 2, 3 amino acid sequences are set forth in SEQ ID NOs: 29-31, respectively;
(29) the selected VH1 CDRs 1, 2, 3 amino acid sequences are set forth in SEQ ID NOs: 10-12, respectively, and the selected VL1 CDRs 1, 2, 3 amino acid sequences are set forth in SEQ ID NOs: 29-31, respectively, and the selected VH2 CDRs 1, 2, 3 amino acid sequences are set forth in SEQ ID NOs: 48-50, respectively, and the selected VL2 CDRs 1, 2, 3 amino acid sequences are set forth in SEQ ID NOs: 29-31, respectively;
(30) the selected VH1 CDRs 1, 2, 3 amino acid sequences are set forth in SEQ ID NOs: 10-12, respectively, and the selected VL1 CDRs 1, 2, 3 amino acid sequences are set forth in SEQ ID NOs: 29-31, respectively, and the selected VH2 CDRs 1, 2, 3 amino acid sequences are set forth in SEQ ID NOs: 51-53, respectively, and the selected VL2 CDRs 1, 2, 3 amino acid sequences are set forth in SEQ ID NOs: 29-31, respectively;
(31) the selected VH1 CDRs 1, 2, 3 amino acid sequences are set forth in SEQ ID NOs: 10-12, respectively, and the selected VL1 CDRs 1, 2, 3 amino acid sequences are set forth in SEQ ID NOs: 29-31, respectively, and the selected VH2 CDRs 1, 2, 3 amino acid sequences are set forth in SEQ ID NOs: 54-56, respectively, and the selected VL2 CDRs 1, 2, 3 amino acid sequences are set forth in SEQ ID NOs: 29-31, respectively;
(32) the selected VH1 CDRs 1, 2, 3 amino acid sequences are set forth in SEQ ID NOs: 10-12, respectively, and the selected VL1 CDRs 1, 2, 3 amino acid sequences are set forth in SEQ ID NOs: 29-31, respectively, and the selected VH2 CDRs 1, 2, 3 amino acid sequences are set forth in SEQ ID NOs: 57-59, respectively, and the selected VL2 CDRs 1, 2, 3 amino acid sequences are set forth in SEQ ID NOs: 29-31, respectively;
(33) the selected VH1 CDRs 1, 2, 3 amino acid sequences are set forth in SEQ ID NOs: 13-15, respectively, and the selected VL1 CDRs 1, 2, 3 amino acid sequences are set forth in SEQ ID NOs: 32-34, respectively, and the selected VH2 CDRs 1, 2, 3 amino acid sequences are set forth in SEQ ID NOs: 60-62, respectively, and the selected VL2 CDRs 1, 2, 3 amino acid sequences are set forth in SEQ ID NOs: 32-34, respectively;
(34) the selected VH1 CDRs 1, 2, 3 amino acid sequences are set forth in SEQ ID NOs: 13-15, respectively, and the selected VL1 CDRs 1, 2, 3 amino acid sequences are set forth in SEQ ID NOs: 32-34, respectively, and the selected VH2 CDRs 1, 2, 3 amino acid sequences are set forth in SEQ ID NOs: 63-65, respectively, and the selected VL2 CDRs 1, 2, 3 amino acid sequences are set forth in SEQ ID NOs: 32-34, respectively;
(35) the selected VH1 CDRs 1, 2, 3 amino acid sequences are set forth in SEQ ID NOs: 13-15, respectively, and the selected VL1 CDRs 1, 2, 3 amino acid sequences are set forth in SEQ ID NOs: 32-34, respectively, and the selected VH2 CDRs 1, 2, 3 amino acid sequences are set forth in SEQ ID NOs: 66-68, respectively, and the selected VL2 CDRs 1, 2, 3 amino acid sequences are set forth in SEQ ID NOs: 32-34, respectively;
(36) the selected VH1 CDRs 1, 2, 3 amino acid sequences are set forth in SEQ ID NOs: 13-15, respectively, and the selected VL1 CDRs 1, 2, 3 amino acid sequences are set forth in SEQ ID NOs: 32-34, respectively, and the selected VH2 CDRs 1, 2, 3 amino acid sequences are set forth in SEQ ID NOs: 69-71, respectively, and the selected VL2 CDRs 1, 2, 3 amino acid sequences are set forth in SEQ ID NOs: 32-34, respectively;
(37) the selected VH1 CDRs 1, 2, 3 amino acid sequences are set forth in SEQ ID NOs: 13-15, respectively, and the selected VL1 CDRs 1, 2, 3 amino acid sequences are set forth in SEQ ID NOs: 32-34, respectively, and the selected VH2 CDRs 1, 2, 3 amino acid sequences are set forth in SEQ ID NOs: 72-74, respectively, and the selected VL2 CDRs 1, 2, 3 amino acid sequences are set forth in SEQ ID NOs: 32-34, respectively;
(38) the selected VH1 CDRs 1, 2, 3 amino acid sequences are set forth in SEQ ID NOs: 13-15, respectively, and the selected VL1 CDRs 1, 2, 3 amino acid sequences are set forth in SEQ ID NOs: 32-34, respectively, and the selected VH2 CDRs 1, 2, 3 amino acid sequences are set forth in SEQ ID NOs: 75-77, respectively, and the selected VL2 CDRs 1, 2, 3 amino acid sequences are set forth in SEQ ID NOs: 32-34, respectively;
(39) the selected VH1 CDRs 1, 2, 3 amino acid sequences are set forth in SEQ ID NOs: 13-15, respectively, and the selected VL1 CDRs 1, 2, 3 amino acid sequences are set forth in SEQ ID NOs: 32-34, respectively, and the selected VH2 CDRs 1, 2, 3 amino acid sequences are set forth in SEQ ID NOs: 78-80, respectively, and the selected VL2 CDRs 1, 2, 3 amino acid sequences are set forth in SEQ ID NOs: 32-34, respectively;
(40) the selected VH1 CDRs 1, 2, 3 amino acid sequences are set forth in SEQ ID NOs: 13-15, respectively, and the selected VL1 CDRs 1, 2, 3 amino acid sequences are set forth in SEQ ID NOs: 32-34, respectively, and the selected VH2 CDRs 1, 2, 3 amino acid sequences are set forth in SEQ ID NOs: 81-83, respectively, and the selected VL2 CDRs 1, 2, 3 amino acid sequences are set forth in SEQ ID NOs: 32-34, respectively;
(41) the selected VH1 CDRs 1, 2, 3 amino acid sequences are set forth in SEQ ID NOs: 16-18, respectively, and the selected VL1 CDRs 1, 2, 3 amino acid sequences are set forth in SEQ ID NOs: 32-34, respectively, and the selected VH2 CDRs 1, 2, 3 amino acid sequences are set forth in SEQ ID NOs: 60-62, respectively, and the selected VL2 CDRs 1, 2, 3 amino acid sequences are set forth in SEQ ID NOs: 32-34, respectively;
(42) the selected VH1 CDRs 1, 2, 3 amino acid sequences are set forth in SEQ ID NOs: 16-18, respectively, and the selected VL1 CDRs 1, 2, 3 amino acid sequences are set forth in SEQ ID NOs: 32-34, respectively, and the selected VH2 CDRs 1, 2, 3 amino acid sequences are set forth in SEQ ID NOs: 63-65, respectively, and the selected VL2 CDRs 1, 2, 3 amino acid sequences are set forth in SEQ ID NOs: 32-34, respectively;
(43) the selected VH1 CDRs 1, 2, 3 amino acid sequences are set forth in SEQ ID NOs: 16-18, respectively, and the selected VL1 CDRs 1, 2, 3 amino acid sequences are set forth in SEQ ID NOs: 32-34, respectively, and the selected VH2 CDRs 1, 2, 3 amino acid sequences are set forth in SEQ ID NOs: 66-68, respectively, and the selected VL2 CDRs 1, 2, 3 amino acid sequences are set forth in SEQ ID NOs: 32-34, respectively;
(44) the selected VH1 CDRs 1, 2, 3 amino acid sequences are set forth in SEQ ID NOs: 16-18, respectively, and the selected VL1 CDRs 1, 2, 3 amino acid sequences are set forth in SEQ ID NOs: 32-34, respectively, and the selected VH2 CDRs 1, 2, 3 amino acid sequences are set forth in SEQ ID NOs: 69-71, respectively, and the selected VL2 CDRs 1, 2, 3 amino acid sequences are set forth in SEQ ID NOs: 32-34, respectively;
(45) the selected VH1 CDRs 1, 2, 3 amino acid sequences are set forth in SEQ ID NOs: 16-18, respectively, and the selected VL1 CDRs 1, 2, 3 amino acid sequences are set forth in SEQ ID NOs: 32-34, respectively, and the selected VH2 CDRs 1, 2, 3 amino acid sequences are set forth in SEQ ID NOs: 72-74, respectively, and the selected VL2 CDRs 1, 2, 3 amino acid sequences are set forth in SEQ ID NOs: 32-34, respectively;
(46) the selected VH1 CDRs 1, 2, 3 amino acid sequences are set forth in SEQ ID NOs: 16-18, respectively, and the selected VL1 CDRs 1, 2, 3 amino acid sequences are set forth in SEQ ID NOs: 32-34, respectively, and the selected VH2 CDRs 1, 2, 3 amino acid sequences are set forth in SEQ ID NOs: 75-77, respectively, and the selected VL2 CDRs 1, 2, 3 amino acid sequences are set forth in SEQ ID NOs: 32-34, respectively;
(47) the selected VH1 CDRs 1, 2, 3 amino acid sequences are set forth in SEQ ID NOs: 16-18, respectively, and the selected VL1 CDRs 1, 2, 3 amino acid sequences are set forth in SEQ ID NOs: 32-34, respectively, and the selected VH2 CDRs 1, 2, 3 amino acid sequences are set forth in SEQ ID NOs: 78-80, respectively, and the selected VL2 CDRs 1, 2, 3 amino acid sequences are set forth in SEQ ID NOs: 32-34, respectively;
(48) the selected VH1 CDRs 1, 2, 3 amino acid sequences are set forth in SEQ ID NOs: 16-18, respectively, and the selected VL1 CDRs 1, 2, 3 amino acid sequences are set forth in SEQ ID NOs: 32-34, respectively, and the selected VH2 CDRs 1, 2, 3 amino acid sequences are set forth in SEQ ID NOs: 81-83, respectively, and the selected VL2 CDRs 1, 2, 3 amino acid sequences are set forth in SEQ ID NOs: 32-34, respectively;
(49) the selected VH1 CDRs 1, 2, 3 amino acid sequences are set forth in SEQ ID NOs: 19-21, respectively, and the selected VL1 CDRs 1, 2, 3 amino acid sequences are set forth in SEQ ID NOs: 32-34, respectively, and the selected VH2 CDRs 1, 2, 3 amino acid sequences are set forth in SEQ ID NOs: 60-62, respectively, and the selected VL2 CDRs 1, 2, 3 amino acid sequences are set forth in SEQ ID NOs: 32-34, respectively;
(50) the selected VH1 CDRs 1, 2, 3 amino acid sequences are set forth in SEQ ID NOs: 19-21, respectively, and the selected VL1 CDRs 1, 2, 3 amino acid sequences are set forth in SEQ ID NOs: 32-34, respectively, and the selected VH2 CDRs 1, 2, 3 amino acid sequences are set forth in SEQ ID NOs: 63-65, respectively, and the selected VL2 CDRs 1, 2, 3 amino acid sequences are set forth in SEQ ID NOs: 32-34, respectively;
(51) the selected VH1 CDRs 1, 2, 3 amino acid sequences are set forth in SEQ ID NOs: 19-21, respectively, and the selected VL1 CDRs 1, 2, 3 amino acid sequences are set forth in SEQ ID NOs: 32-34, respectively, and the selected VH2 CDRs 1, 2, 3 amino acid sequences are set forth in SEQ ID NOs: 66-68, respectively, and the selected VL2 CDRs 1, 2, 3 amino acid sequences are set forth in SEQ ID NOs: 32-34, respectively;
(52) the selected VH1 CDRs 1, 2, 3 amino acid sequences are set forth in SEQ ID NOs: 19-21, respectively, and the selected VL1 CDRs 1, 2, 3 amino acid sequences are set forth in SEQ ID NOs: 32-34, respectively, and the selected VH2 CDRs 1, 2, 3 amino acid sequences are set forth in SEQ ID NOs: 69-71, respectively, and the selected VL2 CDRs 1, 2, 3 amino acid sequences are set forth in SEQ ID NOs: 32-34, respectively;
(53) the selected VH1 CDRs 1, 2, 3 amino acid sequences are set forth in SEQ ID NOs: 19-21, respectively, and the selected VL1 CDRs 1, 2, 3 amino acid sequences are set forth in SEQ ID NOs: 32-34, respectively, and the selected VH2 CDRs 1, 2, 3 amino acid sequences are set forth in SEQ ID NOs: 72-74, respectively, and the selected VL2 CDRs 1, 2, 3 amino acid sequences are set forth in SEQ ID NOs: 32-34, respectively;
(54) the selected VH1 CDRs 1, 2, 3 amino acid sequences are set forth in SEQ ID NOs: 19-21, respectively, and the selected VL1 CDRs 1, 2, 3 amino acid sequences are set forth in SEQ ID NOs: 32-34, respectively, and the selected VH2 CDRs 1, 2, 3 amino acid sequences are set forth in SEQ ID NOs: 75-77, respectively, and the selected VL2 CDRs 1, 2, 3 amino acid sequences are set forth in SEQ ID NOs: 32-34, respectively;
(55) the selected VH1 CDRs 1, 2, 3 amino acid sequences are set forth in SEQ ID NOs: 19-21, respectively, and the selected VL1 CDRs 1, 2, 3 amino acid sequences are set forth in SEQ ID NOs: 32-34, respectively, and the selected VH2 CDRs 1, 2, 3 amino acid sequences are set forth in SEQ ID NOs: 78-80, respectively, and the selected VL2 CDRs 1, 2, 3 amino acid sequences are set forth in SEQ ID NOs: 32-34, respectively;
(56) the selected VH1 CDRs 1, 2, 3 amino acid sequences are set forth in SEQ ID NOs: 19-21, respectively, and the selected VL1 CDRs 1, 2, 3 amino acid sequences are set forth in SEQ ID NOs: 32-34, respectively, and the selected VH2 CDRs 1, 2, 3 amino acid sequences are set forth in SEQ ID NOs: 81-83, respectively, and the selected VL2 CDRs 1, 2, 3 amino acid sequences are set forth in SEQ ID NOs: 32-34, respectively;
(57) the selected VH1 CDRs 1, 2, 3 amino acid sequences are set forth in SEQ ID NOs: 22-24, respectively, and the selected VL1 CDRs 1, 2, 3 amino acid sequences are set forth in SEQ ID NOs: 32-34, respectively, and the selected VH2 CDRs 1, 2, 3 amino acid sequences are set forth in SEQ ID NOs: 60-62, respectively, and the selected VL2 CDRs 1, 2, 3 amino acid sequences are set forth in SEQ ID NOs: 32-34, respectively;
(58) the selected VH1 CDRs 1, 2, 3 amino acid sequences are set forth in SEQ ID NOs: 22-24, respectively, and the selected VL1 CDRs 1, 2, 3 amino acid sequences are set forth in SEQ ID NOs: 32-34, respectively, and the selected VH2 CDRs 1, 2, 3 amino acid sequences are set forth in SEQ ID NOs: 63-65, respectively, and the selected VL2 CDRs 1, 2, 3 amino acid sequences are set forth in SEQ ID NOs: 32-34, respectively;
(59) the selected VH1 CDRs 1, 2, 3 amino acid sequences are set forth in SEQ ID NOs: 22-24, respectively, and the selected VL1 CDRs 1, 2, 3 amino acid sequences are set forth in SEQ ID NOs: 32-34, respectively, and the selected VH2 CDRs 1, 2, 3 amino acid sequences are set forth in SEQ ID NOs: 66-68, respectively, and the selected VL2 CDRs 1, 2, 3 amino acid sequences are set forth in SEQ ID NOs: 32-34, respectively;
(60) the selected VH1 CDRs 1, 2, 3 amino acid sequences are set forth in SEQ ID NOs: 22-24, respectively, and the selected VL1 CDRs 1, 2, 3 amino acid sequences are set forth in SEQ ID NOs: 32-34, respectively, and the selected VH2 CDRs 1, 2, 3 amino acid sequences are set forth in SEQ ID NOs: 69-71, respectively, and the selected VL2 CDRs 1, 2, 3 amino acid sequences are set forth in SEQ ID NOs: 32-34, respectively;
(61) the selected VH1 CDRs 1, 2, 3 amino acid sequences are set forth in SEQ ID NOs: 22-24, respectively, and the selected VL1 CDRs 1, 2, 3 amino acid sequences are set forth in SEQ ID NOs: 32-34, respectively, and the selected VH2 CDRs 1, 2, 3 amino acid sequences are set forth in SEQ ID NOs: 72-74, respectively, and the selected VL2 CDRs 1, 2, 3 amino acid sequences are set forth in SEQ ID NOs: 32-34, respectively;
(62) the selected VH1 CDRs 1, 2, 3 amino acid sequences are set forth in SEQ ID NOs: 22-24, respectively, and the selected VL1 CDRs 1, 2, 3 amino acid sequences are set forth in SEQ ID NOs: 32-34, respectively, and the selected VH2 CDRs 1, 2, 3 amino acid sequences are set forth in SEQ ID NOs: 75-77, respectively, and the selected VL2 CDRs 1, 2, 3 amino acid sequences are set forth in SEQ ID NOs: 32-34, respectively;
(63) the selected VH1 CDRs 1, 2, 3 amino acid sequences are set forth in SEQ ID NOs: 22-24, respectively, and the selected VL1 CDRs 1, 2, 3 amino acid sequences are set forth in SEQ ID NOs: 32-34, respectively, and the selected VH2 CDRs 1, 2, 3 amino acid sequences are set forth in SEQ ID NOs: 78-80, respectively, and the selected VL2 CDRs 1, 2, 3 amino acid sequences are set forth in SEQ ID NOs: 32-34, respectively; and
(64) the selected VH1 CDRs 1, 2, 3 amino acid sequences are set forth in SEQ ID NOs: 22-24, respectively, and the selected VL1 CDRs 1, 2, 3 amino acid sequences are set forth in SEQ ID NOs: 32-34, respectively, and the selected VH2 CDRs 1, 2, 3 amino acid sequences are set forth in SEQ ID NOs: 81-83, respectively, and the selected VL2 CDRs 1, 2, 3 amino acid sequences are set forth in SEQ ID NOs: 32-34, respectively.

In some embodiments, the first heavy chain variable region comprises a sequence that is at least 80%, 85%, 90%, or 95% identical to SEQ ID NO: 25, the first light chain variable region comprises a sequence that is at least 80%, 85%, 90%, or 95% identical to SEQ ID NO: 35, the second heavy chain variable region comprises a sequence that is at least 80%, 85%, 90%, or 95% identical to SEQ ID NO: 84, and the second light chain variable region comprises a sequence that is at least 80%, 85%, 90%, or 95% identical to SEQ ID NO: 35.

In some embodiments, the first heavy chain variable region comprises a sequence that is at least 80%, 85%, 90%, or 95% identical to SEQ ID NO: 25, the first light chain variable region comprises a sequence that is at least 80%, 85%, 90%, or 95% identical to SEQ ID NO: 35, the second heavy chain variable region comprises a sequence that is at least 80%, 85%, 90%, or 95% identical to SEQ ID NO: 85, and the second light chain variable region comprises a sequence that is at least 80%, 85%, 90%, or 95% identical to SEQ ID NO: 35.

In some embodiments, the first heavy chain variable region comprises a sequence that is at least 80%, 85%, 90%, or 95% identical to SEQ ID NO: 25, the first light chain variable region comprises a sequence that is at least 80%, 85%, 90%, or 95% identical to SEQ ID NO: 35, the second heavy chain variable region comprises a sequence that is at least 80%, 85%, 90%, or 95% identical to SEQ ID NO: 86, and the second light chain variable region comprises a sequence that is at least 80%, 85%, 90%, or 95% identical to SEQ ID NO: 35.

In some embodiments, the first heavy chain variable region comprises a sequence that is at least 80%, 85%, 90%, or 95% identical to SEQ ID NO: 25, the first light chain variable region comprises a sequence that is at least 80%, 85%, 90%, or 95% identical to SEQ ID NO: 35, the second heavy chain variable region comprises a sequence that is at least 80%, 85%, 90%, or 95% identical to SEQ ID NO: 87, and the second light chain variable region comprises a sequence that is at least 80%, 85%, 90%, or 95% identical to SEQ ID NO: 35.

In some embodiments, the first heavy chain variable region comprises a sequence that is at least 80%, 85%, 90%, or 95% identical to SEQ ID NO: 25, the first light chain variable region comprises a sequence that is at least 80%, 85%, 90%, or 95% identical to SEQ ID NO: 35, the second heavy chain variable region comprises a sequence that is at least 80%, 85%, 90%, or 95% identical to SEQ ID NO: 88, and the second light chain variable region comprises a sequence that is at least 80%, 85%, 90%, or 95% identical to SEQ ID NO: 35.

In some embodiments, the first heavy chain variable region comprises a sequence that is at least 80%, 85%, 90%, or 95% identical to SEQ ID NO: 25, the first light chain variable region comprises a sequence that is at least 80%, 85%, 90%, or 95% identical to SEQ ID NO: 35, the second heavy chain variable region comprises a sequence that is at least 80%, 85%, 90%, or 95% identical to SEQ ID NO: 89, and the second light chain variable region comprises a sequence that is at least 80%, 85%, 90%, or 95% identical to SEQ ID NO: 35.

In some embodiments, the first heavy chain variable region comprises a sequence that is at least 80%, 85%, 90%, or 95% identical to SEQ ID NO: 25, the first light chain variable region comprises a sequence that is at least 80%, 85%, 90%, or 95% identical to SEQ ID NO: 35, the second heavy chain variable region comprises a sequence that is at least 80%, 85%, 90%, or 95% identical to SEQ ID NO: 90, and the second light chain variable region comprises a sequence that is at least 80%, 85%, 90%, or 95% identical to SEQ ID NO: 35.

In some embodiments, the first heavy chain variable region comprises a sequence that is at least 80%, 85%, 90%, or 95% identical to SEQ ID NO: 25, the first light chain variable region comprises a sequence that is at least 80%, 85%, 90%, or 95% identical to SEQ ID NO: 35, the second heavy chain variable region comprises a sequence that is at least 80%, 85%, 90%, or 95% identical to SEQ ID NO: 91, and the second light chain variable region comprises a sequence that is at least 80%, 85%, 90%, or 95% identical to SEQ ID NO: 35.

In some embodiments, the first heavy chain variable region comprises a sequence that is at least 80%, 85%, 90%, or 95% identical to SEQ ID NO: 26, the first light chain variable region comprises a sequence that is at least 80%, 85%, 90%, or 95% identical to SEQ ID NO: 35, the second heavy chain variable region comprises a sequence that is at least 80%, 85%, 90%, or 95% identical to SEQ ID NO: 84, and the second light chain variable region comprises a sequence that is at least 80%, 85%, 90%, or 95% identical to SEQ ID NO: 35.

In some embodiments, the first heavy chain variable region comprises a sequence that is at least 80%, 85%, 90%, or 95% identical to SEQ ID NO: 26, the first light chain variable region comprises a sequence that is at least 80%, 85%, 90%, or 95% identical to SEQ ID NO: 35, the second heavy chain variable region comprises a sequence that is at least 80%, 85%, 90%, or 95% identical to SEQ ID NO: 85, and the second light chain variable region comprises a sequence that is at least 80%, 85%, 90%, or 95% identical to SEQ ID NO: 35.

In some embodiments, the first heavy chain variable region comprises a sequence that is at least 80%, 85%, 90%, or 95% identical to SEQ ID NO: 26, the first light chain variable region comprises a sequence that is at least 80%, 85%, 90%, or 95% identical to SEQ ID NO: 35, the second heavy chain variable region comprises a sequence that is at least 80%, 85%, 90%, or 95% identical to SEQ ID NO: 86, and the second light chain variable region comprises a sequence that is at least 80%, 85%, 90%, or 95% identical to SEQ ID NO: 35.

In some embodiments, the first heavy chain variable region comprises a sequence that is at least 80%, 85%, 90%, or 95% identical to SEQ ID NO: 26, the first light chain variable region comprises a sequence that is at least 80%, 85%, 90%, or 95% identical to SEQ ID NO: 35, the second heavy chain variable region comprises a sequence that is at least 80%, 85%, 90%, or 95% identical to SEQ ID NO: 87, and the second light chain variable region comprises a sequence that is at least 80%, 85%, 90%, or 95% identical to SEQ ID NO: 35.

In some embodiments, the first heavy chain variable region comprises a sequence that is at least 80%, 85%, 90%, or 95% identical to SEQ ID NO: 26, the first light chain variable region comprises a sequence that is at least 80%, 85%, 90%, or 95% identical to SEQ ID NO: 35, the second heavy chain variable region comprises a sequence that is at least 80%, 85%, 90%, or 95% identical to SEQ ID NO: 88, and the second light chain variable region comprises a sequence that is at least 80%, 85%, 90%, or 95% identical to SEQ ID NO: 35.

In some embodiments, the first heavy chain variable region comprises a sequence that is at least 80%, 85%, 90%, or 95% identical to SEQ ID NO: 26, the first light chain variable region comprises a sequence that is at least 80%, 85%, 90%, or 95% identical to SEQ ID NO: 35, the second heavy chain variable region comprises a sequence that is at least 80%, 85%, 90%, or 95% identical to SEQ ID NO: 89, and the second light chain variable region comprises a sequence that is at least 80%, 85%, 90%, or 95% identical to SEQ ID NO: 35.

In some embodiments, the first heavy chain variable region comprises a sequence that is at least 80%, 85%, 90%, or 95% identical to SEQ ID NO: 26, the first light chain variable region comprises a sequence that is at least 80%, 85%, 90%, or 95% identical to SEQ ID NO: 35, the second heavy chain variable region comprises a sequence that is at least 80%, 85%, 90%, or 95% identical to SEQ ID NO: 90, and the second light chain variable region comprises a sequence that is at least 80%, 85%, 90%, or 95% identical to SEQ ID NO: 35.

In some embodiments, the first heavy chain variable region comprises a sequence that is at least 80%, 85%, 90%, or 95% identical to SEQ ID NO: 26, the first light chain variable region comprises a sequence that is at least 80%, 85%, 90%, or 95% identical to SEQ ID NO: 35, the second heavy chain variable region comprises a sequence that is at least 80%, 85%, 90%, or 95% identical to SEQ ID NO: 91, and the second light chain variable region comprises a sequence that is at least 80%, 85%, 90%, or 95% identical to SEQ ID NO: 35.

In some embodiments, the first heavy chain variable region comprises a sequence that is at least 80%, 85%, 90%, or 95% identical to SEQ ID NO: 27, the first light chain variable region comprises a sequence that is at least 80%, 85%, 90%, or 95% identical to SEQ ID NO: 35, the second heavy chain variable region comprises a sequence that is at least 80%, 85%, 90%, or 95% identical to SEQ ID NO: 84, and the second light chain variable region comprises a sequence that is at least 80%, 85%, 90%, or 95% identical to SEQ ID NO: 35.

In some embodiments, the first heavy chain variable region comprises a sequence that is at least 80%, 85%, 90%, or 95% identical to SEQ ID NO: 27, the first light chain variable region comprises a sequence that is at least 80%, 85%, 90%, or 95% identical to SEQ ID NO: 35, the second heavy chain variable region comprises a sequence that is at least 80%, 85%, 90%, or 95% identical to SEQ ID NO: 85, and the second light chain variable region comprises a sequence that is at least 80%, 85%, 90%, or 95% identical to SEQ ID NO: 35.

In some embodiments, the first heavy chain variable region comprises a sequence that is at least 80%, 85%, 90%, or 95% identical to SEQ ID NO: 27, the first light chain variable region comprises a sequence that is at least 80%, 85%, 90%, or 95% identical to SEQ ID NO: 35, the second heavy chain variable region comprises a sequence that is at least 80%, 85%, 90%, or 95% identical to SEQ ID NO: 86, and the second light chain variable region comprises a sequence that is at least 80%, 85%, 90%, or 95% identical to SEQ ID NO: 35.

In some embodiments, the first heavy chain variable region comprises a sequence that is at least 80%, 85%, 90%, or 95% identical to SEQ ID NO: 27, the first light chain variable region comprises a sequence that is at least 80%, 85%, 90%, or 95% identical to SEQ ID NO: 35, the second heavy chain variable region comprises a sequence that is at least 80%, 85%, 90%, or 95% identical to SEQ ID NO: 87, and the second light chain variable region comprises a sequence that is at least 80%, 85%, 90%, or 95% identical to SEQ ID NO: 35.

In some embodiments, the first heavy chain variable region comprises a sequence that is at least 80%, 85%, 90%, or 95% identical to SEQ ID NO: 27, the first light chain variable region comprises a sequence that is at least 80%, 85%, 90%, or 95% identical to SEQ ID NO: 35, the second heavy chain variable region comprises a sequence that is at least 80%, 85%, 90%, or 95% identical to SEQ ID NO: 88, and the second light chain variable region comprises a sequence that is at least 80%, 85%, 90%, or 95% identical to SEQ ID NO: 35.

In some embodiments, the first heavy chain variable region comprises a sequence that is at least 80%, 85%, 90%, or 95% identical to SEQ ID NO: 27, the first light chain variable region comprises a sequence that is at least 80%, 85%, 90%, or 95% identical to SEQ ID NO: 35, the second heavy chain variable region comprises a sequence that is at least 80%, 85%, 90%, or 95% identical to SEQ ID NO: 89, and the second light chain variable region comprises a sequence that is at least 80%, 85%, 90%, or 95% identical to SEQ ID NO: 35.

In some embodiments, the first heavy chain variable region comprises a sequence that is at least 80%, 85%, 90%, or 95% identical to SEQ ID NO: 27, the first light chain variable region comprises a sequence that is at least 80%, 85%, 90%, or 95% identical to SEQ ID NO: 35, the second heavy chain variable region comprises a sequence that is at least 80%, 85%, 90%, or 95% identical to SEQ ID NO: 90, and the second light chain variable region comprises a sequence that is at least 80%, 85%, 90%, or 95% identical to SEQ ID NO: 35.

In some embodiments, the first heavy chain variable region comprises a sequence that is at least 80%, 85%, 90%, or 95% identical to SEQ ID NO: 27, the first light chain variable region comprises a sequence that is at least 80%, 85%, 90%, or 95% identical to SEQ ID NO: 35, the second heavy chain variable region comprises a sequence that is at least 80%, 85%, 90%, or 95% identical to SEQ ID NO: 91, and the second light chain variable region comprises a sequence that is at least 80%, 85%, 90%, or 95% identical to SEQ ID NO: 35.

In some embodiments, the first heavy chain variable region comprises a sequence that is at least 80%, 85%, 90%, or 95% identical to SEQ ID NO: 28, the first light chain variable region comprises a sequence that is at least 80%, 85%, 90%, or 95% identical to SEQ ID NO: 35, the second heavy chain variable region comprises a sequence that is at least 80%, 85%, 90%, or 95% identical to SEQ ID NO: 84, and the second light chain variable region comprises a sequence that is at least 80%, 85%, 90%, or 95% identical to SEQ ID NO: 35.

In some embodiments, the first heavy chain variable region comprises a sequence that is at least 80%, 85%, 90%, or 95% identical to SEQ ID NO: 28, the first light chain variable region comprises a sequence that is at least 80%, 85%, 90%, or 95% identical to SEQ ID NO: 35, the second heavy chain variable region comprises a sequence that is at least 80%, 85%, 90%, or 95% identical to SEQ ID NO: 85, and the second light chain variable region comprises a sequence that is at least 80%, 85%, 90%, or 95% identical to SEQ ID NO: 35.

In some embodiments, the first heavy chain variable region comprises a sequence that is at least 80%, 85%, 90%, or 95% identical to SEQ ID NO: 28, the first light chain variable region comprises a sequence that is at least 80%, 85%, 90%, or 95% identical to SEQ ID NO: 35, the second heavy chain variable region comprises a sequence that is at least 80%, 85%, 90%, or 95% identical to SEQ ID NO: 86, and the second light chain variable region comprises a sequence that is at least 80%, 85%, 90%, or 95% identical to SEQ ID NO: 35.

In some embodiments, the first heavy chain variable region comprises a sequence that is at least 80%, 85%, 90%, or 95% identical to SEQ ID NO: 28, the first light chain variable region comprises a sequence that is at least 80%, 85%, 90%, or 95% identical to SEQ ID NO: 35, the second heavy chain variable region comprises a sequence that is at least 80%, 85%, 90%, or 95% identical to SEQ ID NO: 87, and the second light chain variable region comprises a sequence that is at least 80%, 85%, 90%, or 95% identical to SEQ ID NO: 35.

In some embodiments, the first heavy chain variable region comprises a sequence that is at least 80%, 85%, 90%, or 95% identical to SEQ ID NO: 28, the first light chain variable region comprises a sequence that is at least 80%, 85%, 90%, or 95% identical to SEQ ID NO: 35, the second heavy chain variable region comprises a sequence that is at least 80%, 85%, 90%, or 95% identical to SEQ ID NO: 88, and the second light chain variable region comprises a sequence that is at least 80%, 85%, 90%, or 95% identical to SEQ ID NO: 35.

In some embodiments, the first heavy chain variable region comprises a sequence that is at least 80%, 85%, 90%, or 95% identical to SEQ ID NO: 28, the first light chain variable region comprises a sequence that is at least 80%, 85%, 90%, or 95% identical to SEQ ID NO: 35, the second heavy chain variable region comprises a sequence that is at least 80%, 85%, 90%, or 95% identical to SEQ ID NO: 89, and the second light chain variable region comprises a sequence that is at least 80%, 85%, 90%, or 95% identical to SEQ ID NO: 35.

In some embodiments, the first heavy chain variable region comprises a sequence that is at least 80%, 85%, 90%, or 95% identical to SEQ ID NO: 28, the first light chain variable region comprises a sequence that is at least 80%, 85%, 90%, or 95% identical to SEQ ID NO: 35, the second heavy chain variable region comprises a sequence that is at least 80%, 85%, 90%, or 95% identical to SEQ ID NO: 90, and the second light chain variable region comprises a sequence that is at least 80%, 85%, 90%, or 95% identical to SEQ ID NO: 35.

In some embodiments, the first heavy chain variable region comprises a sequence that is at least 80%, 85%, 90%, or 95% identical to SEQ ID NO: 28, the first light chain variable region comprises a sequence that is at least 80%, 85%, 90%, or 95% identical to SEQ ID NO: 35, the second heavy chain variable region comprises a sequence that is at least 80%, 85%, 90%, or 95% identical to SEQ ID NO: 91, and the second light chain variable region comprises a sequence that is at least 80%, 85%, 90%, or 95% identical to SEQ ID NO: 35.

In some embodiments, the antigen-binding protein construct is a multi-specific antibody (e.g., a bispecific antibody). In some embodiments, the first antigen-binding domain is a single-chain variable fragment (scFV); and/or the second antigen-binding domain is a scFv.

In some embodiments, the first light chain variable region and the second light chain variable region are identical.

In one aspect, the disclosure is related to a vector comprising one or more of the nucleic acids as described herein, a nucleic acid encoding the antibody or antigen-binding fragment thereof as described herein, or a nucleic acid encoding the antigen-binding protein construct as described herein.

In one aspect, the disclosure is related to a cell comprising the vector as described herein. In some embodiments, the cell is a CHO cell.

In one aspect, the disclosure is related to a cell comprising one or more of the nucleic acids as described herein, a nucleic acid encoding the antibody or antigen-binding fragment thereof as described herein, or a nucleic acid encoding the antigen-binding protein construct as described herein.

In one aspect, the disclosure is related to a method of producing an antibody or an antigen-binding fragment thereof, or an antigen-binding protein construct, the method comprising (a) culturing the cell as described herein under conditions sufficient for the cell to produce the antibody or the antigen-binding fragment thereof, or the antigen-binding protein construct; and (b) collecting the antibody or the antigen-binding fragment thereof, or the antigen-binding protein construct produced by the cell.

In one aspect, the disclosure is related to an antibody-drug conjugate comprising a therapeutic agent covalently bound to: (a) the antibody or antigen-binding fragment thereof as described herein; or (b) the antigen-binding protein construct as described herein. In some embodiments, the therapeutic agent is a cytotoxic or cytostatic agent.

In one aspect, the disclosure is related to a method of treating a subject having cancer, the method comprising administering a therapeutically effective amount of a composition comprising the antibody or antigen-binding fragment thereof as described herein, the antigen-binding protein construct as described herein, or the antibody-drug conjugate as described herein, to the subject. In some embodiments, the subject has a solid tumor. In some embodiments, the cancer is breast cancer, oropharyngeal cancer, ovarian cancer, bladder cancer, colon cancer, pancreas cancer, B cell lymphoma, hepatocellular carcinoma, or Non-Hodgkin's lymphoma. In some embodiments, the subject is a human. In some embodiments, the method further comprises administering an anti-PD1 antibody to the subject. In some embodiments, the method further comprises administering a chemotherapy to the subject.

In one aspect, the disclosure is related to a method of decreasing the rate of tumor growth, the method comprising contacting a tumor cell with an effective amount of a composition comprising the antibody or antigen-binding fragment thereof, the antigen-binding protein construct, or the antibody-drug conjugate as described herein.

In one aspect, the disclosure is related to a method of killing a tumor cell, the method comprising contacting a tumor cell with an effective amount of a composition comprising the antibody or antigen-binding fragment thereof, the antigen-binding protein construct, or the antibody-drug conjugate as described herein.

In one aspect, the disclosure is related to a pharmaceutical composition comprising a pharmaceutically acceptable carrier and (a) the antibody or antigen-binding fragment thereof as described herein, (b) the antigen-binding protein construct as described herein, or (c) the antibody-drug conjugate as described herein.

In one aspect, the disclosure provides a nucleic acid comprising a polynucleotide encoding any polypeptide as described herein. In some embodiments, the nucleic acid encodes a bispecific antibody. In some embodiments, the nucleic acid is cDNA.

In one aspect, the disclosure provides a vector comprising one or more of the nucleic acids described herein.

In one aspect, the disclosure provides a cell comprising the vector described herein. In some embodiments, the cell is a CHO cell. In one aspect, the disclosure provides a cell comprising one or more of the nucleic acids described herein.

As used herein, the term “antigen-binding protein construct” is (i) a single polypeptide that includes at least two different antigen-binding domains or (ii) a complex of two or more polypeptides (e.g., the same or different polypeptides) that together form at least two different antigen-binding domains. Non-limiting examples and aspects of antigen-binding protein constructs are described herein. Additional examples and aspects of antigen-binding protein constructs are known in the art.

As used herein, the term “antigen-binding domain” refers to one or more protein domain(s) (e.g., formed from amino acids from a single polypeptide or formed from amino acids from two or more polypeptides (e.g., the same or different polypeptides) that is capable of specifically binding to one or more different antigen(s) (e.g., an effector antigen or control antigen). In some examples, an antigen-binding domain can bind to an antigen or epitope with specificity and affinity similar to that of naturally-occurring antibodies. In some embodiments, the antigen-binding domain can be an antibody or a fragment thereof. One example of an antigen-binding domain is an antigen-binding domain formed by a VH-VL dimer. In some embodiments, an antigen-binding domain can include an alternative scaffold. In some embodiments, the antigen-binding domain is a VHH. Non-limiting examples of antigen-binding domains are described herein. Additional examples of antigen-binding domains are known in the art. In some examples, an antigen-binding domain can bind to a single antigen (e.g., one of an effector antigen and a control antigen). In other examples, an antigen-binding domain can bind to two different antigens (e.g., an effector antigen and a control antigen).

The term “antibody” is used herein in its broadest sense and includes certain types of immunoglobulin molecules that include one or more antigen-binding domains that specifically bind to an antigen or epitope. An antibody specifically includes, e.g., intact antibodies (e.g., intact immunoglobulins), antibody fragments, bispecific antibodies, and multi-specific antibodies. One example of an antibody is a protein complex that includes two heavy chains and two light chains. Additional examples of an antibody are described herein.

As used herein, the term “multispecific antigen-binding protein construct” is an antigen-binding protein construct that includes two or more different antigen-binding domains that collectively specifically bind two or more different epitopes. The two or more different epitopes may be epitopes on the same antigen (e.g., a single polypeptide present on the surface of a cell) or on different antigens (e.g., different proteins present on the surface of the same cell or present on the surface of different cells). In some aspects, a multi-specific antigen-binding protein construct binds two different epitopes (i.e., a “bispecific antigen-binding protein construct”). In some aspects, a multi-specific antigen-binding protein construct binds three different epitopes (i.e., a “trispecific antigen-binding protein construct”). In some aspects, a multi-specific antigen-binding protein construct binds four different epitopes (i.e., a “quadspecific antigen-binding protein construct”). In some aspects, a multi-specific antigen-binding protein construct binds five different epitopes (i.e., a “quintspecific antigen-binding protein construct”). Each binding specificity may be present in any suitable valency. Non-limiting examples of multispecific antigen-binding protein constructs are described herein.

As used herein, the term “bispecific antibody” refers to an antibody that binds to two different epitopes. The epitopes can be on the same antigen or on different antigens.

As used herein, a “VHH” refers to the variable domain of a heavy chain antibody. In some embodiments, the VHH is a humanized VHH.

As used herein, the term “common light chain” refers to a light chain that can interact with two or more different heavy chains, forming different antigen-binding sites, wherein these different antigen-binding sites can specifically bind to different antigens or epitopes. Similarly, the term “common light chain variable region” refers to a light chain variable region that can interact with two or more different heavy chain variable regions, forming different antigen-binding sites, wherein these different antigen-binding sites can specifically bind to different antigens or epitopes. In some embodiments, the antigen-binding construct can have a common light chain. In some embodiments, the antigen-binding construct can have a common light chain variable region.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 is a schematic diagram showing a bispecific antibody having the knobs-into-holes structure with common light chain.

FIG. 2 is a graph showing the average tumor volume in different groups of hCTLA4/hOX40 mice that were injected with colon adenocarcinoma cells MC38, and were treated with anti-CTLA4/OX40 bispecific antibody OX-2C11-CT-3D2-IgG1-SI treatment (G2), OX-2F2-CT-3D2-IgG1-SI (G3), CT-5F9-OX-2F2-IgG1-SI (G4), CT-5F9-OX-2G11-IgG1-SI (G5), or OX-2G11-CT-4E12-IgG1-SI (G6). PBS was injected as a control (G1).

FIG. 3 is a graph showing the average tumor volume in different groups of hCTLA4/hOX40 mice that were injected with colon adenocarcinoma cells MC38, and were treated with anti-CTLA4/OX40 bispecific antibody CT-5E3-OX-2F2-IgG1-SI (G2), CT-5E3-OX-2G11-IgG1-SI (G3), OX-2C11-CT-4E12-IgG1-SI (G4) or OX-2F2-CT-4E12-IgG1-SI (G5). PBS was injected as a control (G1).

FIG. 4 is a graph showing the average tumor volume in different groups of hCTLA4/hOX40 mice that were injected with colon adenocarcinoma cells MC38, and were treated with anti-CTLA4/OX40 bispecific antibody OX-2G11-CT-3D2-IgG1-SI (G2), CT-5F9-OX-2C11-IgG1-SI (G3) or CT-5E3-OX-2C11-IgG1-SI (G4). PBS was injected as a control (G1).

FIG. 5 is a graph showing the average tumor volume in different groups of hCTLA4/hOX40 mice that were injected with colon adenocarcinoma cells MC38, and were treated with an Ipilimumab analog (G2), anti-CTLA4/OX40 bispecific antibody OX-1F9-CT-3D2-IgG1-SI (G3), OX-2F5-CT-3D2-IgG1-SI (G4), OX-H7-CT-3D2-IgG1-SI (G5), OX-H8-CT-3D2-IgG1-SI (G6), or OX-4C8-CT-3D2-IgG1-SI (G7). PBS was injected as a control (G1).

FIG. 6 is a graph showing the average tumor volume in different groups of hCTLA4/hOX40 mice that were injected with colon adenocarcinoma cells MC38, and were treated with anti-CTLA4/OX40 bispecific antibody OX-H7-CT-3D2-IgG1-SI at a dose level of 0.3 mg/kg (G2), 1 mg/kg (G3), 3 mg/kg (G4), or 10 mg/kg (G5). PBS was injected as a control (G1).

FIG. 7 is a graph showing the average tumor volume in different groups of hCTLA4/hOX40 mice that were injected with colon adenocarcinoma cells MC38, and were treated with anti-CTLA4/OX40 bispecific antibody OX-H7-CT-3D2-IgG1-SI (G2), OX-4C8-CT-3D2-IgG1-SI (G3), anti-CTLA4 antibody CT-3D2-IgG1-SI (G4), anti-OX40 antibody OX-4C8-IgG1-SI (G5), OX-H7-IgG1-SI (G6), or an Ipilimumab analog (G7). PBS was injected as a control (G1).

FIG. 8A shows the binding curves of anti-CTLA4/OX40 bispecific antibody OX-H7-CT-3D2-IgG1-SI, anti-CTLA4 antibody CT-3D2-IgG1, an Ipilimumab analog, and anti-OX40 antibodies OX-H7-IgG1 to HEK293T-hCTLA4-hOX40 cells.

FIG. 8B shows the binding curves of anti-CTLA4/OX40 bispecific antibody OX-H8-CT-3D2-IgG1-SI, anti-CTLA4 antibody CT-3D2-IgG1, an Ipilimumab analog, and anti-OX40 antibodies OX-H8-IgG1 to HEK293T-hCTLA4-hOX40 cells.

FIG. 8C shows the binding curves of anti-CTLA4/OX40 bispecific antibody OX-4C8-CT-3D2-IgG1-SI, anti-CTLA4 antibody CT-3D2-IgG1, an Ipilimumab analog, and anti-OX40 antibodies OX-4C8-IgG1 to HEK293T-hCTLA4-hOX40 cells.

FIG. 9 shows the fluorescence signal of effector cells Jurkat-luc-hCTLA4 that were co-cultured with target cells Raji-aAPC-CD80/86 and serially diluted antibodies.

FIG. 10 shows the fluorescence signal of effector cells Jurkat-luc-hOX40 that were co-cultured with target cells Jurkat-hCTLA4 and serially diluted antibodies, or effector cells Jurkat-luc-hOX40 alone with serially diluted antibodies.

FIG. 11 shows the fluorescence signal of effector cells Jurkat-luc-hCD16A that were co-cultured with Jurkat-hCTLA4-hOX40 cells and serially diluted antibodies.

FIG. 12 shows the CD3+CD8+ T cell-binding avidity in human PBMCs of serially diluted antibodies.

FIG. 13 shows the CD3+CD4+ T cell-binding avidity in human PBMCs of serially diluted antibodies.

FIG. 14 a graph showing the body weight in different groups of hCTLA4/hOX40 mice that were treated with anti-CTLA4/OX40 bispecific antibody OX-H7-CT-3D2-IgG1-SI (G2) or OX-4C8-CT-3D2-IgG1-SI (G3). PBS was injected as a control (G1).

FIG. 15 show the ALT level 4 days after grouping.

FIG. 16 show the AST level 4 days after grouping.

FIG. 17 show the ALT level 7 days after grouping.

FIG. 18 show the AST level 7 days after grouping.

FIG. 19 shows the cell sorting strategy for TILs analysis.

FIGS. 20A-20B show the ratio of CTL cells in tumor and normal (spleen) tissues, respectively.

FIGS. 20C-20D show the ratio of Treg cells in tumor and normal (spleen) tissues, respectively.

FIGS. 20E-20F show the ratio of CTL cells to Treg cells in tumor and normal (spleen) tissues, respectively.

FIGS. 21A-21I show the expression level of activation markers expressed on T cells in tumor tissues. FIGS. 21A-21C show the expression level of activation marker mICOS on CTL cells, Th cells and Treg cells, respectively. FIGS. 21D-21E show the expression level of activation marker mKi67 on CTL cell and Tregs cell, respectively. FIGS. 21F-21G show the expression level of activation marker mTNF-α on CTL cells and Th cells, respectively. FIGS. 21H-21I show the expression level of activation marker mIFN-γ on CTL cells and Th cells, respectively.

FIG. 22 lists heavy chain CDR sequences of anti-CTLA4 antibodies CT-3D2, CT-4E12, CT-5E3, and CT-5F9 as defined by Kabat numbering.

FIG. 23 lists heavy chain CDR sequences of anti-CTLA4 antibodies CT-3D2, CT-4E12, CT-5E3, and CT-5F9 as defined by Chothia numbering.

FIG. 24 lists heavy chain CDR sequences of anti-OX40 antibodies OX-1F9, OX-2C11, OX-2F2, OX-2F5, OX-2G11, OX-4C8, OX-H7, and OX-H8 as defined by Kabat numbering.

FIG. 25 lists heavy chain CDR sequences of anti-OX40 antibodies OX-1F9, OX-2C11, OX-2F2, OX-2F5, OX-2G11, OX-4C8, OX-H7, and OX-H8 as defined by Chothia numbering.

FIG. 26 lists light chain CDR sequences for the common light chain, monoclonal anti-OX40 antibodies OX-H7 and OX-H8 as defined by Kabat numbering.

FIG. 27 lists light chain CDR sequences for the common light chain, monoclonal anti-OX40 antibodies OX-H7 and OX-H8 as defined by Chothia numbering.

FIG. 28 lists antibody heavy chain and light chain variable region sequences discussed in the disclosure.

FIG. 29 lists the knob heavy chain, hole heavy chain, and common light chain sequences of anti-CTLA4/OX40 bispecific antibodies discussed in the disclosure.

FIG. 30 lists additional amino acid sequences discussed in the disclosure.

FIG. 31 shows the fluorescence signal of effector cells FcR-TANK that were co-cultured with human Treg cells and serially diluted antibodies.

FIG. 32 shows the human IL2 secretion in human PBMCs of serially diluted antibodies.

FIGS. 33A-33C show the ratio of CTL cells, Treg cells and CTL/Treg in tumor, respectively.

FIG. 34 is a graph showing the average tumor volume in different groups of hCTLA4/hOX40 mice that were injected with colon adenocarcinoma cells MC38, and were treated with anti-CTLA4/OX40 bispecific antibody OX-H7-CT-3D2-IgG1-SI (G2 and G3), combination of CT-3D2-IgG1-SI and OX-H7-IgG1-SI (G4), mPD1-Ab (G5) or combination of OX-H7-CT-3D2-IgG1-SI and mPD1-Ab (G6). PBS was injected as a control (G1).

FIG. 35 is a graph showing the average tumor volume in different groups of hCTLA4/hOX40 mice that were injected with B16-F10-OVA melanoma cells, and were treated with OX-H7-CT-3D2-IgG1-SI (G2), Atezolizumab analog (G3), or combination of OX-H7-CT-3D2-IgG1-SI and Atezolizumab analog (G4). PBS was injected as a control (G1).

FIG. 36 is a graph showing the average tumor volume in different groups of hPD1/hCD40/hCTLA4/hOX40 mice that were injected with colon adenocarcinoma cells MC38, and were treated with PD1/CD40-Ab (G2 and G3), OX-H7-CT-3D2-IgG1-SI (G4), or combination of PD1/CD40-Ab and OX-H7-CT-3D2-IgG1-SI (G5). PBS was injected as a control (G1).

### DETAILED DESCRIPTION

A bispecific antibody or antigen-binding fragment thereof is an artificial protein that can simultaneously bind to two different epitopes (e.g., on two different antigens). In some embodiments, a bispecific antibody or antigen-binding fragment thereof can have two arms (Arms A and B). Each arm has one heavy chain variable region and one light chain variable region, forming an antigen-binding domain (or an antigen-binding region). In some embodiments, the bispecific antibody has a common light chain.

The present disclosure relates to anti-CTLA4 antibodies or antigen binding fragments thereof, anti-OX40 antibodies or antigen binding fragments thereof, antigen-binding protein constructs (e.g., bispecific antibodies or antigen-binding fragments thereof) that specifically bind to two different antigens (e.g., CTLA4 and OX40), and antibody drug conjugates.

### Anti-CTLA4/OX40 Antigen-Binding Protein Construct

The immune system can differentiate between normal cells in the body and those it sees as “foreign,” which allows the immune system to attack the foreign cells while leaving the normal cells alone. This mechanism sometimes involves proteins called immune checkpoints. Immune checkpoints are molecules in the immune system that either turn up a signal (co-stimulatory molecules) or turn down a signal.

The immune checkpoint pathway involves a series of cellular interactions that prevents excessive effector activity by T cells under normal conditions. Cytotoxic T-lymphocyte antigen-4 (CTLA4 or CD152) is involved in this pathway. CTLA4 is a member of the immunoglobulin superfamily that is expressed exclusively on T-cells. CTLA4 acts to inhibit T-cell activation and is reported to inhibit helper T-cell activity and enhance regulatory T-cell immunosuppressive activity. Thus, CTLA-4 acts as an “off” switch, and turns down the immune response. Once a cytotoxic T cell becomes active, it expresses CTLA-4 on its cell surface, which then competes with the costimulatory molecule CD28 for their mutually shared ligands, B7-1 (CD80) or B7-2 (CD86) on the APC. This “yin-yang” balance holds cytotoxic activity in check, while allowing T-cell function to proceed in a self-limited manner (Creelan, Benjamin C. “Update on immune checkpoint inhibitors in lung cancer.” Cancer Control 21.1 (2014): 80-89).

In 2011, the first anti-CTLA4 antibody inhibitor Ipilimumab (trade name YERVOY®) was approved for treatment of melanoma. However, YERVOY® presented strong immunotherapy-related side effects (Larkin, James, et al. “Combined nivolumab and ipilimumab or monotherapy in untreated melanoma.” New England Journal of Medicine 373.1 (2015): 23-34), such as anemia, myocarditis, pneumonia, hepatitis, kidney and other multi-organ inflammations, which limited the clinical use of anti-CTLA4 antibodies.

The anti-tumor effects and the side effects of anti-CTLA4 antibody are not necessarily concomitant. The side effects may arise from the expansion of autoreactive T cells and the activation of systemic T cells (or blocking effect). In recent years, some studies have found that anti-CTLA4 antibody Ipilimumab may not exert anti-tumor effect by blocking CTLA4/B7 interaction, but by antibody Fc domain-mediated effector function to eliminate Regulatory T cells (Tregs) (Selby, Mark J., et al. “Anti-CTLA-4 antibodies of IgG2a isotype enhance antitumor activity through reduction of intratumoral regulatory T cells.” Cancer Immunology Research 1.1 (2013): 32-42).

Thus, it is hypothesized that the therapeutic effects can be increased if the blocking effects of the anti-CTLA4 antibody is attenuated and the targeting and killing activity of Treg in the tumor microenvironment is enhanced.

An antigen-binding protein construct that target OX40 and CTLA4 can be developed for this purpose. Tumor necrosis factor receptor superfamily, member 4 (TNFRSF4), also known as CD134 and OX40, is a member of the TNFR-superfamily of receptors which is not constitutively expressed on resting naïve T cells. OX40 is a secondary co-stimulatory immune checkpoint molecule, expressed after 24 to 72 hours following activation; its ligand, OX40L, is also not expressed on resting antigen presenting cells, but is following their activation. The expression of OX40 on the surface of mouse T-cells typically occurs between 24 hours and 96 hours after cognate antigen recognition. Engagement of the OX40 receptor on T-cells (in vitro), using anti-OX40 agonistic antibodies, directly promotes an increase in survival of different effector T-cell subsets. Moreover, the immunosuppressive subset of CD4+ T-cells called regulatory T-cells (Tregs) also express high levels of OX40. Tregs can inhibit effector T-cells through the secretion of immunosuppressive cytokines such as transforming growth factor-beta (TGFb) and interleukin-10 (IL-10). These negative regulators can be counter balanced by the stimulation of OX40 on effector T-cells and other TNFRSF co-stimulatory receptors such as 41BB (CD137) and glucocorticoid-induced tumor necrosis factor receptor (GITR) (CD357).

Both OX40 and CTLA4 are highly expressed specifically in Tregs from tumor infiltrating lymphocytes (TIL) (Vargas, Frederick Arce, et al. “Fc effector function contributes to the activity of human anti-CTLA-4 antibodies.” Cancer Cell 33.4 (2018): 649-663). The elimination effect of Tregs is also regarded as a mechanism for anti-OX40 antibody in cancer immunotherapy. Accordingly, an antigen-binding protein construct (e.g., bispecific antibody) that target OX40 and CTLA4 can effectively eliminate Tregs, increase the anti-tumor effects, and reduce potential side effects.

The anti-CTLA4 antibodies (e.g., CT-3D2 (“3D2”), CT-4E12 (“4E12”), CT-5E3 (“5E3”), CT-5F9 (“5F9”)) and anti-OX40 antibodies (e.g., OX-1F9 (“1F9”), OX-2C11 (“2C11”), OX-2F2 (“2F2”), OX-2F5 (“2F5”), OX-2G11 (“2G11”), OX-4C8 (“4C8”), OX-H7 (“H7”), and OX-H8 (“H8”)) in the present disclosure are human antibodies produced in RenLite® mice. Because these antibodies have an identical fully-humanized common light chain, anti-CTLA4/OX40 bispecific antibodies were generated having a heavy chain variable region targeting CTLA4 (e.g., any one of the VH targeting CTLA4 described herein), a heavy chain variable region targeting OX40 (e.g., any one of the VH targeting OX40 described herein), and two identical common light chain variable regions.

The bispecific antibody described herein can be designed to have an IgG1 subtype structure with knobs-into-holes (KIH) mutations and SI mutations. The KIH mutations can promote heterodimerization, and the SI mutations can enhance the antibody affinity to FcγRIIIA. The bispecific antibodies showed good avidity to human T cells and can effectively increase immune response by blocking the CTLA4 signaling pathway. The bispecific antibodies can also activate the OX40 signaling pathway, but only in the presence of CTLA4. This property can reduce the chance of non-specific activation of the OX40 pathway, and may explain the low toxicity of the bispecific antibodies.

In particular, the bispecific antibodies also showed stronger ADCC effects and more effective Treg cell depletion than the corresponding monoclonal antibodies.

In some embodiments, the bispecific antibody or antigen-binding fragment thereof described herein has a common light chain.

### Anti-OX40 Antibodies and Antigen-Binding Fragments

The disclosure provides antibodies and antigen-binding fragments thereof that specifically bind to OX40. The anti-CTLA4/OX40 antigen-binding protein construct (e.g., bispecific antibodies) can include an antigen binding region that is derived from these antibodies.

The antibodies and antigen-binding fragments described herein are capable of binding to OX40. The disclosure provides anti-OX40 antibodies OX-1F9 (“1F9”), OX-2C11 (“2C11”), OX-2F2 (“2F2”), OX-2F5 (“2F5”), OX-2G11 (“2G11”), OX-4C8 (“4C8”), OX-H7 (“H7”), and OX-H8 (“H8”), and the antibodies derived therefrom.

The CDR sequences for 1F9, and 1F9 derived antibodies (e.g., human antibodies) include CDRs of the heavy chain variable domain, SEQ ID NOs: 36-38, and CDRs of the light chain variable domain, SEQ ID NOs: 29-31 as defined by Kabat numbering. The CDRs can also be defined by Chothia system. Under the Chothia numbering, the CDR sequences of the heavy chain variable domain are set forth in SEQ ID NOs: 60-62, and CDR sequences of the light chain variable domain are set forth in SEQ ID NOs: 32-34.

The CDR sequences for 2C11, and 2C11 derived antibodies include CDRs of the heavy chain variable domain, SEQ ID NOs: 39-41, and CDRs of the light chain variable domain, SEQ ID NOs: 29-31, as defined by Kabat numbering. Under Chothia numbering, the CDR sequences of the heavy chain variable domain are set forth in SEQ ID NOs: 63-65, and CDRs of the light chain variable domain are set forth in SEQ ID NOs: 32-34.

The CDR sequences for 2F2, and 2F2 derived antibodies include CDRs of the heavy chain variable domain, SEQ ID NOs: 42-44, and CDRs of the light chain variable domain, SEQ ID NOs: 29-31, as defined by Kabat numbering. Under Chothia numbering, the CDR sequences of the heavy chain variable domain are set forth in SEQ ID NOs: 66-68, and CDRs of the light chain variable domain are set forth in SEQ ID NOs: 32-34.

The CDR sequences for 2F5, and 2F5 derived antibodies include CDRs of the heavy chain variable domain, SEQ ID NOs: 45-47, and CDRs of the light chain variable domain, SEQ ID NOs: 29-31, as defined by Kabat numbering. Under Chothia numbering, the CDR sequences of the heavy chain variable domain are set forth in SEQ ID NOs: 69-71, and CDRs of the light chain variable domain are set forth in SEQ ID NOs: 32-34.

The CDR sequences for 2G11, and 2G11 derived antibodies include CDRs of the heavy chain variable domain, SEQ ID NOs: 48-50, and CDRs of the light chain variable domain, SEQ ID NOs: 29-31, as defined by Kabat numbering. Under Chothia numbering, the CDR sequences of the heavy chain variable domain are set forth in SEQ ID NOs: 72-74, and CDRs of the light chain variable domain are set forth in SEQ ID NOs: 32-34.

The CDR sequences for 4C8, and 4C8 derived antibodies include CDRs of the heavy chain variable domain, SEQ ID NOs: 51-53, and CDRs of the light chain variable domain, SEQ ID NOs: 29-31, as defined by Kabat numbering. Under Chothia numbering, the CDR sequences of the heavy chain variable domain are set forth in SEQ ID NOs: 75-77, and CDRs of the light chain variable domain are set forth in SEQ ID NOs: 32-34.

The CDR sequences for H7, and H7 derived antibodies include CDRs of the heavy chain variable domain, SEQ ID NOs: 54-56, and CDRs of the light chain variable domain, SEQ ID NOs: 29-31, or SEQ ID NOs: 149-151, as defined by Kabat numbering. Under Chothia numbering, the CDR sequences of the heavy chain variable domain are set forth in SEQ ID NOs: 78-80, and CDRs of the light chain variable domain are set forth in SEQ ID NOs: 32-34, or SEQ ID NOs: 155-157.

The CDR sequences for H8, and H8 derived antibodies include CDRs of the heavy chain variable domain, SEQ ID NOs: 57-59, and CDRs of the light chain variable domain, SEQ ID NOs: 29-31, or SEQ ID NOs: 152-154, as defined by Kabat numbering. Under Chothia numbering, the CDR sequences of the heavy chain variable domain are set forth in SEQ ID NOs: 81-83, and CDRs of the light chain variable domain are set forth in SEQ ID NOs: 32-34, or SEQ ID NOs: 158-160.

Furthermore, in some embodiments, the antibodies or antigen-binding fragments thereof described herein can also contain one, two, or three heavy chain variable region CDRs selected from the group of SEQ ID NOs: 36-38, SEQ ID NOs: 39-41, SEQ ID NOs: 42-44, SEQ ID NOs: 45-47, SEQ ID NOs: 48-50, SEQ ID NOs: 51-53, SEQ ID NOs: 54-56, SEQ ID NOs: 57-59, SEQ ID NOs: 60-62, SEQ ID NOs: 63-65, SEQ ID NOs: 66-68, SEQ ID NOs: 69-71, SEQ ID NOs: 72-74, SEQ ID NOs: 75-77, SEQ ID NOs: 78-80, and SEQ ID NOs: 81-83; and/or one, two, or three light chain variable region CDRs selected from the group of SEQ ID NOs: 29-31, SEQ ID NOs: 32-34, SEQ ID NOs: 149-151, SEQ ID NOs: 152-154, SEQ ID NOs: 155-157, and SEQ ID NOs: 158-160.

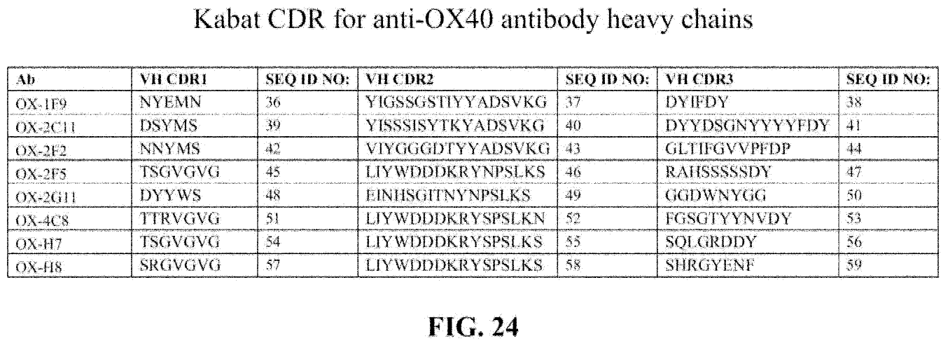

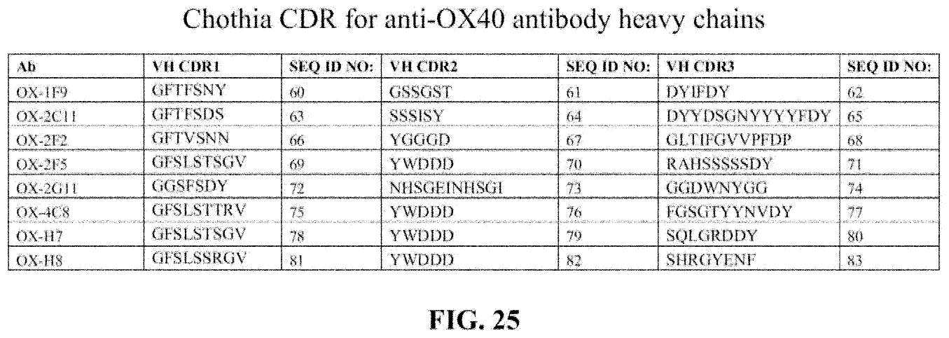

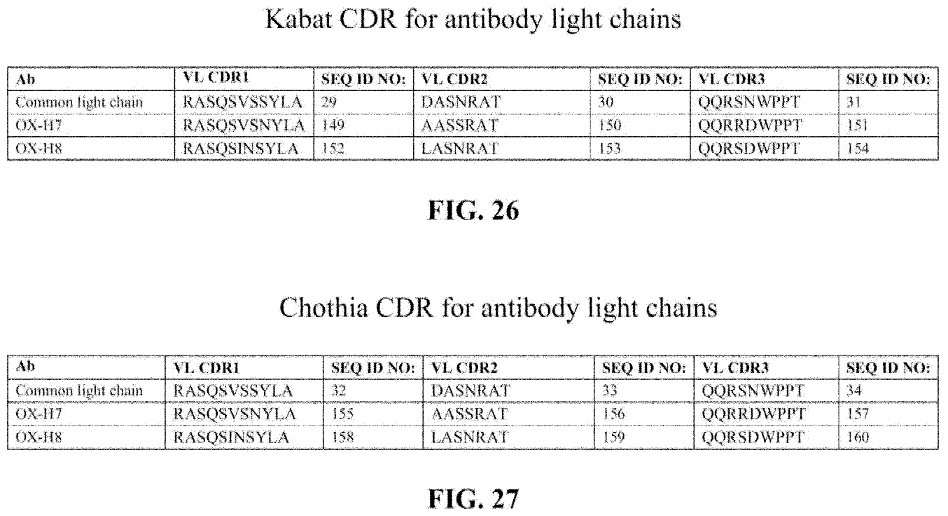

In some embodiments, the antibodies can have a heavy chain variable region (VH) comprising complementarity determining regions (CDRs) 1, 2, 3, wherein the CDR1 region comprises or consists of an amino acid sequence that is at least 80%, 85%, 90%, or 95% identical to a selected VH CDR1 amino acid sequence, the CDR2 region comprises or consists of an amino acid sequence that is at least 80%, 85%, 90%, or 95% identical to a selected VH CDR2 amino acid sequence, and the CDR3 region comprises or consists of an amino acid sequence that is at least 80%, 85%, 90%, or 95% identical to a selected VH CDR3 amino acid sequence, and a light chain variable region (VL) comprising CDRs 1, 2, 3, wherein the CDR1 region comprises or consists of an amino acid sequence that is at least 80%, 85%, 90%, or 95% identical to a selected VL CDR1 amino acid sequence, the CDR2 region comprises or consists of an amino acid sequence that is at least 80%, 85%, 90%, or 95% identical to a selected VL CDR2 amino acid sequence, and the CDR3 region comprises or consists of an amino acid sequence that is at least 80%, 85%, 90%, or 95% identical to a selected VL CDR3 amino acid sequence. The selected VH CDRs 1, 2, 3 amino acid sequences and the selected VL CDRs, 1, 2, 3 amino acid sequences are shown in FIGS. 24 and 26 (Kabat CDR) and FIGS. 25 and 27 (Chothia CDR).

In some embodiments, the antibody or an antigen-binding fragment described herein can contain a heavy chain variable domain containing one, two, or three of the CDRs of SEQ ID NO: 36 with zero, one or two amino acid insertions, deletions, or substitutions; SEQ ID NO: 37 with zero, one or two amino acid insertions, deletions, or substitutions; SEQ ID NO: 38 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 39 with zero, one or two amino acid insertions, deletions, or substitutions; SEQ ID NO: 40 with zero, one or two amino acid insertions, deletions, or substitutions; SEQ ID NO: 41 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 42 with zero, one or two amino acid insertions, deletions, or substitutions; SEQ ID NO: 43 with zero, one or two amino acid insertions, deletions, or substitutions; SEQ ID NO: 44 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 45 with zero, one or two amino acid insertions, deletions, or substitutions; SEQ ID NO: 46 with zero, one or two amino acid insertions, deletions, or substitutions; SEQ ID NO: 47 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 48 with zero, one or two amino acid insertions, deletions, or substitutions; SEQ ID NO: 49 with zero, one or two amino acid insertions, deletions, or substitutions; SEQ ID NO: 50 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 51 with zero, one or two amino acid insertions, deletions, or substitutions; SEQ ID NO: 52 with zero, one or two amino acid insertions, deletions, or substitutions; SEQ ID NO: 53 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 54 with zero, one or two amino acid insertions, deletions, or substitutions; SEQ ID NO: 55 with zero, one or two amino acid insertions, deletions, or substitutions; SEQ ID NO: 56 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 57 with zero, one or two amino acid insertions, deletions, or substitutions; SEQ ID NO: 58 with zero, one or two amino acid insertions, deletions, or substitutions; SEQ ID NO: 59 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 60 with zero, one or two amino acid insertions, deletions, or substitutions; SEQ ID NO: 61 with zero, one or two amino acid insertions, deletions, or substitutions; SEQ ID NO: 62 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 63 with zero, one or two amino acid insertions, deletions, or substitutions; SEQ ID NO: 64 with zero, one or two amino acid insertions, deletions, or substitutions; SEQ ID NO: 65 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 66 with zero, one or two amino acid insertions, deletions, or substitutions; SEQ ID NO:67 with zero, one or two amino acid insertions, deletions, or substitutions; SEQ ID NO: 68 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 69 with zero, one or two amino acid insertions, deletions, or substitutions; SEQ ID NO: 70 with zero, one or two amino acid insertions, deletions, or substitutions; SEQ ID NO: 71 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 72 with zero, one or two amino acid insertions, deletions, or substitutions; SEQ ID NO: 73 with zero, one or two amino acid insertions, deletions, or substitutions; SEQ ID NO: 74 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 75 with zero, one or two amino acid insertions, deletions, or substitutions; SEQ ID NO: 76 with zero, one or two amino acid insertions, deletions, or substitutions; SEQ ID NO: 77 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 78 with zero, one or two amino acid insertions, deletions, or substitutions; SEQ ID NO: 79 with zero, one or two amino acid insertions, deletions, or substitutions; SEQ ID NO: 80 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 81 with zero, one or two amino acid insertions, deletions, or substitutions; SEQ ID NO: 82 with zero, one or two amino acid insertions, deletions, or substitutions; SEQ ID NO: 83 with zero, one or two amino acid insertions, deletions, or substitutions.

In some embodiments, the antibody or an antigen-binding fragment described herein can contain a light chain variable domain containing one, two, or three of the CDRs of SEQ ID NO: 29 with zero, one or two amino acid insertions, deletions, or substitutions; SEQ ID NO: 30 with zero, one or two amino acid insertions, deletions, or substitutions; SEQ ID NO: 31 with zero, one or two amino acid insertions, deletions, or substitutions.

In some embodiments, the antibody or an antigen-binding fragment described herein can contain a light chain variable domain containing one, two, or three of the CDRs of SEQ ID NO: 32 with zero, one or two amino acid insertions, deletions, or substitutions; SEQ ID NO: 33 with zero, one or two amino acid insertions, deletions, or substitutions; SEQ ID NO: 34 with zero, one or two amino acid insertions, deletions, or substitutions.

In some embodiments, the antibody or an antigen-binding fragment described herein can contain a light chain variable domain containing one, two, or three of the CDRs of SEQ ID NO: 149 with zero, one or two amino acid insertions, deletions, or substitutions; SEQ ID NO: 150 with zero, one or two amino acid insertions, deletions, or substitutions; SEQ ID NO: 151 with zero, one or two amino acid insertions, deletions, or substitutions.

In some embodiments, the antibody or an antigen-binding fragment described herein can contain a light chain variable domain containing one, two, or three of the CDRs of SEQ ID NO: 152 with zero, one or two amino acid insertions, deletions, or substitutions; SEQ ID NO: 153 with zero, one or two amino acid insertions, deletions, or substitutions; SEQ ID NO: 154 with zero, one or two amino acid insertions, deletions, or substitutions.

In some embodiments, the antibody or an antigen-binding fragment described herein can contain a light chain variable domain containing one, two, or three of the CDRs of SEQ ID NO: 155 with zero, one or two amino acid insertions, deletions, or substitutions; SEQ ID NO: 156 with zero, one or two amino acid insertions, deletions, or substitutions; SEQ ID NO: 157 with zero, one or two amino acid insertions, deletions, or substitutions.

In some embodiments, the antibody or an antigen-binding fragment described herein can contain a light chain variable domain containing one, two, or three of the CDRs of SEQ ID NO: 158 with zero, one or two amino acid insertions, deletions, or substitutions; SEQ ID NO: 159 with zero, one or two amino acid insertions, deletions, or substitutions; SEQ ID NO: 160 with zero, one or two amino acid insertions, deletions, or substitutions.

The insertions, deletions, and substitutions can be within the CDR sequence, or at one or both terminal ends of the CDR sequence.

The disclosure also provides antibodies or antigen-binding fragments thereof that binds to OX40. The antibodies or antigen-binding fragments thereof contain a heavy chain variable region (VH) comprising or consisting of an amino acid sequence that is at least 80%, 85%, 90%, or 95% identical to a selected VH sequence, and a light chain variable region (VL) comprising or consisting of an amino acid sequence that is at least 80%, 85%, 90%, or 95% identical to a selected VL sequence. In some embodiments, the selected VH sequence is SEQ ID NO: 84, 85, 86, 87, 88, 89, 90, or 91 and the selected VL sequence is SEQ ID NO: 35, 161, or 162.

In some embodiments, the antibody or antigen binding fragments thereof can have 3 VH CDRs that are identical to the CDRs of any VH sequences as described herein. In some embodiments, the antibody or antigen binding fragments thereof can have 3 VL CDRs that are identical to the CDRs of any VL sequences as described herein.

The disclosure also provides nucleic acid comprising a polynucleotide encoding a polypeptide comprising an immunoglobulin heavy chain or an immunoglobulin heavy chain.

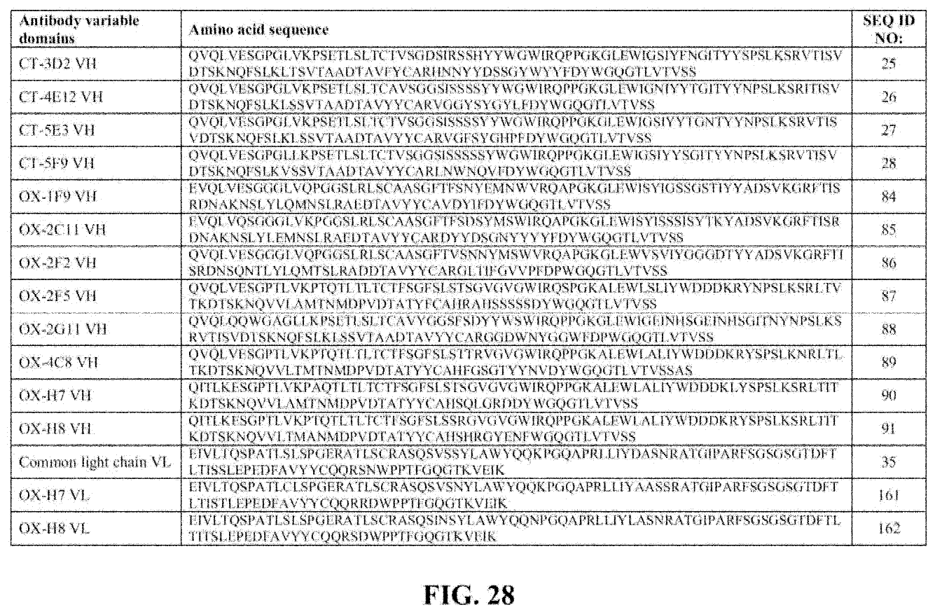

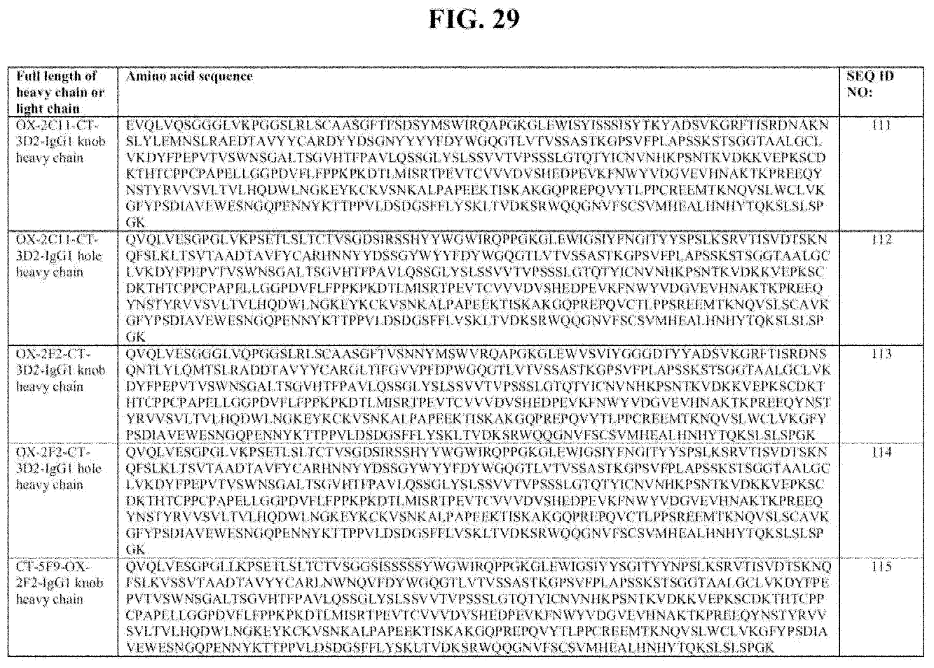

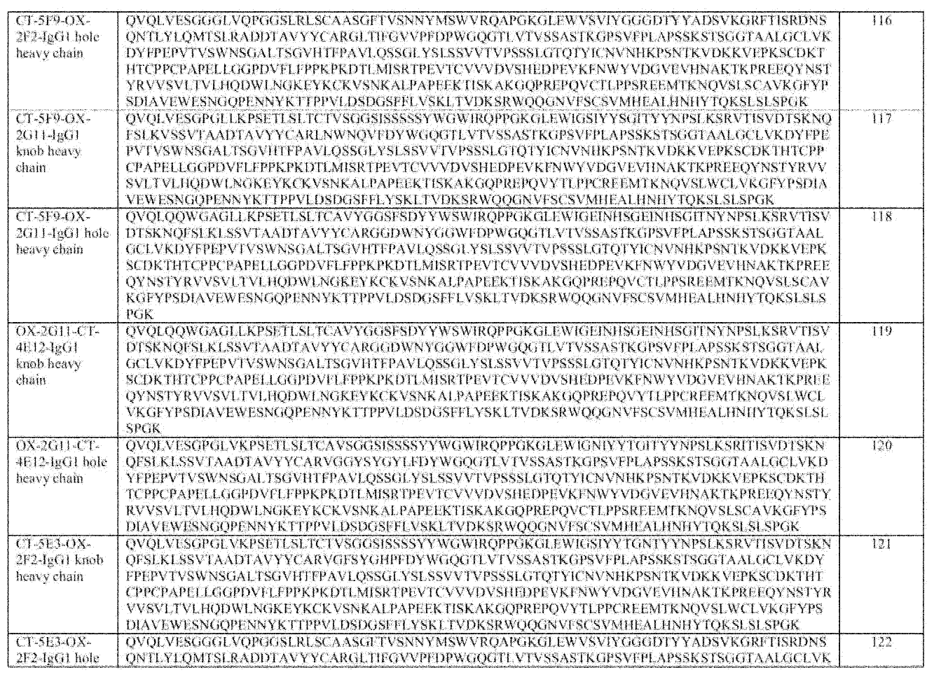

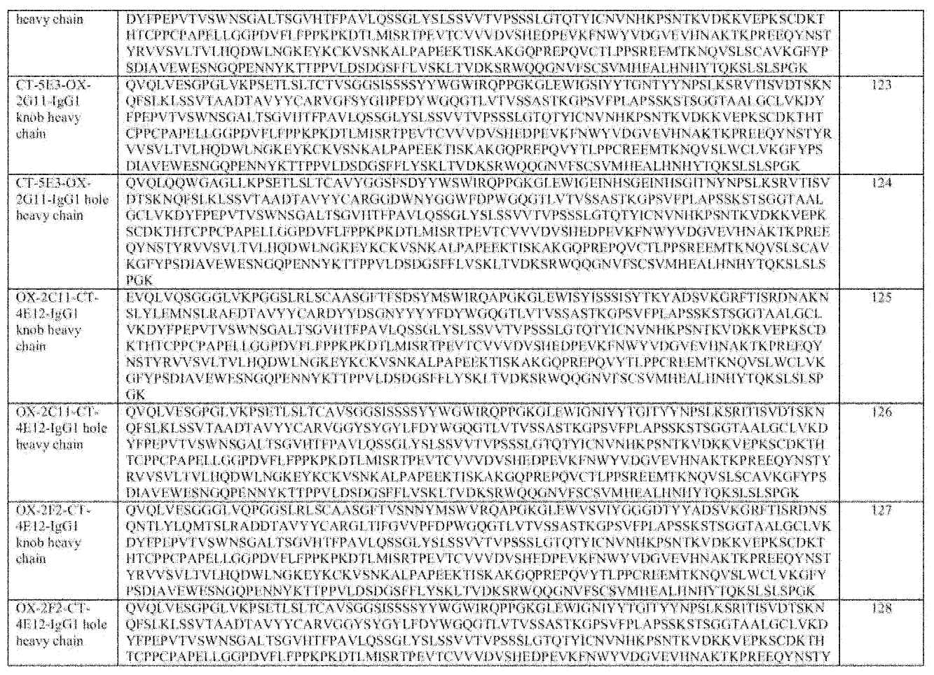

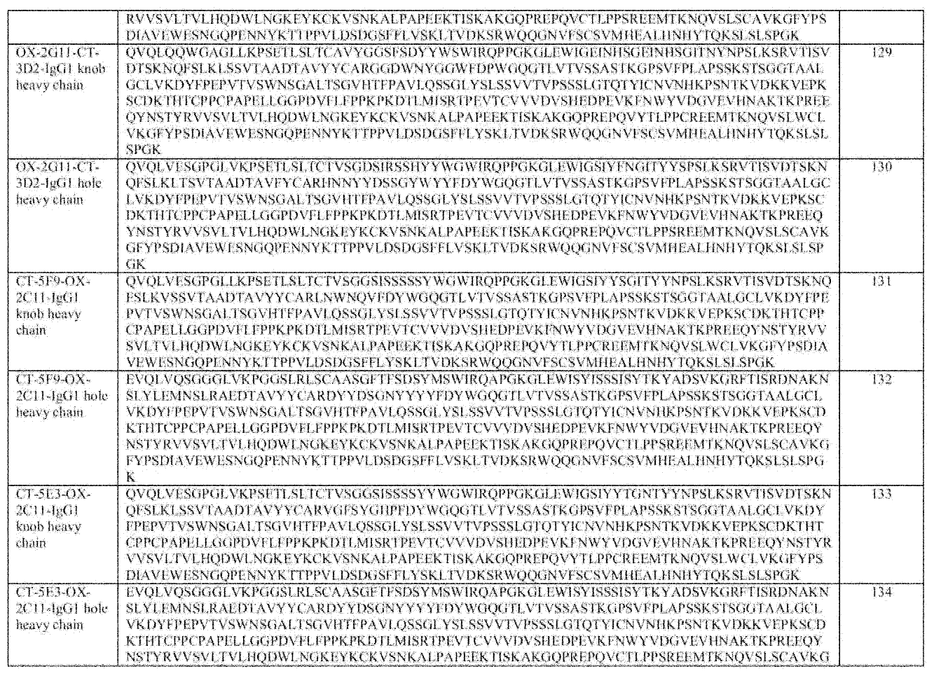

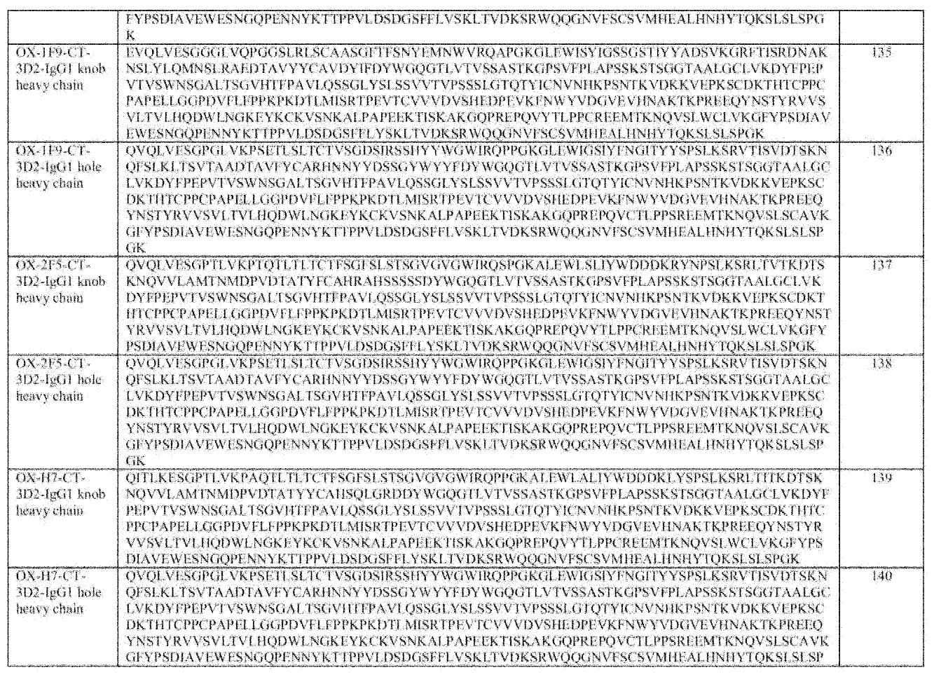

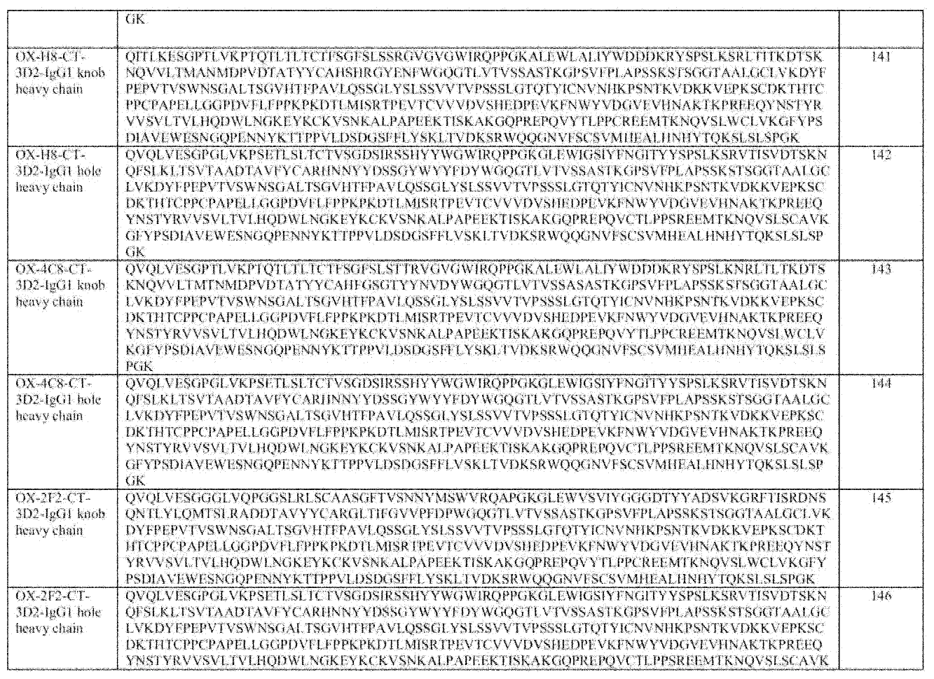

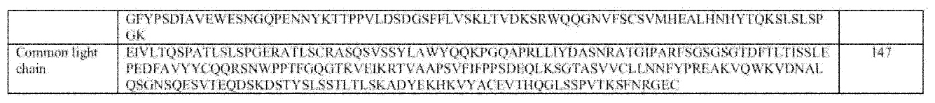

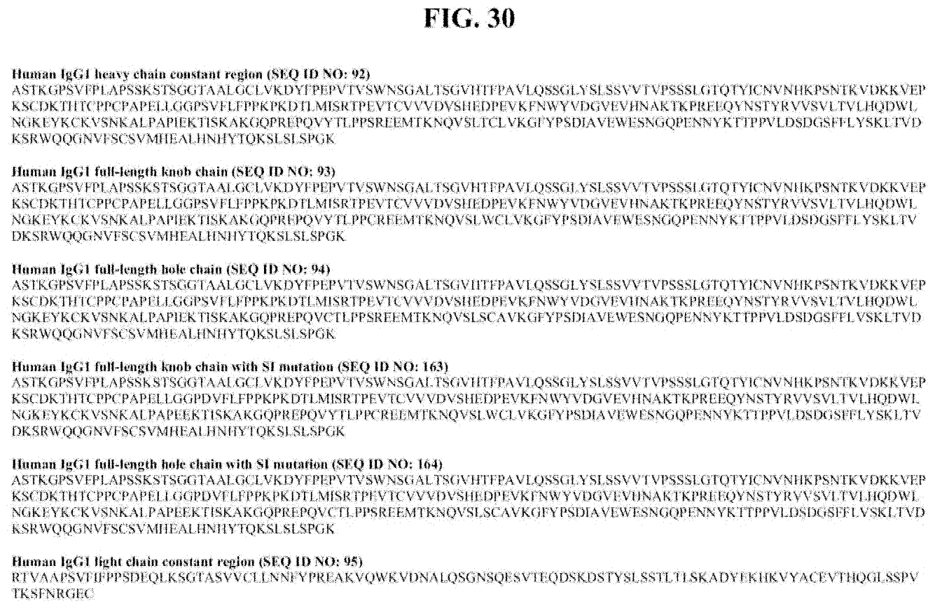

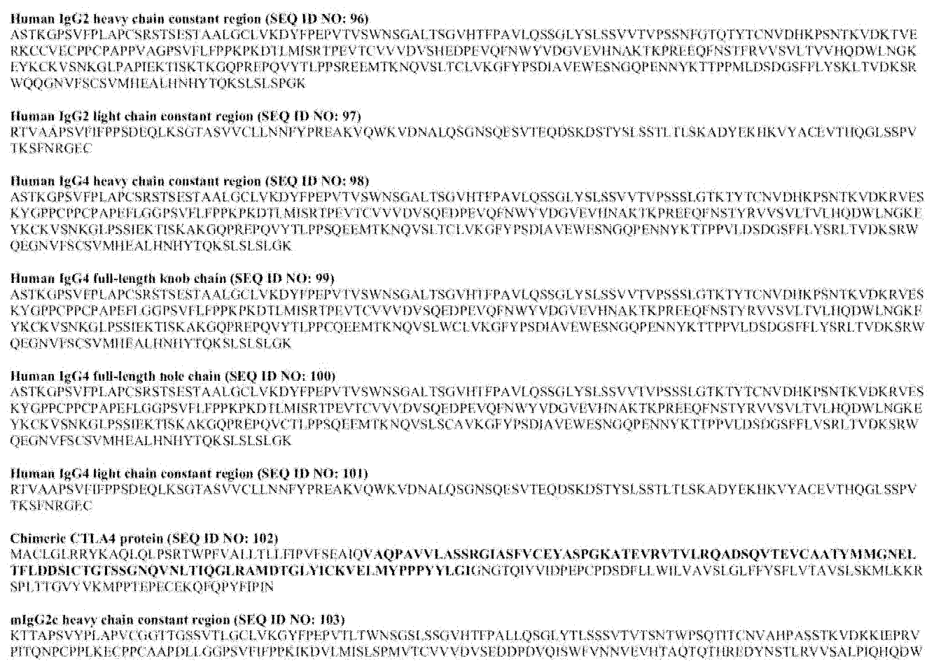

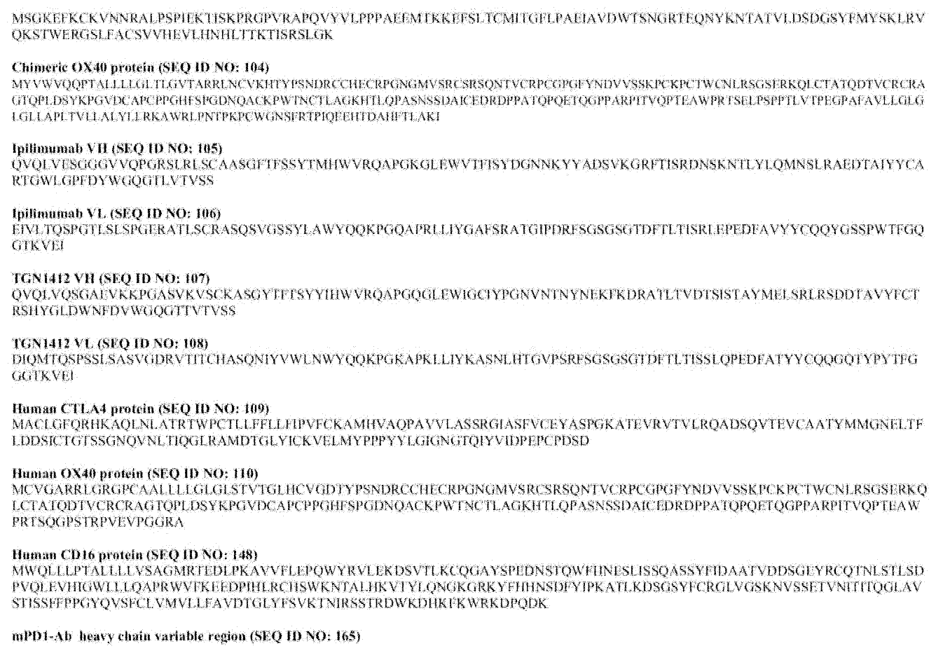

The immunoglobulin heavy chain or immunoglobulin light chain comprises CDRs as shown in FIG. 24, FIG. 25, FIG. 26, or FIG. 27, or have sequences as shown in FIG. 28. When the polypeptides are paired with corresponding polypeptide (e.g., a corresponding heavy chain variable region or a corresponding light chain variable region), the paired polypeptides bind to OX40.

The anti-OX40 antibodies and antigen-binding fragments can also be antibody variants (including derivatives and conjugates) of antibodies or antibody fragments and multi-specific (e.g., bispecific) antibodies or antibody fragments. Additional antibodies provided herein are polyclonal, monoclonal, multi-specific (multimeric, e.g., bispecific), human antibodies, chimeric antibodies (e.g., human-mouse chimera), single-chain antibodies, intracellularly-made antibodies (i.e., intrabodies), and antigen-binding fragments thereof. The antibodies or antigen-binding fragments thereof can be of any type (e.g., IgG, IgE, IgM, IgD, IgA, and IgY), class (e.g., IgG1, IgG2, IgG3, IgG4, IgA1, and IgA2), or subclass. In some embodiments, the antibody or antigen-binding fragment thereof is an IgG antibody or antigen-binding fragment thereof.

Fragments of antibodies are suitable for use in the methods provided so long as they retain the desired affinity and specificity of the full-length antibody. Thus, a fragment of an antibody that binds to OX40 will retain an ability to bind to OX40. An Fv fragment is an antibody fragment which contains a complete antigen recognition and binding site. This region consists of a dimer of one heavy and one light chain variable domain in tight association, which can be covalent in nature, for example in scFv. It is in this configuration that the three CDRs of each variable domain interact to define an antigen binding site on the surface of the VH-VL dimer. Collectively, the six CDRs or a subset thereof confer antigen binding specificity to the antibody. However, even a single variable domain (or half of an Fv comprising only three CDRs specific for an antigen) can have the ability to recognize and bind antigen, although usually at a lower affinity than the entire binding site.

### Anti-CTLA4 Antibodies and Antigen-Binding Fragments

The disclosure provides several antibodies and antigen-binding fragments thereof that specifically bind to CTLA4. In some embodiments, the anti-CTLA4/OX40 antigen-binding protein constructs (e.g., bispecific antibodies) can include an antigen binding region that is derived from these antibodies.

The antibodies and antigen-binding fragments described herein are capable of binding to CTLA4. The disclosure provides e.g., anti-CTLA4 antibodies CT-3D2 (“3D2”), CT-4E12 (“4E12”), CT-5E3 (“5E3”), CT-5F9 (“5F9”), and the antibodies derived therefrom.

The CDR sequences for 3D2, and 3D2 derived antibodies (e.g., human antibodies) include CDRs of the heavy chain variable domain, SEQ ID NOs: 1-3, and CDRs of the light chain variable domain, SEQ ID NOs: 29-31 as defined by Kabat numbering. The CDRs can also be defined by Chothia system. Under the Chothia numbering, the CDR sequences of the heavy chain variable domain are set forth in SEQ ID NOs: 13-15, and CDR sequences of the light chain variable domain are set forth in SEQ ID NOs: 32-34.

The CDR sequences for 4E12, and 4E12 derived antibodies (e.g., human antibodies) include CDRs of the heavy chain variable domain, SEQ ID NOs: 4-6, and CDRs of the light chain variable domain, SEQ ID NOs: 29-31, as defined by Kabat numbering. Under Chothia numbering, the CDR sequences of the heavy chain variable domain are set forth in SEQ ID NOs: 16-18, and CDRs of the light chain variable domain are set forth in SEQ ID NOs: 32-34.

The CDR sequences for 5E3, and 5E3 derived antibodies (e.g., human antibodies) include CDRs of the heavy chain variable domain, SEQ ID NOs: 7-9, and CDRs of the light chain variable domain, SEQ ID NOs: 29-31, as defined by Kabat numbering. Under Chothia numbering, the CDR sequences of the heavy chain variable domain are set forth in SEQ ID NOs: 19-21, and CDRs of the light chain variable domain are set forth in SEQ ID NOs: 32-34.

The CDR sequences for 5F9, and 5F9 derived antibodies (e.g., human antibodies) include CDRs of the heavy chain variable domain, SEQ ID NOs: 10-12, and CDRs of the light chain variable domain, SEQ ID NOs: 29-31, as defined by Kabat numbering. Under Chothia numbering, the CDR sequences of the heavy chain variable domain are set forth in SEQ ID NOs: 22-24, and CDRs of the light chain variable domain are set forth in SEQ ID NOs: 32-34.

Furthermore, in some embodiments, the antibodies or antigen-binding fragments thereof described herein can also contain one, two, or three heavy chain variable region CDRs selected from the group of SEQ ID NOs: 1-3, SEQ ID NOs: 4-6, SEQ ID NOs: 7-9, SEQ ID NOs: 10-12, SEQ ID NOs: 13-15, SEQ ID NOs: 16-18, SEQ ID NOs: 19-21, SEQ ID NOs: 22-24; and/or one, two, or three light chain variable region CDRs selected from the group of SEQ ID NOs: 29-31, and SEQ ID NOs: 32-34.

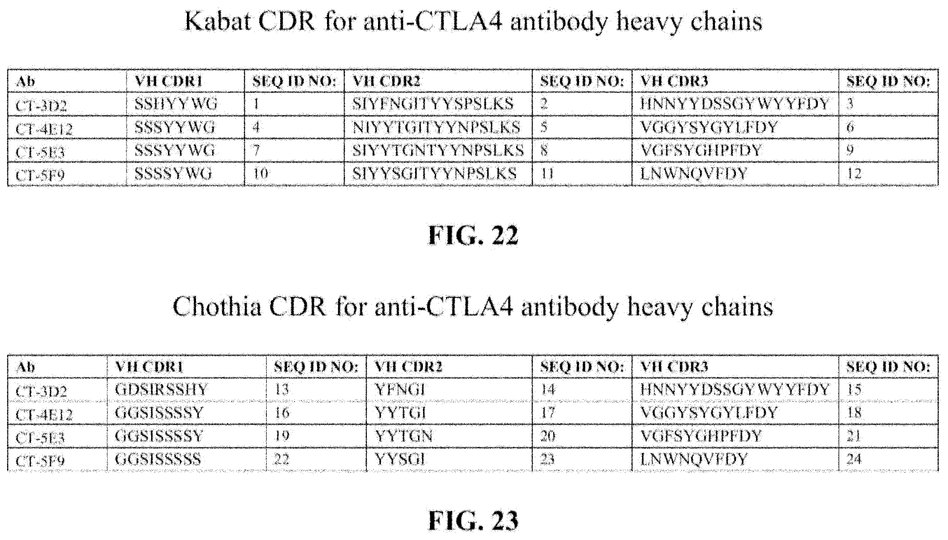

In some embodiments, the antibodies can have a heavy chain variable region (VH) comprising complementarity determining regions (CDRs) 1, 2, 3, wherein the CDR1 region comprises or consists of an amino acid sequence that is at least 80%, 85%, 90%, or 95% identical to a selected VH CDR1 amino acid sequence, the CDR2 region comprises or consists of an amino acid sequence that is at least 80%, 85%, 90%, or 95% identical to a selected VH CDR2 amino acid sequence, and the CDR3 region comprises or consists of an amino acid sequence that is at least 80%, 85%, 90%, or 95% identical to a selected VH CDR3 amino acid sequence, and a light chain variable region (VL) comprising CDRs 1, 2, 3, wherein the CDR1 region comprises or consists of an amino acid sequence that is at least 80%, 85%, 90%, or 95% identical to a selected VL CDR1 amino acid sequence, the CDR2 region comprises or consists of an amino acid sequence that is at least 80%, 85%, 90%, or 95% identical to a selected VL CDR2 amino acid sequence, and the CDR3 region comprises or consists of an amino acid sequence that is at least 80%, 85%, 90%, or 95% identical to a selected VL CDR3 amino acid sequence. The selected VH CDRs 1, 2, 3 amino acid sequences and the selected VL CDRs, 1, 2, 3 amino acid sequences are shown in FIGS. 22 and 26 (Kabat CDR) and FIGS. 23 and 27 (Chothia CDR).

In some embodiments, the antibody or an antigen-binding fragment described herein can contain a heavy chain variable domain containing one, two, or three of the CDRs of SEQ ID NO: 1 with zero, one or two amino acid insertions, deletions, or substitutions; SEQ ID NO: 2 with zero, one or two amino acid insertions, deletions, or substitutions; SEQ ID NO: 3 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 4 with zero, one or two amino acid insertions, deletions, or substitutions; SEQ ID NO: 5 with zero, one or two amino acid insertions, deletions, or substitutions; SEQ ID NO: 6 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 7 with zero, one or two amino acid insertions, deletions, or substitutions; SEQ ID NO: 8 with zero, one or two amino acid insertions, deletions, or substitutions; SEQ ID NO: 9 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 10 with zero, one or two amino acid insertions, deletions, or substitutions; SEQ ID NO: 11 with zero, one or two amino acid insertions, deletions, or substitutions; SEQ ID NO: 12 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 13 with zero, one or two amino acid insertions, deletions, or substitutions; SEQ ID NO: 14 with zero, one or two amino acid insertions, deletions, or substitutions; SEQ ID NO: 15 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 16 with zero, one or two amino acid insertions, deletions, or substitutions; SEQ ID NO: 17 with zero, one or two amino acid insertions, deletions, or substitutions; SEQ ID NO: 18 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 19 with zero, one or two amino acid insertions, deletions, or substitutions; SEQ ID NO: 20 with zero, one or two amino acid insertions, deletions, or substitutions; SEQ ID NO: 21 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 22 with zero, one or two amino acid insertions, deletions, or substitutions; SEQ ID NO: 23 with zero, one or two amino acid insertions, deletions, or substitutions; SEQ ID NO: 24 with zero, one or two amino acid insertions, deletions, or substitutions.

In some embodiments, the antibody or an antigen-binding fragment described herein can contain a light chain variable domain containing one, two, or three of the CDRs of SEQ ID NO: 29 with zero, one or two amino acid insertions, deletions, or substitutions; SEQ ID NO: 30 with zero, one or two amino acid insertions, deletions, or substitutions; SEQ ID NO: 31 with zero, one or two amino acid insertions, deletions, or substitutions.

In some embodiments, the antibody or an antigen-binding fragment described herein can contain a light chain variable domain containing one, two, or three of the CDRs of SEQ ID NO: 32 with zero, one or two amino acid insertions, deletions, or substitutions; SEQ ID NO: 33 with zero, one or two amino acid insertions, deletions, or substitutions; SEQ ID NO: 34 with zero, one or two amino acid insertions, deletions, or substitutions.

The insertions, deletions, and substitutions can be within the CDR sequence, or at one or both terminal ends of the CDR sequence.

The disclosure also provides antibodies or antigen-binding fragments thereof that bind to CTLA4. The antibodies or antigen-binding fragments thereof contain a heavy chain variable region (VH) comprising or consisting of an amino acid sequence that is at least 80%, 85%, 90%, or 95% identical to a selected VH sequence, and a light chain variable region (VL) comprising or consisting of an amino acid sequence that is at least 80%, 85%, 90%, or 95% identical to a selected VL sequence. In some embodiments, the selected VH sequence is SEQ ID NOs: 25, 26, 27, or 28, and the selected VL sequence is SEQ ID NOs: 35.

In some embodiments, the antibody or antigen binding fragment thereof can have 3 VH CDRs that are identical to the CDRs of any VH sequences as described herein. In some embodiments, the antibody or antigen binding fragment thereof can have 3 VL CDRs that are identical to the CDRs of any VL sequences as described herein.

The disclosure also provides nucleic acid comprising a polynucleotide encoding a polypeptide comprising an immunoglobulin heavy chain or an immunoglobulin light chain. The immunoglobulin heavy chain or immunoglobulin light chain comprises CDRs as shown in FIG. 22, FIG. 23, FIG. 26, or FIG. 27, or have sequences as shown in FIG. 28. When the polypeptides are paired with corresponding polypeptide (e.g., a corresponding heavy chain variable region or a corresponding light chain variable region), the paired polypeptides bind to CTLA4 (e.g., human CTLA4).

The anti-CTLA4 antibodies and antigen-binding fragments can also be antibody variants (including derivatives and conjugates) of antibodies or antibody fragments and multi-specific (e.g., bispecific) antibodies or antibody fragments. Additional antibodies provided herein are polyclonal, monoclonal, multi-specific (multimeric, e.g., bispecific), human antibodies, chimeric antibodies (e.g., human-mouse chimera), single-chain antibodies, intracellularly-made antibodies (i.e., intrabodies), and antigen-binding fragments thereof. The antibodies or antigen-binding fragments thereof can be of any type (e.g., IgG, IgE, IgM, IgD, IgA, and IgY), class (e.g., IgG1, IgG2, IgG3, IgG4, IgA1, and IgA2), or subclass. In some embodiments, the antibody or antigen-binding fragment thereof is an IgG antibody or antigen-binding fragment thereof.

Fragments of antibodies are suitable for use in the methods provided so long as they retain the desired affinity and specificity of the full-length antibody. Thus, a fragment of an antibody that binds to CTLA4 will retain an ability to bind to CTLA4. An Fv fragment is an antibody fragment which contains a complete antigen recognition and binding site. This region consists of a dimer of one heavy and one light chain variable domain in tight association, which can be covalent in nature, for example in scFv. It is in this configuration that the three CDRs of each variable domain interact to define an antigen binding site on the surface of the VH-VL dimer. Collectively, the six CDRs or a subset thereof confer antigen binding specificity to the antibody. However, even a single variable domain (or half of an Fv comprising only three CDRs specific for an antigen) can have the ability to recognize and bind antigen, although usually at a lower affinity than the entire binding site.

### Antibodies, Antigen Binding Fragments and Antigen Binding Protein Constructs

The present disclosure provides antibodies, the antigen-binding fragments thereof, or the antigen-binding protein constructs (e.g., bispecific antibodies). The antigen-binding protein construct (e.g., bispecific antibody) can comprise an anti-CTLA4 antibody or antigen-binding fragment thereof, and anti-OX40 antibody or antigen-binding fragment thereof. These antigen-binding protein constructs (e.g., bispecific antibody), anti-CTLA4 antibodies, anti-OX40 antibodies, and antigen-binding fragments thereof can have various forms.

In general, antibodies (also called immunoglobulins) can be made up of two classes of polypeptide chains, light chains and heavy chains. A non-limiting antibody of the present disclosure can be an intact, four immunoglobulin chain antibody comprising two heavy chains and two light chains. The heavy chain of the antibody can be of any isotype including IgM, IgG, IgE, IgA, or IgD or sub-isotype including IgG1, IgG2, IgG2a, IgG2b, IgG3, IgG4, IgE1, IgE2, etc. The light chain can be a kappa light chain or a lambda light chain. An antibody can comprise two identical copies of a light chain and/or two identical copies of a heavy chain. The heavy chains, which each contain one variable domain (or variable region, VH) and multiple constant domains (or constant regions), bind to one another via disulfide bonding within their constant domains to form the “stem” of the antibody. The light chains, which each contain one variable domain (or variable region, VL) and one constant domain (or constant region), each bind to one heavy chain via disulfide binding. The variable region of each light chain is aligned with the variable region of the heavy chain to which it is bound. The variable regions of both the light chains and heavy chains contain three hypervariable regions sandwiched between more conserved framework regions (FR).

These hypervariable regions, known as the complementary determining regions (CDRs), form loops that comprise the principle antigen binding surface of the antibody. The four framework regions largely adopt a beta-sheet conformation and the CDRs form loops connecting, and in some cases forming part of, the beta-sheet structure. The CDRs in each chain are held in close proximity by the framework regions and, with the CDRs from the other chain, contribute to the formation of the antigen-binding region.

Methods for identifying the CDR regions of an antibody by analyzing the amino acid sequence of the antibody are well known, and a number of definitions of the CDRs are commonly used. The Kabat definition is based on sequence variability, and the Chothia definition is based on the location of the structural loop regions. These methods and definitions are described in, e.g., Martin, “Protein sequence and structure analysis of antibody variable domains,” Antibody engineering, Springer Berlin Heidelberg, 2001. 422-439; Abhinandan, et al. “Analysis and improvements to Kabat and structurally correct numbering of antibody variable domains,” Molecular immunology 45.14 (2008): 3832-3839; Wu, T. T. and Kabat, E. A. (1970) J. Exp. Med. 132: 211-250; Martin et al., Methods Enzymol. 203:121-53 (1991); Morea et al., Biophys Chem. 68(1-3):9-16 (October 1997); Morea et al., J Mol Biol. 275(2):269-94 (January 1998); Chothia et al., Nature 342(6252):877-83 (December 1989); Ponomarenko and Bourne, BMC Structural Biology 7:64 (2007); each of which is incorporated herein by reference in its entirety.

The CDRs are important for recognizing an epitope of an antigen. As used herein, an “epitope” is the smallest portion of a target molecule capable of being specifically bound by the antigen binding domain of an antibody. The minimal size of an epitope may be about three, four, five, six, or seven amino acids, but these amino acids need not be in a consecutive linear sequence of the antigen's primary structure, as the epitope may depend on an antigen's three-dimensional configuration based on the antigen's secondary and tertiary structure.

In some embodiments, the antibody is an intact immunoglobulin molecule (e.g., IgG1, IgG2a, IgG2b, IgG3, IgM, IgD, IgE, IgA). The IgG subclasses (IgG1, IgG2, IgG3, and IgG4) are highly conserved, differ in their constant region, particularly in their hinges and upper CH2 domains. The sequences and differences of the IgG subclasses are known in the art, and are described, e.g., in Vidarsson, et al, “IgG subclasses and allotypes: from structure to effector functions.” Frontiers in immunology 5 (2014); Irani, et al. “Molecular properties of human IgG subclasses and their implications for designing therapeutic monoclonal antibodies against infectious diseases.” Molecular immunology 67.2 (2015): 171-182; Shakib, Farouk, ed. The human IgG subclasses: molecular analysis of structure, function and regulation. Elsevier, 2016; each of which is incorporated herein by reference in its entirety.

The antibody can also be an immunoglobulin molecule that is derived from any species (e.g., human, rodent, mouse, rat, camelid). Antibodies disclosed herein also include, but are not limited to, polyclonal, monoclonal, monospecific, polyspecific antibodies, and chimeric antibodies that include an immunoglobulin binding domain fused to another polypeptide. The antigen binding domain or antigen binding fragment is a portion of an antibody that retains specific binding activity of the intact antibody, i.e., any portion of an antibody that is capable of specific binding to an epitope on the intact antibody's target molecule. It includes, e.g., Fab, Fab′, F(ab′)₂, and variants of these fragments. Thus, in some embodiments, an antibody or an antigen binding fragment thereof can be, e.g., a scFv, a Fv, a Fd, a dAb, a bispecific antibody, a bispecific scFv, a diabody, a linear antibody, a single-chain antibody molecule, a multi-specific antibody formed from antibody fragments, and any polypeptide that includes a binding domain which is, or is homologous to, an antibody binding domain. Non-limiting examples of antigen binding domains include, e.g., the heavy chain and/or light chain CDRs of an intact antibody, the heavy and/or light chain variable regions of an intact antibody, full length heavy or light chains of an intact antibody, or an individual CDR from either the heavy chain or the light chain of an intact antibody.

In some embodiments, the scFV has two heavy chain variable domains, and two light chain variable domains. In some embodiments, the scFV has two antigen binding regions (Antigen binding regions: A and B), and the two antigen binding regions can bind to the respective target antigens with different affinities.

In some embodiments, the antigen binding fragment can form a part of a chimeric antigen receptor (CAR). In some embodiments, the chimeric antigen receptor are fusions of single-chain variable fragments (scFv) as described herein, fused to CD3-zeta transmembrane- and endodomain. In some embodiments, the chimeric antigen receptor also comprises intracellular signaling domains from various costimulatory protein receptors (e.g., CD28, 41BB, ICOS). In some embodiments, the chimeric antigen receptor comprises multiple signaling domains, e.g., CD3z-CD28-41BB or CD3z-CD28-OX40, to increase potency. Thus, in one aspect, the disclosure further provides cells (e.g., T cells) that express the chimeric antigen receptors as described herein.

In some embodiments, the antibodies, the antigen-binding fragments thereof, or the antigen-binding protein constructs (e.g., bispecific antibodies) can bind to two different antigens or two different epitopes.

In some embodiments, the antibodies, the antigen-binding fragments thereof, or the antigen-binding protein constructs (e.g., bispecific antibodies) can comprises one, two, or three heavy chain variable region CDRs selected from FIGS. 22, 23, 24, and 25. In some embodiments, the antibodies, the antigen-binding fragments thereof, or the antigen-binding protein constructs (e.g., bispecific antibodies) can comprises one, two, or three light chain variable region CDRs selected from FIGS. 26, and 27.

In some embodiments, the antibodies, the antigen-binding fragments thereof, or the antigen-binding protein constructs (e.g., bispecific antibodies) described herein can be conjugated to a therapeutic agent. The antibody-drug conjugate comprising the antibody or antigen-binding fragment thereof can covalently or non-covalently bind to a therapeutic agent.

In some embodiments, the therapeutic agent is a cytotoxic or cytostatic agent (e.g., monomethyl auristatin E, monomethyl auristatin F, cytochalasin B, gramicidin D, ethidium bromide, emetine, mitomycin, etoposide, tenoposide, vincristine, vinblastine, colchicin, doxorubicin, daunorubicin, dihydroxy anthracin, maytansinoids such as DM-1 and DM-4, dione, mitoxantrone, mithramycin, actinomycin D, 1-dehydrotestosterone, glucocorticoids, procaine, tetracaine, lidocaine, propranolol, puromycin, epirubicin, and cyclophosphamide and analogs).

Multimerization of antibodies may be accomplished through natural aggregation of antibodies or through chemical or recombinant linking techniques known in the art. For example, some percentage of purified antibody preparations (e.g., purified IgG1 molecules) spontaneously form protein aggregates containing antibody homodimers and other higher-order antibody multimers.

In some embodiments, the multi-specific antibody is a bispecific antibody. Bispecific antibodies can be made by engineering the interface between a pair of antibody molecules to maximize the percentage of heterodimers that are recovered from recombinant cell culture. For example, the interface can contain at least a part of the CH3 domain of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers. This method is described, e.g., in WO 96/27011, which is incorporated by reference in its entirety.

Any of the antibodies, the antigen-binding fragments thereof, or the antigen-binding protein constructs (e.g., bispecific antibodies) described herein may be conjugated to a stabilizing molecule (e.g., a molecule that increases the half-life of the antibody or antigen-binding fragment thereof in a subject or in solution). Non-limiting examples of stabilizing molecules include: a polymer (e.g., a polyethylene glycol) or a protein (e.g., serum albumin, such as human serum albumin). The conjugation of a stabilizing molecule can increase the half-life or extend the biological activity of an antibody or an antigen-binding fragment in vitro (e.g., in tissue culture or when stored as a pharmaceutical composition) or in vivo (e.g., in a human).

The antibodies, the antigen-binding fragments thereof, or the antigen-binding protein constructs (e.g., bispecific antibodies) can also have various forms. Many different formats of antigen binding constructs are known in the art, and are described e.g., in Suurs, et al. “A review of bispecific antibodies and antibody constructs in oncology and clinical challenges,” Pharmacology & therapeutics (2019), which is incorporated herein by reference in the entirety.

In some embodiments, the antigen-binding protein construct is a BiTe, a (scFv)2, a nanobody, a nanobody-HSA, a DART, a TandAb, a scDiabody, a scDiabody-CH3, scFv-CH-CL-scFv, a HSAbody, scDiabody-HAS, or a tandem-scFv. In some embodiments, the antigen-binding protein construct is a VHH-scAb, a VHH-Fab, a Dual scFab, a F(ab′)2, a diabody, a crossMab, a DAF (two-in-one), a DAF (four-in-one), a DutaMab, a DT-IgG, a knobs-in-holes common light chain, a knobs-in-holes assembly, a charge pair, a Fab-arm exchange, a SEEDbody, a LUZ-Y, a Fcab, a KX-body, an orthogonal Fab, a DVD-IgG, a IgG(H)-scFv, a scFv-(H)IgG, IgG(L)-scFv, scFv-(L)IgG, IgG(L,H)-Fv, IgG(H)-V, V(H)-IgG, IgG(L)-V, V(L)-IgG, KIH IgG-scFab, 2scFv-IgG, IgG-2scFv, scFv4-Ig, Zybody, DVI-IgG, Diabody-CH3, a triple body, a miniantibody, a minibody, a TriBi minibody, scFv-CH3 KIH, Fab-scFv, a F(ab′)₂-scFv2, a scFv-KIH, a Fab-scFv-Fc, a tetravalent HCAb, a scDiabody-Fc, a Diabody-Fc, a tandem scFv-Fc, an Intrabody, a dock and lock, a lmmTAC, an IgG-IgG conjugate, a Cov-X-Body, or a scFv1-PEG-scFv2.

In some embodiments, the antigen-binding protein construct can be a TrioMab. In a TrioMab, the two heavy chains are from different species, wherein different sequences restrict the heavy-light chain pairing.

In some embodiments, the antigen-binding protein construct has two different heavy chains and one common light chain. Heterodimerization of heavy chains can be based on the knob-in-holes or some other heavy chain pairing technique.

In some embodiments, CrossMAb technique can be used produce bispecific antibodies. CrossMAb technique can be used enforce correct light chain association in bispecific heterodimeric IgG antibodies, this technique allows the generation of various bispecific antibody formats, including bi-(1+1), tri-(2+1) and tetra-(2+2) valent bispecific antibodies, as well as non-Fc tandem antigen-binding fragment (Fab)-based antibodies. These formats can be derived from any existing antibody pair using domain crossover, without the need for the identification of common light chains, post-translational processing/in vitro chemical assembly or the introduction of a set of mutations enforcing correct light chain association. The method is described in Klein et al., “The use of CrossMAb technology for the generation of bi- and multispecific antibodies.” MAbs. Vol. 8. No. 6. Taylor & Francis, 2016, which is incorporated by reference in its entirety. In some embodiments, the CH1 in the heavy chain and the CL domain in the light chain are swapped.

The antigen-binding protein construct can be a Duobody. The Fab-exchange mechanism naturally occurring in IgG4 antibodies is mimicked in a controlled matter in IgG1 antibodies, a mechanism called controlled Fab exchange. This format can ensure specific pairing between the heavy-light chains.

In Dual-variable-domain antibody (DVD-Ig), additional VH and variable light chain (VL) domain are added to each N-terminus for bispecific targeting. This format resembles the IgG-scFv, but the added binding domains are bound individually to their respective N-termini instead of a scFv to each heavy chain N-terminus.

In scFv-IgG, the two scFv are connected to the C-terminus of the heavy chain (CH3). The scFv-IgG format has two different bivalent binding sites and is consequently also called tetravalent. There are no heavy-chain and light-chain pairing problem in the scFv-IgG.

In some embodiments, the antigen-binding protein construct can be have a IgG-IgG format. Two intact IgG antibodies are conjugated by chemically linking the C-terminals of the heavy chains.

The antigen-binding protein construct can also have a Fab-scFv-Fc format. In Fab-scFv-Fc format, a light chain, heavy chain and a third chain containing the Fc region and the scFv are assembled. It can ensure efficient manufacturing and purification.

In some embodiments, antigen-binding protein construct can be a TF. Three Fab fragments are linked by disulfide bridges. Two fragments target the tumor associated antigen (TAA) and one fragment targets a hapten. The TF format does not have an Fc region.

ADAPTIR has two scFvs bound to each sides of an Fc region. It abandons the intact IgG as a basis for its construct, but conserves the Fc region to extend the half-life and facilitate purification.

Bispecific T cell Engager (“BiTE”) consists of two scFvs, VLA VHA and VHB VLB on one peptide chain. It has only binding domains, no Fc region.

In BiTE-Fc, an Fc region is fused to the BiTE construct. The addition of Fc region enhances half-life leading to longer effective concentrations, avoiding continuous IV.

Dual affinity retargeting (DART) has two peptide chains connecting the opposite fragments, thus VLA with VHB and VLB with VHA, and a sulfur bond at their C-termini fusing them together. In DART, the sulfur bond can improve stability over BiTEs.

In DART-Fe, an Fe region is attached to the DART structure. It can be generated by assembling three chains, two via a disulfide bond, as with the DART. One chain contains half of the Fc region which will dimerize with the third chain, only expressing the Fc region. The addition of Fc region enhances half-life leading to longer effective concentrations, avoiding continuous IV.

In tetravalent DART, four peptide chains are assembled. Basically, two DART molecules are created with half an Fe region and will dimerize. This format has bivalent binding to both targets, thus it is a tetravalent molecule.

Tandem diabody (TandAb) comprises two diabodies. Each diabody consists of an VHA and VLB fragment and a VHA and VLB fragment that are covalently associated. The two diabodies are linked with a peptide chain. It can improve stability over the diabody consisting of two scFvs. It has two bivalent binding sites.

The ScFv-scFv-toxin includes toxin and two scFv with a stabilizing linker. It can be used for specific delivery of payload.

In modular scFv-scFv-scFv, one scFv directed against the TAA is tagged with a short recognizable peptide is assembled to a bsAb consisting of two scFvs, one directed against CD3 and one against the recognizable peptide.

In lmmTAC, a stabilized and soluble T cell receptor is fused to a scFv recognizing CD3. By using a TCR, the lmmTAC is suitable to target processed, e.g. intracellular, proteins.

Tri-specific nanobody has two single variable domains (nanobodies) with an additional module for half-life extension. The extra module is added to enhance half-life.

In Trispecific Killer Engager (TriKE), two scFvs are connected via polypeptide linkers incorporating human IL-15. The linker to IL-15 is added to increase survival and proliferation of NKs.

In some embodiments, the antigen-binding protein construct is a bispecific antibody. In some embodiments, the bispecific antibody in present disclosure is designed to be 1+1 (monovalent for each target) and has an IgG1 subtype structure. This can reduce the avidity to cells with low expression levels of CTLA4 and OX40, and increase the avidity to cells that co-express CTLA4 and OX40, to achieve enhanced targeting function. Mutations S239D and/or 1332E (SI mutations) can also be introduced in antibody heavy chains to enhance the antibody affinity to FcγRIIIA.

In some embodiments, the antibodies, the antigen-binding fragments thereof, the antigen-binding protein constructs (e.g., the anti-OX40 antibody, the anti-CTLA4 antibody, or the bispecific antibody) have a light chain constant region that is at least 80%, 85%, 86%, 87%, 88%, 89%, 90%, 91%, 92%, 93%, 94%, 95%, 96%, 97%, 98%, 99% or 100% identical to SEQ ID NO: 95, 97, or 101, and a heavy chain constant region that is at least 80%, 85%, 86%, 87%, 88%, 89%, 90%, 91%, 92%, 93%, 94%, 95%, 96%, 97%, 98%, 99% or 100% identical to any one of SEQ ID NOs: 92-94, 96, 98-100, 163, and 164.

In some embodiments, the anti-CTLA4/OX40 antigen-binding protein construct (e.g., antibodies, bispecific antibodies, or antibody fragments thereof) include KIH mutations and/or SI mutations. In some embodiments, the antigen-binding protein construct includes a first antigen-binding domain that specifically binds to CTLA4, and a second antigen-binding domain that specially binds to OX40. In some embodiments, the first antigen-binding domain includes a heavy chain that including one or more knob mutations (a knob heavy chain), and the second antigen-binding domain includes a heavy chain including one or more hole mutations (a hole heavy chain). In some embodiments, the first antigen-binding domain includes a heavy chain that including one or more hole mutations (a hole heavy chain), and the second antigen-binding domain includes a heavy chain including one or more knob mutations (a knob heavy chain). In some embodiments, the anti-CTLA4/OX40 antigen-binding protein construct includes a knob heavy chain that is at least 80%, 85%, 86%, 87%, 88%, 89%, 90%, 91%, 92%, 93%, 94%, 95%, 96%, 97%, 98%, 99% or 100% identical to any one of SEQ ID NOs: 111, 113, 115, 117, 119, 121, 123, 125, 127, 129, 131, 133, 135, 137, 139, 141, 143, or 145. In some embodiments, the anti-CTLA4/OX40 antigen-binding protein construct includes a hole heavy chain that is at least 80%, 85%, 86%, 87%, 88%, 89%, 90%, 91%, 92%, 93%, 94%, 95%, 96%, 97%, 98%, 99% or 100% identical to any one of SEQ ID NOs: 112, 114, 116, 118, 120, 122, 124, 126, 128, 130, 132, 134, 136, 138, 140, 142, 144, or 146.

In some embodiments, the anti-CTLA4/OX40 antigen-binding protein construct described herein includes a knob heavy chain that is at least 80%, 85%, 90%, or 95% identical to SEQ ID NO: 111, and a hole heavy chain that is at least 80%, 85%, 90%, or 95% identical to SEQ ID NO: 112. In some embodiments, the anti-CTLA4/OX40 antigen-binding protein construct described herein includes a knob heavy chain that is at least 80%, 85%, 90%, or 95% identical to SEQ ID NO: 113, and a hole heavy chain that is at least 80%, 85%, 90%, or 95% identical to SEQ ID NO: 114. In some embodiments, the anti-CTLA4/OX40 antigen-binding protein construct described herein includes a knob heavy chain that is at least 80%, 85%, 90%, or 95% identical to SEQ ID NO: 115, and a hole heavy chain that is at least 80%, 85%, 90%, or 95% identical to SEQ ID NO: 116. In some embodiments, the anti-CTLA4/OX40 antigen-binding protein construct described herein includes a knob heavy chain that is at least 80%, 85%, 90%, or 95% identical to SEQ ID NO: 117, and a hole heavy chain that is at least 80%, 85%, 90%, or 95% identical to SEQ ID NO: 118. In some embodiments, the anti-CTLA4/OX40 antigen-binding protein construct described herein includes a knob heavy chain that is at least 80%, 85%, 90%, or 95% identical to SEQ ID NO: 119, and a hole heavy chain that is at least 80%, 85%, 90%, or 95% identical to SEQ ID NO: 120. In some embodiments, the anti-CTLA4/OX40 antigen-binding protein construct described herein includes a knob heavy chain that is at least 80%, 85%, 90%, or 95% identical to SEQ ID NO: 121, and a hole heavy chain that is at least 80%, 85%, 90%, or 95% identical to SEQ ID NO: 122. In some embodiments, the anti-CTLA4/OX40 antigen-binding protein construct described herein includes a knob heavy chain that is at least 80%, 85%, 90%, or 95% identical to SEQ ID NO: 123, and a hole heavy chain that is at least 80%, 85%, 90%, or 95% identical to SEQ ID NO: 124. In some embodiments, the anti-CTLA4/OX40 antigen-binding protein construct described herein includes a knob heavy chain that is at least 80%, 85%, 90%, or 95% identical to SEQ ID NO: 125, and a hole heavy chain that is at least 80%, 85%, 90%, or 95% identical to SEQ ID NO: 126. In some embodiments, the anti-CTLA4/OX40 antigen-binding protein construct described herein includes a knob heavy chain that is at least 80%, 85%, 90%, or 95% identical to SEQ ID NO: 127, and a hole heavy chain that is at least 80%, 85%, 90%, or 95% identical to SEQ ID NO: 128. In some embodiments, the anti-CTLA4/OX40 antigen-binding protein construct described herein includes a knob heavy chain that is at least 80%, 85%, 90%, or 95% identical to SEQ ID NO: 129, and a hole heavy chain that is at least 80%, 85%, 90%, or 95% identical to SEQ ID NO: 130. In some embodiments, the anti-CTLA4/OX40 antigen-binding protein construct described herein includes a knob heavy chain that is at least 80%, 85%, 90%, or 95% identical to SEQ ID NO: 131, and a hole heavy chain that is at least 80%, 85%, 90%, or 95% identical to SEQ ID NO: 132. In some embodiments, the anti-CTLA4/OX40 antigen-binding protein construct described herein includes a knob heavy chain that is at least 80%, 85%, 90%, or 95% identical to SEQ ID NO: 133, and a hole heavy chain that is at least 80%, 85%, 90%, or 95% identical to SEQ ID NO: 134. In some embodiments, the anti-CTLA4/OX40 antigen-binding protein construct described herein includes a knob heavy chain that is at least 80%, 85%, 90%, or 95% identical to SEQ ID NO: 135, and a hole heavy chain that is at least 80%, 85%, 90%, or 95% identical to SEQ ID NO: 136. In some embodiments, the anti-CTLA4/OX40 antigen-binding protein construct described herein includes a knob heavy chain that is at least 80%, 85%, 90%, or 95% identical to SEQ ID NO: 137, and a hole heavy chain that is at least 80%, 85%, 90%, or 95% identical to SEQ ID NO: 138. In some embodiments, the anti-CTLA4/OX40 antigen-binding protein construct described herein includes a knob heavy chain that is at least 80%, 85%, 90%, or 95% identical to SEQ ID NO: 139, and a hole heavy chain that is at least 80%, 85%, 90%, or 95% identical to SEQ ID NO: 140. In some embodiments, the anti-CTLA4/OX40 antigen-binding protein construct described herein includes a knob heavy chain that is at least 80%, 85%, 90%, or 95% identical to SEQ ID NO: 141, and a hole heavy chain that is at least 80%, 85%, 90%, or 95% identical to SEQ ID NO: 142. In some embodiments, the anti-CTLA4/OX40 antigen-binding protein construct described herein includes a knob heavy chain that is at least 80%, 85%, 90%, or 95% identical to SEQ ID NO: 143, and a hole heavy chain that is at least 80%, 85%, 90%, or 95% identical to SEQ ID NO: 144. In some embodiments, the anti-CTLA4/OX40 antigen-binding protein construct described herein includes a knob heavy chain that is at least 80%, 85%, 90%, or 95% identical to SEQ ID NO: 145, and a hole heavy chain that is at least 80%, 85%, 90%, or 95% identical to SEQ ID NO: 146. In some embodiments, the anti-CTLA4/OX40 antigen-binding protein construct described herein includes a common light chain that is at least 80%, 85%, 90%, or 95% identical to SEQ ID NO: 147.

In some embodiments, the bispecific antibody or antigen-binding fragment thereof described herein has a common light chain.

### Antibody Characteristics

The anti-CTLA4/OX40 antigen-binding protein construct (e.g., antibodies, bispecific antibodies, or antibody fragments thereof) can include an antigen-binding region that is derived from any anti-CTLA4 antibody or any antigen-binding fragment thereof as described herein.

The anti-CTLA4 antibodies, or antigen-binding fragments thereof described herein can bind to CTLA4, and block the binding between CTLA4 and CD80, and/or the binding between CTLA4 and CD86. By blocking the binding between CTLA4 and CD80, and/or the binding between CTLA4 and CD86, the anti-CTLA4 antibodies disrupts the CTLA4 inhibitory pathway and upregulates the immune response. In some embodiments, the anti-CTLA4 antibodies, or antigen-binding fragments thereof can bind to Treg that express CTLA4, and induce ADCC.

General techniques can be used to measure the affinity of an antibody for an antigen include, e.g., ELISA, RIA, and surface plasmon resonance (SPR). Affinities can be deduced from the quotient of the kinetic rate constants (KD=koff/kon). In some implementations, the antibodies, the antigen-binding fragments thereof, or the antigen-binding protein construct (e.g., bispecific antibody), can bind to CTLA4 (e.g., human CTLA4, monkey CTLA4, mouse CTLA4, and/or chimeric CTLA4) with a dissociation rate (koff) of less than 0.1 s⁻¹, less than 0.01 s⁻¹, less than 0.001 s⁻¹, less than 0.0001 s⁻¹, or less than 0.00001 s⁻¹. In some embodiments, the dissociation rate (koff) is greater than 0.01 s⁻¹, greater than 0.001 s⁻¹, greater than 0.0001 s⁻¹, greater than 0.00001 s⁻¹, or greater than 0.000001 s⁻¹.

In some embodiments, kinetic association rates (kon) is greater than 1×10²/Ms, greater than 1×10³/Ms, greater than 1×10⁴/Ms, greater than 1×10⁵/Ms, or greater than 1×10⁶/Ms. In some embodiments, kinetic association rates (kon) is less than 1×10⁵/Ms, less than 1×10⁶/Ms, or less than 1×10⁷/Ms.

In some embodiments, the antibodies, the antigen-binding fragments thereof, or the antigen-binding protein construct (e.g., bispecific antibody) can bind to CTLA4 (e.g., human CTLA4, monkey CLTA4, mouse CTLA4, and/or chimeric CTLA4) with a KD of less than 1×10⁻⁶ M, less than 1×10⁻⁷ M, less than 1×10⁻⁸ M, less than 1×10⁻⁹ M, or less than 1×10⁻¹⁰ M. In some embodiments, the KD is less than 30 nM, 20 nM, 15 nM, 10 nM, 9 nM, 8 nM, 7 nM, 6 nM, 5 nM, 4 nM, 3 nM, 2 nM, or 1 nM. In some embodiments, KD is greater than 1×10⁻⁷ M, greater than 1×10⁻⁸ M, greater than 1×10⁻⁹ M, or greater than 1×10⁻¹⁰ M.

The anti-CTLA4/OX40 antigen-binding protein construct (e.g., bispecific antibodies) can also include an antigen-binding region that is derived from any anti-OX40 antibody or antigen-binding fragment thereof as described herein. The anti-OX40 antibodies or antigen-binding fragments thereof described herein can block the binding between OX40 and OX40L. In some embodiments, by binding to OX40, the antibody can also promote OX40 signaling pathway and upregulates the immune response. Thus, in some embodiments, the antibodies or antigen-binding fragments thereof as described herein are OX40 agonist. In some embodiments, the antibodies or antigen-binding fragments thereof are OX40 antagonist.

In some implementations, the antibodies, the antigen-binding fragments thereof, or the antigen-binding protein constructs (e.g., bispecific antibody) can bind to OX40 (e.g., human OX40, monkey OX40, mouse OX40, and/or chimeric OX40) with a dissociation rate (koff) of less than 0.1 s⁻¹, less than 0.01 s⁻¹, less than 0.001 s⁻¹, less than 0.0001 s⁻¹, or less than 0.00001 s⁻¹. In some embodiments, the dissociation rate (koff) is greater than 0.01 s⁻¹, greater than 0.001 s⁻¹, greater than 0.0001 s⁻¹, greater than 0.00001 s⁻¹, or greater than 0.000001 s⁻¹.

In some embodiments, kinetic association rates (kon) is greater than 1×10²/Ms, greater than 1×10³/Ms, greater than 1×10⁴/Ms, greater than 1×10⁵/Ms, or greater than 1×10⁶/Ms. In some embodiments, kinetic association rates (kon) is less than 1×10⁵/Ms, less than 1×10⁶/Ms, or less than 1×10⁷/Ms.

Affinities can be deduced from the quotient of the kinetic rate constants (KD=koff/kon). In some embodiments, KD is less than 1×10⁻⁶ M, less than 1×10⁻⁷ M, less than 1×10⁻⁸ M, less than 1×10⁻⁹ M, or less than 1×10⁻¹⁰ M. In some embodiments, the KD is less than 30 nM, 20 nM, 15 nM, 10 nM, 9 nM, 8 nM, 7 nM, 6 nM, 5 nM, 4 nM, 3 nM, 2 nM, or 1 nM. In some embodiments, KD is greater than 1×10⁻⁷ M, greater than 1×10⁻⁸ M, greater than 1×10⁻⁹ M, or greater than 1×10⁻¹⁰ M.

In some embodiments, the antibodies, the antigen-binding fragments thereof, or the antigen-binding protein constructs (e.g., bispecific antibody) as described herein can increase immune response, activity of OX40, activity or number of T cells (e.g., CD8+ and/or CD4+ cells) by at least 10%, 20%, 30%, 40%, 50%, 60%, 70%, 80%, 90%, 100%, 2 folds, 3 folds, 5 folds, 10 folds, or 20 folds. In some embodiments, the antibodies, the antigen-binding fragments thereof, or the antigen-binding protein constructs (e.g., bispecific antibody) as described herein can decrease the activity or number of Treg by at least 10%, 20%, 30%, 40%, 50%, 60%, 70%, 80%, 90%, 100%, 2 folds, 3 folds, 5 folds, 10 folds, or 20 folds.

In some embodiments, the antibodies, the antigen-binding fragments thereof, or the antigen-binding protein constructs (e.g., bispecific antibody) as described herein are OX40 agonist. In some embodiments, the antibodies, the antigen-binding fragments thereof, or the antigen-binding protein constructs (e.g., bispecific antibody) can increase OX40 signal transduction in a target cell (e.g., T cell) that expresses OX40. In some embodiments, OX40 signal transduction is detected by monitoring NFkB downstream signaling.

Because the antigen-binding protein construct (e.g., bispecific antibody) binds to both OX40 and CTLA4, for cells that express both OX40 and CTLA4, the antigen-binding protein construct has a higher binding affinity to these cells. Avidity can be used to measure the binding affinity of an antigen-binding protein construct to these cells. Avidity is the accumulated strength of multiple affinities of individual non-covalent binding interactions.

Thermal stabilities can also be determined. The antibodies, the antigen-binding fragments thereof, or the antigen-binding protein constructs (e.g., bispecific antibody) as described herein can have a Tm greater than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C. As IgG can be described as a multi-domain protein, the melting curve sometimes shows two transitions, with a first denaturation temperature, Tm D1, and a second denaturation temperature Tm D2. The presence of these two peaks often indicate the denaturation of the Fc domains (Tm D1) and Fab domains (Tm D2), respectively. When there are two peaks, Tm usually refers to Tm D2. Thus, in some embodiments, the antibodies or antigen binding fragments as described herein has a Tm D1 greater than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C. In some embodiments, the antibodies or antigen binding fragments as described herein has a Tm D2 greater than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C. In some embodiments, Tm, Tm D1, Tm D2 are less than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C.

In some embodiments, the antibodies, the antigen-binding fragments thereof, the antigen-binding protein constructs (e.g., bispecific antibody), can bind to human CTLA4 or monkey CTLA4. In some embodiments, the antibodies, the antigen-binding fragments thereof, the antigen-binding protein constructs (e.g., bispecific antibody), cannot bind to human CTLA4 or monkey CTLA4. In some embodiments, the antibodies, the antigen-binding fragments thereof, the antigen-binding protein constructs (e.g., bispecific antibody), can bind to human OX40 or monkey OX40. In some embodiments, the antibodies, the antigen-binding fragments thereof, the antigen-binding protein constructs (e.g., bispecific antibody), cannot bind to human OX40 or monkey OX40.

In some embodiments, the antibodies, the antigen-binding fragments thereof, the antigen-binding protein constructs (e.g., the anti-OX40 antibody, the anti-CTLA4 antibody, or the bispecific antibody) has a purity that is greater than 30%, 40%, 50%, 60%, 70%, 72.5%, 75%, 77.5%, 80%, 82.5%, 85%, 87.5%, 90%, 91%, 92%, 93%, 94%, 95%, 96%, 97%, 98%, or 99%, e.g., as measured by HPLC. In some embodiments, the purity is less than 30%, 40%, 50%, 60%, 70%, 72.5%, 75%, 77.5%, 80%, 82.5%, 85%, 87.5%, 90%, 91%, 92%, 93%, 94%, 95%, 96%, 97%, 98%, or 99%, e.g., as measured by HPLC.

In some embodiments, the antibodies, the antigen-binding fragments thereof, the antigen-binding protein constructs (e.g., the anti-OX40 antibody, the anti-CTLA4 antibody, or the bispecific antibody) has a yield that is greater than 20, 30, 40, 50, 60, 70, 80, 90, 100, 200, 300, 400, 500, 600, or 700 (g/mL). In some embodiments, the yield is less than 20, 30, 40, 50, 60, 70, 80, 90, 100, 200, 300, 400, 500, 600, or 700 (g/mL).

In some embodiments, the antibodies, the antigen-binding fragments thereof, or the antigen-binding protein constructs (e.g., the anti-OX40 antibody, the anti-CTLA4 antibody, or the bispecific antibody) has a tumor growth inhibition rate or percentage (TGI_{TV}%) that is greater than 10%, 20%, 30%, 40%, 50%, 60%, 70%, 80%, 90%, 100%, 110%, 120%, 130%, 140%, 150%, 160%, 170%, 180%, 190%, or 200%. In some embodiments, the antibody has a tumor growth inhibition percentage that is less than 60%, 70%, 80%, 90%, 100%, 110%, 120%, 130%, 140%, 150%, 160%, 170%, 180%, 190%, or 200%. The TGI_{TV}% can be determined, e.g., at 3, 4, 5, 6, 7, 8, 9, 10, 11, 12, 13, 14, 15, 16, 17, 18, 19, 20, 21, 22, 23, 24, 25, 26, 27, 28, 29, or 30 days after the treatment starts, or 1, 2, 3, 4, 5, 6, 7, 8, 9, 10, 11, or 12 months after the treatment starts. As used herein, the tumor growth inhibition rate or percentage (TGI %) is calculated using the following formula:

$$TGI ( \% ) = [ 1 - ( Ti - T 0 ) / ( Vi - V 0 ) ] \times 100$$

Ti is the average tumor volume in the treatment group on day i. T0 is the average tumor volume in the treatment group on day zero. Vi is the average tumor volume in the control group on day i. V0 is the average tumor volume in the control group on day zero.

In some embodiments, the antibody, the antigen-binding fragment thereof, or the antigen-binding protein construct (e.g., bispecific antibody) has a functional Fc region. In some embodiments, effector function of a functional Fc region is antibody-dependent cell-mediated cytotoxicity (ADCC). In some embodiments, effector function of a functional Fc region is phagocytosis. In some embodiments, effector function of a functional Fc region is ADCC and phagocytosis. In some embodiments, the Fc region is human IgG1, human IgG2, human IgG3, or human IgG4. In some embodiments, one or both mutations S239D and/or 1332E (SI mutations) are introduced in antibody Fc region to enhance the antibody affinity to FcγRIIIA, thereby increasing ADCC effects. A detailed description of SI mutations can be found in U.S. Pat. No. 7,662,925B2, which is incorporated by reference in its entirety.

In some embodiments, the antibody, the antigen-binding fragment thereof, or the antigen-binding protein construct (e.g., bispecific antibody) does not have a functional Fc region. For example, the antibodies or antigen binding fragments are Fab, Fab′, F(ab′)₂, and Fv fragments. In some embodiments, the protein constructs as described herein have an Fc region without effector function. In some embodiments, the Fc is a human IgG4 Fc. In some embodiments, the Fe does not have a functional Fc region. For example, the Fc region has LALA mutations (L234A and L235A mutations in EU numbering), or LALA-PG mutations (L234A, L235A, P329G mutations in EU numbering).

Some other modifications to the Fc region can be made. For example, a cysteine residue(s) can be introduced into the Fe region, thereby allowing interchain disulfide bond formation in this region. The homodimeric fusion protein thus generated may have any increased half-life in vitro and/or in vivo.

In some embodiments, the IgG4 has S228P mutation (EU numbering). The S228P mutation prevents in vivo and in vitro IgG4 Fab-arm exchange.

In some embodiments, Fc regions are provided having a carbohydrate structure that lacks fucose attached (directly or indirectly) to an Fe region. For example, the amount of fucose in such Fe region composition may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues; or position 314 in Kabat numbering); however, Asn297 may also be located about ±3 amino acids upstream or downstream of position 297, i.e., between positions 294 and 300, due to minor sequence variations in Fc region sequences. Such fucosylation variants may have improved ADCC function. In some embodiments, to reduce glycan heterogeneity, the Fc region can be further engineered to replace the Asparagine at position 297 with Alanine (N297A).

In some embodiments, the main peak of HPLC-SEC accounts for at least 80%, 85%, 90%, 95%, 96%, 97%, 98%, 99%, or 99.5% of the protein complex described herein after purification by protein A-based affinity chromatography and/or size-exclusive chromatography.

### Methods of Making Antigen-Binding Protein Constructs

An isolated fragment of human protein can be used as an immunogen to generate antibodies using standard techniques for polyclonal and monoclonal antibody preparation. Polyclonal antibodies can be raised in animals by multiple injections (e.g., subcutaneous or intraperitoneal injections) of an antigenic peptide or protein. In some embodiments, the antigenic peptide or protein is injected with at least one adjuvant. In some embodiments, the antigenic peptide or protein can be conjugated to an agent that is immunogenic in the species to be immunized. Animals can be injected with the antigenic peptide or protein more than one time (e.g., twice, three times, or four times).

The full-length polypeptide or protein can be used or, alternatively, antigenic peptide fragments thereof can be used as immunogens. The antigenic peptide of a protein comprises at least 8 (e.g., at least 10, 15, 20, or 30) amino acid residues of the amino acid sequence of the protein and encompasses an epitope of the protein such that an antibody raised against the peptide forms a specific immune complex with the protein.

An immunogen typically is used to prepare antibodies by immunizing a suitable subject (e.g., human or transgenic animal expressing at least one human immunoglobulin locus). An appropriate immunogenic preparation can contain, for example, a recombinantly-expressed or a chemically-synthesized polypeptide. The preparation can further include an adjuvant, such as Freund's complete or incomplete adjuvant, or a similar immunostimulatory agent.

Polyclonal antibodies can be prepared as described above by immunizing a suitable subject with a polypeptide, or an antigenic peptide thereof (e.g., part of the protein) as an immunogen. The antibody titer in the immunized subject can be monitored over time by standard techniques, such as with an enzyme-linked immunosorbent assay (ELISA) using the immobilized polypeptide or peptide. If desired, the antibody molecules can be isolated from the mammal (e.g., from the blood) and further purified by well-known techniques, such as protein A of protein G chromatography to obtain the IgG fraction. At an appropriate time after immunization, e.g., when the specific antibody titers are highest, antibody-producing cells can be obtained from the subject and used to prepare monoclonal antibodies by standard techniques, such as the hybridoma technique originally described by Kohler et al. (Nature 256:495-497, 1975), the human B cell hybridoma technique (Kozbor et al., Immunol. Today 4:72, 1983), the EBV-hybridoma technique (Cole et al., Monoclonal Antibodies and Cancer Therapy, Alan R. Liss, Inc., pp. 77-96, 1985), or trioma techniques. The technology for producing hybridomas is well known (see, generally, Current Protocols in Immunology, 1994, Coligan et al. (Eds.), John Wiley & Sons, Inc., New York, NY). Hybridoma cells producing a monoclonal antibody are detected by screening the hybridoma culture supernatants for antibodies that bind the polypeptide or epitope of interest, e.g., using a standard ELISA assay.

Variants of the antibodies or antigen-binding fragments described herein can be prepared by introducing appropriate nucleotide changes into the DNA encoding a human, humanized, or chimeric antibody, or antigen-binding fragment thereof described herein, or by peptide synthesis. Such variants include, for example, deletions, insertions, or substitutions of residues within the amino acids sequences that make-up the antigen-binding site of the antibody or an antigen-binding domain. In a population of such variants, some antibodies or antigen-binding fragments will have increased affinity for the target protein. Any combination of deletions, insertions, and/or combinations can be made to arrive at an antibody or antigen-binding fragment thereof that has increased binding affinity for the target. The amino acid changes introduced into the antibody or antigen-binding fragment can also alter or introduce new post-translational modifications into the antibody or antigen-binding fragment, such as changing (e.g., increasing or decreasing) the number of glycosylation sites, changing the type of glycosylation site (e.g., changing the amino acid sequence such that a different sugar is attached by enzymes present in a cell), or introducing new glycosylation sites.

Antibodies disclosed herein can be derived from any species of animal, including mammals. Non-limiting examples of native antibodies include antibodies derived from humans, primates, e.g., monkeys and apes, cows, pigs, horses, sheep, camelids (e.g., camels and llamas), chicken, goats, and rodents (e.g., rats, mice, hamsters and rabbits), including transgenic rodents genetically engineered to produce human antibodies.

Phage display (panning) can be used to optimize antibody sequences with desired binding affinities. In this technique, a gene encoding single chain Fv (comprising VH or VL) can be inserted into a phage coat protein gene, causing the phage to “display” the scFv on its outside while containing the gene for the protein on its inside, resulting in a connection between genotype and phenotype. These displaying phages can then be screened against target antigens, in order to detect interaction between the displayed antigen binding sites and the target antigen. Thus, large libraries of proteins can be screened and amplified in a process called in vitro selection, and antibodies sequences with desired binding affinities can be obtained.

Human and humanized antibodies include antibodies having variable and constant regions derived from (or having the same amino acid sequence as those derived from) human germline immunoglobulin sequences. Human antibodies may include amino acid residues not encoded by human germline immunoglobulin sequences (e.g., mutations introduced by random or site-specific mutagenesis in vitro or by somatic mutation in vivo), for example in the CDRs.

A humanized antibody, typically has a human framework (FR) grafted with non-human CDRs. Thus, a humanized antibody has one or more amino acid sequence introduced into it from a source which is non-human. These non-human amino acid residues are often referred to as “import” residues, which are typically taken from an “import” variable domain. Humanization can be essentially performed by e.g., substituting rodent CDRs or CDR sequences for the corresponding sequences of a human antibody. These methods are described in e.g., Jones et al., Nature, 321:522-525 (1986); Riechmann et al., Nature, 332:323-327 (1988); Verhoeyen et al., Science, 239:1534-1536 (1988); each of which is incorporated by reference herein in its entirety. Accordingly, “humanized” antibodies are chimeric antibodies wherein substantially less than an intact human V domain has been substituted by the corresponding sequence from a non-human species. In practice, humanized antibodies are typically mouse antibodies in which some CDR residues and some FR residues are substituted by residues from analogous sites in human antibodies.

It is further important that antibodies be humanized with retention of high specificity and affinity for the antigen and other favorable biological properties. To achieve this goal, humanized antibodies can be prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved.

Identity or homology with respect to an original sequence is usually the percentage of amino acid residues present within the candidate sequence that are identical with a sequence present within the human, humanized, or chimeric antibody or fragment, after aligning the sequences and introducing gaps, if necessary, to achieve the maximum percent sequence identity, and not considering any conservative substitutions as part of the sequence identity.

In some embodiments, a covalent modification can be made to the antibodies, the antigen-binding fragments thereof, or the antigen-binding protein constructs (e.g., bispecific antibodies). These covalent modifications can be made by chemical or enzymatic synthesis, or by enzymatic or chemical cleavage. Other types of covalent modifications of the antibody or antibody fragment are introduced into the molecule by reacting targeted amino acid residues of the antibody or fragment with an organic derivatization agent that is capable of reacting with selected side chains or the N- or C-terminal residues.

In some embodiments, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues; or position 314 in Kabat numbering); however, Asn297 may also be located about ±3 amino acids upstream or downstream of position 297, i.e., between positions 294 and 300, due to minor sequence variations in antibodies. Such fucosylation variants may have improved ADCC function. In some embodiments, to reduce glycan heterogeneity, the Fc region of the antibody can be further engineered to replace the Asparagine at position 297 with Alanine (N297A).

In some embodiments, to facilitate production efficiency by avoiding Fab-arm exchange, the Fc region of the antibodies was further engineered to replace the serine at position 228 (EU numbering) of IgG4 with proline (S228P). A detailed description regarding S228 mutation is described, e.g., in Silva et al. “The S228P mutation prevents in vivo and in vitro IgG4 Fab-arm exchange as demonstrated using a combination of novel quantitative immunoassays and physiological matrix preparation.” Journal of Biological Chemistry 290.9 (2015): 5462-5469, which is incorporated by reference in its entirety.

In some embodiments, the methods described here are designed to make a bispecific antibody. Bispecific antibodies can be made by engineering the interface between a pair of antibody molecules to maximize the percentage of heterodimers that are recovered from recombinant cell culture. For example, the interface can contain at least a part of the CH3 domain of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers. This method is described, e.g., in WO 96/27011, which is incorporated by reference in its entirety.

In some embodiments, knobs-into-holes (KIH) technology can be used, which involves engineering CH3 domains to create either a “knob” or a “hole” in each heavy chain to promote heterodimerization. The KIH technique is described e.g., in Xu, Yiren, et al. “Production of bispecific antibodies in ‘knobs-into-holes’ using a cell-free expression system.” MAbs. Vol. 7. No. 1. Taylor & Francis, 2015, which is incorporated by reference in its entirety. In some embodiments, one heavy chain has a T366W, and/or S354C (knob) substitution (EU numbering), and the other heavy chain has an Y349C, T366S, L368A, and/or Y407V (hole) substitution (EU numbering). In some embodiments, one heavy chain has one or more of the following substitutions Y349C and T366W (EU numbering). The other heavy chain can have one or more the following substitutions E356C, T366S, L368A, and Y407V (EU numbering). Furthermore, a substitution (-ppcpScp-->-ppcpPcp-) can also be introduced at the hinge regions of both substituted IgG.

Furthermore, an anion-exchange chromatography can be used to purify bispecific antibodies. Anion-exchange chromatography is a process that separates substances based on their charges using an ion-exchange resin containing positively charged groups, such as diethyl-aminoethyl groups (DEAE). In solution, the resin is coated with positively charged counter-ions (cations). Anion exchange resins will bind to negatively charged molecules, displacing the counter-ion. Anion exchange chromatography can be used to purify proteins based on their isoelectric point (pI). The isoelectric point is defined as the pH at which a protein has no net charge. When the pH>pI, a protein has a net negative charge and when the pH<pI, a protein has a net positive charge. Thus, in some embodiments, different amino acid substitution can be introduced into two heavy chains, so that the pI for the homodimer comprising two Arm A and the pI for the homodimer comprising two Arm B is different. The pI for the bispecific antibody having Arm A and Arm B will be somewhere between the two pIs of the homodimers. Thus, the two homodimers and the bispecific antibody can be released at different pH conditions. The present disclosure shows that a few amino acid residue substitutions can be introduced to the heavy chains to adjust pI.

Thus, in some embodiments, the amino acid residue at Kabat numbering position 83 is lysine, arginine, or histidine. In some embodiments, the amino acid residues at one or more of the positions 1, 6, 43, 81, and 105 (Kabat numbering) is aspartic acid or glutamic acid. In some embodiments, the amino acid residues at one or more of the positions 13 and 105 (Kabat numbering) is aspartic acid or glutamic acid. In some embodiments, the amino acid residues at one or more of the positions 13 and 42 (Kabat numbering) is lysine, arginine, histidine, or glycine.

Bispecific antibodies can also include e.g., cross-linked or “heteroconjugate” antibodies. For example, one of the antibodies in the heteroconjugate can be coupled to avidin and the other to biotin. Heteroconjugate antibodies can also be made using any convenient cross-linking methods. Suitable cross-linking agents and cross-linking techniques are well known in the art and are disclosed in U.S. Pat. No. 4,676,980, which is incorporated herein by reference in its entirety.

Methods for generating bispecific antibodies from antibody fragments are also known in the art. For example, bispecific antibodies can be prepared using chemical linkage. Brennan et al. (Science 229:81, 1985) describes a procedure where intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′ TNB derivatives is then reconverted to the Fab′ thiol by reduction with mercaptoethylamine, and is mixed with an equimolar amount of another Fab′ TNB derivative to form the bispecific antibody.

### Recombinant Vectors

The present disclosure also provides recombinant vectors (e.g., expression vectors) that include an isolated polynucleotide disclosed herein (e.g., a polynucleotide that encodes a polypeptide disclosed herein), host cells into which are introduced the recombinant vectors (i.e., such that the host cells contain the polynucleotide and/or a vector comprising the polynucleotide), and the production of recombinant antibody polypeptides or fragments thereof by recombinant techniques.

As used herein, a “vector” is any construct capable of delivering one or more polynucleotide(s) of interest to a host cell when the vector is introduced to the host cell. An “expression vector” is capable of delivering and expressing the one or more polynucleotide(s) of interest as an encoded polypeptide in a host cell into which the expression vector has been introduced. Thus, in an expression vector, the polynucleotide of interest is positioned for expression in the vector by being operably linked with regulatory elements such as a promoter, enhancer, and/or a poly-A tail, either within the vector or in the genome of the host cell at or near or flanking the integration site of the polynucleotide of interest such that the polynucleotide of interest will be translated in the host cell introduced with the expression vector.

A vector can be introduced into the host cell by methods known in the art, e.g., electroporation, chemical transfection (e.g., DEAE-dextran), transformation, transfection, and infection and/or transduction (e.g., with recombinant virus). Thus, non-limiting examples of vectors include viral vectors (which can be used to generate recombinant virus), naked DNA or RNA, plasmids, cosmids, phage vectors, and DNA or RNA expression vectors associated with cationic condensing agents.

In some implementations, a polynucleotide disclosed herein (e.g., a polynucleotide that encodes a polypeptide disclosed herein) is introduced using a viral expression system (e.g., vaccinia or other pox virus, retrovirus, or adenovirus), which may involve the use of a non-pathogenic (defective), replication competent virus, or may use a replication defective virus. In the latter case, viral propagation generally will occur only in complementing virus packaging cells. Suitable systems are disclosed, for example, in Fisher-Hoch et al., 1989, Proc. Natl. Acad. Sci. USA 86:317-321; Flexner et al., 1989, Ann. N.Y. Acad Sci. 569:86-103; Flexner et al., 1990, Vaccine, 8:17-21; U.S. Pat. Nos. 4,603,112, 4,769,330, and 5,017,487; WO 89/01973; U.S. Pat. No. 4,777,127; GB 2,200,651; EP 0,345,242; WO 91/02805; Berkner-Biotechniques, 6:616-627, 1988; Rosenfeld et al., 1991, Science, 252:431-434; Kolls et al., 1994, Proc. Natl. Acad. Sci. USA, 91:215-219; Kass-Eisler et al., 1993, Proc. Natl. Acad. Sci. USA, 90:11498-11502; Guzman et al., 1993, Circulation, 88:2838-2848; and Guzman et al., 1993, Cir. Res., 73:1202-1207. Techniques for incorporating DNA into such expression systems are well known to those of ordinary skill in the art. The DNA may also be “naked,” as described, for example, in Ulmer et al., 1993, Science, 259:1745-1749, and Cohen, 1993, Science, 259:1691-1692. The uptake of naked DNA may be increased by coating the DNA onto biodegradable beads that are efficiently transported into the cells.

For expression, the DNA insert comprising an antibody-encoding or polypeptide-encoding polynucleotide disclosed herein can be operatively linked to an appropriate promoter (e.g., a heterologous promoter), such as the phage lambda PL promoter, the E. coli lac, trp and tac promoters, the SV40 early and late promoters and promoters of retroviral LTRs, to name a few. Other suitable promoters are known to the skilled artisan. The expression constructs can further contain sites for transcription initiation, termination and, in the transcribed region, a ribosome binding site for translation. The coding portion of the mature transcripts expressed by the constructs may include a translation initiating at the beginning and a termination codon (UAA, UGA, or UAG) appropriately positioned at the end of the polypeptide to be translated.

As indicated, the expression vectors can include at least one selectable marker. Such markers include dihydrofolate reductase or neomycin resistance for eukaryotic cell culture and tetracycline or ampicillin resistance genes for culturing in E. coli and other bacteria. Representative examples of appropriate hosts include, but are not limited to, bacterial cells, such as E. coli, Streptomyces, and Salmonella typhimurium cells; fungal cells, such as yeast cells; insect cells such as Drosophila S2 and Spodoptera Sf9 cells; animal cells such as CHO, COS, Bowes melanoma, and HK 293 cells; and plant cells. Appropriate culture mediums and conditions for the host cells described herein are known in the art.

Non-limiting vectors for use in bacteria include pQE70, pQE60 and pQE-9, available from Qiagen; pBS vectors, Phagescript vectors, Bluescript vectors, pNH8A, pNH16a, pNH18A, pNH46A, available from Stratagene; and ptrc99a, pKK223-3, pKK233-3, pDR540, pRIT5 available from Pharmacia. Non-limiting eukaryotic vectors include pWLNEO, pSV2CAT, pOG44, pXT1 and pSG available from Stratagene; and pSVK3, pBPV, pMSG and pSVL available from Pharmacia. Other suitable vectors will be readily apparent to the skilled artisan.

Non-limiting bacterial promoters suitable for use include the E. coli lacI and lacZ promoters, the T3 and T7 promoters, the gpt promoter, the lambda PR and PL promoters and the trp promoter. Suitable eukaryotic promoters include the CMV immediate early promoter, the HSV thymidine kinase promoter, the early and late SV40 promoters, the promoters of retroviral LTRs, such as those of the Rous sarcoma virus (RSV), and metallothionein promoters, such as the mouse metallothionein-I promoter.

In the yeast Saccharomyces cerevisiae, a number of vectors containing constitutive or inducible promoters such as alpha factor, alcohol oxidase, and PGH may be used. For reviews, see Ausubel et al. (1989) Current Protocols in Molecular Biology, John Wiley & Sons, New York, N.Y, and Grant et al., Methods Enzymol., 153: 516-544 (1997).

Introduction of the construct into the host cell can be effected by calcium phosphate transfection, DEAE-dextran mediated transfection, cationic lipid-mediated transfection, electroporation, transduction, infection or other methods. Such methods are described in many standard laboratory manuals, such as Davis et al., Basic Methods In Molecular Biology (1986), which is incorporated herein by reference in its entirety.

Transcription of DNA encoding an antibody of the present disclosure by higher eukaryotes may be increased by inserting an enhancer sequence into the vector. Enhancers are cis-acting elements of DNA, usually about from 10 to 300 bp that act to increase transcriptional activity of a promoter in a given host cell-type. Examples of enhancers include the SV40 enhancer, which is located on the late side of the replication origin at base pairs 100 to 270, the cytomegalovirus early promoter enhancer, the polyoma enhancer on the late side of the replication origin, and adenovirus enhancers.

For secretion of the translated protein into the lumen of the endoplasmic reticulum, into the periplasmic space or into the extracellular environment, appropriate secretion signals may be incorporated into the expressed polypeptide. The signals may be endogenous to the polypeptide or they may be heterologous signals.

The polypeptide (e.g., antibody) can be expressed in a modified form, such as a fusion protein (e.g., a GST-fusion) or with a histidine-tag, and may include not only secretion signals, but also additional heterologous functional regions. For instance, a region of additional amino acids, particularly charged amino acids, may be added to the N-terminus of the polypeptide to improve stability and persistence in the host cell, during purification, or during subsequent handling and storage. Also, peptide moieties can be added to the polypeptide to facilitate purification. Such regions can be removed prior to final preparation of the polypeptide. The addition of peptide moieties to polypeptides to engender secretion or excretion, to improve stability and to facilitate purification, among others, are familiar and routine techniques in the art.

The disclosure also provides a nucleic acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any nucleotide sequence as described herein, and an amino acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any amino acid sequence as described herein.

The disclosure also provides a nucleic acid sequence that has a homology of at least 1%, 2%, 3%, 4%, 5%, 6%, 7%, 8%, 9%, 10%, 15%, 20%, 25%, 30%, 3%, 40%, 45%, 50%, 55%, 60%, 65%, 70%, 75%, 80%, 85%, 90%, 91%, 92%, 93%, 94%, 95%, 96%, 97%, 98%, 99% to any nucleotide sequence as described herein, and an amino acid sequence that has a homology of at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% to any amino acid sequence as described herein.

In some embodiments, the disclosure relates to nucleotide sequences encoding any peptides that are described herein, or any amino acid sequences that are encoded by any nucleotide sequences as described herein. In some embodiments, the nucleic acid sequence is less than 10, 20, 30, 40, 50, 60, 70, 80, 90, 100, 110, 120, 130, 150, 200, 250, 300, 350, 400, 500, or 600 nucleotides. In some embodiments, the amino acid sequence is less than 5, 6, 7, 8, 9, 10, 20, 30, 40, 50, 60, 70, 80, 90, 100, 110, 120, 130, 140, 150, 160, 170, 180, 190, 200, 250, 300, 350, or 400 amino acid residues.

In some embodiments, the amino acid sequence (i) comprises an amino acid sequence; or (ii) consists of an amino acid sequence, wherein the amino acid sequence is any one of the sequences as described herein.

In some embodiments, the nucleic acid sequence (i) comprises a nucleic acid sequence; or (ii) consists of a nucleic acid sequence, wherein the nucleic acid sequence is any one of the sequences as described herein.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences. For example, the comparison of sequences and determination of percent identity between two sequences can be accomplished using a Blossum 62 scoring matrix with a gap penalty of 12, a gap extend penalty of 4, and a frameshift gap penalty of 5.

The percentage of sequence homology (e.g., amino acid sequence homology or nucleic acid homology) can also be determined. How to determine percentage of sequence homology is known in the art. In some embodiments, amino acid residues conserved with similar physicochemical properties (percent homology), e.g. leucine and isoleucine, can be used to measure sequence similarity. Families of amino acid residues having similar physicochemical properties have been defined in the art. These families include e.g., amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). The homology percentage, in many cases, is higher than the identity percentage.

The disclosure provides one or more nucleic acid encoding any of the polypeptides as described herein. In some embodiments, the nucleic acid (e.g., cDNA) includes a polynucleotide encoding a polypeptide of a heavy chain as described herein. In some embodiments, the nucleic acid includes a polynucleotide encoding a polypeptide of a light chain as described herein. In some embodiments, the nucleic acid includes a polynucleotide encoding a scFv polypeptide as described herein.

In some embodiments, the vector can have two of the nucleic acids as described herein, wherein the vector encodes the VL region and the VH region that together bind to CTLA4. In some embodiments, a pair of vectors is provided, wherein each vector comprises one of the nucleic acids as described herein, wherein together the pair of vectors encodes the VL region and the VH region that together bind to CTLA4.

In some embodiments, the vector includes two of the nucleic acids as described herein, wherein the vector encodes the VL region and the VH region that together bind to OX40. In some embodiments, a pair of vectors is provided, wherein each vector comprises one of the nucleic acids as described herein, wherein together the pair of vectors encodes the VL region and the VH region that together bind to OX40.

Vectors can also be constructed to express specific antibodies or polypeptides. In some embodiments, a vector can be constructed to co-express anti-CTLA4 antibody light chain (CTLA4-K) and heavy chain (CTLA4-H). In some embodiments, a vector can contain sequences of, from 5′ end to 3′ end, cytomegalovirus promotor (CMV), CTLA4-K, polyadenylation (PolyA), CMV, CTLA4-H, PolyA, simian vacuolating virus 40 terminator (SV40) and glutamine synthetase marker (GS). In some embodiments, a vector can be constructed to co-express anti-OX40 antibody light chain (OX40-K) and anti-OX40 antibody heavy chain (OX40-H). In some embodiments, a vector can contain sequences of, from 5′ end to 3′ end, CMV, OX40-K, PolyA, CMV, OX40-H, SV40 and GS. In some embodiments, a vector can be constructed to express anti-OX40 antibody scFv polypeptide chain. In some embodiments, a first vector expressing antibody heavy chains (e.g., any of the heavy chains described herein) and a second vector expressing antibody light chains (e.g., any of the light chains described herein) are used to co-transfect cells (e.g., CHO cells) to produce the monoclonal antibody or antigen-binding fragment thereof described herein. In some embodiments, a first vector expressing an anti-CTLA4 antibody heavy chain (e.g., any of the anti-CTLA4 antibody heavy chains described herein), a second vector expressing an anti-OX40 antibody heavy chain (e.g., any of the anti-OX40 antibody heavy chains described herein), and a third vector expressing a common light chain (e.g., any of the common light chains described herein) are used to co-transfect cells (e.g., CHO cells) to produce the antigen-binding protein construct described herein (e.g., any of the anti-CTLA4/OX40 bispecific antibodies described herein).

### Methods of Treatment

The methods described herein include methods for the treatment of disorders associated with cancer. Generally, the methods include administering a therapeutically effective amount of engineered antibodies, the antigen-binding fragments thereof, or the antigen-binding protein constructs (e.g., bispecific antibodies) as described herein, to a subject who is in need of, or who has been determined to be in need of, such treatment.

As used in this context, to “treat” means to ameliorate at least one symptom of the disorder associated with cancer. Often, cancer results in death; thus, a treatment can result in an increased life expectancy (e.g., by at least 1, 2, 3, 4, 5, 6, 7, 8, 9, 10, 11, 12 months, or by at least 1, 2, 3, 4, 5, 6, 7, 8, 9, 10 years). Administration of a therapeutically effective amount of an agent described herein for the treatment of a condition associated with cancer will result in decreased number of cancer cells and/or alleviated symptoms.

As used herein, the term “cancer” refers to cells having the capacity for autonomous growth, i.e., an abnormal state or condition characterized by rapidly proliferating cell growth. The term is meant to include all types of cancerous growths or oncogenic processes, metastatic tissues or malignantly transformed cells, tissues, or organs, irrespective of histopathologic type or stage of invasiveness. The term “tumor” as used herein refers to cancerous cells, e.g., a mass of cancerous cells. Cancers that can be treated or diagnosed using the methods described herein include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus. In some embodiments, the agents described herein are designed for treating or diagnosing a carcinoma in a subject. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the cancer is renal carcinoma or melanoma. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation.

In some embodiments, the cancer is a chemotherapy resistant cancer.

In one aspect, the disclosure also provides methods for treating a cancer in a subject, methods of reducing the rate of the increase of volume of a tumor in a subject over time, methods of reducing the risk of developing a metastasis, or methods of reducing the risk of developing an additional metastasis in a subject. In some embodiments, the treatment can halt, slow, retard, or inhibit progression of a cancer. In some embodiments, the treatment can result in the reduction of in the number, severity, and/or duration of one or more symptoms of the cancer in a subject.

In one aspect, the disclosure features methods that include administering a therapeutically effective amount of antibodies, the antigen-binding fragments thereof, or the antigen-binding protein constructs (e.g., bispecific antibodies), or an antibody drug conjugates disclosed herein to a subject in need thereof, e.g., a subject having, or identified or diagnosed as having, a cancer, e.g., breast cancer (e.g., triple-negative breast cancer), carcinoid cancer, cervical cancer, endometrial cancer, glioma, head and neck cancer, liver cancer, lung cancer, small cell lung cancer, lymphoma, melanoma, ovarian cancer, pancreatic cancer, prostate cancer, renal cancer, colorectal cancer, gastric cancer, testicular cancer, thyroid cancer, bladder cancer, urethral cancer, or hematologic malignancy.

As used herein, the terms “subject” and “patient” are used interchangeably throughout the specification and describe an animal, human or non-human, to whom treatment according to the methods of the present invention is provided. Veterinary and non-veterinary applications are contemplated by the present invention. Human patients can be adult humans or juvenile humans (e.g., humans below the age of 18 years old). In addition to humans, patients include but are not limited to mice, rats, hamsters, guinea-pigs, rabbits, ferrets, cats, dogs, and primates. Included are, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

In some embodiments, the cancer is bladder cancer, colon cancer, or pancreas cancer. In some embodiments, the cancer is non-small cell lung cancer (NSCLC), renal cell carcinoma (RCC), urothelial carcinoma or prostate cancer. In some embodiments, the subject has solid tumor or neoplasms. In some embodiments, the subject has hepatocellular carcinoma.

In some embodiments, the compositions and methods disclosed herein can be used for treatment of patients at risk for a cancer. Patients with cancer can be identified with various methods known in the art.

As used herein, by an “effective amount” is meant an amount or dosage sufficient to effect beneficial or desired results including halting, slowing, retarding, or inhibiting progression of a disease, e.g., a cancer. An effective amount will vary depending upon, e.g., an age and a body weight of a subject to which the antibody, antigen binding fragment, antibody-drug conjugates, antibody-encoding polynucleotide, vector comprising the polynucleotide, and/or compositions thereof is to be administered, a severity of symptoms and a route of administration, and thus administration can be determined on an individual basis.

An effective amount can be administered in one or more administrations. By way of example, an effective amount of an antibody, an antigen binding fragment, or an antibody-drug conjugate is an amount sufficient to ameliorate, stop, stabilize, reverse, inhibit, slow and/or delay progression of an autoimmune disease or a cancer in a patient or is an amount sufficient to ameliorate, stop, stabilize, reverse, slow and/or delay proliferation of a cell (e.g., a biopsied cell, any of the cancer cells described herein, or cell line (e.g., a cancer cell line)) in vitro. As is understood in the art, an effective amount of an antibody, antigen binding fragment, or antibody-drug conjugate may vary, depending on, inter alia, patient history as well as other factors such as the type (and/or dosage) of antibody used.

Effective amounts and schedules for administering the antibodies, antibody-encoding polynucleotides, antibody-drug conjugates, and/or compositions disclosed herein may be determined empirically, and making such determinations is within the skill in the art. Those skilled in the art will understand that the dosage that must be administered will vary depending on, for example, the mammal that will receive the antibodies, antibody-encoding polynucleotides, antibody-drug conjugates, and/or compositions disclosed herein, the route of administration, the particular type of antibodies, antibody-encoding polynucleotides, antigen binding fragments, antibody-drug conjugates, and/or compositions disclosed herein used and other drugs being administered to the mammal. Guidance in selecting appropriate doses for antibody or antigen binding fragment can be found in the literature on therapeutic uses of antibodies and antigen binding fragments, e.g., Handbook of Monoclonal Antibodies, Ferrone et al., eds., Noges Publications, Park Ridge, N.J., 1985, ch. 22 and pp. 303-357; Smith et al., Antibodies in Human Diagnosis and Therapy, Haber et al., eds., Raven Press, New York, 1977, pp. 365-389.

A typical daily dosage of an effective amount of an antibody, the antigen-binding fragment thereof, or the antigen-binding protein construct (e.g., a bispecific antibody) is 0.01 mg/kg to 100 mg/kg. In some embodiments, the dosage can be less than 100 mg/kg, 30 mg/kg, 20 mg/kg, 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, or 0.1 mg/kg. In some embodiments, the dosage can be greater than 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, 0.1 mg/kg, 0.05 mg/kg, or 0.01 mg/kg. In some embodiments, the dosage is about or at least 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.9 mg/kg, 0.8 mg/kg, 0.7 mg/kg, 0.6 mg/kg, 0.5 mg/kg, 0.4 mg/kg, 0.3 mg/kg, 0.2 mg/kg, or 0.1 mg/kg.

In any of the methods described herein, the at least one antibody, the antigen-binding fragment thereof, or the antigen-binding protein construct (e.g., a bispecific antibody), antibody-drug conjugates, or pharmaceutical composition (e.g., any of the antibodies, antigen-binding fragments, antibody-drug conjugates, or pharmaceutical compositions described herein) and, optionally, at least one additional therapeutic agent can be administered to the subject at least once a week (e.g., once a week, twice a week, three times a week, four times a week, once a day, twice a day, or three times a day). In some embodiments, at least two different antibodies and/or antigen-binding fragments are administered in the same composition (e.g., a liquid composition). In some embodiments, at least one antibody, the antigen-binding fragment thereof, the antigen-binding protein construct (e.g., a bispecific antibody), or antibody-drug conjugate, and at least one additional therapeutic agent are administered in the same composition (e.g., a liquid composition). In some embodiments, the at least one antibody or antigen-binding fragment and the at least one additional therapeutic agent are administered in two different compositions (e.g., a liquid composition containing at least one antibody or antigen-binding fragment and a solid oral composition containing at least one additional therapeutic agent). In some embodiments, the at least one additional therapeutic agent is administered as a pill, tablet, or capsule. In some embodiments, the at least one additional therapeutic agent is administered in a sustained-release oral formulation.

In some embodiments, the one or more additional therapeutic agents can be administered to the subject prior to, or after administering the at least one antibody, antigen-binding antibody fragment, antibody-drug conjugate, or pharmaceutical composition (e.g., any of the antibodies, antigen-binding antibody fragments, or pharmaceutical compositions described herein). In some embodiments, the one or more additional therapeutic agents and the at least one antibody, antigen-binding antibody fragment, antibody-drug conjugate, or pharmaceutical composition (e.g., any of the antibodies, antigen-binding antibody fragments, or pharmaceutical compositions described herein) are administered to the subject such that there is an overlap in the bioactive period of the one or more additional therapeutic agents and the at least one antibody or antigen-binding fragment (e.g., any of the antibodies or antigen-binding fragments described herein) in the subject.

In some embodiments, the subject can be administered the at least one antibody, antigen-binding antibody fragment, antibody-drug conjugate, or pharmaceutical composition (e.g., any of the antibodies, antigen-binding antibody fragments, or pharmaceutical compositions described herein) over an extended period of time (e.g., over a period of at least 1 week, 2 weeks, 3 weeks, 1 month, 2 months, 3 months, 4 months, 5 months, 6 months, 7 months, 8 months, 9 months, 10 months, 11 months, 12 months, 1 year, 2 years, 3 years, 4 years, or 5 years). A skilled medical professional may determine the length of the treatment period using any of the methods described herein for diagnosing or following the effectiveness of treatment (e.g., the observation of at least one symptom of cancer). As described herein, a skilled medical professional can also change the identity and number (e.g., increase or decrease) of antibodies or antigen-binding antibody fragments, antibody-drug conjugates (and/or one or more additional therapeutic agents) administered to the subject and can also adjust (e.g., increase or decrease) the dosage or frequency of administration of at least one antibody or antigen-binding antibody fragment (and/or one or more additional therapeutic agents) to the subject based on an assessment of the effectiveness of the treatment (e.g., using any of the methods described herein and known in the art).

In some embodiments, one or more additional therapeutic agents can be administered to the subject. The additional therapeutic agent can comprise one or more inhibitors selected from the group consisting of an inhibitor of B-Raf, an EGFR inhibitor, an inhibitor of a MEK, an inhibitor of ERK, an inhibitor of K-Ras, an inhibitor of c-Met, an inhibitor of anaplastic lymphoma kinase (ALK), an inhibitor of a phosphatidylinositol 3-kinase (PI3K), an inhibitor of an Akt, an inhibitor of mTOR, a dual PI3K/mTOR inhibitor, an inhibitor of Bruton's tyrosine kinase (BTK), and an inhibitor of Isocitrate dehydrogenase 1 (IDH1) and/or Isocitrate dehydrogenase 2 (IDH2). In some embodiments, the additional therapeutic agent is an inhibitor of indoleamine 2,3-dioxygenase-1) (IDO1) (e.g., epacadostat).

In some embodiments, the additional therapeutic agent can comprise one or more inhibitors selected from the group consisting of an inhibitor of HER3, an inhibitor of LSD1, an inhibitor of MDM2, an inhibitor of BCL2, an inhibitor of CHK1, an inhibitor of activated hedgehog signaling pathway, and an agent that selectively degrades the estrogen receptor.

In some embodiments, the additional therapeutic agent can comprise one or more therapeutic agents selected from the group consisting of Trabectedin, nab-paclitaxel, Trebananib, Pazopanib, Cediranib, Palbociclib, everolimus, fluoropyrimidine, IFL, regorafenib, Reolysin, Alimta, Zykadia, Sutent, temsirolimus, axitinib, everolimus, sorafenib, Votrient, Pazopanib, IMA-901, AGS-003, cabozantinib, Vinflunine, an Hsp90 inhibitor, Ad-GM-CSF, Temazolomide, IL-2, IFNa, vinblastine, Thalomid, dacarbazine, cyclophosphamide, lenalidomide, azacytidine, lenalidomide, bortezomid, amrubicine, carfilzomib, pralatrexate, and enzastaurin.

In some embodiments, the additional therapeutic agent can comprise one or more therapeutic agents selected from the group consisting of an adjuvant, a TLR agonist, tumor necrosis factor (TNF) alpha, IL-1, HMGB1, an IL-10 antagonist, an IL-4 antagonist, an IL-13 antagonist, an IL-17 antagonist, an HVEM antagonist, an ICOS agonist, a treatment targeting CX3CL1, a treatment targeting CXCL9, a treatment targeting CXCL10, a treatment targeting CCL5, an LFA-1 agonist, an ICAM1 agonist, and a Selectin agonist.

In some embodiments, carboplatin, nab-paclitaxel, paclitaxel, cisplatin, pemetrexed, gemcitabine, FOLFOX, or FOLFIRI are administered to the subject.

In some embodiments, the additional therapeutic agent is an anti-PD-1 antibody, an anti-PD-L1 antibody, an anti-LAG-3 antibody, an anti-TIGIT antibody, an anti-BTLA antibody, an anti-CD40 antibody, an anti-PD1/CD40 bispecific antibody, or an anti-GITR antibody.

### Pharmaceutical Compositions and Routes of Administration

Also provided herein are pharmaceutical compositions that contain at least one (e.g., one, two, three, or four) of the antigen-binding protein constructs, antibodies (e.g., bispecific antibodies), antigen-binding fragments, or antibody-drug conjugates described herein. Two or more (e.g., two, three, or four) of any of the antigen-binding protein constructs, antibodies, antigen-binding fragments, or antibody-drug conjugates described herein can be present in a pharmaceutical composition in any combination. The pharmaceutical compositions may be formulated in any manner known in the art.

Pharmaceutical compositions are formulated to be compatible with their intended route of administration (e.g., intravenous, intraarterial, intramuscular, intradermal, subcutaneous, or intraperitoneal). The compositions can include a sterile diluent (e.g., sterile water or saline), a fixed oil, polyethylene glycol, glycerine, propylene glycol or other synthetic solvents, antibacterial or antifungal agents, such as benzyl alcohol or methyl parabens, chlorobutanol, phenol, ascorbic acid, thimerosal, and the like, antioxidants, such as ascorbic acid or sodium bisulfite, chelating agents, such as ethylenediaminetetraacetic acid, buffers, such as acetates, citrates, or phosphates, and isotonic agents, such as sugars (e.g., dextrose), polyalcohols (e.g., mannitol or sorbitol), or salts (e.g., sodium chloride), or any combination thereof. Liposomal suspensions can also be used as pharmaceutically acceptable carriers (see, e.g., U.S. Pat. No. 4,522,811). Preparations of the compositions can be formulated and enclosed in ampules, disposable syringes, or multiple dose vials. Where required (as in, for example, injectable formulations), proper fluidity can be maintained by, for example, the use of a coating, such as lecithin, or a surfactant. Absorption of the antibody or antigen-binding fragment thereof can be prolonged by including an agent that delays absorption (e.g., aluminum monostearate and gelatin). Alternatively, controlled release can be achieved by implants and microencapsulated delivery systems, which can include biodegradable, biocompatible polymers (e.g., ethylene vinyl acetate, polyanhydrides, polyglycolic acid, collagen, polyorthoesters, and polylactic acid; Alza Corporation and Nova Pharmaceutical, Inc.).

Compositions containing one or more of any of the antigen-binding protein constructs, antibodies, antigen-binding fragments, antibody-drug conjugates described herein can be formulated for parenteral (e.g., intravenous, intraarterial, intramuscular, intradermal, subcutaneous, or intraperitoneal) administration in dosage unit form (i.e., physically discrete units containing a predetermined quantity of active compound for ease of administration and uniformity of dosage).

Toxicity and therapeutic efficacy of compositions can be determined by standard pharmaceutical procedures in cell cultures or experimental animals (e.g., monkeys). One can determine the LD50 (the dose lethal to 50% of the population) and the ED50 (the dose therapeutically effective in 50% of the population): the therapeutic index being the ratio of LD50:ED50. Agents that exhibit high therapeutic indices are preferred. Where an agent exhibits an undesirable side effect, care should be taken to minimize potential damage (i.e., reduce unwanted side effects). Toxicity and therapeutic efficacy can be determined by other standard pharmaceutical procedures.

Data obtained from cell culture assays and animal studies can be used in formulating an appropriate dosage of any given agent for use in a subject (e.g., a human). A therapeutically effective amount of the one or more (e.g., one, two, three, or four) antigen-binding protein constructs, antibodies or antigen-binding fragments thereof (e.g., any of the antibodies or antibody fragments described herein) will be an amount that treats the disease in a subject (e.g., kills cancer cells) in a subject (e.g., a human subject identified as having cancer), or a subject identified as being at risk of developing the disease (e.g., a subject who has previously developed cancer but now has been cured), decreases the severity, frequency, and/or duration of one or more symptoms of a disease in a subject (e.g., a human). The effectiveness and dosing of any of the antigen-binding protein constructs, antibodies or antigen-binding fragments described herein can be determined by a health care professional or veterinary professional using methods known in the art, as well as by the observation of one or more symptoms of disease in a subject (e.g., a human). Certain factors may influence the dosage and timing required to effectively treat a subject (e.g., the severity of the disease or disorder, previous treatments, the general health and/or age of the subject, and the presence of other diseases).

Exemplary doses include milligram or microgram amounts of any of the antigen-binding protein constructs, antibodies or antigen-binding fragments, or antibody-drug conjugates described herein per kilogram of the subject's weight (e.g., about 1 μg/kg to about 500 mg/kg; about 100 μg/kg to about 500 mg/kg; about 100 μg/kg to about 50 mg/kg; about 10 μg/kg to about 5 mg/kg; about 10 μg/kg to about 0.5 mg/kg; or about 0.1 mg/kg to about 0.5 mg/kg). While these doses cover a broad range, one of ordinary skill in the art will understand that therapeutic agents, including antigen-binding protein constructs, antibodies and antigen-binding fragments thereof, vary in their potency, and effective amounts can be determined by methods known in the art. Typically, relatively low doses are administered at first, and the attending health care professional or veterinary professional (in the case of therapeutic application) or a researcher (when still working at the development stage) can subsequently and gradually increase the dose until an appropriate response is obtained. In addition, it is understood that the specific dose level for any particular subject will depend upon a variety of factors including the activity of the specific compound employed, the age, body weight, general health, gender, and diet of the subject, the time of administration, the route of administration, the rate of excretion, and the half-life of the antibody or antibody fragment in vivo.

The pharmaceutical compositions can be included in a container, pack, or dispenser together with instructions for administration. The disclosure also provides methods of manufacturing the antibodies or antigen binding fragments thereof, or antibody-drug conjugates for various uses as described herein.

### EXAMPLES

The invention is further described in the following examples, which do not limit the scope of the invention described in the claims.

### Example 1. Preparation of Anti-CTLA4 Antibodies

### Generating Anti-CTLA4 Antibodies in RenLite® Mice

Human CTLA4 protein having a His-Tag (hCTLA4-His, Beijing ACROBiosystems Co., LTD., Cat #: CT4-H5229) or a DNA fragment encoding the protein was emulsified with adjuvant to immunize RenLite® mice (obtained from Biocytogen Pharmaceuticals (Beijing) Co., Ltd.). The RenLite® mouse is a genetically-engineered model with complete humanization in the variable region of the heavy chains, while maintaining a fully-humanized common light chain strategically engineered into the antibody gene. Details of RenLite® mice can be found, e.g., in PCT/CN2021/097652, which is incorporated herein by reference in its entirety. Orbital blood collected before immunization was used as a negative control. The mice were immunized for a total of 4 times: complete Freund's adjuvant (CFA) was used for the first immunization, and incomplete Freund's adjuvant (IFA) was used for the second to fourth immunization. The interval between the first and second immunization was two weeks, and the interval between other immunizations was 1 week. Orbital blood was collected 1 week after the four immunization, and analyzed for antibody titer using ELISA. 1 week later, mice with high titers were selected and injected with hCTLA4-His via tail vein. The mice were also injected (by intraperitoneal administration) with CHO-S-hCTLA4 (transfected CHO-S cells expressing human CTLA4 protein with amino acid sequence set forth in SEQ ID NO: 109) for pulse immunization.

Antigen-specific immune cells were isolated from immunized mice. Further, anti-CTLA4 antibodies or the variable region sequences of the antibody heavy and light chains were obtained. For example, single-cell technologies (e.g., using Beacon® Optofluidic System, Berkeley Lights Inc.) can be used to screen and identify plasma cells that secrete antigen-specific monoclonal antibodies. Then, reverse transcription and PCR-based sequencing were used to obtain antibody variable region sequences. According to the sequences, antibodies were expressed and FACS (fluorescence-activated cell sorting) was used to determine the binding specificity of the expressed anti-CTLA4 antibodies. These antibodies included essentially identical light chain variable domains, and exemplary antibodies obtained by this method include: CT-3D2, CT-4E12, CT-5E3 and CT-5F9.

The antibodies were named following the rules below. For example, the heavy chain variable region (VH) and light chain variable region (VL) of CT-3D2 were linked to the IgG1 constant regions, and the obtained antibody was named CT-3D2-IgG1. Similarly, CT-3D2-IgG2 and CT-3D2-IgG4 were generated, which included the same VH and VL sequences of CT-3D2-IgG1, but the constant regions were from the IgG2 and IgG4 subtypes, respectively. The constant regions can also include mutations. For example, LALA mutations (L234A and L235A according to EU numbering) or SI mutations (S239D and 1332E according to EU numbering) were introduced into the Fc region of CT-3D2-IgG1, generating CT-3D2-IgG1-LALA or CT-3D2-IgG1-SI, respectively. Antibodies with different constant region sequences were also produced for CT-4E12, CT-5E3 and CT-5F9.

The heavy chain CDR1, CDR2, and CDR3 amino acid sequences for CT-3D2 are shown in SEQ ID NOs: 1-3 (Kabat numbering) or SEQ ID NOs: 13-15 (Chothia numbering). The antibody's human heavy chain variable region is shown in SEQ ID NO: 25.

The heavy chain CDR1, CDR2, and CDR3 amino acid sequences for CT-4E12 are shown in SEQ ID NOs: 4-6 (Kabat numbering) or SEQ ID NOs: 16-18 (Chothia numbering). The antibody's human heavy chain variable region is shown in SEQ ID NO: 26.

The heavy chain CDR1, CDR2, and CDR3 amino acid sequences for CT-5E3 are shown in SEQ ID NOs: 7-9 (Kabat numbering) or SEQ ID NOs: 19-21 (Chothia numbering). The antibody's human heavy chain variable region is shown in SEQ ID NO: 27.

The heavy chain CDR1, CDR2, and CDR3 amino acid sequences for CT-5F9 are shown in SEQ ID NOs: 10-12 (Kabat numbering) or SEQ ID NOs: 22-24 (Chothia numbering). The antibody's human heavy chain variable region is shown in SEQ ID NO: 28.

The light chain CDR1, CDR2, and CDR3 amino acid sequences for these anti-CTLA4 antibodies are shown in SEQ ID NOs: 29-31 (Kabat numbering) or SEQ ID NOs: 32-34 (Chothia numbering). The antibody's human light chain variable region is shown in SEQ ID NO: 35.

### Binding Affinity of Anti-CTLA4 Antibodies to Human CTLA4

The binding affinity of the anti-CTLA4 antibodies to human CTLA4 (hCTLA4) were measured using surface plasmon resonance (SPR) using Biacore™ (Biacore, Inc., Piscataway N.J.) 8K biosensor equipped with pre-immobilized Protein A sensor chips.

hCTLA4-His were diluted to 1 μg/ml and then injected into the Biacore™ 8K biosensor at 10 μL/min for about 50 seconds to achieve a desired protein density (e.g., about 50 response units (RU)). Purified anti-CTLA4 antibodies at concentrations of 200, 100, 50, 25, 6.25 or 1.56 nM were then injected at 30 μL/min for 120 seconds. Dissociation was monitored for 600 seconds. The chip was regenerated after the last injection of each titration with Glycine (pH 2.0, 30 μL/min for 30 seconds).

Kinetic association rates (kon) and dissociation rates (koff) were obtained simultaneously by fitting the data globally to a 1:1 Langmuir binding model (Karlsson, R. Roos, H. Fagerstam, L. Petersson, B., 1994. Methods Enzymology 6. 99-110) using Biacore™ 8K Evaluation Software 3.0. Affinities were deduced from the quotient of the kinetic rate constants (KD=koff/kon).

As a person of ordinary skill in the art would understand, the same method with appropriate adjustments for parameters (e.g., antibody concentration) was performed for each tested antibody. The results for the tested antibodies are summarized in the table below.

The results showed that the tested anti-CTLA4 antibodies (CT-3D2-IgG1, CT-4E12-IgG1, CT-5E3-IgG1, and CT-5F9-IgG1) exhibited a high binding affinity to human CTLA4.

TABLE 1

| Capture 1 | Analyte 1 | | | |
|---|---|---|---|---|
| Solution | Solution | kon (1/Ms) | koff (1/s) | KD (M) |
| CT-3D2-IgG1 | hCTLA4-His | 4.62E+04 | 7.90E−04 | 1.71E−08 |
| CT-4E12-IgG1 | hCTLA4-His | 2.48E+04 | 4.22E−04 | 1.70E−08 |
| CT-5E3-IgG1 | hCTLA4-His | 3.09E+04 | 6.74E−04 | 2.18E−08 |
| CT-5F9-IgG1 | hCTLA4-His | 1.64E+05 | 1.23E−03 | 7.48E−09 |

### Example 2. Preparation of Anti-OX40 Antibodies

### Generating Anti-OX40 Antibodies in RenLite® Mice

Human OX40 protein having a His-Tag (hOX40-His, Beijing ACROBiosystems CO.LTD., Cat #: OX0-H5224) or a DNA fragment encoding the protein was emulsified with adjuvant to immunize RenLite® mice. Orbital blood collected before immunization was used as a negative control. The mice were immunized for a total of 4 times: complete Freund's adjuvant (CFA) was used for the first immunization, and incomplete Freund's adjuvant (IFA) was used for the second to fourth immunization. The interval between the first and second immunization was two weeks, and the interval between other immunizations was 1 week. Orbital blood was collected 1 week after the four immunization, and the serum titer of human OX40 was detected by ELISA. 1 week later, mice with high titers were selected and injected with hOX40-His via tail vein. The mice were also injected (by intraperitoneal administration) with CHO-S-hOX40 (transfected CHO-S cells expressing human OX40 protein with amino acid sequence set forth in SEQ ID NO: 110) for pulse immunization.

Antigen-specific immune cells were isolated from immunized mice. Further, anti-OX40 antibodies or the variable region sequences of the antibody heavy and light chains were obtained. For example, single-cell technologies (e.g., using Beacon® Optofluidic System, Berkeley Lights Inc.) can be used to screen and identify plasma cells that secrete antigen-specific monoclonal antibodies. Then, reverse transcription and PCR-based sequencing were used to obtain antibody variable region sequences. According to the sequences, antibodies were expressed and FACS was used to determine the binding specificity of the expressed anti-OX40 antibodies. These antibodies included identical light chain variable domains of the anti-CTLA4 antibodies obtained in Example 1, and exemplary antibodies obtained by this method include: OX-1F9, OX-2C11, OX-2F2, OX-2F5, OX-2G11, OX-4C8, OX-H7, and OX-H8.

The antibodies were named following the rules below. For example, the heavy chain variable region (VH) and light chain variable region (VL) of OX-1F9 were linked to the IgG1 constant regions, and the obtained antibody was named OX-1F9-IgG1. Similarly, OX-1F9-IgG2 and OX-1F9-IgG4 were generated, which included the same VH and VL sequences of OX-1F9-IgG1, but the constant regions were from the IgG2 and IgG4 subtypes, respectively. The constant regions can also include mutations. For example, LALA mutations or SI mutations were introduced into the Fc region of OX-1F9-IgG1, generating OX-1F9-IgG1-LALA or OX-1F9-IgG1-SI, respectively. Antibodies with different constant region sequences were also produced for OX-2C11, OX-2F2, OX-2F5, OX-2G11, OX-4C8, OX-H7, and OX-H8.

The heavy chain CDR1, CDR2, and CDR3 amino acid sequences for OX-1F9 are shown in SEQ ID NOs: 36-38 (Kabat numbering) or SEQ ID NOs: 60-62 (Chothia numbering). The antibody's human heavy chain variable region is shown in SEQ ID NO: 84.

The heavy chain CDR1, CDR2, and CDR3 amino acid sequences for OX-2C11 are shown in SEQ ID NOs: 39-41 (Kabat numbering) or SEQ ID NOs: 63-65 (Chothia numbering). The antibody's human heavy chain variable region is shown in SEQ ID NO: 85.

The heavy chain CDR1, CDR2, and CDR3 amino acid sequences for OX-2F2 are shown in SEQ ID NOs: 42-44 (Kabat numbering) or SEQ ID NOs: 66-68 (Chothia numbering). The antibody's human heavy chain variable region is shown in SEQ ID NO: 86.

The heavy chain CDR1, CDR2, and CDR3 amino acid sequences for OX-2F5 are shown in SEQ ID NOs: 45-47 (Kabat numbering) or SEQ ID NOs: 69-71 (Chothia numbering). The antibody's human heavy chain variable region is shown in SEQ ID NO: 87.

The heavy chain CDR1, CDR2, and CDR3 amino acid sequences for OX-2G11 are shown in SEQ ID NOs: 48-50 (Kabat numbering) or SEQ ID NOs: 72-74 (Chothia numbering). The antibody's human heavy chain variable region is shown in SEQ ID NO: 88.

The heavy chain CDR1, CDR2, and CDR3 amino acid sequences for OX-4C8 are shown in SEQ ID NOs: 51-53 (Kabat numbering) or SEQ ID NOs: 75-77 (Chothia numbering). The antibody's human heavy chain variable region is shown in SEQ ID NO: 89.

The light chain CDR1, CDR2, and CDR3 amino acid sequences for the anti-OX40 antibodies above are shown in SEQ ID NOs: 29-31 (Kabat numbering) or SEQ ID NOs: 32-34 (Chothia numbering). The antibody's human light chain variable region is shown in SEQ ID NO: 35.

The heavy chain CDR1, CDR2, and CDR3 amino acid sequences for monoclonal anti-OX40 antibody OX-H7 are shown in SEQ ID NOs: 54-56 (Kabat numbering) or SEQ ID NOs: 78-80 (Chothia numbering). The antibody's human heavy chain variable region is shown in SEQ ID NO: 90. The light chain CDR1, CDR2, and CDR3 amino acid sequences for OX-H7 are shown in SEQ ID NOs: 149-151 (Kabat numbering) or SEQ ID NOs: 155-157 (Chothia numbering). The antibody's human light chain variable region is shown in SEQ ID NO: 161.

The heavy chain CDR1, CDR2, and CDR3 amino acid sequences for monoclonal anti-OX40 antibody OX-H8 are shown in SEQ ID NOs: 57-59 (Kabat numbering) or SEQ ID NOs: 81-83 (Chothia numbering). The antibody's human heavy chain variable region is shown in SEQ ID NO: 91. The light chain CDR1, CDR2, and CDR3 amino acid sequences for OX-H8 are shown in SEQ ID NOs: 152-154 (Kabat numbering) or SEQ ID NOs: 158-160 (Chothia numbering). The antibody's human light chain variable region is shown in SEQ ID NO: 162.

In addition, the light chain CDR1, CDR2, and CDR3 amino acid sequences for OX-H7 and OX-H8 can be set forth in SEQ ID NOs: 29-31 (Kabat numbering) or SEQ ID NOs: 32-34 (Chothia numbering). The human light chain variable region of OX-H7 and OX-H8 can be set forth in SEQ ID NO: 35.

### Binding Affinity of Anti-OX40 Antibodies to Human OX40

The binding affinity of the anti-OX40 antibodies to human OX40 (hOX40) were measured using surface plasmon resonance (SPR) using Biacore™ (Biacore, Inc., Piscataway N.J.) 8K biosensor equipped with pre-immobilized Protein A sensor chips.

hOX40-His were diluted to 1 μg/ml and then injected into the Biacore™ 8K biosensor at 10 μL/min for about 50 seconds to achieve a desired protein density (e.g., about 50 response units (RU)). Purified anti-OX40 antibodies at concentrations of 200, 100, 50, 25, 6.25 or 1.56 nM were then injected at 30 L/min for 120 seconds. Dissociation was monitored for 600 seconds. The chip was regenerated after the last injection of each titration with Glycine (pH 2.0, 30 L/min for 30 seconds).

Kinetic association rates (kon) and dissociation rates (koff) were obtained simultaneously by fitting the data globally to a 1:1 Langmuir binding model (Karlsson, R. Roos, H. Fagerstam, L. Petersson, B., 1994. Methods Enzymology 6. 99-110) using Biacore™ 8K Evaluation Software 3.0. Affinities were deduced from the quotient of the kinetic rate constants (KD=koff/kon).

As a person of ordinary skill in the art would understand, the same method with appropriate adjustments for parameters (e.g., antibody concentration) was performed for each tested antibody. The results for the tested antibodies are summarized in the table below.

The results show that the tested anti-OX40 antibodies (OX-1F9-IgG1, OX-2C11-IgG1, OX-2F2-IgG1, OX-2F5-IgG1, OX-2G11-IgG1, OX-4C8-IgG1, OX-H7-IgG1 (VH: SEQ ID NO: 90; VL: SEQ ID NO: 161) and OX-H8-IgG1 (VH: SEQ ID NO: 91; VL: SEQ ID NO: 162)) have a high binding affinity with human OX40.

TABLE 2

| Capture 1 | Analyte 1 | | | |
|---|---|---|---|---|
| Solution | Solution | kon (1/Ms) | koff (1/s) | KD (M) |
| OX-1F9-IgG1 | hOX40-His | 3.30E+05 | 7.44E−03 | 2.26E−08 |
| OX-2C11-IgG1 | hOX40-His | 5.44E+04 | 4.12E−04 | 7.59E−09 |
| OX-2F2-IgG1 | hOX40-His | 1.09E+06 | 2.17E−02 | 2.00E−08 |
| OX-2F5-IgG1 | hOX40-His | 7.53E+05 | 3.43E−02 | 4.56E−08 |
| OX-2G11-IgG1 | hOX40-His | 3.08E+06 | 6.64E−03 | 2.16E−09 |
| OX-4C8-IgG1 | hOX40-His | 1.13E+05 | 7.38E−03 | 6.54E−08 |
| OX-H7-IgG1 | hOX40-His | 5.98E+05 | 8.24E−03 | 1.38E−08 |
| OX-H8-IgG1 | hOX40-His | 5.08E+05 | 4.41E−03 | 8.68E−09 |

### Example 3. Anti-CTLA4/OX40 Bispecific Antibody

### Preparation of Anti-CTLA4/OX40 Bispecific Antibody

Different bispecific antibodies can be produced having the heavy chain and light chain sequences derived from the anti-CTLA4 antibodies (e.g., CT-3D2, CT-4E12, CT-5E3, or CT-5F9) and the anti-OX40 antibodies (e.g., OX-1F9, OX-2C11, OX-2F2, OX-2F5, OX-2G11, OX-4C8, OX-H7, or OX-H8) described herein. A first vector expressing an anti-CTLA4 antibody heavy chain, a second vector expressing an anti-OX40 antibody heavy chain, and a third vector expressing a common light chain were constructed. CHO-S cells were co-transfected with the three vectors. After 14 days of culture, cell supernatant was collected and purified by Protein A affinity chromatography.

Many methods can be used to reduce the chance of wrong pairing between the heavy chain and the light chain, and between the two heavy chains of a bispecific antibody. In the Fc region, knobs-into-holes mutations were introduced to the anti-CTLA4 arm heavy chain and the anti-OX40 arm heavy chain. Exemplary bispecific antibodies obtained include: CT-5E3-OX-2C11, OX-4C8-CT-3D2, OX-2C11-CT-3D2, OX-2F2-CT-3D2, CT-5F9-OX-2F2, CT-5F9-OX-2G11, OX-2G11-CT-4E12, CT-5E3-OX-2F2, CT-5E3-OX-2G11, OX-2C11-CT-4E12, OX-2F2-CT-4E12, OX-2G11-CT-3D2, CT-5F9-OX-2C11, OX-1F9-CT-3D2, OX-2F5-CT-3D2, OX-H7-CT-3D2, and OX-H8-CT-3D2.

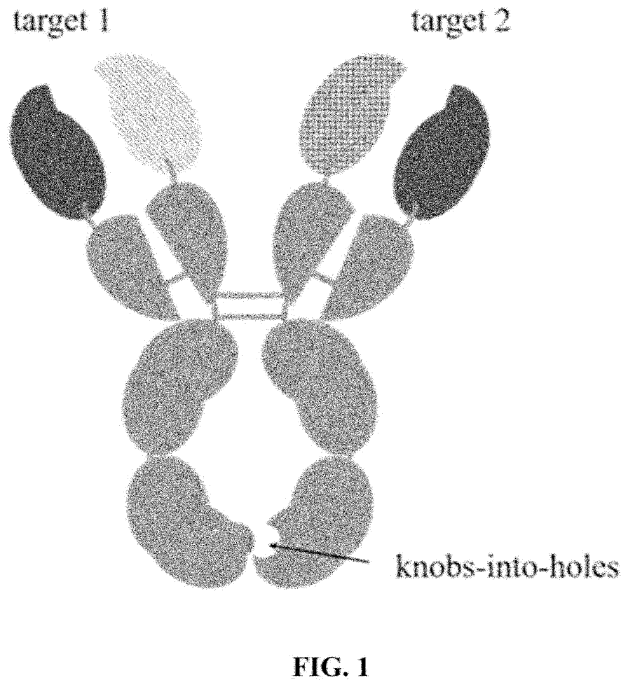

These bispecific antibodies all have the knobs-into-holes structure shown in FIG. 1. For example, in CT-5E3-OX-2C11, the heavy chain constant domain 3 (CH3) of the CT-5E3 arm includes knob mutations, and the CH3 of the OX-2C11 arm includes the corresponding hole mutations; in OX-2C11-CT-5E3, the CH3 of the OX-2C11 arm includes knob mutations, and the CH3 of the CT-5E3 arm includes the corresponding hole mutations.

The bispecific antibodies can also have different IgG subtypes. For example, the VH and VL of CT-5E3-OX-2C11 were linked to the IgG1 constant regions, and the obtained antibody was named CT-5E3-OX-2C11-IgG1. The constant regions can also include mutations. For example, LALA mutations or SI mutations were introduced into the Fc region of CT-5E3-OX-2C11-IgG1, generating CT-5E3-OX-2C11-IgG1-LALA or CT-5E3-OX-2C11-IgG1-SI, respectively.

Sequences of the human IgG (e.g., IgG1, IgG2, and IgG4) heavy chain constant regions, human IgG (e.g., IgG1, IgG2, and IgG4) light chain constant regions, and human IgG (e.g., IgG1 and IgG4) heavy chain constant regions with knob or hole mutations (i.e., knob or hole chains) are listed in SEQ ID NOs: 92-101. SI mutations can also be introduced to the human IgG1 knob and hole chains with amino acid sequences set forth in SEQ ID NOs: 163 and 164, respectively.

Purified anti-CTLA4/OX40 bispecific antibodies were analyzed by SEC-HPLC (Size Exclusion Chromatography-High Performance Liquid Chromatography). In the SEC-HPLC method, the antibody samples were diluted to 1 mg/mL with purified water and an Agilent 1290 chromatograph system (connected with XBridge™ Protein BEH SEC column (200 Å, Waters Corporation)) was used. The following parameters were used: mobile phase: 25 mmol/L phosphate buffer (PB)+300 mmol/L NaCl, pH 6.8; flow rate: 1.8 mL/min; column temperature: 25° C.; detection wavelength: 280 nm; injection volume: 10 μL; sample tray temperature: about 4° C.; and running time: 7 minutes. The results for the tested antibodies are summarized in the table below.

TABLE 3

| Antibody Name | SEC-HPLC (%) |
|---|---|
| OX-1F9-CT-3D2-IgG1-SI | 97.4 |
| OX-2C11-CT-3D2-IgG1-SI | 90.6 |
| OX-2C11-CT-4E12-IgG1-SI | 98.6 |
| OX-2F2-CT-3D2-IgG1-SI | 96.0 |
| OX-2F2-CT-4E12-IgG1-SI | 98.3 |
| OX-2F5-CT-3D2-IgG1-SI | 94.2 |
| OX-2G11-CT-3D2-IgG1-SI | 98.2 |
| OX-2G11-CT-4E12-IgG1-SI | 97.0 |
| OX-H7-CT-3D2-IgG1-SI | 96.2 |
| OX-H8-CT-3D2-IgG1-SI | 98.4 |
| OX-4C8-CT-3D2-IgG1-SI | 94.3 |
| CT-5E3-OX-2C11-IgG1-SI | 95.2 |
| CT-5E3-OX-2F2-IgG1-SI | 94.9 |
| CT-5E3-OX-2G11-IgG1-SI | 96.0 |
| CT-5F9-OX-2C11-IgG1-SI | 97.8 |
| CT-5F9-OX-2F2-IgG1-SI | 96.0 |
| CT-5F9-OX-2G11-IgG1-SI | 98.5 |

### Cross-Species Binding Analyses of Anti-CTLA4/OX40 Bispecific Antibody

The binding affinity of the anti-CTLA4/OX40 bispecific antibodies to hCTLA4-His or fasCTLA4-His (Cynomolgus/Rhesus macaque CTLA-4 Protein, His Tag, Beijing ACROBiosystems CO.LTD., Cat #: CT4-C5227) was measured by surface plasmon resonance (SPR) using Biacore™ (Biacore, Inc., Piscataway N.J.) 8K biosensor equipped with pre-immobilized Protein A sensor chips. The experiment was performed using a similar protocol as described above.

The results for the tested antibodies are summarized in the table below. The results showed that each one of the anti-CTLA4/OX40 bispecific antibodies OX-2C11-CT-4E12-IgG1-SI, CT-5F9-OX-2G11-IgG1-SI, CT-5E3-OX-2F2-IgG1-SI and OX-2F2-CT-3D2-IgG1-SI had a high binding affinity to both human and monkey CTLA4.

TABLE 4

| Capture 1 Solution | Analyte 1 Solution | kon (1/Ms) | koff (1/s) | KD (M) |
|---|---|---|---|---|
| OX-2C11-CT-4E12-IgG1-SI | hCTLA4-His | 2.24E+04 | 2.30E−04 | 1.02E−08 |
| OX-2C11-CT-4E12-IgG1-SI | fasCTLA4-His | 3.78E+04 | 4.22E−04 | 1.12E−08 |
| CT-5F9-OX-2G11-IgG1-SI | hCTLA4-His | 3.30E+04 | 3.10E−03 | 9.40E−08 |
| CT-5F9-OX-2G11-IgG1-SI | fasCTLA4-His | 5.71E+05 | 1.19E−02 | 2.09E−08 |
| CT-5E3-OX-2F2-IgG1-SI | hCTLA4-His | 3.27E+04 | 9.09E−04 | 2.78E−08 |
| CT-5E3-OX-2F2-IgG1-SI | fasCTLA4-His | 1.26E+05 | 1.05E−03 | 8.36E−09 |
| OX-2F2-CT-3D2-IgG1-SI | hCTLA4-His | 5.46E+04 | 7.74E−04 | 1.42E−08 |
| OX-2F2-CT-3D2-IgG1-SI | FasCTLA4-His | 1.28E+05 | 6.12E−04 | 4.77E−09 |

In a similar experiment, the binding affinity of anti-CTLA4/OX40 bispecific antibodies to hOX40-His or fasOX40-His (Cynomolgus/Rhesus macaque OX40/TNFRSF4/CD134 Protein, His Tag, Beijing ACROBiosystems CO.LTD., Cat #: OX0-C5220) was measured. The results showed that each one of OX-2C11-CT-4E12-IgG1-SI, CT-5F9-OX-2G11-IgG1-SI, CT-5E3-OX-2F2-IgG1-SI, OX-4C8-CT-3D2-IgG1-SI, OX-H7-CT-3D2-IgG1-SI, OX-H8-CT-3D2-IgG1-SI, OX-2F5-CT-3D2-IgG1-SI and OX-1F9-CT-3D2-IgG1-SI had a high binding affinity to both human and monkey OX40.

TABLE 5

| Capture 1 Solution | Analyte 1 Solution | kon (1/Ms) | koff (1/s) | KD (M) |
|---|---|---|---|---|
| OX-2C11-CT-4E12-IgG1-SI | hOX40-His | 3.75E+04 | 6.26E−04 | 1.67E−08 |
| OX-2C11-CT-4E12-IgG1-SI | fasOX40-His | 1.85E+04 | 6.85E−04 | 3.70E−08 |
| CT-5F9-OX-2G11-IgG1-SI | hOX40-His | 6.47E+04 | 7.24E−03 | 1.12E−07 |
| CT-5F9-OX-2G11-IgG1-SI | fasOX40-His | 1.37E+04 | 4.89E−03 | 3.57E−07 |
| CT-5E3-OX-2F2-IgG1-SI | hOX40-His | 2.70E+05 | 1.36E−02 | 5.02E−08 |
| CT-5E3-OX-2F2-IgG1-SI | fasOX40-His | 4.18E+04 | 2.05E−04 | 4.91E−09 |
| OX-4C8-CT-3D2-IgG1-SI | hOX40-His | 1.47E+05 | 2.58E−03 | 1.76E−08 |
| OX-4C8-CT-3D2-IgG1-SI | fasOX40-His | 5.78E+05 | 3.70E−03 | 6.40E−09 |
| OX-H7-CT-3D2-IgG1-SI | hOX40-His | 9.26E+03 | 1.69E−03 | 1.82E−07 |
| OX-H7-CT-3D2-IgG1-SI | fasOX40-His | 6.17E+03 | 1.00E−03 | 1.63E−07 |
| OX-H8-CT-3D2-IgG1-SI | hOX40-His | 4.90E+05 | 7.84E−03 | 1.60E−08 |
| OX-H8-CT-3D2-IgG1-SI | fasOX40-His | 5.10E+05 | 9.66E−03 | 1.89E−08 |
| OX-2F5-CT-3D2-IgG1-SI | hOX40-His | 1.36E+05 | 9.47E−03 | 6.96E−08 |
| OX-2F5-CT-3D2-IgG1-SI | fasOX40-His | 8.45E+05 | 2.57E−02 | 3.04E−08 |
| OX-1F9-CT-3D2-IgG1-SI | hOX40-His | 3.06E+05 | 6.98E−03 | 2.28E−08 |
| OX-1F9-CT-3D2-IgG1-SI | fasOX40-His | 1.50E+05 | 3.71E−04 | 2.48E−09 |

### Example 4. In Vivo Testing of Anti-CTLA4 or Anti-OX40 Antibodies

In Vivo Efficacy of 1 mg/kg Anti-CTLA4 Antibodies in hCTLA4 Mice

A hCTLA4 mouse model (obtained from Biocytogen Pharmaceuticals (Beijing) Co., Ltd., Cat #: 110011) was engineered to express a chimeric CTLA4 protein. The chimeric CTLA4 protein (SEQ ID NO: 102) includes a replacement of a portion of mouse CTLA4 extracellular region with the corresponding human CTLA4 extracellular region. The humanized mouse model (hCTLA4 mice) provides a tool for testing new therapeutics in a clinical setting by significantly decreasing the difference between clinical outcome in human and in laboratory mice expressing mouse CTLA4. A detailed description regarding the humanized CTLA4 mouse model can be found in U.S. patent application Ser. No. 16/329,275, which is incorporated herein by reference in its entirety.

The hCTLA4 mice (5-9 weeks) were subcutaneously injected with 5×10⁵ colon adenocarcinoma cell line MC38, and when the tumor volume reached about 100-150 mm³, the mice were placed into a control group and three treatment groups based on tumor size (6 mice per group). The treatment group mice were randomly selected for intraperitoneal (i.p.) administration of anti-CTLA4 antibodies CT-3D2-IgG1 (G2), CT-5E3-IgG1 (G3), or CT-5F9-IgG1 (G4) at a dose level of 1 mg/kg. The control group mice (G1) were injected with an equal volume of phosphate buffered saline (PBS). The frequency of administration was twice a week (4 times of administrations in total). The tumor volume was measured twice a week and the body weight of the mice was weighed as well. Euthanasia was performed when the tumor volume of the mouse reached 3000 mm³. Details of the administration scheme are shown in the table below.

TABLE 6

| | No. of | | Dosage | | | Total No. of |
|---|---|---|---|---|---|---|
| Group | mice | Antibodies | (mg/kg) | Route | Frequency | administration |
| G1 | 6 | PBS | — | i.p. | BIW | 4 |
| G2 | 6 | CT-3D2-IgG1 | 1 | i.p. | BIW | 4 |
| G3 | 6 | CT-5E3-IgG1 | 1 | i.p. | BIW | 4 |
| G4 | 6 | CT-5F9-IgG1 | 1 | i.p. | BIW | 4 |

Body weight of the mice was monitored during the entire treatment period. The weight of mice in different groups all increased, with no statistically significant differences (P>0.05) between groups. The results showed that the anti-CTLA4 antibodies were well tolerated and not toxic to the mice.

The Tumor Growth Inhibition rate based on tumor volume (TGI_{TV}%) on Day 18 (18 days after grouping) for each treatment group was also calculated as shown in the table below. The results indicate that all the tested human anti-hCTLA4 antibodies can inhibit tumor growth. Among them, CT-5F9-IgG1 (G4) had the highest TGI_{TV}%.

TABLE 7

| Group | Antibodies | TGI_{TV} % |
|---|---|---|
| G1 | PBS | — |
| G2 | CT-3D2-IgG1 | 82.2% |
| G3 | CT-5E3-IgG1 | 86.1% |
| G4 | CT-5F9-IgG1 | 96.1% |

In order to verify the in vivo efficacy of CT-4E12, a heavy chain-encoding plasmid CT-4E12-mIgG2c-H (in which the amino acid sequence of mIgG2c is shown in SEQ ID NO: 103) and a light chain-encoding plasmid CT-4E12-L were constructed. Both plasmids included a CAG promotor for heavy or light chain expression.

hCTLA4 humanized mouse (5-9 weeks) were subcutaneously injected with 5×10⁵ colon adenocarcinoma cell line MC38, and when the tumor volume reached about 100 mm³, the mice were placed into a control group and a treatment group based on tumor size (6 mice per group). The treatment group mice were administered with a mixture of 50 μg/mouse CT-4E12-mIgG2c-H and 50 μg/mouse CT-4E12-L by intramuscular (i.m.) injection. The control group mice were injected with an equal volume of phosphate buffered saline (PBS). Only one administration was performed in total. The tumor volume was measured twice a week and the body weight of the mice was weighed as well. Euthanasia was performed when the tumor volume of the mouse reached 3000 mm³.

The weight of the mice was monitored during the entire treatment period. The weight of mice in different groups all increased, with no statistically significant differences (P>0.05) between groups. According to the tumor size measurement results, the TGI_{TV}% rate for the treatment group mice was determined as 44.10%, indicating a good therapeutic effect. The results indicate that the antibody can be expressed from the plasmids in mice. In addition, the expressed antibody was well tolerated and not toxic to the mice.

In Vivo Efficacy of 1 mg/kg Anti-OX40 Antibodies in hOX40 Mice

A hOX40 mouse model (obtained from Biocytogen Pharmaceuticals (Beijing) Co., Ltd., Cat #: 110014) was engineered to express a chimeric OX40 protein. The chimeric OX40 protein (SEQ ID NO: 104) includes a replacement of a portion of mouse OX40 extracellular region with the corresponding human OX40 extracellular region. The humanized mouse model (hOX40 mice) provides a tool for testing new therapeutics in a clinical setting by significantly decreasing the difference between clinical outcome in human and in laboratory mice expressing mouse OX40. A detailed description regarding the humanized OX40 mouse model can be found in U.S. patent application Ser. No. 16/329,269, which is incorporated herein by reference in its entirety.

The hOX40 mice (5-9 weeks) were subcutaneously injected with 5×10⁵ colon adenocarcinoma cell line MC38, and when the tumor volume reached about 100-150 mm³, the mice were placed into a control group and eight treatment groups based on tumor size (6 mice per group). The treatment group mice were randomly selected for intraperitoneal (i.p.) administration of the following anti-OX40 antibodies OX-1F9-IgG1 (G2), OX-2C11-IgG1 (G3), OX-2F2-IgG1 (G4), OX-2F5-IgG1 (G5), OX-2G11-IgG1 (G6), OX-4C8-IgG1 (G7), OX-H7-IgG1 (G8) or OX-H8-IgG1 (G9) at a dose level of 1 mg/kg. The control group mice were injected with an equal volume of PBS (G1). The frequency of administration was twice a week (4 times of administrations in total). The tumor volume was measured twice a week and the body weight of the mice was weighed as well. Euthanasia was performed when the tumor volume of the mouse reached 3000 mm³. Details of the administration scheme are shown in the table below.

TABLE 8

| | No. of | | Dosage | | | Total No. of |
|---|---|---|---|---|---|---|
| Group | mice | Antibodies | (mg/kg) | Route | Frequency | administration |
| G1 | 6 | PBS | — | i.p. | BIW | 4 |
| G2 | 6 | OX-1F9-IgG1 | 1 | i.p. | BIW | 4 |
| G3 | 6 | OX-2C11-IgG1 | 1 | i.p. | BIW | 4 |
| G4 | 6 | OX-2F2-IgG1 | 1 | i.p. | BIW | 4 |
| G5 | 6 | OX-2F5-IgG1 | 1 | i.p. | BIW | 4 |
| G6 | 6 | OX-2G11-IgG1 | 1 | i.p. | BIW | 4 |
| G7 | 6 | OX-4C8-IgG1 | 1 | i.p. | BIW | 4 |
| G8 | 6 | OX-H7-IgG1 | 1 | i.p. | BIW | 4 |
| G9 | 6 | OX-H8-IgG1 | 1 | i.p. | BIW | 4 |

Body weight of the mice was monitored during the entire treatment period. The weight of mice in different groups all increased, with no statistically significant differences (P>0.05) between groups. The results showed that the anti-OX40 antibodies were well tolerated and not toxic to the mice.

The TGI_{TV}%0 on Day 18 (18 days after grouping) for each treatment group was also calculated, which is shown in the table below.

TABLE 9

| Group | Antibodies | TGI_{TV} % |
|---|---|---|
| G1 | PBS | — |
| G2 | OX-1F9-IgG1 | 47.8% |
| G3 | OX-2C11-IgG1 | 74.3% |
| G4 | OX-2F2-IgG1 | 31.2% |
| G5 | OX-2F5-IgG1 | 65.8% |
| G6 | OX-2G11-IgG1 | 75.8% |
| G7 | OX-4C8-IgG1 | 63.4% |
| G8 | OX-H7-IgG1 | 59.4% |
| G9 | OX-H8-IgG1 | 55.7% |

The results indicate that all the tested human anti-hOX40 antibodies can inhibit tumor growth. Among them, OX-2G11-IgG1 (G6) had the highest TGI_{TV}% rate.

### Example 5. In Vivo Testing of Anti-CTLA4/OX40 Antibodies

In Vivo Efficacy of 0.3 mg/kg Bispecific Antibodies in hCTLA4/hOX40 Mice

A hCTLA4/hOX40 mouse model (obtained from Biocytogen Pharmaceuticals (Beijing) Co., Ltd., Cat #: 120531) was engineered to express a chimeric CTLA4 protein and a chimeric OX40 protein. A detailed description regarding the humanized CTLA4 mouse model, the humanized OX40 mouse model, and the double humanized hCTLA4/hOX40 mouse model can be found in PCT Application Nos. PCT/CN2017/099577 and PCT/CN2017/099575, each of which is incorporated herein by reference in its entirety.

The hCTLA4/hOX40 mice (5-9 weeks) were subcutaneously injected with 5×10⁵ colon adenocarcinoma cell line MC38, and when the tumor volume reached about 100-150 mm³, the mice were placed into a control group and five treatment groups based on tumor size (6 mice per group). The treatment group mice were randomly selected for intraperitoneal (i.p.) administration of anti-CTLA4/OX40 bispecific antibody OX-2C11-CT-3D2-IgG1-SI treatment (G2), OX-2F2-CT-3D2-IgG1-SI (G3), CT-5F9-OX-2F2-IgG1-SI (G4), CT-5F9-OX-2G11-IgG1-SI (G5), or OX-2G11-CT-4E12-IgG1-SI (G6) at a dose level of 0.3 mg/kg. The control group mice were injected with an equal volume of PBS (G1). The frequency of administration was twice a week (4 times of administrations in total). The tumor volume was measured twice a week and the body weight of the mice was weighed as well. Euthanasia was performed when the tumor volume of the mouse reached 3000 mm³. Details of the administration scheme are shown in the table below.

TABLE 10

| | No. of | | Dosage | | | Total No. of |
|---|---|---|---|---|---|---|
| Group | mice | Antibodies | (mg/kg) | Route | Frequency | administration |
| G1 | 6 | PBS | — | i.p. | BIW | 4 |
| G2 | 6 | OX-2C11-CT-3D2-IgG1-SI | 0.3 | i.p. | BIW | 4 |
| G3 | 6 | OX-2F2-CT-3D2-IgG1-SI | 0.3 | i.p. | BIW | 4 |
| G4 | 6 | CT-5F9-OX-2F2-IgG1-SI | 0.3 | i.p. | BIW | 4 |
| G5 | 6 | CT-5F9-OX-2G11-IgG1-SI | 0.3 | i.p. | BIW | 4 |
| G6 | 6 | OX-2G11-CT-4E12-IgG1-SI | 0.3 | i.p. | BIW | 4 |

Body weight of the mice was monitored during the entire treatment period. The weight of mice in different groups all increased, with no statistically significant differences (P>0.05) between groups. On the day of grouping (Day 0; or “D0”), the average weight of each group was in a range of 20.2 g-20.5 g. At the end of the experiment (D21; or 21 days after grouping), the average weight of each group was in a range of 22.5 g-24.4 g, and the weight changes was in a range of 111.0%-120.9%. The results showed that the anti-CTLA4/OX40 bispecific antibodies were well tolerated and not toxic to the mice.

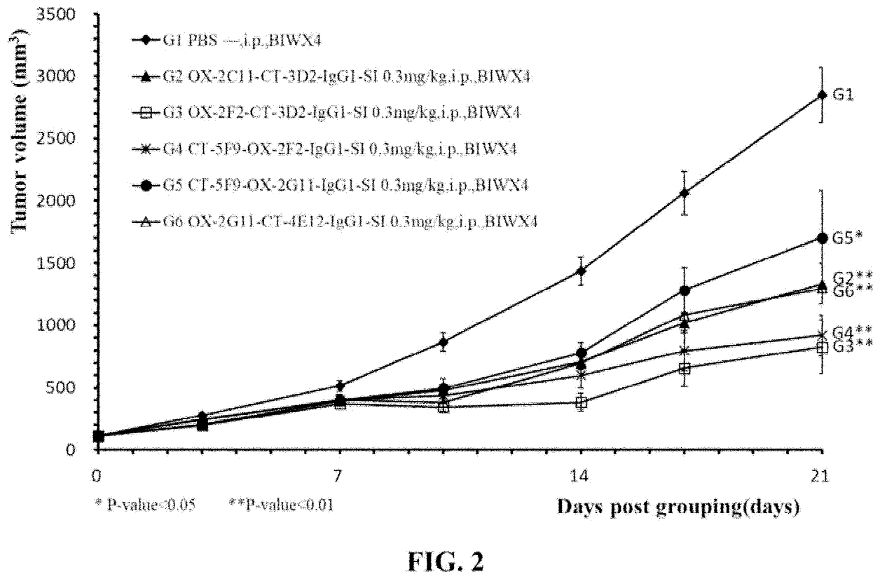

The tumor volumes in groups treated with the bispecific antibodies are shown in FIG. 2. The table below summarizes the results for this experiment, including the tumor volumes on the day of grouping (D0), 10 days after the grouping (D10), and at the end of the experiment (D21); the survival rate of the mice; the Tumor growth inhibition rate (TGI_{TV}%); and the statistical differences (P value) of body weight and tumor volume between the treatment and control groups.

TABLE 11

| | | | | | | | P value | |
|---|---|---|---|---|---|---|---|---|
| | | Tumor volume(mm³) | | | | | Body | Tumor |
| | | D 0 | D 10 | D 21 | Survival | TGI_{TV} % | weight | Volume |
| Control | G1 | 112 ± 3 | 864 ± 73 | 2850 ± 221 | 6/6 | n.a. | n.a. | n.a. |
| Treatment | G2 | 112 ± 3 | 482 ± 29 | 1333 ± 159 | 6/6 | 55.4 | 0.840 | 2.40E−04 |
| | G3 | 112 ± 3 | 343 ± 32 | 827 ± 215 | 6/6 | 73.9 | 0.187 | 6.46E−05 |
| | G4 | 112 ± 3 | 436 ± 54 | 919 ± 162 | 6/6 | 70.5 | 0.628 | 3.55E−05 |
| | G5 | 112 ± 4 | 496 ± 77 | 1704 ± 379 | 6/6 | 41.9 | 0.543 | 0.026 |
| | G6 | 112 ± 5 | 384 ± 41 | 1297 ± 122 | 6/6 | 56.7 | 0.411 | 1.08E−04 |

Throughout the experimental period, the average tumor volume of all treatment group mice (G2-G6) was smaller than that of the control group mice (G1). The results also showed that anti-CTLA4/OX40 bispecific antibodies had different tumor inhibitory effects. Among the tested antibodies, OX-2F2-CT-3D2-IgG1-SI (G3) had the highest tumor growth inhibition rate (TGI_{TV}%).

In Vivo Efficacy of 0.3 mg/kg Bispecific Antibodies in hCTLA4/hOX40 Mice

Similar to the previous experiment, anti-CTLA4/OX40 bispecific antibodies CT-5E3-OX-2F2-IgG1-SI, CT-5E3-OX-2G11-IgG1-SI, OX-2C11-CT-4E12-IgG1-SI and OX-2F2-CT-4E12-IgG1-SI were tested for their in vivo effects on tumor growth in the hCTLA4/hOX40 mouse model that were subcutaneously injected with colon carcinoma cell line MC38. When the tumors in the mice reached a volume of about 100-150 mm³, the mice were randomly placed into different groups (6 mice per group) based on the tumor volume. Details of the administration scheme are shown in the table below.

TABLE 12

| | No. of | | Dosage | | | Total No. of |
|---|---|---|---|---|---|---|
| Group | mice | Antibodies | (mg/kg) | Route | Frequency | administration |
| G1 | 6 | PBS | — | i.p. | BIW | 4 |
| G2 | 6 | CT-5E3-OX-2F2-IgG1-SI | 0.3 | i.p. | BIW | 4 |
| G3 | 6 | CT-5E3-OX-2G11-IgG1-SI | 0.3 | i.p. | BIW | 4 |
| G4 | 6 | OX-2C11-CT-4E12-IgG1-SI | 0.3 | i.p. | BIW | 4 |
| G5 | 6 | OX-2F2-CT-4E12-IgG1-SI | 0.3 | i.p. | BIW | 4 |

Body weight of the mice was monitored throughout the entire experiment. The weight of mice in different groups all increased and there was no significant difference (P>0.05) between groups. At the time of grouping (D0), the average weight of each group was in a range of 20.6 g-21.9 g. At the end of the experiment (D18; or 18 days after grouping), the average weight of each group was in a range of 21.6 g-23.4 g, and the weight change was in a range of 104.9%-111.5%. Similar to the previous experiments, the results showed that the anti-CTLA4/OX40 bispecific antibodies were well tolerated and not toxic to the mice.

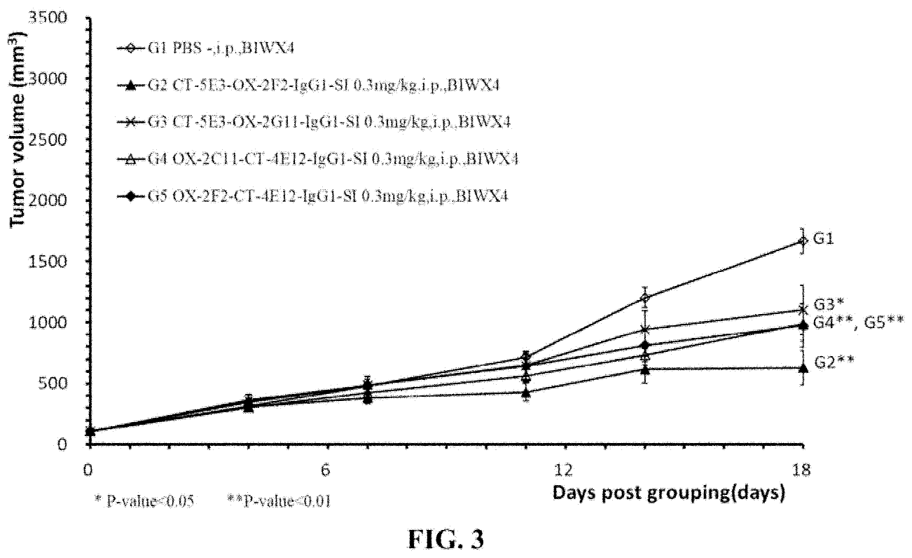

The tumor volumes in groups treated with the bispecific antibodies are shown in FIG. 3. The table below summarizes the results for this experiment, including the tumor volumes on the day of grouping (D0), 11 days after the grouping (D11), and at the end of the experiment (D18); the survival rate of the mice; the Tumor growth inhibition rate (TGI_{TV}%); and the statistical differences (P value) of body weight and tumor volume between the treatment and control groups.

TABLE 13

| | | | | | | | P value | |
|---|---|---|---|---|---|---|---|---|
| | | Tumor volume (mm³) | | | | | Body | Tumor |
| | | D 0 | D 11 | D 18 | Survival | TGI_{TV} % | weight | Volume |
| Control | G1 | 113 ± 3 | 718 ± 15 | 1665 ± 100 | 6/6 | n.a. | n.a. | n.a. |
| Treatment | G2 | 113 ± 3 | 429 ± 76 | 629 ± 139 | 6/6 | 66.7% | 0.041 | 1.21E−04 |
| | G3 | 113 ± 3 | 649 ± 116 | 1101 ± 201 | 6/6 | 36.3% | 0.219 | 0.031 |
| | G4 | 113 ± 4 | 564 ± 51 | 985 ± 136 | 6/6 | 43.8% | 0.952 | 0.002 |
| | G5 | 113 ± 4 | 643 ± 116 | 974 ± 177 | 6/6 | 44.5% | 0.507 | 0.007 |

Throughout the experimental period, the average tumor volume of all treatment group mice (G2-G5) were smaller than that of the control group mice (G1). The results also showed that anti-CTLA4/OX40 bispecific antibodies had different tumor inhibitory effects. Among the tested antibodies, CT-5E3-OX-2F2-IgG1-SI (G2) had the highest tumor growth inhibition rate (TGI_{TV}%).

In Vivo Efficacy of 0.3 mg/kg Bispecific Antibodies in hCTLA4/hOX40 Mice

Similar to the previous experiments, anti-CTLA4/OX40 bispecific antibodies OX-2G11-CT-3D2-IgG1-SI, CT-5F9-OX-2C11-IgG1-SI and CT-5E3-OX-2C11-IgG1-SI were tested for their in vivo effects on tumor growth in the hCTLA4/hOX40 mouse model that were subcutaneously injected with colon carcinoma cell line MC38. When the tumors in the mice reached a volume of about 100-150 mm³, the mice were randomly placed into different groups (6 mice per group) based on the tumor volume. Details of the administration scheme are shown in the table below.

TABLE 14

| | No. of | | Dosage | | | Total No. of |
|---|---|---|---|---|---|---|
| Group | mice | Antibodies | (mg/kg) | Route | Frequency | administration |
| G1 | 6 | PBS | — | i.p. | BIW | 4 |
| G2 | 6 | OX-2G11-CT-3D2-IgG1-SI | 0.3 | i.p. | BIW | 4 |
| G3 | 6 | CT-5F9-OX-2C11-IgG1-SI | 0.3 | i.p. | BIW | 4 |
| G4 | 6 | CT-5E3-OX-2C11-IgG1-SI | 0.3 | i.p. | BIW | 4 |

Body weight of the mice was monitored throughout the entire experiment. The weight of mice in different groups all increased and there was no significant difference (P>0.05) between groups. At the time of grouping (D0), the average weight of each group was in a range of 20.7 g-21.0 g. At the end of the experiment (D21; or 21 days after grouping), the average weight of each group was in a range of 23.2 g-25.6 g, and the weight change was in a range of 112.1%-122.6%. Similar to the previous experiments, the results showed that the anti-CTLA4/OX40 bispecific antibodies were well tolerated and not toxic to the mice.

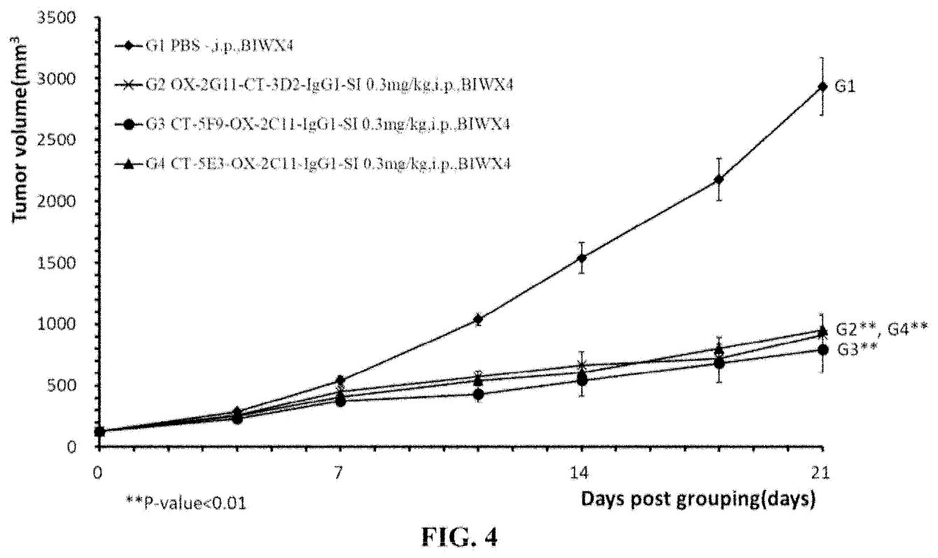

The tumor volumes in groups treated with the bispecific antibodies are shown in FIG. 4. The table below summarizes the results for this experiment, including the tumor volumes on the day of grouping (D0), 11 days after the grouping (D11), and at the end of the experiment (D21); the survival rate of the mice; the Tumor growth inhibition rate (TGI_{TV}%); and the statistical differences (P value) of body weight and tumor volume between the treatment and control groups.

TABLE 15

| | | | | | | | P value | |
|---|---|---|---|---|---|---|---|---|
| | | Tumor volume (mm³) | | | | | Body | Tumor |
| | | D 0 | D 11 | D 21 | Survival | TGI_{TV} % | weight | Volume |
| Control | G1 | 128 ± 3 | 1036 ± 49 | 2937 ± 233 | 6/6 | n.a. | n.a. | n.a. |
| Treatment | G2 | 128 ± 4 | 569 ± 49 | 916 ± 154 | 6/6 | 71.9 | 0.329 | 2.76E−05 |
| | G3 | 128 ± 5 | 431 ± 65 | 793 ± 184 | 6/6 | 76.3 | 0.105 | 2.82E−05 |
| | G4 | 128 ± 5 | 540 ± 33 | 954 ± 133 | 6/6 | 70.6 | 0.240 | 2.32E−05 |

Throughout the experimental period, the average tumor volume of all treatment group mice (G2-G4) was smaller than that of the control group mice (G1). The results also showed that anti-CTLA4/OX40 bispecific antibodies had different tumor inhibitory effects. Among the tested antibodies, CT-5F9-OX-2C11-IgG1-SI (G3) had the highest tumor growth inhibition rate (TGI_{TV}%).

In vivo efficacy of 0.3 mg/kg antibodies in hCTLA4/hOX40 mice Similar to the previous experiments, an analog of the marketed anti-CTLA4 monoclonal antibody Ipilimumab (VH SEQ ID NO: 105; VL SEQ ID NO: 106), and anti-CTLA4/OX40 bispecific antibodies OX-1F9-CT-3D2-IgG1-SI, OX-2F5-CT-3D2-IgG1-SI, OX-H7-CT-3D2-IgG1-SI, OX-H8-CT-3D2-IgG1-SI, and OX-4C8-CT-3D2-IgG1-SI were tested for their in vivo effect on tumor growth in hCTLA4/hOX40 mouse model that were subcutaneously injected with colon carcinoma cell line MC38. When the tumors in the mice reached a volume of about 100-150 mm³, the mice were randomly placed into different groups (6 mice per group) based on the tumor volume. Details of the administration scheme are shown in the table below.

TABLE 16

| | No. of | | Dosage | | | Total No. of |
|---|---|---|---|---|---|---|
| Group | mice | Antibodies | (mg/kg) | Route | Frequency | administration |
| G1 | 6 | PBS | — | i.p. | BIW | 4 |
| G2 | 6 | Ipilimumab analog | 0.3 | i.p. | BIW | 4 |
| G3 | 6 | OX-1F9-CT-3D2-IgG1-SI | 0.3 | i.p. | BIW | 4 |
| G4 | 6 | OX-2F5-CT-3D2-IgG1-SI | 0.3 | i.p. | BIW | 4 |
| G5 | 6 | OX-H7-CT-3D2-IgG1-SI | 0.3 | i.p. | BIW | 4 |
| G6 | 6 | OX-H8-CT-3D2-IgG1-SI | 0.3 | i.p. | BIW | 4 |
| G7 | 6 | OX-4C8-CT-3D2-IgG1-SI | 0.3 | i.p. | BIW | 4 |

Body weight of the mice was monitored throughout the entire experiment. The weight of mice in different groups all increased and there was no significant difference (P>0.05) between groups. At the time of grouping (D0), the average weight of each group was in a range of 19.7 g-19.9 g. At the end of the experiment (D21; or 21 days after grouping), the average weight of each group was in a range of 20.8 g-22.4 g, and the weight change was in the range of 105.1%-110.0%. Similar to the previous experiments, the results showed that the anti-CTLA4/OX40 bispecific antibodies were well tolerated and not toxic to the mice.

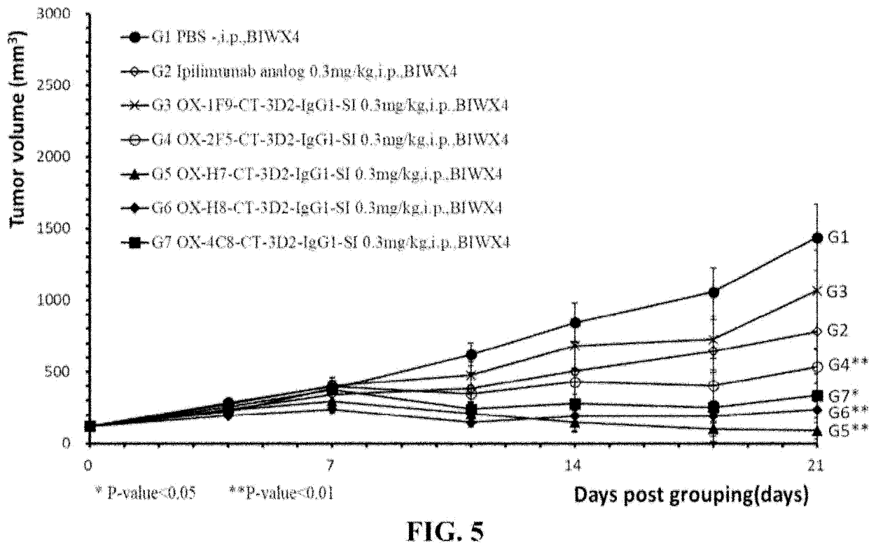

The tumor volumes in groups treated with the antibodies are shown in FIG. 5. The table below summarizes the results for this experiment, including the tumor volumes on the 15 day of grouping (D0), 11 days after the grouping (D11), and at the end of the experiment (D 21); the survival rate of the mice; the number of tumor-free mice; the Tumor growth inhibition rate (TGI_{TV}%); and the statistical differences (P value) of body weight and tumor volume between the treatment and control groups.

TABLE 17

| | | | | | | | | P value | |
|---|---|---|---|---|---|---|---|---|---|
| | | Tumor volume (mm³) | | | | Tumor- | TGI_{TV} | Body | Tumor |
| | | D 0 | D 11 | D 21 | Survival | free | % | weight | Volume |
| Control | G1 | 121 ± 3 | 622 ± 79 | 1438 ± 231 | 6/6 | 0 | n.a. | n.a. | n.a. |
| Treat | G2 | 121 ± 3 | 383 ± 110 | 783 ± 298 | 6/6 | 0 | 49.8 | 0.639 | 0.113 |
| | G3 | 121 ± 4 | 477 ± 93 | 1067 ± 283 | 6/6 | 0 | 28.2 | 0.797 | 0.334 |
| | G4 | 121 ± 5 | 349 ± 56 | 538 ± 116 | 6/6 | 0 | 68.4 | 0.831 | 0.006 |
| | G5 | 121 ± 5 | 209 ± 76 | 92 ± 57 | 6/6 | 2 | 102.2 | 0.044 | 2.13E−04 |
| | G6 | 121 ± 6 | 148 ± 31 | 236 ± 56 | 6/6 | 1 | 91.2 | 0.155 | 0.001 |
| | G7 | 121 ± 6 | 242 ± 111 | 336 ± 331 | 6/6 | 4 | 83.7 | 0.196 | 0.021 |

The results showed that, all the tested anti-CTLA4/OX40 bispecific antibodies had different tumor inhibitory effects. Under the same treatment conditions (e.g., administration dosage and frequency), the inhibitory effects of the anti-CTLA4/OX40 bispecific antibodies (G4-G7) were better than Ipilimumab analog (G2). In particular, all mice in the G5, G6, and G7 group survived with tumor completely disappeared in 2 mice of group G5, 1 mouse in group G6, and 4 mice in group G7.

In Vivo Efficacy of 0.3-10 mg/kg Bispecific Antibodies in hCTLA4/hOX40 Mice

The hCTLA4/hOX40 mice (5-9 weeks) were subcutaneously injected 5×10⁵ colon adenocarcinoma cell line MC38, and when the tumor volume reached about 400 mm³, the mice were placed into a control group and four treatment groups based on tumor size (6 mice per group). The treatment group mice were randomly selected for intraperitoneal (i.p.) administration of anti-CTLA4/OX40 bispecific antibody OX-H7-CT-3D2-IgG1-SI at a dose level of 0.3 mg/kg (G2), 1 mg/kg (G3), 3 mg/kg (G4), or 10 mg/kg (G5). The control group mice were injected with an equal volume of PBS. The frequency of administration was once a week (3 times of administrations in total). The tumor volume was measured twice a week and the body weight of the mice was weighed as well. Euthanasia was performed when the tumor volume of the mouse reached 3000 mm³. Details of the administration scheme are shown in the table below.

TABLE 18

| | No. of | | Dosage | | | Total No. of |
|---|---|---|---|---|---|---|
| Group | mice | Antibodies | (mg/kg) | Route | Frequency | administration |
| G1 | 6 | PBS | — | i.p. | QW | 3 |
| G2 | 6 | OX-H7-CT-3D2-IgG1-SI | 0.3 | i.p. | QW | 3 |
| G3 | 6 | OX-H7-CT-3D2-IgG1-SI | 1 | i.p. | QW | 3 |
| G4 | 6 | OX-H7-CT-3D2-IgG1-SI | 3 | i.p. | QW | 3 |
| G5 | 6 | OX-H7-CT-3D2-IgG1-SI | 10 | i.p. | QW | 3 |

Body weight of the mice was monitored during the entire treatment period. The weight of mice in different groups all increased, with no statistically significant differences (P>0.05) between groups. On the day of grouping (Day 0; or “DO”), the average weight of each group was in a range of 20.6 g-21.4 g. At the end of the experiment (D23; or 23 days after grouping), the average weight of each group was in a range of 22.4 g-26.0 g, and the weight change was in the range of 108.3%-122.8%. The results showed that the anti-CTLA4/OX40 bispecific antibodies were well tolerated and not toxic to the mice.

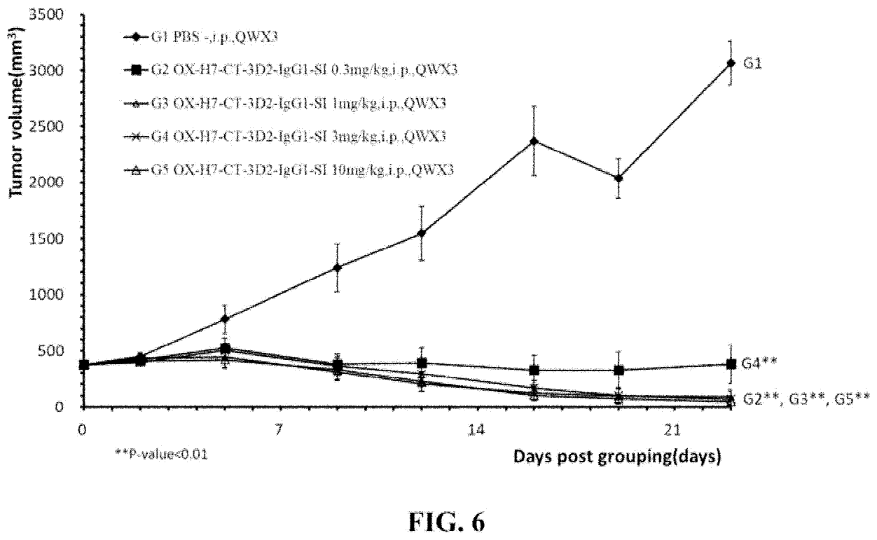

The tumor volumes in groups treated with the bispecific antibodies are shown in FIG. 6. The table below summarizes the results for this experiment, including the tumor volumes on the day of grouping (D0), 12 days after the grouping (D12), and at the end of the experiment (D23); the survival rate of the mice; the number of tumor-free mice; the Tumor growth inhibition rate (TGI_{TV}%); and the statistical differences (P value) of body weight and tumor volume between the treatment and control groups.

TABLE 19

| | | | | | | | | P value | |
|---|---|---|---|---|---|---|---|---|---|
| | | Tumor volume (mm³) | | | | Tumor- | | Body | Tumor |
| | | D 0 | D 12 | D 23 | Survival | free | TGI_{TV} % | weight | Volume |
| Control | G1 | 374 ± 13 | 1548 ± 244 | 3066 ± 195 | 4/6 | 0 | n.a. | n.a. | n.a. |
| Treat | G2 | 374 ± 13 | 392 ± 136 | 379 ± 175 | 6/6 | 1 | 99.8 | 0.050 | 8.16E−06 |
| | G3 | 374 ± 13 | 202 ± 63 | 89 ± 65 | 6/6 | 3 | 110.6 | 0.002 | 1.38E−07 |
| | G4 | 374 ± 13 | 293 ± 79 | 70 ± 65 | 6/6 | 3 | 111.3 | 4.52E−04 | 1.32E−07 |
| | G5 | 374 ± 15 | 223 ± 87 | 49 ± 36 | 6/6 | 3 | 112.1 | 3.97E−04 | 6.59E−08 |

The results showed that the anti-CTLA4/OX40 bispecific antibody OX-H7-CT-3D2-IgG1-SI can effectively inhibit tumor growth (with a TGI_{TV}% higher than 99%) at a dose level between 0.3 mg/kg to 10 mg/kg. The tumor inhibition also showed a correlation trend with an increasing dose. In addition, on Day 23 (23 days post grouping), all mice survived in groups G2-G5, with tumor disappeared in 1 mouse in group G2, and 3 mice in groups G3-G5.

In Vivo Efficacy of 0.3 mg/kg Antibodies in hCTLA4/hOX40 Mice

Similar to the previous experiments, anti-CTLA4/OX40 bispecific antibodies OX-H7-CT-3D2-IgG1-SI, OX-4C8-CT-3D2-IgG1-SI; anti-CTLA4 antibody CT-3D2-IgG1-SI; anti-OX40 antibodies OX-4C8-IgG1-SI, OX-H7-IgG1-SI; and an analog of the anti-CTLA4 monoclonal antibody Ipilimumab were tested for their in vivo effect on tumor growth in hCTLA4/hOX40 mouse model that were subcutaneously injected with colon carcinoma cell line MC38. When the tumors in the mice reached a volume of about 100-150 mm³, the mice were randomly placed into different groups (6 mice per group) based on the tumor volume. Details of the administration scheme are shown in the table below.

TABLE 20

| | No. of | | Dosage | | | Total No. of |
|---|---|---|---|---|---|---|
| Group | mice | Antibodies | (mg/kg) | Route | Frequency | administration |
| G1 | 6 | PBS | — | i.p. | QW | 4 |
| G2 | 6 | OX-H7-CT-3D2-IgG1-SI | 0.3 | i.p. | QW | 4 |
| G3 | 6 | OX-4C8-CT-3D2-IgG1-SI | 0.3 | i.p. | QW | 4 |
| G4 | 6 | CT-3D2-IgG1-SI | 0.3 | i.p. | QW | 4 |
| G5 | 6 | OX-4C8-IgG1-SI | 0.3 | i.p. | QW | 4 |
| G6 | 6 | OX-H7-IgG1-SI | 0.3 | i.p. | QW | 4 |
| G7 | 6 | Ipilimumab analog | 0.3 | i.p. | QW | 4 |

Body weight of the mice was monitored throughout the entire experiment. The weight of mice in different groups all increased and there was no significant difference (P>0.05) between groups. At the time of grouping (D0), the average weight of each group was in a range of 20.5 g-20.7 g. At the end of the experiment (D16; or 16 days after grouping), the average weight of each group was in a range of 22.4 g-25.5 g, and the weight change was in a range of 109.3%-123.4%. Similar to the previous experiments, the results showed that the tested antibodies were well tolerated and not toxic to the mice.

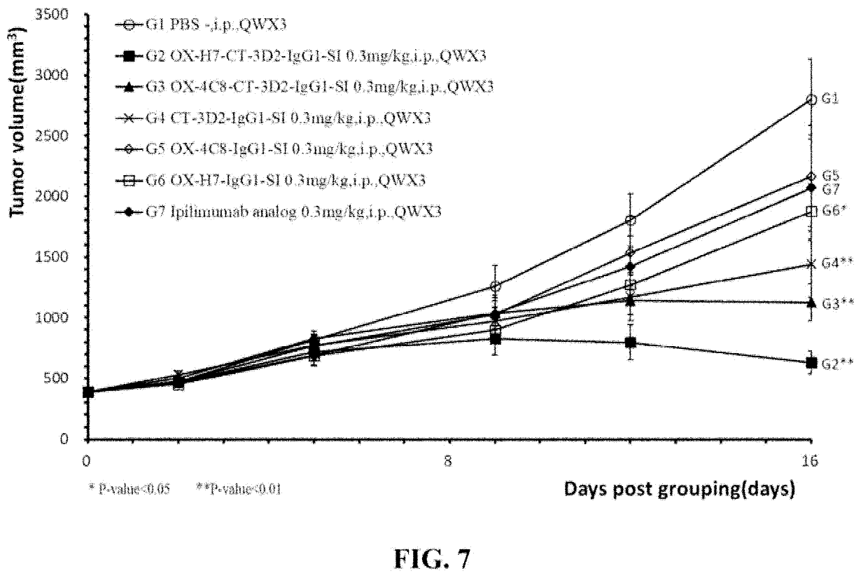

The tumor volumes in groups treated with the antibodies are shown in FIG. 7. The table below summarizes the results for this experiment, including the tumor volumes on the day of grouping (D0), 9 days after the grouping (D9), and at the end of the experiment (D16); the survival rate of the mice; the Tumor growth inhibition rate (TGI_{TV}%); and the statistical differences (P value) of body weight and tumor volume between the treatment and control groups.

TABLE 21

| | | | | | | | P value | |
|---|---|---|---|---|---|---|---|---|
| | | Tumor volume (mm³) | | | | | Body | Tumor |
| | | D 0 | D 9 | D 16 | Survival | TGI_{TV} % | weight | Volume |
| Control | G1 | 388 ± 11 | 1258 ± 176 | 2801 ± 327 | 6/6 | n.a. | n.a. | n.a. |
| Treatment | G2 | 388 ± 12 | 830 ± 138 | 639 ± 93 | 6/6 | 90.0% | 0.022 | 8.08E−05 |
| | G3 | 388 ± 12 | 1037 ± 128 | 1126 ± 152 | 6/6 | 69.4% | 0.137 | 0.001 |
| | G4 | 388 ± 15 | 971 ± 121 | 1440 ± 273 | 6/6 | 56.4% | 0.277 | 0.010 |
| | G5 | 388 ± 16 | 1026 ± 160 | 2167 ± 418 | 6/6 | 26.3% | 0.902 | 0.260 |
| | G6 | 389 ± 19 | 897 ± 87 | 1876 ± 224 | 6/6 | 38.3% | 0.461 | 0.042 |
| | G7 | 388 ± 19 | 1037 ± 105 | 2071 ± 438 | 6/6 | 30.3% | 0.326 | 0.211 |

Throughout the experimental period, the average tumor volume of all treatment group mice (G2-G7) was smaller than that of the control group mice (G1). The results also showed that, OX-H7-CT-3D2-IgG1-SI (G2) and OX-4C8-CT-3D2-IgG1-SI (G3) can inhibit tumor growth with a higher TGI_{TV}% (e.g., on Day 16) than that of the monoclonal antibodies CT-3D2-IgG1-SI (G4), OX-4C8-IgG1-SI (G5) or OX-H7-IgG1-SI (G6), as well as the Ipilimumab analog (G7).

In another similar experiment, hCTLA4/hOX40 mice were subcutaneously injected with glioma cell line GL261. When the tumors in the mice reached a volume of about 100-150 mm³, the mice were randomly placed into different groups (6 mice per group) based on the tumor volume. The treatment groups were randomly selected for OX-H7-CT-3D2-IgG1-SI treatment at a dosage of 1 mg/kg (G2), 3 mg/kg (G3) or 10 mg/kg (G4). The control group were injected with PBS (G1). The frequency of administration was once a week (3 administrations in total). The tumor volume was measured twice a week and the body weights of the mice were measured twice a week. Euthanasia was performed when the tumor volume of a mouse reached 3000 mm³.

On Day 19 post grouping, the tumor volume of the control group G1 and treatment groups G2-G4 were 1476±392 mm³, 32±9 mm³, 39±7 mm³ and 62±13 mm³, respectively. OX-H7-CT-3D2-IgG1-SI treatment groups obtained better anti-tumor effect compared with the control group, with a TGI % of 105.8% (G2), 105.3% (G3) and 103.6% (G4) respectively.

### Example 6. Binding Activity of Anti-CTLA4/OX40 Bispecific Antibodies to Cell Lines

The binding activity to HEK293T-hCTLA4-hOX40 cells (transfected HEK293T cells expressing human CTLA4 and human OX40) of anti-CTLA4/OX40 bispecific antibodies OX-H7-CT-3D2-IgG1-SI, OX-H8-CT-3D2-IgG1-SI, OX-4C8-CT-3D2-IgG1-SI; anti-CTLA4 antibody CT-3D2-IgG1; anti-OX40 antibodies OX-H7-IgG1, OX-H8-IgG1, OX-4C8-IgG1; or the Ipilimumab analog was measured by flow cytometry. Specifically, the tested antibodies were serially diluted (2-fold) with the highest concentration of 60 μg/ml. HEK293T-hCTLA4-hOX40 cells were seeded in a 96-well plate (cell density 1×10⁵ cells/well). The cells and the antibodies were incubated at 4° C. for 30 minutes. Afterwards, the cells were washed with 200 μL PBS and then centrifuged at 1200 rpm for 3 minutes. Anti-hIgG-Fc-Alexa Fluor™ 647 (Jackson ImmunoResearch, Cat #: 109-606-170) at a 1:500 dilution ratio was added to each well, and the plate was incubated at 4° C. for 15 minutes, followed by a PBS wash. The cells were centrifuged and then resuspended in 30 μL PBS. Signals for Alexa Fluor™ 647 were detected by flow cytometry.

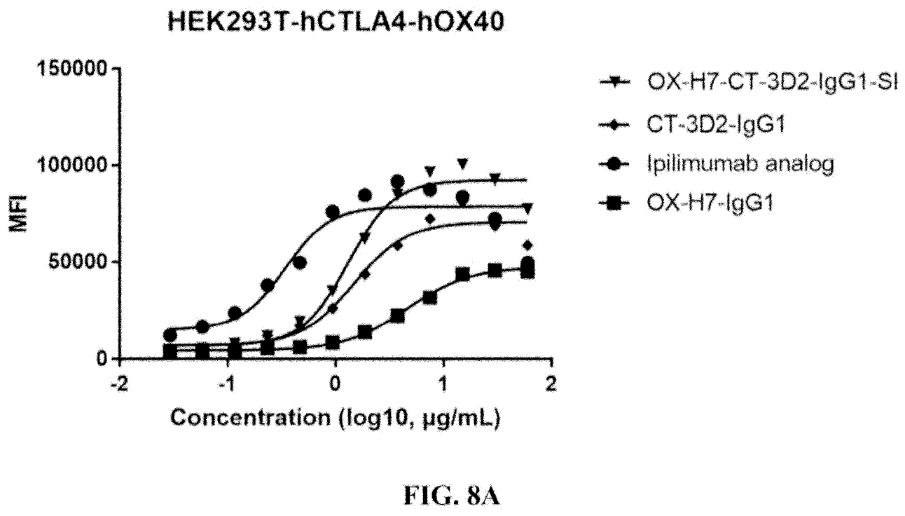

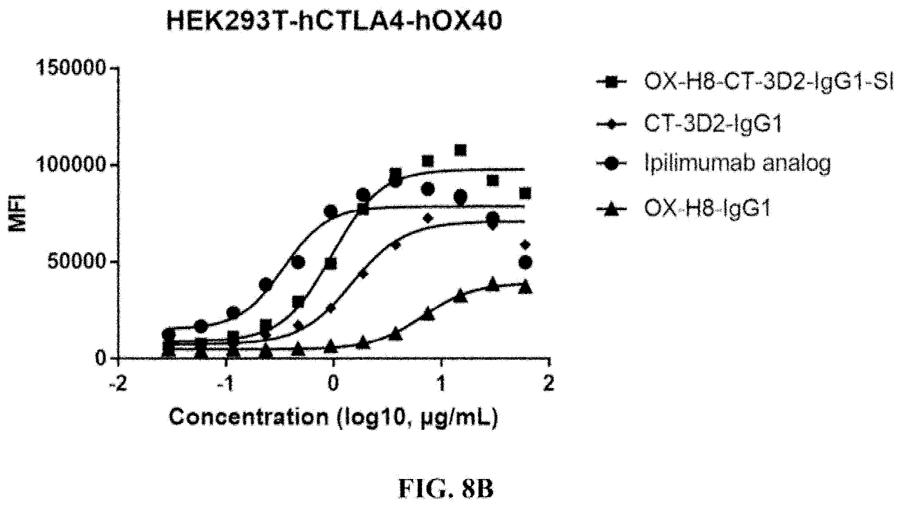

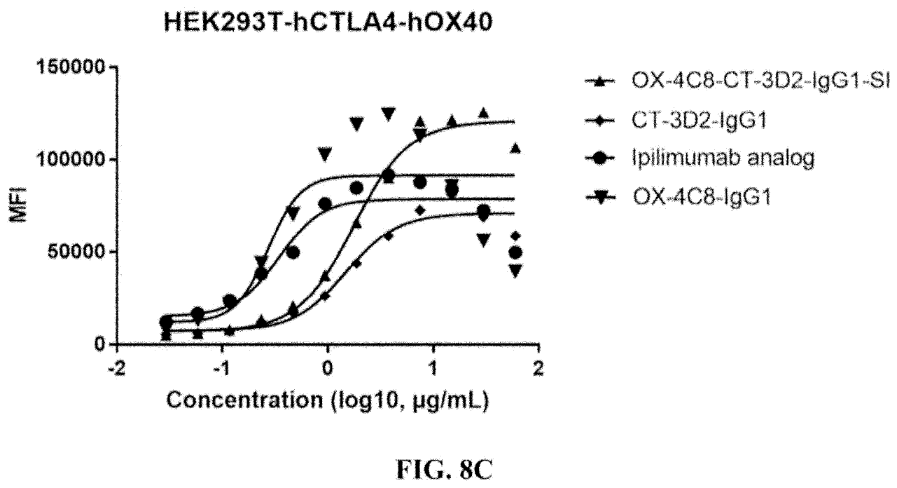

As shown in FIGS. 8A-8C, all tested antibodies can bind to HEK293T-hCTLA4-hOX40 cells. In particular, all anti-CTLA4/OX40 bispecific antibodies exhibited a higher binding activity than their corresponding monoclonal antibodies.

### Example 7. Jurkat-Luc-hCTLA4 Reporter Cell Activation Assays

The experiment was performed to test whether anti-CTLA4/OX40 bispecific antibodies OX-H7-CT-3D2-IgG1-SI, OX-4C8-CT-3D2-IgG1-SI; anti-CTLA4 monoclonal antibody CT-3D2-IgG1; and the Ipilimumab analog can block the CTLA4 signaling pathway.

The experiment was performed as follows. Effector cells Jurkat-luc-hCTLA4 (Promega, Cat #: CA186903) and target cells Raji-aAPC-CD80/86 (Promega, Cat #: CS186905) were cultured in RPMI 1640 medium supplemented with 10% fetal bovine serum (FBS). The tested antibodies were serially diluted (3-fold) with the highest concentration at 100 μg/ml. Effector cells Jurkat-luc-hCTLA4 were seeded in a 96-well plate (cell density 1×10⁵ cells/well), and then 25 μL target cells Raji-aAPC-CD80/86 together with 25 μL of the serially diluted antibody solution were added to each well. The above 96-well plate was incubated in a 37° C. incubator for 6 hours. After the incubation, the plate was taken out, and 75 L of Bio-Lite™ Luciferase Assay Reagent (Vazyme Biotech Co., Ltd., Cat #: DD1201-02-AB) was added to each well. The plate was incubated at room temperature for 5-10 minutes, and then placed in a luminescence detector to detect the fluorescence signal.

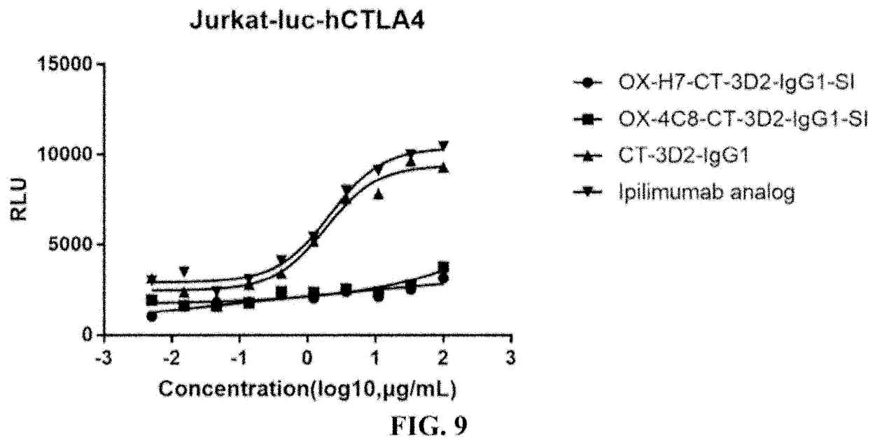

As shown in FIG. 9, the results showed that the activity induced by the bispecific antibodies in CTLA4 reporter cells was lower than the activity induced by the monoclonal antibodies. It is likely that the lower activity of bispecific antibodies was caused by their single anti-CTLA4 arm, as compared to the two anti-CTLA4 arms of the monoclonal antibodies.

### Example 8. Jurkat-Luc-hOX40 Reporter Cell Activation Assays

The experiment was performed to test whether anti-CTLA4/OX40 bispecific antibodies OX-H7-CT-3D2-IgG1-SI, OX-4C8-CT-3D2-IgG1-SI; and anti-OX40 monoclonal antibody 040-H7-IgG1 can activate the OX40 signaling pathway.

The experiment was performed as follows. Effector cells Jurkat-luc-hOX40 (Promega, Cat #: CS197703) and target cells Jurkat-hCTLA4 (Promega, Cat #: CA186903) were plated in a 96-well flat bottom plate (cell density 5×10⁴ cells/well). Alternatively, only effector cells Jurkat-luc-hOX40 were plated in a 96-well flat bottom plate (cell density 5×10⁴ cells/well). The tested antibodies were serially diluted (3-fold) with the highest concentration at 100 μg/ml. 25 μL of the serially diluted antibody solution was added to each well, and the plate was incubated at 37° C. for 6 hours. After the incubation, the plate was taken out, and 25 L of the Bio-Lite™ Luciferase Assay Reagent was added, followed by an incubation at room temperature for 5 minutes in dark. The plate was then placed in a luminescence detector to detect the fluorescence signal.

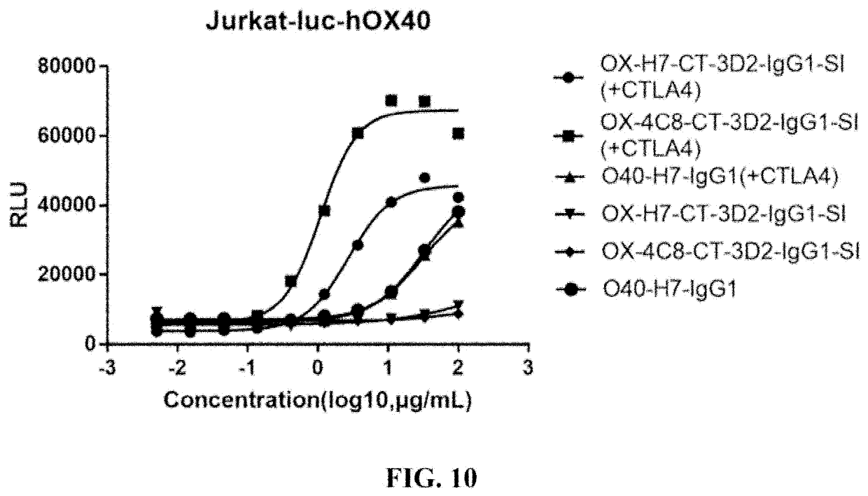

As shown in FIG. 10, when the assay only contained effector cells Jurkat-luc-hOX40, the anti-OX40 monoclonal antibody 040-H7-IgG1, but not the bispecific antibodies, can activate the OX40 signaling pathway. However, when the assay contained both effector cells Jurkat-luc-hOX40 and target cells Jurkat-hCTLA4, bispecific antibodies OX-H7-CT-3D2-IgG1-SI and OX-4C8-CT-3D2-IgG1-SI can also activate the OX40 signaling pathway. The results indicate that anti-CTLA4/OX40 bispecific antibodies can effectively activate the OX40 pathway only in the presence of CTLA4-expressing cells.

### Example 9. ADCC Activity Assays

The experiment was performed to test ADCC (antibody-dependent cellular cytotoxicity) activity. The experiment was performed as follows. The bispecific antibodies OX-H7-CT-3D2-IgG1-SI, OX-4C8-CT-3D2-IgG1-SI; anti-CTLA4 monoclonal antibody CT-3D2-IgG1-SI; and the Ipilimumab analog were serially diluted (3-fold) with the highest concentration at 15 μg/ml. Effector cells Jurkat-luc-hCD16A (transfected Jurkat-luc cells expressing human CD16 (hCD16) with an amino acid sequence shown in SEQ ID NO: 148) were seeded in a 96-well plate (cell density 8×10⁴ cells/well), and then 25 μL target cells Jurkat-hCTLA4-hOX40 (transfected Jurkat cells expressing human CTLA4 and human OX40) together with 25 μL of the serially diluted antibody solution were added to each well (cell density 2×10⁴ cells/well). The above 96-well plate was incubated at 37° C. for 6 hours. After the incubation, the plate was taken out, and 75 μL of Bio-Lite™ Luciferase Assay Reagent was added to each well. The plate was incubated at room temperature for 5-10 minutes, and then placed in a luminescence detector to detect the fluorescence signal emitted from the effector cells.

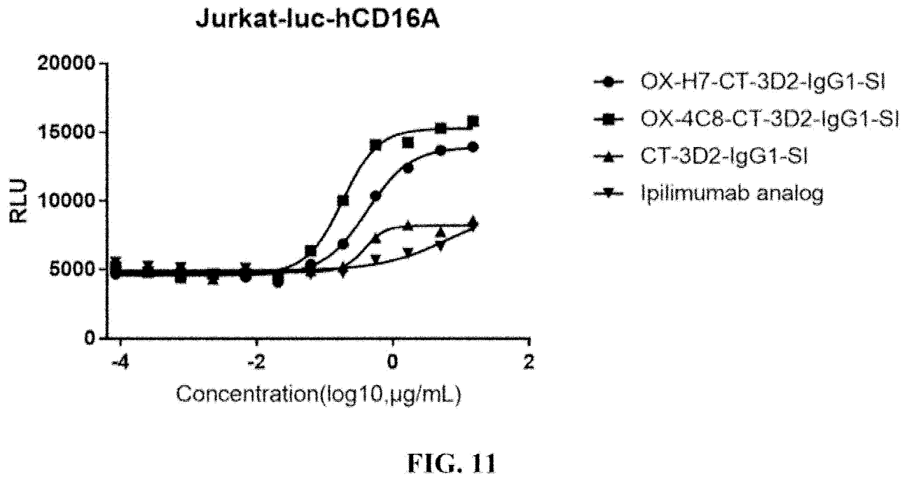

As shown in FIG. 11, all tested antibodies exhibited ADCC effects. In particular, bispecific antibodies OX-H7-CT-3D2-IgG1 and OX-4C8-CT-3D2-IgG1 exhibited stronger ADCC effects than the monoclonal antibody CT-3D2-IgG1-SI. It is likely that the anti-CTLA4/OX40 bispecific antibodies can target both CTLA4 and OX40 on the target cells, thereby having a stronger ADCC effect than the monoclonal antibodies, which can target either one of CTLA4 or OX40.

In another similar experiment, target cells human Treg cells (Oricell®, including donor 1 and donar 2) and effector cells FcR-TANK (ImmuneOnco Biopharmaceuticals (Shanghai) Co., Ltd.) were used to evaluate ADCC effects of the bispecific antibody OX-H7-CT-3D2-IgG1-SI and anti-CTLA4 monoclonal antibody Ipilimumab analog. Human IgG1 (hIgG1) was used for isotype control. After incubating at 37° C. for 4 hours, the cells above were detected by flow cytometry.

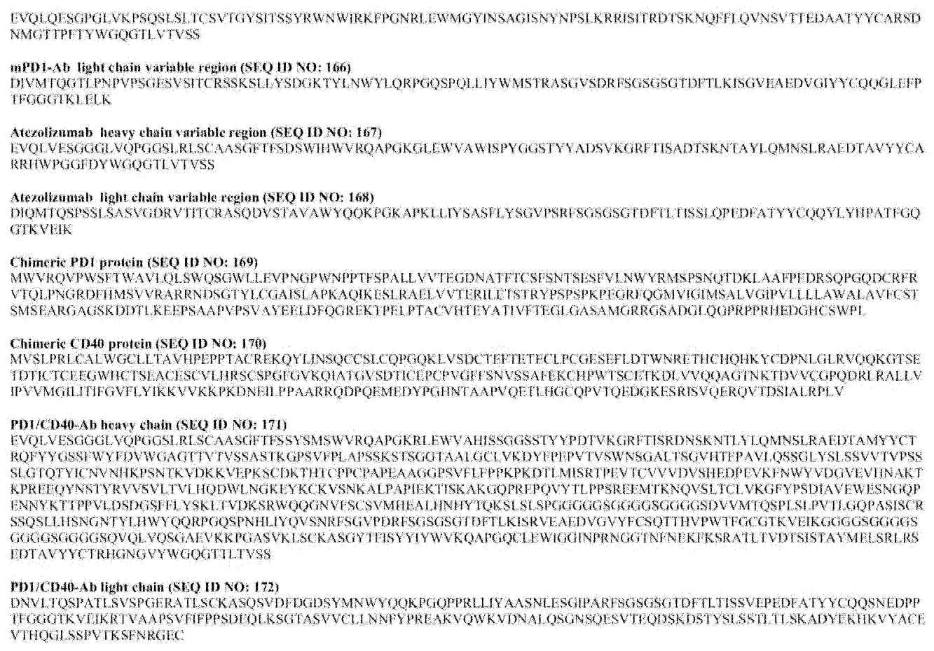

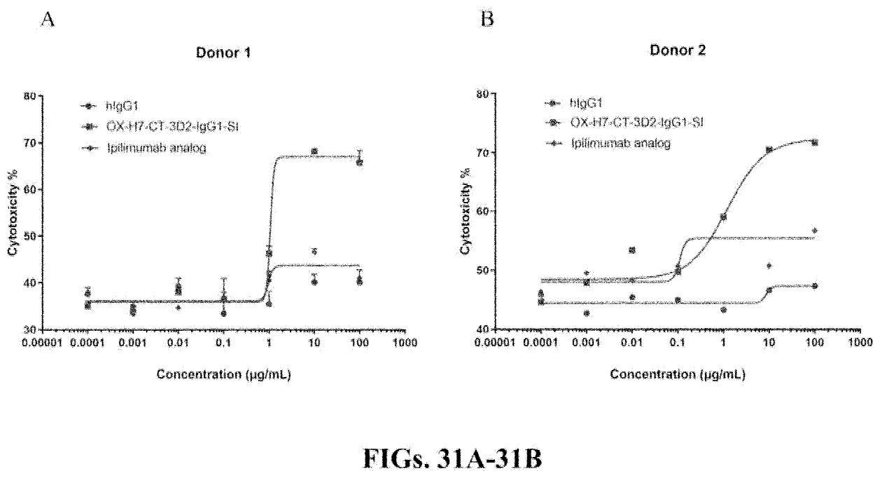

As shown in FIGS. 31A-31B, OX-H7-CT-3D2-IgG1-SI exhibited better ADCC effects in vitro than the monoclonal antibody Ipilimumab analog both in donor 1 and donor 2.

### Example 10. Activity Detection of Anti-CTLA4/OX40 Bispecific Antibodies in Human PBMCs

### Binding to T Cells

TGN1412 (VH SEQ ID NO: 107, VL SEQ ID NO: 108), a monoclonal antibody targeting CD28, was developed by TeGenero. TGN1412 can activate Treg cells and is mainly used for the treatment of autoimmune diseases (e.g., rheumatoid arthritis). Anti-CD28 monoclonal antibody CD28-TGN1412-hIgG4-S228P was produced, which includes human IgG4 constant regions (and a S228P mutation according to EU numbering).

The following experiment was performed to verify the human PBMC-binding avidity of anti-CTLA4/OX40 bispecific antibodies OX-H7-CT-3D2-IgG1-SI, OX-4C8-CT-3D2-IgG1-SI; anti-OX40 antibody OX-H7-IgG1; and anti-CTLA4 antibody CT-3D2-IgG1. The antibodies were serially diluted (2-fold) with the highest concentration at 60 μg/ml. CD28-TGN1412-hIgG4-S228P and Monoclonal Anti-Human CD3 Antibody, Mouse IgG2a (Beijing ACROBiosystems Co.Ltd., Cat #: CDE-M120a) were diluted with PBS to a final concentration of 2 μg/mL and 10 μg/mL, respectively. The diluted anti-CD28 and anti-CD3 antibodies were mixed, and 100 μL of the mixed antibody solution was used to coat each well of a 96-well flat bottom plate overnight at 4° C. After coating, PBS was aspirated in the plate, and each well was washed twice by 200 μl PBS. PBMCs were seeded in the 96-well plate (cell density 2×10⁵ cells/well), and the plate was incubated at 37° C. for 72 hours. After the incubation, cells were collected in a 15 ml centrifuge tube by centrifugation at 1200 rpm for 10 minutes. After centrifugation, the supernatant was discarded, and PBS was added to resuspend the cells. 20 μL of the resuspended cell solution was mixed with 10 μL of the serially diluted antibody solution. The cells were then add to a 96-well plate, followed by an incubation at 4° C. for 30 minutes. After the incubation, anti-CD3 antibody APC/Cyanine7 anti-human CD3 Antibody (BioLegend, Cat #: 300318), anti-CD8a antibody FITC anti-human CD8a Antibody (BioLegend, Cat #: 300906), and anti-CD4 antibody FITC anti-human CD4 Antibody (BioLegend, Cat #: 30050) were respectively added, followed by an incubation for 30 minutes. Afterwards, each well was washed twice with 200 μl PBS, and the plate was centrifuged at 1200 rpm for 5 minutes after each PBS wash. Finally, 50 μl PBS was added to resuspend the cells for flow cytometry detection.

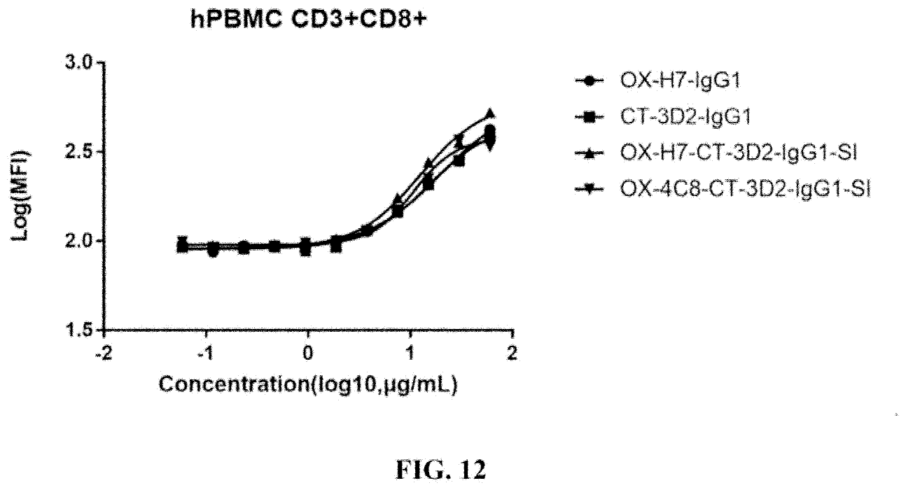

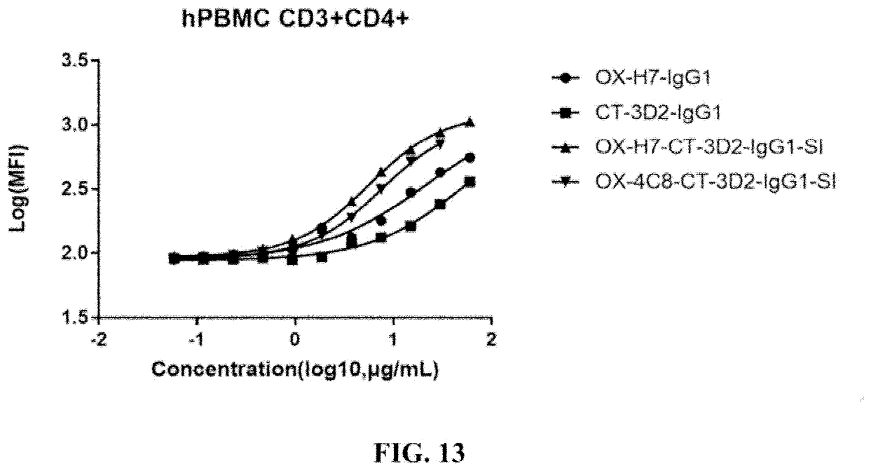

As shown in FIG. 12 and FIG. 13, all tested antibodies showed good avidity to CD8+ T cells and CD4+ T cells in human PBMCs. In particular, anti-CTLA4/OX40 bispecific antibodies OX-H7-CT-3D2-IgG1-SI and OX-4C8-CT-3D2-IgG1-SI showed a slightly higher CD4+ T cell-binding avidity than the monoclonal antibodies.

### IL2 Secretion Determination

Thawed PBMCs (2×10⁶/mL) were stimulated with SEB (Toxin Technology), and plated into 96-well cell culture plates (2×10⁵ cells/well). Then serial dilutions of the anti-CTLA4/OX40 bispecific antibody OX-H7-CT-3D2-IgG1-SI or anti-CTLA4 monoclonal antibody Ipilimumab analog was added (with a final concentration of 0 μg/mL, 0.0001 μg/mL, 0.001 μg/mL, 0.01 μg/mL, 0.1 μg/mL, 1 μg/mL, and 10 μg/mL), and incubated for 72 hours at 37° C. The supernatant was collected to detect the express level of human IL2 using Human IL-2 ELISA kit (Biolegend, Cat #: 431807). Human IgG1 (hIgG1) was used as negative control.

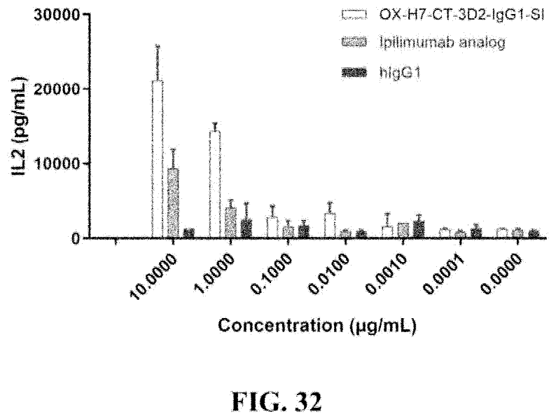

As shown in FIG. 32, compared with the positive control Ipilimumab analog and the negative control hIgG1, OX-H7-CT-3D2-IgG1-SI enhanced IL2 secretion in response to SEB using PBMCs.

### Example 11. In Vivo Toxicity Experiment (Non-Tumor Bearing Model)

The hCTLA4/hOX40 mice (6-8 weeks) were randomly placed into a control group and two treatment group (5 mice in each group) according to their body weight. The treatment group mice were injected with anti-CTLA4/OX40 bispecific antibody OX-H7-CT-3D2-IgG1-SI (G2) or OX-4C8-CT-3D2-IgG1-SI (G3) at a dose level of 30 mg/kg. The control group mice were injected with an equal volume of PBS. The administration frequency was 3 times a week for a total of 3 administrations after grouping. On Day 4 and Day 7 after grouping, blood biochemical analyses were performed to monitor levels of aspartate aminotransferase (AST) and alanine aminotransferase (ALT).

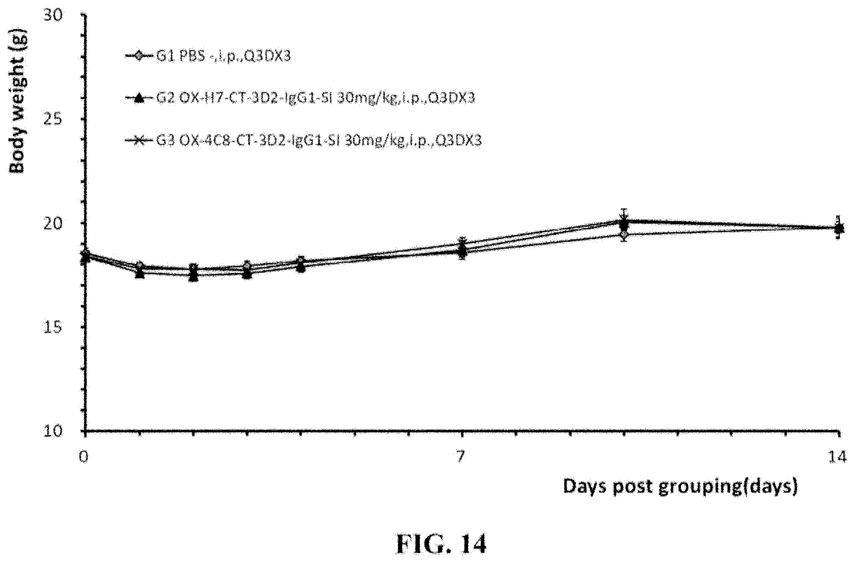

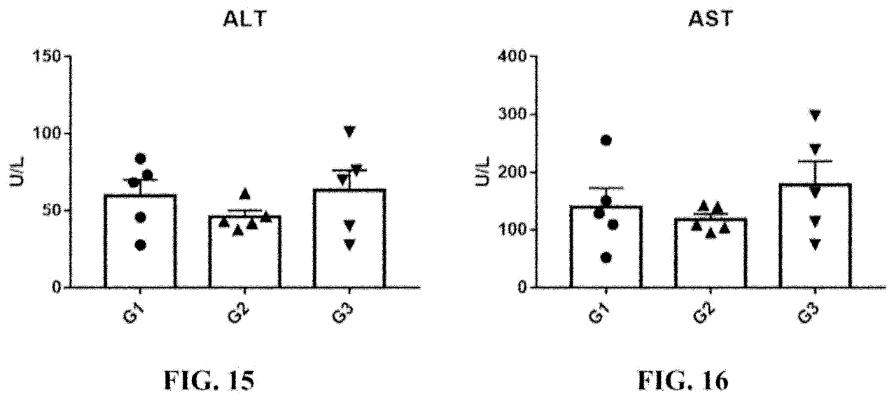

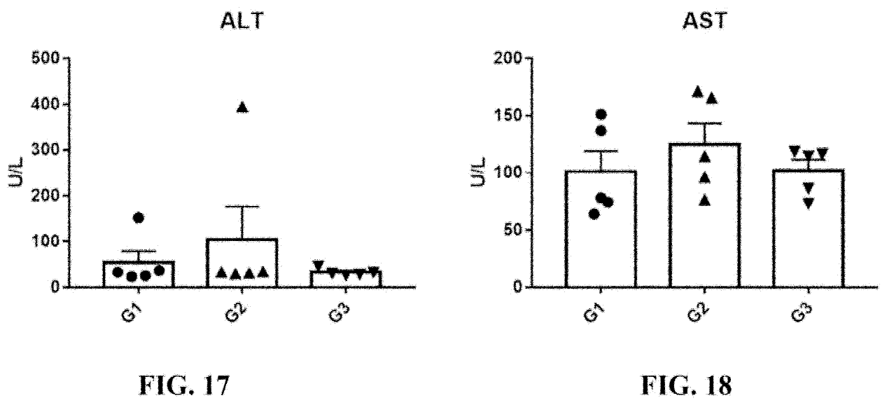

The results in FIG. 14 showed that the body weight of all mice in the control group and the treatment groups increased throughout the experimental period, and there was no significant differences (P>0.05) between groups. FIGS. 15-16 show the levels of ALT and AST on Day 4, respectively; and FIGS. 17-18 show the levels of ALT and AST on Day 7, respectively. No significant difference between groups G1-G3 was detected. Similar to the previous results, the in vivo toxicity results showed that the anti-CTLA4/OX40 bispecific antibodies were well tolerated and not toxic to the mice. In addition, serum cytokine levels on Day 1 and Day 4 were also detected (data not shown), and no obvious cytokine storm was observed.

### Example 12. In Vivo Efficacy in hCTLA4/hOX40 Mice with TILs Analysis

The hCTLA4/hOX40 mice (5-9 weeks) were subcutaneously injected with MC38 (5×10⁵), and when the tumor volume reached about 100-150 mm³, the mice were placed into a control group and three treatment groups based on tumor size (5 mice per group). The treatment group mice were randomly selected for intraperitoneal (i.p.) administration of anti-CTLA4/OX40 bispecific antibodies OX-H7-CT-3D2-IgG1-SI (G2), OX-4C8-CT-3D2-IgG1-SI (G3), or the Ipilimumab analog (G4) at a dose level of 0.3 mg/kg. The control group mice were injected with an equal volume of PBS. The administration was performed on Day 7 (7 days after grouping) and Day 11 (11 days after grouping). Euthanasia was performed when the tumor volume of the mouse reached 3000 mm³. Details of the administration scheme are shown in the table below.

TABLE 22

| | No. of | | Dosage | | Total No. of |
|---|---|---|---|---|---|
| Group | mice | Antibodies | (mg/kg) | Route | administration |
| G1 | 5 | PBS | — | i.p. | 2 |
| G2 | 5 | OX-H7-CT- | 0.3 | i.p. | 2 |
| | | 3D2-IgG1-SI | | | |
| G3 | 5 | OX-4C8-CT- | 0.3 | i.p. | 2 |
| | | 3D2-IgG1-SI | | | |
| G4 | 5 | Ipilimumab | 0.3 | i.p. | 2 |
| | | analog | | | |

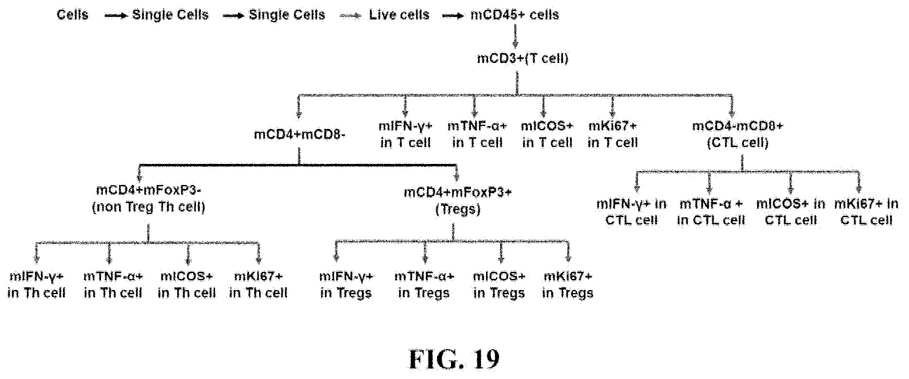

At the end of the experiment, the tumor tissue and mouse spleen were collected, digested, and then re-suspended as a single cell suspension for flow cytometry analysis. Anti-mouse CD45 antibody APC/Cyanine7 anti-mouse CD45 Antibody (BioLegend, Cat #: 103116), anti-mouse CD16/32 antibody Purified anti-mouse CD16/32 antibody (BioLegend, Cat #: 101302), anti-mouse CD3 antibody Alexa Fluor® 700 anti-mouse CD3 Antibody (BioLegend, Cat #: 100216), anti-mouse CD4 antibody Brilliant Violet 421™ anti-mouse CD4 Antibody (BioLegend, Cat #: 100438), anti-mouse CD8a antibody Brilliant Violet 605™ anti-mouse CD8a Antibody (BioLegend, Cat #: 100744), anti-CD278 antibody FITC anti-human/mouse/rat CD278 (ICOS) Antibody (BioLegend, Cat #: 313506), anti-mouse TNF-α antibody APC anti-mouse TNF-α antibody (BioLegend, Cat #: 506308), anti-mouse IFN-γ antibody PE anti-mouse IFN-γ Antibody (BioLegend, Cat #: 505808), anti-mouse Ki-67 antibody PerCP/Cyanine5.5 (BioLegend, Cat #: 652424). anti-mouse Ki-67 Antibody (eBioscience, Cat #: 25-5773-82) were used for cell staining before flow cytometry. The cell sorting strategy for TILs (tumor-infiltrating lymphocytes) analysis is shown in FIG. 19.

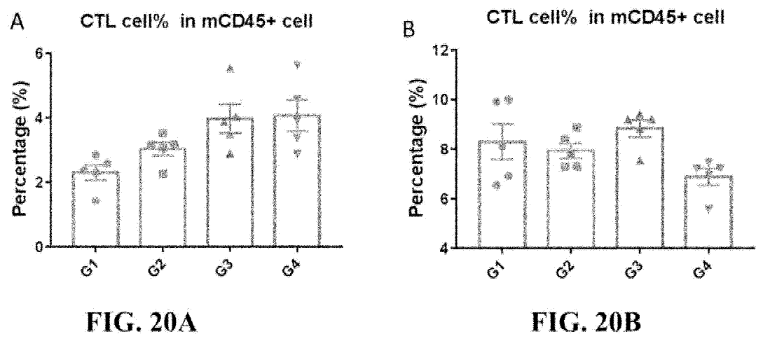

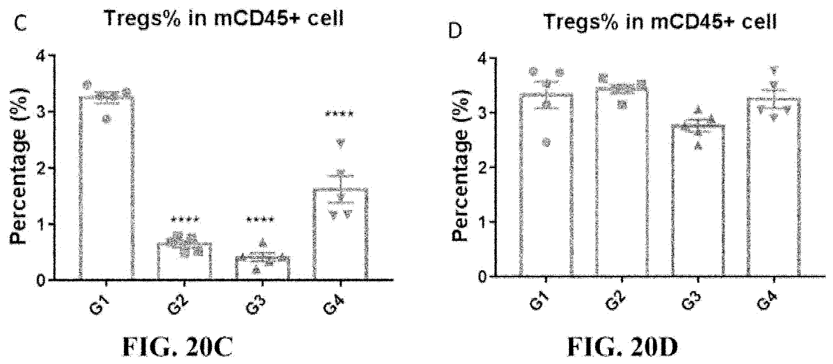

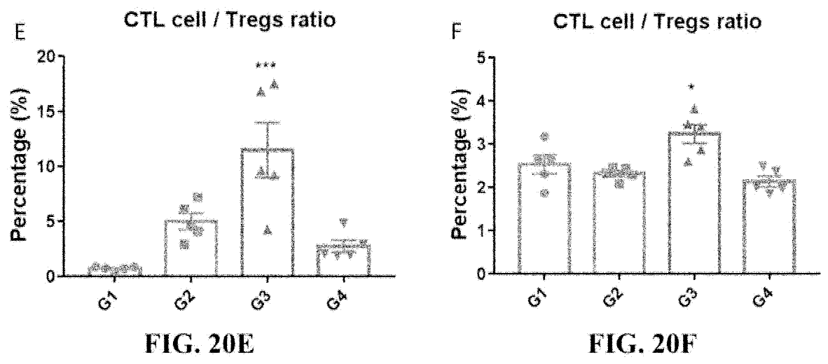

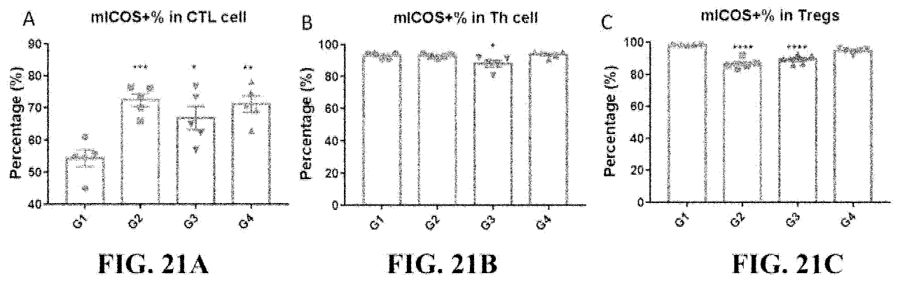

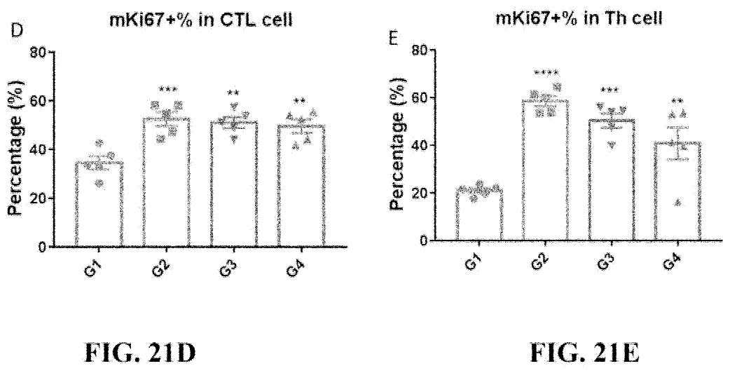

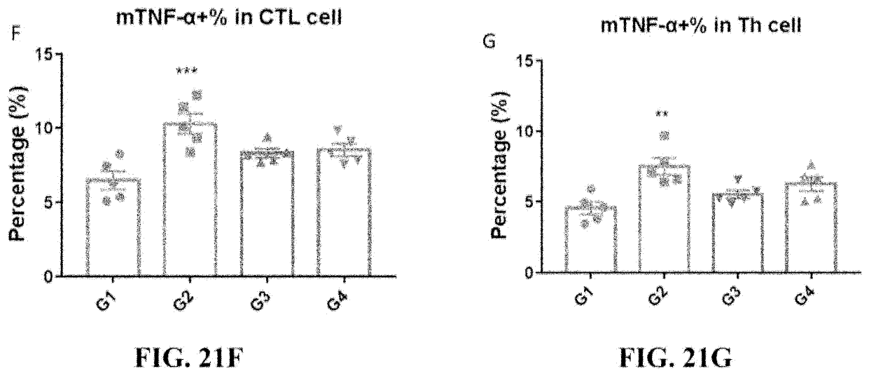

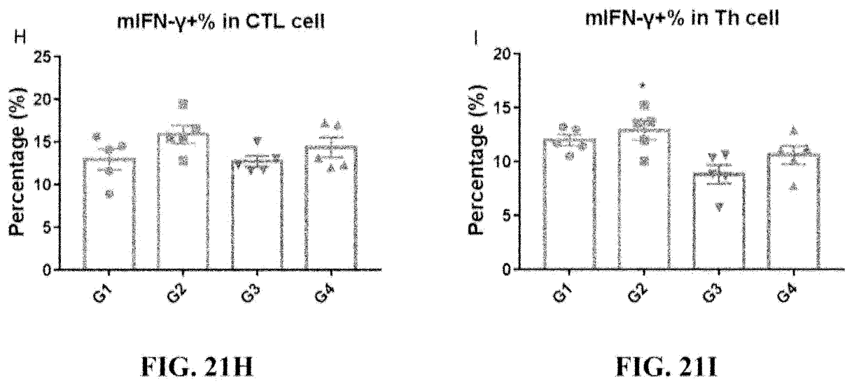

As shown in FIGS. 20A-20F and FIGS. 21A-21I, no significant difference of the immune cell numbers was observed in normal tissues (spleen). However, bispecific antibody OX-H7-CT-3D2-IgG1-SI (G2) and OX-4C8-CT-3D2-IgG1-SI (G3) can deplete Treg cells in tumor tissues more effectively than the Ipilimumab analog (G4). In addition, the bispecific antibodies can effectively promote the activation and proliferation of effector T cells (CTL).

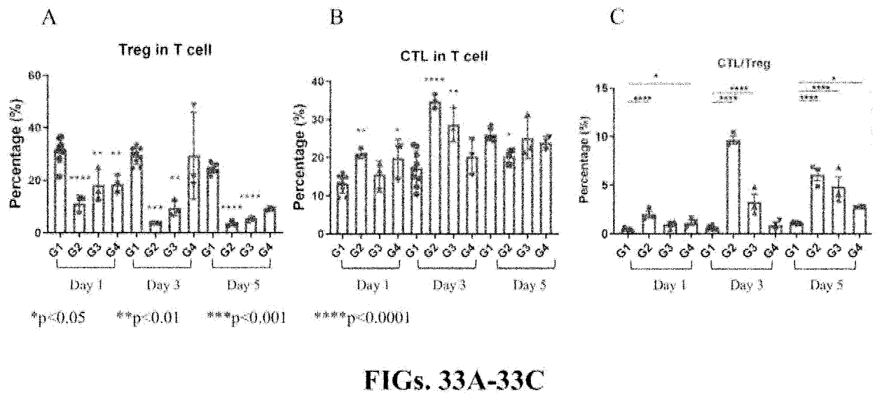

In another similar experiment, the hCTLA4/hOX40 mice were subcutaneously injected with MC38 cells (5×10⁵), and when the tumor volume reached about 180-240 mm³, the mice were placed into a control group and three treatment groups based on tumor size (9 mice per group). The treatment group mice were randomly selected for intravenous (i.v.) administration of OX-H7-CT-3D2-IgG1-SI (G2), CT-3D2-IgG1-SI (G3), or Ipilimumab (from Bristol-Myers Squibb) (G4) at a dose level of 3 mg/kg. The control group mice were injected with an equal volume of PBS (G1). The frequency of administration was once a week (1 administration in total). The tumor tissues were collected on Day 1 (1 day post grouping), Day 3 (3 days post grouping) and Day 5 (5 days post grouping) for TILs analysis. The test results are shown in FIGS. 33A-33C, which showed that compared with the control group G1 and G4, OX-H7-CT-3D2-IgG1-SI significantly deplete Treg cells, and effectively promoted the activation and proliferation of effector T cells in tumor tissues.

### Example 13. Combination Therapeutic Efficacy In Vivo

### Combination of Anti-CTLA4/OX40 Bispecific Antibody and Anti-PD1 Antibody

The hCTLA4/hOX40 mice were subcutaneously injected with MC38 (5×10⁵), and when the tumor volume reached about 400 mm, the mice were placed into a control group and five treatment groups based on tumor size (7 mice per group). The treatment group mice were randomly selected for intraperitoneal (i.p.) administration of OX-H7-CT-3D2-IgG1-SI, combination of CT-3D2-IgG1-SI and OX-H7-IgG1-SI, mPD1-Ab or combination of OX-H7-CT-3D2-IgG1-SI and mPD1-Ab. The control group mice were injected with an equal volume of PBS. Euthanasia was performed when the tumor volume of the mouse reached 3000 mm³. Details of the administration scheme are shown in the table below.

TABLE 23

| | No. of | | | | | | Total No. of |
|---|---|---|---|---|---|---|---|
| Group | mice | Antibodies | Dosage | | Route | Frequency | administration |
| G1 | 7 | PBS | — | | i.p. | QW | 3 |
| G2 | 7 | OX-H7-CT-3D2-IgG1-SI | 0.3 | mg/kg | i.p. | QW | 3 |
| G3 | 7 | OX-H7-CT-3D2-IgG1-SI | 0.6 | mg/kg | i.p. | QW | 3 |
| G4 | 7 | CT-3D2-IgG1-SI | 0.3 | mg/kg | i.p. | QW | 3 |
| | | OX-H7-IgG1-SI | 0.3 | mg/kg | i.p. | QW | 3 |
| G5 | 7 | mPD1-Ab | 3 | mg/kg | i.p. | QW | 3 |
| G6 | 7 | OX-H7-CT-3D2-IgG1-SI | 0.3 | mg/kg | i.p. | QW | 3 |
| | | mPD1-Ab | 3 | mg/kg | i.p. | QW | 3 |

mPD1-Ab is a mouse monoclonal IgG1 antibody targeting PD1, with LALA mutations in the heavy chain constant region. The VH and VL sequences are shown in SEQ ID NOs: 165-166.

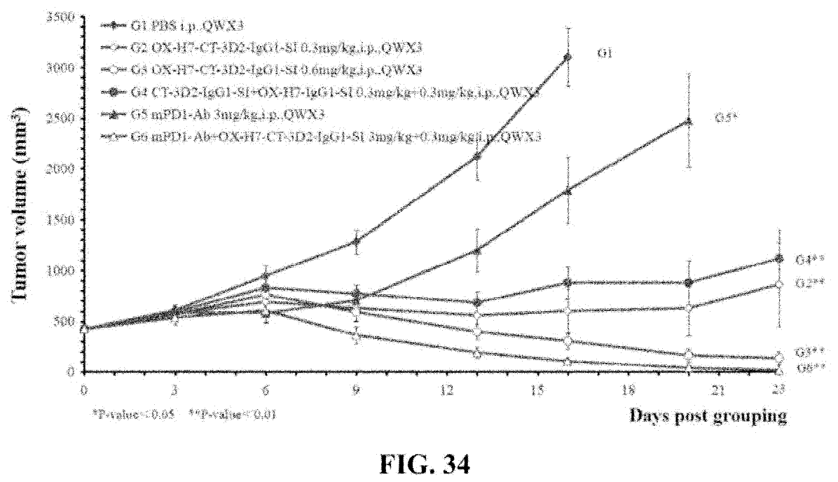

The tumor volumes in groups treated with the antibodies are shown in FIG. 34. The table below summarizes the results for this experiment, including the tumor volumes on the day of grouping (D0), 16 days after grouping (D16) and at the end of the experiment (D23); the TGI_{TV}%; P value of tumor volume between the treatment and control groups; and the survival rate of the mice at the end of the experiment (D23). As all mice in the control group and mPD1-Ab treatment group died, P value for D23 was not available.

TABLE 24

| | | | | | | | Tumor- |
|---|---|---|---|---|---|---|---|
| | Tumor volume (mm³) | | | TGI_{TV} % | P value | Survival | free |
| Group | D 0 | D 16 | D 23 | (D 16) | (D 16) | (D 23) | (D 23) |
| G1 | 424 ± 31 | 3100 ± 288 | n.a. | n.a. | n.a. | 0/7 | n.a. |
| G2 | 423 ± 32 | 598 ± 222 | 863 ± 413 | 93.5% | 1.69E−05 | 7/7 | 2/7 |
| G3 | 423 ± 32 | 305 ± 81 | 135 ± 69 | 104.4% | 7.30E−07 | 7/7 | 1/7 |
| G4 | 423 ± 33 | 879 ± 159 | 1116 ± 289 | 83.0% | 2.02E−05 | 7/7 | 0/7 |
| G5 | 424 ± 35 | 1791 ± 323 | n.a. | 48.9% | 0.011 | 0/7 | n.a. |
| G6 | 424 ± 33 | 105 ± 34 | 16 ± 10 | 111.9% | 2.48E−07 | 7/7 | 5/7 |

Throughout the experimental period, the average tumor volume of all treatment group mice (G2-G6) was smaller than that of the control group mice (G1). Among the treatment groups, OX-H7-CT-3D2-IgG1-SI at a dosage of 0.6 mg/kg (G3) showed better anti-tumor activity than that of the combination of CT-3D2-IgG1-SI and OX-H7-IgG1-SI (G4). In addition, the combination of OX-H7-CT-3D2-IgG1-SI and mPD1-Ab (G6) significantly improved tumor inhibition efficacy, compared with that of OX-H7-CT-3D2-IgG1-SI (G2) or mPD1-Ab (G5) alone.

### Combination of Anti-CTLA4/OX40 Bispecific Antibody and Anti-PDL1 Antibody

The hCTLA4/hOX40 mice were subcutaneously injected with B16-F10-OVA melanoma cells (2×10⁵), and when the tumor volume reached about 60-90 mm³, the mice were placed into a control group and three treatment groups based on tumor size (6 mice per group). The treatment group mice were randomly selected for intraperitoneal (i.p.) administration of 1 mg/kg OX-H7-CT-3D2-IgG1-SI (G2), 10 mg/kg Atezolizumab analog (G3), or combination of 1 mg/kg OX-H7-CT-3D2-IgG1-SI and 10 mg/kg Atezolizumab analog (G4). The control group mice were injected with an equal volume of PBS (G1). The frequency of administration was twice a week (3 administrations in total). The tumor volume and body weights of the mice were measured twice a week. Euthanasia was performed when the tumor volume of a mouse reached 3000 mm³.

Atezolizumab is a humanized anti-PD-L1 monoclonal antibody developed by Genentech, and the VH and VL sequences are shown in SEQ ID NOs: 167-168.

Body weight of the mice was monitored throughout the entire experiment. At the time of grouping (D0), the average weight of each group was in a range of 20.1 g-20.4 g. At the end of the experiment (D7), the average weight of each group was in a range of 22.0 g-23.2 g, and the weight change was in a range of 107.8%-115.7%. The tested antibodies were well tolerated and not toxic to the mice.

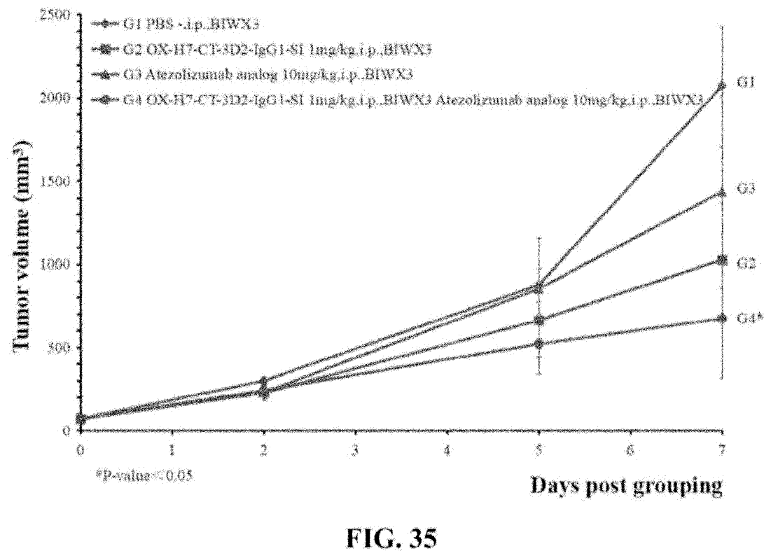

The tumor volumes in groups treated with the antibodies are shown in FIG. 35. Similar to the previous experiment, the results showed that the combination of anti-CTLA4/OX40 bispecific antibody OX-H7-CT-3D2-IgG1-SI and anti-PDL1 antibody Atezolizumab analog (G4) obtained better tumor inhibition efficacy with a TGI_{TV}% of 69.9% than that of OX-H7-CT-3D2-IgG1-SI (G2, 52.2%) or Atezolizumab analog (G3, 31.7%) alone.

### Combination of Anti-CTLA4/OX40 Bispecific Antibody and Anti-PD1/CD40 Bispecific Antibody

A hPD1/hCD40/hCTLA4/hOX40 mouse model (obtained from Biocytogen Pharmaceuticals (Beijing) Co., Ltd.) was engineered to express a chimeric CTLA4 protein, a chimeric OX40 protein, a chimeric PD1 protein (SEQ ID NO: 169), and a chimeric CD40 protein (SEQ ID NO: 170). A detailed description regarding the hPD1 mouse model, the hCD40 mouse model, and the double humanized hPD1/hCD40 mouse model can be found in PCT Application Nos. PCT/CN2017/090320 and PCT/CN2018/091845, each of which is incorporated herein by reference in its entirety.

The hPD1/hCD40/hCTLA4/hOX40 mice were subcutaneously injected with MC38 (5×10⁵), and when the tumor volume reached about 250-300 mm³, the mice were placed into a control group and three treatment groups based on tumor size (5 mice per group). The treatment group mice were randomly selected for intraperitoneal (i.p.) administration of anti-PD1/CD40 bispecific antibody PD1/CD40-Ab, anti-CTLA4/OX40 bispecific antibodies OX-H7-CT-3D2-IgG1-SI, or combination of PD1/CD40-Ab and OX-H7-CT-3D2-IgG1-SI. The control group mice were injected with an equal volume of PBS. Euthanasia was performed when the tumor volume of the mouse reached 3000 mm³. Details of the administration scheme are shown in the table below.

TABLE 25

| | No. of | | | | | | Total No. of |
|---|---|---|---|---|---|---|---|
| Group | mice | Antibodies | Dosage | | Route | Frequency | administration |
| G1 | 7 | PBS | — | | i.p. | BIW | 4 |
| G2 | 7 | PD1/CD40-Ab | 1 | mg/kg | i.p. | BIW | 4 |
| G3 | 7 | PD1/CD40-Ab | 3 | mg/kg | i.p. | BIW | 4 |
| G4 | 7 | OX-H7-CT-3D2-IgG1-SI | 0.3 | mg/kg | i.p. | QW | 2 |
| G5 | 7 | PD1/CD40-Ab | 1 | mg/kg | i.p. | BIW | 4 |
| | | OX-H7-CT-3D2-IgG1-SI | 0.3 | mg/kg | i.p. | QW | 2 |

PD1/CD40-Ab is an humanized bispecific antibody targeting PD1 and CD40, which was obtained by conventional methods. The heavy chain sequence and light chain sequence are shown in SEQ ID NOs: 171-172.

Body weight of the mice was monitored throughout the entire experiment. At the time of grouping (D0), the average weight of each group was in a range of 22.7 g-23.2 g. 24 days post grouping (D24), the average weight of each group was in a range of 24.0 g-26.4 g, and the weight change was in a range of 104.6%-119.7%. The tested antibodies were well tolerated and not toxic to the mice.

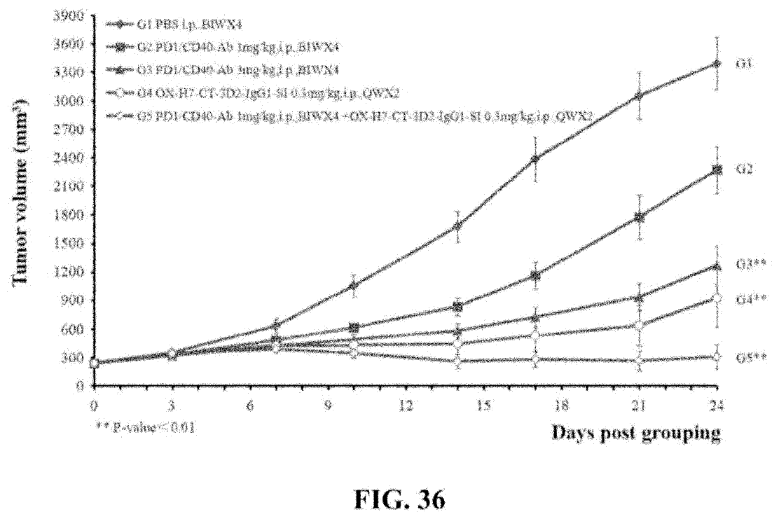

The tumor volumes in groups treated with the antibodies are shown in FIG. 36. The table below summarizes the results for this experiment, including the tumor volumes on the day of grouping (D0), 14 days after grouping (D14) and 24 days after grouping (D24); the TGI_{TV}%; P value of tumor volume between the treatment and control groups; and the survival rate of the mice on D24.

TABLE 26

| | Tumor volume (mm³) | | | | | | Tumor- |
|---|---|---|---|---|---|---|---|
| Group | D 0 | D 14 | D 24 | TGI_{TV} % | P value | Survival | free |
| G1 | 238 ± 15 | 1677 ± 163 | 3387 ± 274 | n.a. | n.a. | 2/7 | 0/7 |
| G2 | 238 ± 15 | 832 ± 93 | 2272 ± 247 | 35.4% | 0.061 | 7/7 | 0/7 |
| G3 | 238 ± 17 | 581 ± 72 | 1267 ± 206 | 67.3% | 0.002 | 7/7 | 0/7 |
| G4 | 238 ± 19 | 439 ± 114 | 922 ± 307 | 78.3% | 0.005 | 7/7 | 0/7 |
| G5 | 238 ± 16 | 257 ± 73 | 304 ± 128 | 97.9% | 1.06E−05 | 7/7 | 1/7 |

Similar to the previous experiments, the results showed that the combination of anti-CTLA4/OX40 bispecific antibody OX-H7-CT-3D2-IgG1-SI and anti-PD1/CD40 bispecific antibody PD1/CD40-Ab obtained better tumor inhibition efficacy with a TGI_{TV}% of 97.9% than that of OX-H7-CT-3D2-IgG1-SI (78.3%) or Atezolizumab analog (35.4%) alone.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An antibody or antigen-binding fragment thereof that binds to CTLA4 (Cytotoxic T-lymphocyte antigen-4) comprising:
a heavy chain variable region (VH) comprising complementarity determining regions (CDRs) 1, 2, and 3, wherein the VH CDR1 region comprises an amino acid sequence that is at least 80% identical to a selected VH CDR1 amino acid sequence, the VH CDR2 region comprises an amino acid sequence that is at least 80% identical to a selected VH CDR2 amino acid sequence, and the VH CDR3 region comprises an amino acid sequence that is at least 80% identical to a selected VH CDR3 amino acid sequence; and
a light chain variable region (VL) comprising CDRs 1, 2, and 3, wherein the VL CDR1 region comprises an amino acid sequence that is at least 80% identical to a selected VL CDR1 amino acid sequence, the VL CDR2 region comprises an amino acid sequence that is at least 80% identical to a selected VL CDR2 amino acid sequence, and the VL CDR3 region comprises an amino acid sequence that is at least 80% identical to a selected VL CDR3 amino acid sequence,
wherein the selected VH CDRs 1, 2, and 3 amino acid sequences and the selected VL CDRs, 1, 2, and 3 amino acid sequences are one of the following:
(1) the selected VH CDRs 1, 2, 3 amino acid sequences are set forth in SEQ ID NOs: 1-3, respectively, and the selected VL CDRs 1, 2, 3 amino acid sequences are set forth in SEQ ID NOs: 29-31, respectively;
(2) the selected VH CDRs 1, 2, 3 amino acid sequences are set forth in SEQ ID NOs: 4-6, respectively, and the selected VL CDRs 1, 2, 3 amino acid sequences are set forth in SEQ ID NOs: 29-31, respectively;
(3) the selected VH CDRs 1, 2, 3 amino acid sequences are set forth in SEQ ID NOs: 7-9, respectively, and the selected VL CDRs 1, 2, 3 amino acid sequences are set forth in SEQ ID NOs: 29-31, respectively;
(4) the selected VH CDRs 1, 2, 3 amino acid sequences are set forth in SEQ ID NOs: 10-12, respectively, and the selected VL CDRs 1, 2, 3 amino acid sequences are set forth in SEQ ID NOs: 29-31, respectively;
(5) the selected VH CDRs 1, 2, 3 amino acid sequences are set forth in SEQ ID NOs: 13-15, respectively, and the selected VL CDRs 1, 2, 3 amino acid sequences are set forth in SEQ ID NOs: 32-34, respectively;
(6) the selected VH CDRs 1, 2, 3 amino acid sequences are set forth in SEQ ID NOs: 16-18, respectively, and the selected VL CDRs 1, 2, 3 amino acid sequences are set forth in SEQ ID NOs: 32-34, respectively;
(7) the selected VH CDRs 1, 2, 3 amino acid sequences are set forth in SEQ ID NOs: 19-21, respectively, and the selected VL CDRs 1, 2, 3 amino acid sequences are set forth in SEQ ID NOs: 32-34, respectively; and
(8) the selected VH CDRs 1, 2, 3 amino acid sequences are set forth in SEQ ID NOs: 22-24, respectively, and the selected VL CDRs 1, 2, 3 amino acid sequences are set forth in SEQ ID NOs: 32-34, respectively.

2. The antibody or antigen-binding fragment thereof of claim 1, wherein the VH comprises CDRs 1, 2, 3 with the amino acid sequences set forth in SEQ ID NOs: 1-3, respectively, and the VL comprises CDRs 1, 2, 3 with the amino acid sequences set forth in SEQ ID NOs: 29-31, respectively, according to the Kabat numbering scheme.

3. The antibody or antigen-binding fragment thereof of claim 1, wherein the VH comprises CDRs 1, 2, 3 with the amino acid sequences set forth in SEQ ID NOs: 4-6, respectively, and the VL comprises CDRs 1, 2, 3 with the amino acid sequences set forth in SEQ ID NOs: 29-31, respectively, according to the Kabat numbering scheme.

4. The antibody or antigen-binding fragment thereof of claim 1, wherein the VH comprises CDRs 1, 2, 3 with the amino acid sequences set forth in SEQ ID NOs: 7-9, respectively, and the VL comprises CDRs 1, 2, 3 with the amino acid sequences set forth in SEQ ID NOs: 29-31, respectively, according to the Kabat numbering scheme.

5. The antibody or antigen-binding fragment thereof of claim 1, wherein the VH comprises CDRs 1, 2, 3 with the amino acid sequences set forth in SEQ ID NOs: 10-12, respectively, and the VL comprises CDRs 1, 2, 3 with the amino acid sequences set forth in SEQ ID NOs: 29-31, respectively, according to the Kabat numbering scheme.

6. The antibody or antigen-binding fragment thereof of claim 1, wherein the VH comprises CDRs 1, 2, 3 with the amino acid sequences set forth in SEQ ID NOs: 13-15, respectively, and the VL comprises CDRs 1, 2, 3 with the amino acid sequences set forth in SEQ ID NOs: 32-34, respectively, according to the Chothia numbering scheme.

7. The antibody or antigen-binding fragment thereof of claim 1, wherein the VH comprises CDRs 1, 2, 3 with the amino acid sequences set forth in SEQ ID NOs: 16-18, respectively, and the VL comprises CDRs 1, 2, 3 with the amino acid sequences set forth in SEQ ID NOs: 32-34, respectively, according to the Chothia numbering scheme.

8. The antibody or antigen-binding fragment thereof of claim 1, wherein the VH comprises CDRs 1, 2, 3 with the amino acid sequences set forth in SEQ ID NOs: 19-21, respectively, and the VL comprises CDRs 1, 2, 3 with the amino acid sequences set forth in SEQ ID NOs: 32-34, respectively, according to the Chothia numbering scheme.

9. The antibody or antigen-binding fragment thereof of claim 1, wherein the VH comprises CDRs 1, 2, 3 with the amino acid sequences set forth in SEQ ID NOs: 22-24, respectively, and the VL comprises CDRs 1, 2, 3 with the amino acid sequences set forth in SEQ ID NOs: 32-34, respectively, according to the Chothia numbering scheme.

10. An antibody or antigen-binding fragment thereof that binds to CTLA4 comprising
a heavy chain variable region (VH) comprising an amino acid sequence that is at least 90% identical to a selected VH sequence, and a light chain variable region (VL) comprising an amino acid sequence that is at least 90% identical to a selected VL sequence, wherein the selected VH sequence and the selected VL sequence are one of the following:
(1) the selected VH sequence is SEQ ID NO: 25, and the selected VL sequence is SEQ ID NO: 35;
(2) the selected VH sequence is SEQ ID NO: 26, and the selected VL sequence is SEQ ID NO: 35;
(3) the selected VH sequence is SEQ ID NO: 27, and the selected VL sequence is SEQ ID NO: 35; and
(4) the selected VH sequence is SEQ ID NO: 28, and the selected VL sequence is SEQ ID NO: 35.

11. The antibody or antigen-binding fragment thereof of claim 10, wherein the VH comprises the sequence of SEQ ID NO: 25 and the VL comprises the sequence of SEQ ID NO: 35.

12. The antibody or antigen-binding fragment thereof of claim 10, wherein the VH comprises the sequence of SEQ ID NO: 26 and the VL comprises the sequence of SEQ ID NO: 35.

13. The antibody or antigen-binding fragment thereof of claim 10, wherein the VH comprises the sequence of SEQ ID NO: 27 and the VL comprises the sequence of SEQ ID NO: 35.

14. The antibody or antigen-binding fragment thereof of claim 10, wherein the VH comprises the sequence of SEQ ID NO: 28 and the VL comprises the sequence of SEQ ID NO: 35.

15. An antibody or antigen-binding fragment thereof that binds to CTLA4 comprising
a heavy chain variable region (VH) comprising VH CDR1, VH CDR2, and VH CDR3 that are identical to VH CDR1, VH CDR2, and VH CDR3 of a selected VH sequence; and a light chain variable region (VL) comprising VL CDR1, VL CDR2, and VL CDR3 that are identical to VL CDR1, VL CDR2, and VL CDR3 of a selected VL sequence,
wherein the selected VH sequence and the selected VL sequence are one of the following:
(1) the selected VH sequence is SEQ ID NO: 25, and the selected VL sequence is SEQ ID NO: 35;
(2) the selected VH sequence is SEQ ID NO: 26, and the selected VL sequence is SEQ ID NO: 35;
(3) the selected VH sequence is SEQ ID NO: 27, and the selected VL sequence is SEQ ID NO: 35; and
(4) the selected VH sequence is SEQ ID NO: 28, and the selected VL sequence is SEQ ID NO: 35.

16. The antibody or antigen-binding fragment thereof of any one of claims 1-15, wherein the antibody or antigen-binding fragment thereof specifically binds to human or monkey CTLA4.

17. The antibody or antigen-binding fragment thereof of any one of claims 1-16, wherein the antibody or antigen-binding fragment is a human or humanized antibody or antigen-binding fragment thereof.

18. The antibody or antigen-binding fragment thereof of any one of claims 1-17, wherein the antibody or antigen-binding fragment is a single-chain variable fragment (scFV).

19. The antibody or antigen-binding fragment thereof of any one of claims 1-17, wherein the antibody or antigen-binding fragment thereof is a bispecific or a multispecific antibody or an antigen-binding fragment thereof.

20. The antibody or antigen-binding fragment thereof of claim 19, wherein the antibody or antigen-binding fragment thereof further specifically binds to OX40.

21. An antibody or antigen-binding fragment thereof that cross-competes with the antibody or antigen-binding fragment thereof of any one of claims 1-20.

22. A nucleic acid comprising a polynucleotide encoding a polypeptide comprising:
(1) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 1-3, respectively, and wherein the VH, when paired with a light chain variable region (VL) comprising the amino acid sequence set forth in SEQ ID NO: 35, binds to CTLA4;
(2) an immunoglobulin light chain or a fragment thereof comprising a light chain variable region (VL) comprising complementarity determining regions (CDRs) 1, 2, and 3 comprising the amino acid sequences set forth in SEQ ID NOs: 29-31, respectively, and wherein the VL, when paired with a heavy chain variable region (VH) comprising the amino acid sequence set forth in SEQ ID NO: 25, binds to CTLA4;
(3) an immunoglobulin heavy chain or a fragment thereof comprising a VH comprising CDRs 1, 2, and 3 comprising the amino acid sequences set forth in SEQ ID NOs: 4-6, respectively, and wherein the VH, when paired with a VL comprising the amino acid sequence set forth in SEQ ID NO: 35, binds to CTLA4;
(4) an immunoglobulin light chain or a fragment thereof comprising a VL comprising CDRs 1, 2, and 3 comprising the amino acid sequences set forth in SEQ ID NOs: 29-31, respectively, and wherein the VL, when paired with a VH comprising the amino acid sequence set forth in SEQ ID NO: 26, binds to CTLA4;
(5) an immunoglobulin heavy chain or a fragment thereof comprising a VH comprising CDRs 1, 2, and 3 comprising the amino acid sequences set forth in SEQ ID NOs: 7-9, respectively, and wherein the VH, when paired with a VL comprising the amino acid sequence set forth in SEQ ID NO: 35, binds to CTLA4;
(6) an immunoglobulin light chain or a fragment thereof comprising a VL comprising CDRs 1, 2, and 3 comprising the amino acid sequences set forth in SEQ ID NOs: 29-31, respectively, and wherein the VL, when paired with a VH comprising the amino acid sequence set forth in SEQ ID NO: 27, binds to CTLA4;
(7) an immunoglobulin heavy chain or a fragment thereof comprising a VH comprising CDRs 1, 2, and 3 comprising the amino acid sequences set forth in SEQ ID NOs: 10-12, respectively, and wherein the VH, when paired with a VL comprising the amino acid sequence set forth in SEQ ID NO: 35, binds to CTLA4;
(8) an immunoglobulin light chain or a fragment thereof comprising a VL comprising CDRs 1, 2, and 3 comprising the amino acid sequences set forth in SEQ ID NOs: 29-31, respectively, and wherein the VL, when paired with a VH comprising the amino acid sequence set forth in SEQ ID NO: 28, binds to CTLA4;
(9) an immunoglobulin heavy chain or a fragment thereof comprising a VH comprising CDRs 1, 2, and 3 comprising the amino acid sequences set forth in SEQ ID NOs: 13-15, respectively, and wherein the VH, when paired with a VL comprising the amino acid sequence set forth in SEQ ID NO: 35, binds to CTLA4;
(10) an immunoglobulin light chain or a fragment thereof comprising a VL comprising CDRs 1, 2, and 3 comprising the amino acid sequences set forth in SEQ ID NOs: 32-34, respectively, and wherein the VL, when paired with a VH comprising the amino acid sequence set forth in SEQ ID NO: 25, binds to CTLA4;
(11) an immunoglobulin heavy chain or a fragment thereof comprising a VH comprising CDRs 1, 2, and 3 comprising the amino acid sequences set forth in SEQ ID NOs: 16-18, respectively, and wherein the VH, when paired with a VL comprising the amino acid sequence set forth in SEQ ID NO: 35, binds to CTLA4;
(12) an immunoglobulin light chain or a fragment thereof comprising a VL comprising CDRs 1, 2, and 3 comprising the amino acid sequences set forth in SEQ ID NOs: 32-34, respectively, and wherein the VL, when paired with a VH comprising the amino acid sequence set forth in SEQ ID NO: 26, binds to CTLA4;
(13) an immunoglobulin heavy chain or a fragment thereof comprising a VH comprising CDRs 1, 2, and 3 comprising the amino acid sequences set forth in SEQ ID NOs: 19-21, respectively, and wherein the VH, when paired with a VL comprising the amino acid sequence set forth in SEQ ID NO: 35, binds to CTLA4;
(14) an immunoglobulin light chain or a fragment thereof comprising a VL comprising CDRs 1, 2, and 3 comprising the amino acid sequences set forth in SEQ ID NOs: 32-34, respectively, and wherein the VL, when paired with a VH comprising the amino acid sequence set forth in SEQ ID NO: 27, binds to CTLA4;
(15) an immunoglobulin heavy chain or a fragment thereof comprising a VH comprising CDRs 1, 2, and 3 comprising the amino acid sequences set forth in SEQ ID NOs: 22-24, respectively, and wherein the VH, when paired with a VL comprising the amino acid sequence set forth in SEQ ID NO: 35, binds to CTLA4; and
(16) an immunoglobulin light chain or a fragment thereof comprising a VL comprising CDRs 1, 2, and 3 comprising the amino acid sequences set forth in SEQ ID NOs: 32-34, respectively, and wherein the VL, when paired with a VH comprising the amino acid sequence set forth in SEQ ID NO: 28, binds to CTLA4.

23. The nucleic acid of claim 22, wherein the VH when paired with a VL specifically binds to human or monkey CTLA4.

24. The nucleic acid of claim 22 or 23, wherein the immunoglobulin heavy chain or the fragment thereof is a human or humanized immunoglobulin heavy chain or a fragment thereof.

25. The nucleic acid of any one of claims 22-24, wherein the nucleic acid encodes a single-chain variable fragment (scFv).

26. The nucleic acid of any one of claims 22-25, wherein the nucleic acid is cDNA.

27. An antibody or antigen-binding fragment thereof that binds to OX40 (Tumor necrosis factor receptor superfamily, member 4) comprising:
a heavy chain variable region (VH) comprising complementarity determining regions (CDRs) 1, 2, and 3, wherein the VH CDR1 region comprises an amino acid sequence that is at least 80% identical to a selected VH CDR1 amino acid sequence, the VH CDR2 region comprises an amino acid sequence that is at least 80% identical to a selected VH CDR2 amino acid sequence, and the VH CDR3 region comprises an amino acid sequence that is at least 80% identical to a selected VH CDR3 amino acid sequence; and
a light chain variable region (VL) comprising CDRs 1, 2, and 3, wherein the VL CDR1 region comprises an amino acid sequence that is at least 80% identical to a selected VL CDR1 amino acid sequence, the VL CDR2 region comprises an amino acid sequence that is at least 80% identical to a selected VL CDR2 amino acid sequence, and the VL CDR3 region comprises an amino acid sequence that is at least 80% identical to a selected VL CDR3 amino acid sequence,
wherein the selected VH CDRs 1, 2, and 3 amino acid sequences and the selected VL CDRs, 1, 2, and 3 amino acid sequences are one of the following:
(1) the selected VH CDRs 1, 2, 3 amino acid sequences are set forth in SEQ ID NOs: 36-38, respectively, and the selected VL CDRs 1, 2, 3 amino acid sequences are set forth in SEQ ID NOs: 29-31, respectively;
(2) the selected VH CDRs 1, 2, 3 amino acid sequences are set forth in SEQ ID NOs: 39-41, respectively, and the selected VL CDRs 1, 2, 3 amino acid sequences are set forth in SEQ ID NOs: 29-31, respectively;
(3) the selected VH CDRs 1, 2, 3 amino acid sequences are set forth in SEQ ID NOs: 42-44, respectively, and the selected VL CDRs 1, 2, 3 amino acid sequences are set forth in SEQ ID NOs: 29-31, respectively;
(4) the selected VH CDRs 1, 2, 3 amino acid sequences are set forth in SEQ ID NOs: 45-47, respectively, and the selected VL CDRs 1, 2, 3 amino acid sequences are set forth in SEQ ID NOs: 29-31, respectively;
(5) the selected VH CDRs 1, 2, 3 amino acid sequences are set forth in SEQ ID NOs: 48-50, respectively, and the selected VL CDRs 1, 2, 3 amino acid sequences are set forth in SEQ ID NOs: 29-31, respectively;
(6) the selected VH CDRs 1, 2, 3 amino acid sequences are set forth in SEQ ID NOs: 51-53, respectively, and the selected VL CDRs 1, 2, 3 amino acid sequences are set forth in SEQ ID NOs: 29-31, respectively;
(7) the selected VH CDRs 1, 2, 3 amino acid sequences are set forth in SEQ ID NOs: 54-56, respectively, and the selected VL CDRs 1, 2, 3 amino acid sequences are set forth in SEQ ID NOs: 29-31, respectively; alternatively, the selected VH CDRs 1, 2, 3 amino acid sequences are set forth in SEQ ID NOs: 54-56, respectively, and the selected VL CDRs 1, 2, 3 amino acid sequences are set forth in SEQ ID NOs: 149-151, respectively;
(8) the selected VH CDRs 1, 2, 3 amino acid sequences are set forth in SEQ ID NOs: 57-59, respectively, and the selected VL CDRs 1, 2, 3 amino acid sequences are set forth in SEQ ID NOs: 29-31, respectively; alternatively, the selected VH CDRs 1, 2, 3 amino acid sequences are set forth in SEQ ID NOs: 57-59, respectively, and the selected VL CDRs 1, 2, 3 amino acid sequences are set forth in SEQ ID NOs: 152-154, respectively;
(9) the selected VH CDRs 1, 2, 3 amino acid sequences are set forth in SEQ ID NOs: 60-62, respectively, and the selected VL CDRs 1, 2, 3 amino acid sequences are set forth in SEQ ID NOs: 32-34, respectively;
(10) the selected VH CDRs 1, 2, 3 amino acid sequences are set forth in SEQ ID NOs: 63-65, respectively, and the selected VL CDRs 1, 2, 3 amino acid sequences are set forth in SEQ ID NOs: 32-34, respectively;
(11) the selected VH CDRs 1, 2, 3 amino acid sequences are set forth in SEQ ID NOs: 66-68, respectively, and the selected VL CDRs 1, 2, 3 amino acid sequences are set forth in SEQ ID NOs: 32-34, respectively;
(12) the selected VH CDRs 1, 2, 3 amino acid sequences are set forth in SEQ ID NOs: 69-71, respectively, and the selected VL CDRs 1, 2, 3 amino acid sequences are set forth in SEQ ID NOs: 32-34, respectively;
(13) the selected VH CDRs 1, 2, 3 amino acid sequences are set forth in SEQ ID NOs: 72-74, respectively, and the selected VL CDRs 1, 2, 3 amino acid sequences are set forth in SEQ ID NOs: 32-34, respectively;
(14) the selected VH CDRs 1, 2, 3 amino acid sequences are set forth in SEQ ID NOs: 75-77, respectively, and the selected VL CDRs 1, 2, 3 amino acid sequences are set forth in SEQ ID NOs: 32-34, respectively;
(15) the selected VH CDRs 1, 2, 3 amino acid sequences are set forth in SEQ ID NOs: 78-80, respectively, and the selected VL CDRs 1, 2, 3 amino acid sequences are set forth in SEQ ID NOs: 32-34, respectively; alternatively, the selected VH CDRs 1, 2, 3 amino acid sequences are set forth in SEQ ID NOs: 78-80, respectively, and the selected VL CDRs 1, 2, 3 amino acid sequences are set forth in SEQ ID NOs: 155-157, respectively; and
(16) the selected VH CDRs 1, 2, 3 amino acid sequences are set forth in SEQ ID NOs: 81-83, respectively, and the selected VL CDRs 1, 2, 3 amino acid sequences are set forth in SEQ ID NOs: 32-34, respectively; alternatively, the selected VH CDRs 1, 2, 3 amino acid sequences are set forth in SEQ ID NOs: 81-83, respectively, and the selected VL CDRs 1, 2, 3 amino acid sequences are set forth in SEQ ID NOs: 158-160, respectively.

28. The antibody or antigen-binding fragment thereof of claim 27, wherein the VH comprises CDRs 1, 2, 3 with the amino acid sequences set forth in SEQ ID NOs: 36-38, respectively, and the VL comprises CDRs 1, 2, 3 with the amino acid sequences set forth in SEQ ID NOs: 29-31, respectively, according to the Kabat numbering scheme.

29. The antibody or antigen-binding fragment thereof of claim 27, wherein the VH comprises CDRs 1, 2, 3 with the amino acid sequences set forth in SEQ ID NOs: 39-41, respectively, and the VL comprises CDRs 1, 2, 3 with the amino acid sequences set forth in SEQ ID NOs: 29-31, respectively, according to the Kabat numbering scheme.

30. The antibody or antigen-binding fragment thereof of claim 27, wherein the VH comprises CDRs 1, 2, 3 with the amino acid sequences set forth in SEQ ID NOs: 42-44, respectively, and the VL comprises CDRs 1, 2, 3 with the amino acid sequences set forth in SEQ ID NOs: 29-31, respectively, according to the Kabat numbering scheme.

31. The antibody or antigen-binding fragment thereof of claim 27, wherein the VH comprises CDRs 1, 2, 3 with the amino acid sequences set forth in SEQ ID NOs: 45-47, respectively, and the VL comprises CDRs 1, 2, 3 with the amino acid sequences set forth in SEQ ID NOs: 29-31, respectively, according to the Kabat numbering scheme.

32. The antibody or antigen-binding fragment thereof of claim 27, wherein the VH comprises CDRs 1, 2, 3 with the amino acid sequences set forth in SEQ ID NOs: 48-50, respectively, and the VL comprises CDRs 1, 2, 3 with the amino acid sequences set forth in SEQ ID NOs: 29-31, respectively, according to the Kabat numbering scheme.

33. The antibody or antigen-binding fragment thereof of claim 27, wherein the VH comprises CDRs 1, 2, 3 with the amino acid sequences set forth in SEQ ID NOs: 51-53, respectively, and the VL comprises CDRs 1, 2, 3 with the amino acid sequences set forth in SEQ ID NOs: 29-31, respectively, according to the Kabat numbering scheme.

34. The antibody or antigen-binding fragment thereof of claim 27, wherein (1) the VH comprises CDRs 1, 2, 3 with the amino acid sequences set forth in SEQ ID NOs: 54-56, respectively, and the VL comprises CDRs 1, 2, 3 with the amino acid sequences set forth in SEQ ID NOs: 29-31, respectively, according to the Kabat numbering scheme; or (2) the VH comprises CDRs 1, 2, 3 with the amino acid sequences set forth in SEQ ID NOs: 54-56, respectively, and the VL comprises CDRs 1, 2, 3 with the amino acid sequences set forth in SEQ ID NOs: 149-151, respectively, according to the Kabat numbering scheme.

35. The antibody or antigen-binding fragment thereof of claim 27, wherein (1) the VH comprises CDRs 1, 2, 3 with the amino acid sequences set forth in SEQ ID NOs: 57-59, respectively, and the VL comprises CDRs 1, 2, 3 with the amino acid sequences set forth in SEQ ID NOs: 29-31, respectively, according to the Kabat numbering scheme; or (2) the VH comprises CDRs 1, 2, 3 with the amino acid sequences set forth in SEQ ID NOs: 57-59, respectively, and the VL comprises CDRs 1, 2, 3 with the amino acid sequences set forth in SEQ ID NOs: 152-154, respectively, according to the Kabat numbering scheme.

36. The antibody or antigen-binding fragment thereof of claim 27, wherein the VH comprises CDRs 1, 2, 3 with the amino acid sequences set forth in SEQ ID NOs: 60-62, respectively, and the VL comprises CDRs 1, 2, 3 with the amino acid sequences set forth in SEQ ID NOs: 32-34, respectively, according to the Chothia numbering scheme.

37. The antibody or antigen-binding fragment thereof of claim 27, wherein the VH comprises CDRs 1, 2, 3 with the amino acid sequences set forth in SEQ ID NOs: 63-65, respectively, and the VL comprises CDRs 1, 2, 3 with the amino acid sequences set forth in SEQ ID NOs: 32-34, respectively, according to the Chothia numbering scheme.

38. The antibody or antigen-binding fragment thereof of claim 27, wherein the VH comprises CDRs 1, 2, 3 with the amino acid sequences set forth in SEQ ID NOs: 66-68, respectively, and the VL comprises CDRs 1, 2, 3 with the amino acid sequences set forth in SEQ ID NOs: 32-34, respectively, according to the Chothia numbering scheme.

39. The antibody or antigen-binding fragment thereof of claim 27, wherein the VH comprises CDRs 1, 2, 3 with the amino acid sequences set forth in SEQ ID NOs: 69-71, respectively, and the VL comprises CDRs 1, 2, 3 with the amino acid sequences set forth in SEQ ID NOs: 32-34, respectively, according to the Chothia numbering scheme.

40. The antibody or antigen-binding fragment thereof of claim 27, wherein the VH comprises CDRs 1, 2, 3 with the amino acid sequences set forth in SEQ ID NOs: 72-74, respectively, and the VL comprises CDRs 1, 2, 3 with the amino acid sequences set forth in SEQ ID NOs: 32-34, respectively, according to the Chothia numbering scheme.

41. The antibody or antigen-binding fragment thereof of claim 27, wherein the VH comprises CDRs 1, 2, 3 with the amino acid sequences set forth in SEQ ID NOs: 75-77, respectively, and the VL comprises CDRs 1, 2, 3 with the amino acid sequences set forth in SEQ ID NOs: 32-34, respectively, according to the Chothia numbering scheme.

42. The antibody or antigen-binding fragment thereof of claim 27, wherein (1) the VH comprises CDRs 1, 2, 3 with the amino acid sequences set forth in SEQ ID NOs: 78-80, respectively, and the VL comprises CDRs 1, 2, 3 with the amino acid sequences set forth in SEQ ID NOs: 32-34, respectively, according to the Chothia numbering scheme; or (2) the VH comprises CDRs 1, 2, 3 with the amino acid sequences set forth in SEQ ID NOs: 78-80, respectively, and the VL comprises CDRs 1, 2, 3 with the amino acid sequences set forth in SEQ ID NOs: 155-157, respectively, according to the Chothia numbering scheme.

43. The antibody or antigen-binding fragment thereof of claim 27, wherein (1) the VH comprises CDRs 1, 2, 3 with the amino acid sequences set forth in SEQ ID NOs: 81-83, respectively, and the VL comprises CDRs 1, 2, 3 with the amino acid sequences set forth in SEQ ID NOs: 32-34, respectively, according to the Chothia numbering scheme; or (2) the VH comprises CDRs 1, 2, 3 with the amino acid sequences set forth in SEQ ID NOs: 81-83, respectively, and the VL comprises CDRs 1, 2, 3 with the amino acid sequences set forth in SEQ ID NOs: 158-160, respectively, according to the Chothia numbering scheme.

44. An antibody or antigen-binding fragment thereof that binds to OX40 comprising
a heavy chain variable region (VH) comprising an amino acid sequence that is at least 90% identical to a selected VH sequence, and a light chain variable region (VL) comprising an amino acid sequence that is at least 90% identical to a selected VL sequence, wherein the selected VH sequence and the selected VL sequence are one of the following:
(1) the selected VH sequence is SEQ ID NO: 84, and the selected VL sequence is SEQ ID NO: 35;
(2) the selected VH sequence is SEQ ID NO: 85, and the selected VL sequence is SEQ ID NO: 35;
(3) the selected VH sequence is SEQ ID NO: 86, and the selected VL sequence is SEQ ID NO: 35;
(4) the selected VH sequence is SEQ ID NO: 87, and the selected VL sequence is SEQ ID NO: 35;
(5) the selected VH sequence is SEQ ID NO: 88, and the selected VL sequence is SEQ ID NO: 35;
(6) the selected VH sequence is SEQ ID NO: 89, and the selected VL sequence is SEQ ID NO: 35;
(7) the selected VH sequence is SEQ ID NO: 90, and the selected VL sequence is SEQ ID NO: 35 or 161; and
(8) the selected VH sequence is SEQ ID NO: 91, and the selected VL sequence is SEQ ID NO: 35 or 162.

45. The antibody or antigen-binding fragment thereof of claim 44, wherein the VH comprises the sequence of SEQ ID NO: 84 and the VL comprises the sequence of SEQ ID NO: 35.

46. The antibody or antigen-binding fragment thereof of claim 44, wherein the VH comprises the sequence of SEQ ID NO: 85 and the VL comprises the sequence of SEQ ID NO: 35.

47. The antibody or antigen-binding fragment thereof of claim 44, wherein the VH comprises the sequence of SEQ ID NO: 86 and the VL comprises the sequence of SEQ ID NO: 35.

48. The antibody or antigen-binding fragment thereof of claim 44, wherein the VH comprises the sequence of SEQ ID NO: 87 and the VL comprises the sequence of SEQ ID NO: 35.

49. The antibody or antigen-binding fragment thereof of claim 44, wherein the VH comprises the sequence of SEQ ID NO: 88 and the VL comprises the sequence of SEQ ID NO: 35.

50. The antibody or antigen-binding fragment thereof of claim 44, wherein the VH comprises the sequence of SEQ ID NO: 89 and the VL comprises the sequence of SEQ ID NO: 35.

51. The antibody or antigen-binding fragment thereof of claim 44, wherein the VH comprises the sequence of SEQ ID NO: 90 and the VL comprises the sequence of SEQ ID NO: 35 or 161.

52. The antibody or antigen-binding fragment thereof of claim 44, wherein the VH comprises the sequence of SEQ ID NO: 91 and the VL comprises the sequence of SEQ ID NO: 35 or 162.

53. An antibody or antigen-binding fragment thereof that binds to OX40 comprising
a heavy chain variable region (VH) comprising VH CDR1, VH CDR2, and VH CDR3 that are identical to VH CDR1, VH CDR2, and VH CDR3 of a selected VH sequence; and a light chain variable region (VL) comprising VL CDR1, VL CDR2, and VL CDR3 that are identical to VL CDR1, VL CDR2, and VL CDR3 of a selected VL sequence,
wherein the selected VH sequence and the selected VL sequence are one of the following:
(1) the selected VH sequence is SEQ ID NO: 84, and the selected VL sequence is SEQ ID NO: 35;
(2) the selected VH sequence is SEQ ID NO: 85, and the selected VL sequence is SEQ ID NO: 35;
(3) the selected VH sequence is SEQ ID NO: 86, and the selected VL sequence is SEQ ID NO: 35;
(4) the selected VH sequence is SEQ ID NO: 87, and the selected VL sequence is SEQ ID NO: 35;
(5) the selected VH sequence is SEQ ID NO: 88, and the selected VL sequence is SEQ ID NO: 35;
(6) the selected VH sequence is SEQ ID NO: 89, and the selected VL sequence is SEQ ID NO: 35;
(7) the selected VH sequence is SEQ ID NO: 90, and the selected VL sequence is SEQ ID NO: 35 or 161; and
(8) the selected VH sequence is SEQ ID NO: 91, and the selected VL sequence is SEQ ID NO: 35 or 162.

54. The antibody or antigen-binding fragment thereof of any one of claims 27-53, wherein the antibody or antigen-binding fragment thereof specifically binds to human or monkey OX40.

55. The antibody or antigen-binding fragment thereof of any one of claims 27-54, wherein the antibody or antigen-binding fragment is a human or humanized antibody or antigen-binding fragment thereof.

56. The antibody or antigen-binding fragment thereof of any one of claims 27-55, wherein the antibody or antigen-binding fragment is a single-chain variable fragment (scFV).

57. The antibody or antigen-binding fragment thereof of any one of claims 27-55, wherein the antibody or antigen-binding fragment thereof is a bispecific or a multispecific antibody or an antigen-binding fragment thereof.

58. The antibody or antigen-binding fragment thereof of claim 57, wherein the antibody or antigen-binding fragment thereof further specifically binds to CTLA4.

59. An antibody or antigen-binding fragment thereof that cross-competes with the antibody or antigen-binding fragment thereof of any one of claims 27-58.

60. A nucleic acid comprising a polynucleotide encoding a polypeptide comprising:
(1) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 36-38, respectively, and wherein the VH, when paired with a light chain variable region (VL) comprising the amino acid sequence set forth in SEQ ID NO: 35, binds to OX40;
(2) an immunoglobulin light chain or a fragment thereof comprising a light chain variable region (VL) comprising complementarity determining regions (CDRs) 1, 2, and 3 comprising the amino acid sequences set forth in SEQ ID NOs: 29-31, respectively, and wherein the VL, when paired with a heavy chain variable region (VH) comprising the amino acid sequence set forth in SEQ ID NO: 84, binds to OX40;
(3) an immunoglobulin heavy chain or a fragment thereof comprising a VH comprising CDRs 1, 2, and 3 comprising the amino acid sequences set forth in SEQ ID NOs: 39-41, respectively, and wherein the VH, when paired with a VL comprising the amino acid sequence set forth in SEQ ID NO: 35, binds to OX40;
(4) an immunoglobulin light chain or a fragment thereof comprising a VL comprising CDRs 1, 2, and 3 comprising the amino acid sequences set forth in SEQ ID NOs: 29-31, respectively, and wherein the VL, when paired with a VH comprising the amino acid sequence set forth in SEQ ID NO: 85, binds to OX40;
(5) an immunoglobulin heavy chain or a fragment thereof comprising a VH comprising CDRs 1, 2, and 3 comprising the amino acid sequences set forth in SEQ ID NOs: 42-44, respectively, and wherein the VH, when paired with a VL comprising the amino acid sequence set forth in SEQ ID NO: 35, binds to OX40;
(6) an immunoglobulin light chain or a fragment thereof comprising a VL comprising CDRs 1, 2, and 3 comprising the amino acid sequences set forth in SEQ ID NOs: 29-31, respectively, and wherein the VL, when paired with a VH comprising the amino acid sequence set forth in SEQ ID NO: 86, binds to OX40;
(7) an immunoglobulin heavy chain or a fragment thereof comprising a VH comprising CDRs 1, 2, and 3 comprising the amino acid sequences set forth in SEQ ID NOs: 45-47, respectively, and wherein the VH, when paired with a VL comprising the amino acid sequence set forth in SEQ ID NO: 35, binds to OX40;
(8) an immunoglobulin light chain or a fragment thereof comprising a VL comprising CDRs 1, 2, and 3 comprising the amino acid sequences set forth in SEQ ID NOs: 29-31, respectively, and wherein the VL, when paired with a VH comprising the amino acid sequence set forth in SEQ ID NO: 87, binds to OX40;
(9) an immunoglobulin heavy chain or a fragment thereof comprising a VH comprising CDRs 1, 2, and 3 comprising the amino acid sequences set forth in SEQ ID NOs: 48-50, respectively, and wherein the VH, when paired with a VL comprising the amino acid sequence set forth in SEQ ID NO: 35, binds to OX40;
(10) an immunoglobulin light chain or a fragment thereof comprising a VL comprising CDRs 1, 2, and 3 comprising the amino acid sequences set forth in SEQ ID NOs: 29-31, respectively, and wherein the VL, when paired with a VH comprising the amino acid sequence set forth in SEQ ID NO: 88, binds to OX40;
(11) an immunoglobulin heavy chain or a fragment thereof comprising a VH comprising CDRs 1, 2, and 3 comprising the amino acid sequences set forth in SEQ ID NOs: 51-53, respectively, and wherein the VH, when paired with a VL comprising the amino acid sequence set forth in SEQ ID NO: 35, binds to OX40;
(12) an immunoglobulin light chain or a fragment thereof comprising a VL comprising CDRs 1, 2, and 3 comprising the amino acid sequences set forth in SEQ ID NOs: 29-31, respectively, and wherein the VL, when paired with a VH comprising the amino acid sequence set forth in SEQ ID NO: 89, binds to OX40;
(13) an immunoglobulin heavy chain or a fragment thereof comprising a VH comprising CDRs 1, 2, and 3 comprising the amino acid sequences set forth in SEQ ID NOs: 54-56, respectively, and wherein the VH, when paired with a VL comprising the amino acid sequence set forth in SEQ ID NO: 35 or 161, binds to OX40;
(14) an immunoglobulin light chain or a fragment thereof comprising a VL comprising CDRs 1, 2, and 3 comprising the amino acid sequences set forth in SEQ ID NOs: 29-31 or SEQ ID NOs: 149-151, respectively, and wherein the VL, when paired with a VH comprising the amino acid sequence set forth in SEQ ID NO: 90, binds to OX40;
(15) an immunoglobulin heavy chain or a fragment thereof comprising a VH comprising CDRs 1, 2, and 3 comprising the amino acid sequences set forth in SEQ ID NOs: 57-59, respectively, and wherein the VH, when paired with a VL comprising the amino acid sequence set forth in SEQ ID NO: 35 or 162, binds to OX40;
(16) an immunoglobulin light chain or a fragment thereof comprising a VL comprising CDRs 1, 2, and 3 comprising the amino acid sequences set forth in SEQ ID NOs: 29-31 or SEQ ID NOs: 152-154, respectively, and wherein the VL, when paired with a VH comprising the amino acid sequence set forth in SEQ ID NO: 91, binds to OX40;
(17) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 60-62, respectively, and wherein the VH, when paired with a light chain variable region (VL) comprising the amino acid sequence set forth in SEQ ID NO: 35, binds to OX40;
(18) an immunoglobulin light chain or a fragment thereof comprising a light chain variable region (VL) comprising complementarity determining regions (CDRs) 1, 2, and 3 comprising the amino acid sequences set forth in SEQ ID NOs: 32-34, respectively, and wherein the VL, when paired with a heavy chain variable region (VH) comprising the amino acid sequence set forth in SEQ ID NO: 84, binds to OX40;
(19) an immunoglobulin heavy chain or a fragment thereof comprising a VH comprising CDRs 1, 2, and 3 comprising the amino acid sequences set forth in SEQ ID NOs: 63-65, respectively, and wherein the VH, when paired with a VL comprising the amino acid sequence set forth in SEQ ID NO: 35, binds to OX40;
(20) an immunoglobulin light chain or a fragment thereof comprising a VL comprising CDRs 1, 2, and 3 comprising the amino acid sequences set forth in SEQ ID NOs: 32-34, respectively, and wherein the VL, when paired with a VH comprising the amino acid sequence set forth in SEQ ID NO: 85, binds to OX40;
(21) an immunoglobulin heavy chain or a fragment thereof comprising a VH comprising CDRs 1, 2, and 3 comprising the amino acid sequences set forth in SEQ ID NOs: 66-68, respectively, and wherein the VH, when paired with a VL comprising the amino acid sequence set forth in SEQ ID NO: 35, binds to OX40;
(22) an immunoglobulin light chain or a fragment thereof comprising a VL comprising CDRs 1, 2, and 3 comprising the amino acid sequences set forth in SEQ ID NOs: 32-34, respectively, and wherein the VL, when paired with a VH comprising the amino acid sequence set forth in SEQ ID NO: 86, binds to OX40;
(23) an immunoglobulin heavy chain or a fragment thereof comprising a VH comprising CDRs 1, 2, and 3 comprising the amino acid sequences set forth in SEQ ID NOs: 69-71, respectively, and wherein the VH, when paired with a VL comprising the amino acid sequence set forth in SEQ ID NO: 35, binds to OX40;
(24) an immunoglobulin light chain or a fragment thereof comprising a VL comprising CDRs 1, 2, and 3 comprising the amino acid sequences set forth in SEQ ID NOs: 32-34, respectively, and wherein the VL, when paired with a VH comprising the amino acid sequence set forth in SEQ ID NO: 87, binds to OX40;
(25) an immunoglobulin heavy chain or a fragment thereof comprising a VH comprising CDRs 1, 2, and 3 comprising the amino acid sequences set forth in SEQ ID NOs: 72-74, respectively, and wherein the VH, when paired with a VL comprising the amino acid sequence set forth in SEQ ID NO: 35, binds to OX40;
(26) an immunoglobulin light chain or a fragment thereof comprising a VL comprising CDRs 1, 2, and 3 comprising the amino acid sequences set forth in SEQ ID NOs: 32-34, respectively, and wherein the VL, when paired with a VH comprising the amino acid sequence set forth in SEQ ID NO: 88, binds to OX40;
(27) an immunoglobulin heavy chain or a fragment thereof comprising a VH comprising CDRs 1, 2, and 3 comprising the amino acid sequences set forth in SEQ ID NOs: 75-77, respectively, and wherein the VH, when paired with a VL comprising the amino acid sequence set forth in SEQ ID NO: 35, binds to OX40;
(28) an immunoglobulin light chain or a fragment thereof comprising a VL comprising CDRs 1, 2, and 3 comprising the amino acid sequences set forth in SEQ ID NOs: 32-34, respectively, and wherein the VL, when paired with a VH comprising the amino acid sequence set forth in SEQ ID NO: 89, binds to OX40;
(29) an immunoglobulin heavy chain or a fragment thereof comprising a VH comprising CDRs 1, 2, and 3 comprising the amino acid sequences set forth in SEQ ID NOs: 78-80, respectively, and wherein the VH, when paired with a VL comprising the amino acid sequence set forth in SEQ ID NO: 35 or 161, binds to OX40;
(30) an immunoglobulin light chain or a fragment thereof comprising a VL comprising CDRs 1, 2, and 3 comprising the amino acid sequences set forth in SEQ ID NOs: 32-34 or SEQ ID NOs: 155-157, respectively, and wherein the VL, when paired with a VH comprising the amino acid sequence set forth in SEQ ID NO: 90, binds to OX40;
(31) an immunoglobulin heavy chain or a fragment thereof comprising a VH comprising CDRs 1, 2, and 3 comprising the amino acid sequences set forth in SEQ ID NOs: 81-83, respectively, and wherein the VH, when paired with a VL comprising the amino acid sequence set forth in SEQ ID NO: 35 or 162, binds to OX40; and
(32) an immunoglobulin light chain or a fragment thereof comprising a VL comprising CDRs 1, 2, and 3 comprising the amino acid sequences set forth in SEQ ID NOs: 32-34 or SEQ ID NOs: 158-160, respectively, and wherein the VL, when paired with a VH comprising the amino acid sequence set forth in SEQ ID NO: 91, binds to OX40.

61. The nucleic acid of claim 60, wherein the VH when paired with a VL specifically binds to human or monkey OX40.

62. The nucleic acid of claim 60 or 61, wherein the immunoglobulin heavy chain or the fragment thereof is a human or humanized immunoglobulin heavy chain or a fragment thereof.

63. The nucleic acid of any one of claims 60-62, wherein the nucleic acid encodes a single-chain variable fragment (scFv).

64. The nucleic acid of any one of claims 60-63, wherein the nucleic acid is cDNA.

65. An antigen-binding protein construct, comprising: a first antigen-binding domain that specifically binds to CTLA4; and a second antigen-binding domain that specially binds to OX40.

66. The antigen-binding protein construct of claim 65, wherein the first antigen-binding domain comprises a first heavy chain variable region (VH1) and a first light chain variable region (VL1); and the second antigen-binding domain comprises a second heavy chain variable region (VH2) and a second light chain variable region (VL2).

67. The antigen-binding protein construct of claim 66, wherein
the first heavy chain variable region (VH1) comprises complementarity determining regions (CDRs) 1, 2, and 3, wherein the VH1 CDR1 region comprises an amino acid sequence that is at least 80% identical to a selected VH1 CDR1 amino acid sequence, the VH1 CDR2 region comprises an amino acid sequence that is at least 80% identical to a selected VH1 CDR2 amino acid sequence, and the VH1 CDR3 region comprises an amino acid sequence that is at least 80% identical to a selected VH1 CDR3 amino acid sequence; and
the first light chain variable region (VL1) comprises CDRs 1, 2, and 3, wherein the VL1 CDR1 region comprises an amino acid sequence that is at least 80% identical to a selected VL1 CDR1 amino acid sequence, the VL1 CDR2 region comprises an amino acid sequence that is at least 80% identical to a selected VL1 CDR2 amino acid sequence, and the VL1 CDR3 region comprises an amino acid sequence that is at least 80% identical to a selected VL1 CDR3 amino acid sequence,
wherein the selected VH1 CDRs 1, 2, and 3 amino acid sequences, the selected VL1 CDRs 1, 2, and 3 amino acid sequences are one of the following:
(1) the selected VH1 CDRs 1, 2, 3 amino acid sequences are set forth in SEQ ID NOs: 1-3, respectively, and the selected VL1 CDRs 1, 2, 3 amino acid sequences are set forth in SEQ ID NOs: 29-31, respectively;
(2) the selected VH1 CDRs 1, 2, 3 amino acid sequences are set forth in SEQ ID NOs: 4-6, respectively, and the selected VL1 CDRs 1, 2, 3 amino acid sequences are set forth in SEQ ID NOs: 29-31, respectively;
(3) the selected VH1 CDRs 1, 2, 3 amino acid sequences are set forth in SEQ ID NOs: 7-9, respectively, and the selected VL1 CDRs 1, 2, 3 amino acid sequences are set forth in SEQ ID NOs: 29-31, respectively;
(4) the selected VH1 CDRs 1, 2, 3 amino acid sequences are set forth in SEQ ID NOs: 10-12, respectively, and the selected VL1 CDRs 1, 2, 3 amino acid sequences are set forth in SEQ ID NOs: 29-31, respectively;
(5) the selected VH1 CDRs 1, 2, 3 amino acid sequences are set forth in SEQ ID NOs: 13-15, respectively, and the selected VL1 CDRs 1, 2, 3 amino acid sequences are set forth in SEQ ID NOs: 32-34, respectively;
(6) the selected VH1 CDRs 1, 2, 3 amino acid sequences are set forth in SEQ ID NOs: 16-18, respectively, and the selected VL1 CDRs 1, 2, 3 amino acid sequences are set forth in SEQ ID NOs: 32-34, respectively;
(7) the selected VH1 CDRs 1, 2, 3 amino acid sequences are set forth in SEQ ID NOs: 19-21, respectively, and the selected VL1 CDRs 1, 2, 3 amino acid sequences are set forth in SEQ ID NOs: 32-34, respectively; and
(8) the selected VH1 CDRs 1, 2, 3 amino acid sequences are set forth in SEQ ID NOs: 22-24, respectively, and the selected VL1 CDRs 1, 2, 3 amino acid sequences are set forth in SEQ ID NOs: 32-34, respectively.

68. The antigen-binding protein construct of claim 66 or 67, wherein
the second heavy chain variable region (VH2) comprises CDRs 1, 2, and 3, wherein the VH2 CDR1 region comprises an amino acid sequence that is at least 80% identical to a selected VH2 CDR1 amino acid sequence, the VH2 CDR2 region comprises an amino acid sequence that is at least 80% identical to a selected VH2 CDR2 amino acid sequence, and the VH2 CDR3 region comprises an amino acid sequence that is at least 80% identical to a selected VH2 CDR3 amino acid sequence; and
the second light chain variable region (VL2) comprises CDRs 1, 2, and 3, wherein the VL2 CDR1 region comprises an amino acid sequence that is at least 80% identical to a selected VL2 CDR1 amino acid sequence, the VL2 CDR2 region comprises an amino acid sequence that is at least 80% identical to a selected VL2 CDR2 amino acid sequence, and the VL2 CDR3 region comprises an amino acid sequence that is at least 80% identical to a selected VL2 CDR3 amino acid sequence,
wherein the selected VH2 CDRs 1, 2, and 3 amino acid sequences, and the selected VL2 CDRs 1, 2, and 3 amino acid sequences are one of the following:
(1) the selected VH2 CDRs 1, 2, 3 amino acid sequences are set forth in SEQ ID NOs: 36-38, respectively, and the selected VL2 CDRs 1, 2, 3 amino acid sequences are set forth in SEQ ID NOs: 29-31, respectively;
(2) the selected VH2 CDRs 1, 2, 3 amino acid sequences are set forth in SEQ ID NOs: 39-41, respectively, and the selected VL2 CDRs 1, 2, 3 amino acid sequences are set forth in SEQ ID NOs: 29-31, respectively;
(3) the selected VH2 CDRs 1, 2, 3 amino acid sequences are set forth in SEQ ID NOs: 42-44, respectively, and the selected VL2 CDRs 1, 2, 3 amino acid sequences are set forth in SEQ ID NOs: 29-31, respectively;
(4) the selected VH2 CDRs 1, 2, 3 amino acid sequences are set forth in SEQ ID NOs: 45-47, respectively, and the selected VL2 CDRs 1, 2, 3 amino acid sequences are set forth in SEQ ID NOs: 29-31, respectively;
(5) the selected VH2 CDRs 1, 2, 3 amino acid sequences are set forth in SEQ ID NOs: 48-50, respectively, and the selected VL2 CDRs 1, 2, 3 amino acid sequences are set forth in SEQ ID NOs: 29-31, respectively;
(6) the selected VH2 CDRs 1, 2, 3 amino acid sequences are set forth in SEQ ID NOs: 51-53, respectively, and the selected VL2 CDRs 1, 2, 3 amino acid sequences are set forth in SEQ ID NOs: 29-31, respectively;
(7) the selected VH2 CDRs 1, 2, 3 amino acid sequences are set forth in SEQ ID NOs: 54-56, respectively, and the selected VL2 CDRs 1, 2, 3 amino acid sequences are set forth in SEQ ID NOs: 29-31, respectively; alternatively, the selected VH2 CDRs 1, 2, 3 amino acid sequences are set forth in SEQ ID NOs: 54-56, respectively, and the selected VL2 CDRs 1, 2, 3 amino acid sequences are set forth in SEQ ID NOs: 149-151, respectively;
(8) the selected VH2 CDRs 1, 2, 3 amino acid sequences are set forth in SEQ ID NOs: 57-59, respectively, and the selected VL2 CDRs 1, 2, 3 amino acid sequences are set forth in SEQ ID NOs: 29-31, respectively; alternatively, the selected VH2 CDRs 1, 2, 3 amino acid sequences are set forth in SEQ ID NOs: 57-59, respectively, and the selected VL2 CDRs 1, 2, 3 amino acid sequences are set forth in SEQ ID NOs: 152-154, respectively;
(9) the selected VH2 CDRs 1, 2, 3 amino acid sequences are set forth in SEQ ID NOs: 60-62, respectively, and the selected VL2 CDRs 1, 2, 3 amino acid sequences are set forth in SEQ ID NOs: 32-34, respectively;
(10) the selected VH2 CDRs 1, 2, 3 amino acid sequences are set forth in SEQ ID NOs: 63-65, respectively, and the selected VL2 CDRs 1, 2, 3 amino acid sequences are set forth in SEQ ID NOs: 32-34, respectively;
(11) the selected VH2 CDRs 1, 2, 3 amino acid sequences are set forth in SEQ ID NOs: 66-68, respectively, and the selected VL2 CDRs 1, 2, 3 amino acid sequences are set forth in SEQ ID NOs: 32-34, respectively;
(12) the selected VH2 CDRs 1, 2, 3 amino acid sequences are set forth in SEQ ID NOs: 69-71, respectively, and the selected VL2 CDRs 1, 2, 3 amino acid sequences are set forth in SEQ ID NOs: 32-34, respectively;
(13) the selected VH2 CDRs 1, 2, 3 amino acid sequences are set forth in SEQ ID NOs: 72-74, respectively, and the selected VL2 CDRs 1, 2, 3 amino acid sequences are set forth in SEQ ID NOs: 32-34, respectively;
(14) the selected VH2 CDRs 1, 2, 3 amino acid sequences are set forth in SEQ ID NOs: 75-77, respectively, and the selected VL2 CDRs 1, 2, 3 amino acid sequences are set forth in SEQ ID NOs: 32-34, respectively;
(15) the selected VH2 CDRs 1, 2, 3 amino acid sequences are set forth in SEQ ID NOs: 78-80, respectively, and the selected VL2 CDRs 1, 2, 3 amino acid sequences are set forth in SEQ ID NOs: 32-34, respectively; alternatively, the selected VH2 CDRs 1, 2, 3 amino acid sequences are set forth in SEQ ID NOs: 78-80, respectively, and the selected VL2 CDRs 1, 2, 3 amino acid sequences are set forth in SEQ ID NOs: 155-157, respectively; and
(16) the selected VH2 CDRs 1, 2, 3 amino acid sequences are set forth in SEQ ID NOs: 81-83, respectively, and the selected VL2 CDRs 1, 2, 3 amino acid sequences are set forth in SEQ ID NOs: 32-34, respectively; alternatively, the selected VH2 CDRs 1, 2, 3 amino acid sequences are set forth in SEQ ID NOs: 81-83, respectively, and the selected VL2 CDRs 1, 2, 3 amino acid sequences are set forth in SEQ ID NOs: 158-160, respectively.

69. The antigen-binding protein construct of any one of claims 66-68, wherein
(1) the selected VH1 CDRs 1, 2, 3 amino acid sequences are set forth in SEQ ID NOs: 1-3, respectively, and the selected VL1 CDRs 1, 2, 3 amino acid sequences are set forth in SEQ ID NOs: 29-31, respectively, and the selected VH2 CDRs 1, 2, 3 amino acid sequences are set forth in SEQ ID NOs: 36-38, respectively, and the selected VL2 CDRs 1, 2, 3 amino acid sequences are set forth in SEQ ID NOs: 29-31, respectively;
(2) the selected VH1 CDRs 1, 2, 3 amino acid sequences are set forth in SEQ ID NOs: 1-3, respectively, and the selected VL1 CDRs 1, 2, 3 amino acid sequences are set forth in SEQ ID NOs: 29-31, respectively, and the selected VH2 CDRs 1, 2, 3 amino acid sequences are set forth in SEQ ID NOs: 39-41, respectively, and the selected VL2 CDRs 1, 2, 3 amino acid sequences are set forth in SEQ ID NOs: 29-31, respectively;
(3) the selected VH1 CDRs 1, 2, 3 amino acid sequences are set forth in SEQ ID NOs: 1-3, respectively, and the selected VL1 CDRs 1, 2, 3 amino acid sequences are set forth in SEQ ID NOs: 29-31, respectively, and the selected VH2 CDRs 1, 2, 3 amino acid sequences are set forth in SEQ ID NOs: 42-44, respectively, and the selected VL2 CDRs 1, 2, 3 amino acid sequences are set forth in SEQ ID NOs: 29-31, respectively;
(4) the selected VH1 CDRs 1, 2, 3 amino acid sequences are set forth in SEQ ID NOs: 1-3, respectively, and the selected VL1 CDRs 1, 2, 3 amino acid sequences are set forth in SEQ ID NOs: 29-31, respectively, and the selected VH2 CDRs 1, 2, 3 amino acid sequences are set forth in SEQ ID NOs: 45-47, respectively, and the selected VL2 CDRs 1, 2, 3 amino acid sequences are set forth in SEQ ID NOs: 29-31, respectively;
(5) the selected VH1 CDRs 1, 2, 3 amino acid sequences are set forth in SEQ ID NOs: 1-3, respectively, and the selected VL1 CDRs 1, 2, 3 amino acid sequences are set forth in SEQ ID NOs: 29-31, respectively, and the selected VH2 CDRs 1, 2, 3 amino acid sequences are set forth in SEQ ID NOs: 48-50, respectively, and the selected VL2 CDRs 1, 2, 3 amino acid sequences are set forth in SEQ ID NOs: 29-31, respectively;
(6) the selected VH1 CDRs 1, 2, 3 amino acid sequences are set forth in SEQ ID NOs: 1-3, respectively, and the selected VL1 CDRs 1, 2, 3 amino acid sequences are set forth in SEQ ID NOs: 29-31, respectively, and the selected VH2 CDRs 1, 2, 3 amino acid sequences are set forth in SEQ ID NOs: 51-53, respectively, and the selected VL2 CDRs 1, 2, 3 amino acid sequences are set forth in SEQ ID NOs: 29-31, respectively;
(7) the selected VH1 CDRs 1, 2, 3 amino acid sequences are set forth in SEQ ID NOs: 1-3, respectively, and the selected VL1 CDRs 1, 2, 3 amino acid sequences are set forth in SEQ ID NOs: 29-31, respectively, and the selected VH2 CDRs 1, 2, 3 amino acid sequences are set forth in SEQ ID NOs: 54-56, respectively, and the selected VL2 CDRs 1, 2, 3 amino acid sequences are set forth in SEQ ID NOs: 29-31, respectively;
(8) the selected VH1 CDRs 1, 2, 3 amino acid sequences are set forth in SEQ ID NOs: 1-3, respectively, and the selected VL1 CDRs 1, 2, 3 amino acid sequences are set forth in SEQ ID NOs: 29-31, respectively, and the selected VH2 CDRs 1, 2, 3 amino acid sequences are set forth in SEQ ID NOs: 57-59, respectively, and the selected VL2 CDRs 1, 2, 3 amino acid sequences are set forth in SEQ ID NOs: 29-31, respectively;
(9) the selected VH1 CDRs 1, 2, 3 amino acid sequences are set forth in SEQ ID NOs: 4-6, respectively, and the selected VL1 CDRs 1, 2, 3 amino acid sequences are set forth in SEQ ID NOs: 29-31, respectively, and the selected VH2 CDRs 1, 2, 3 amino acid sequences are set forth in SEQ ID NOs: 36-38, respectively, and the selected VL2 CDRs 1, 2, 3 amino acid sequences are set forth in SEQ ID NOs: 29-31, respectively;
(10) the selected VH1 CDRs 1, 2, 3 amino acid sequences are set forth in SEQ ID NOs: 4-6, respectively, and the selected VL1 CDRs 1, 2, 3 amino acid sequences are set forth in SEQ ID NOs: 29-31, respectively, and the selected VH2 CDRs 1, 2, 3 amino acid sequences are set forth in SEQ ID NOs: 39-41, respectively, and the selected VL2 CDRs 1, 2, 3 amino acid sequences are set forth in SEQ ID NOs: 29-31, respectively;
(11) the selected VH1 CDRs 1, 2, 3 amino acid sequences are set forth in SEQ ID NOs: 4-6, respectively, and the selected VL1 CDRs 1, 2, 3 amino acid sequences are set forth in SEQ ID NOs: 29-31, respectively, and the selected VH2 CDRs 1, 2, 3 amino acid sequences are set forth in SEQ ID NOs: 42-44, respectively, and the selected VL2 CDRs 1, 2, 3 amino acid sequences are set forth in SEQ ID NOs: 29-31, respectively;
(12) the selected VH1 CDRs 1, 2, 3 amino acid sequences are set forth in SEQ ID NOs: 4-6, respectively, and the selected VL1 CDRs 1, 2, 3 amino acid sequences are set forth in SEQ ID NOs: 29-31, respectively, and the selected VH2 CDRs 1, 2, 3 amino acid sequences are set forth in SEQ ID NOs: 45-47, respectively, and the selected VL2 CDRs 1, 2, 3 amino acid sequences are set forth in SEQ ID NOs: 29-31, respectively;
(13) the selected VH1 CDRs 1, 2, 3 amino acid sequences are set forth in SEQ ID NOs: 4-6, respectively, and the selected VL1 CDRs 1, 2, 3 amino acid sequences are set forth in SEQ ID NOs: 29-31, respectively, and the selected VH2 CDRs 1, 2, 3 amino acid sequences are set forth in SEQ ID NOs: 48-50, respectively, and the selected VL2 CDRs 1, 2, 3 amino acid sequences are set forth in SEQ ID NOs: 29-31, respectively;
(14) the selected VH1 CDRs 1, 2, 3 amino acid sequences are set forth in SEQ ID NOs: 4-6, respectively, and the selected VL1 CDRs 1, 2, 3 amino acid sequences are set forth in SEQ ID NOs: 29-31, respectively, and the selected VH2 CDRs 1, 2, 3 amino acid sequences are set forth in SEQ ID NOs: 51-53, respectively, and the selected VL2 CDRs 1, 2, 3 amino acid sequences are set forth in SEQ ID NOs: 29-31, respectively;
(15) the selected VH1 CDRs 1, 2, 3 amino acid sequences are set forth in SEQ ID NOs: 4-6, respectively, and the selected VL1 CDRs 1, 2, 3 amino acid sequences are set forth in SEQ ID NOs: 29-31, respectively, and the selected VH2 CDRs 1, 2, 3 amino acid sequences are set forth in SEQ ID NOs: 54-56, respectively, and the selected VL2 CDRs 1, 2, 3 amino acid sequences are set forth in SEQ ID NOs: 29-31, respectively;
(16) the selected VH1 CDRs 1, 2, 3 amino acid sequences are set forth in SEQ ID NOs: 4-6, respectively, and the selected VL1 CDRs 1, 2, 3 amino acid sequences are set forth in SEQ ID NOs: 29-31, respectively, and the selected VH2 CDRs 1, 2, 3 amino acid sequences are set forth in SEQ ID NOs: 57-59, respectively, and the selected VL2 CDRs 1, 2, 3 amino acid sequences are set forth in SEQ ID NOs: 29-31, respectively;
(17) the selected VH1 CDRs 1, 2, 3 amino acid sequences are set forth in SEQ ID NOs: 7-9, respectively, and the selected VL1 CDRs 1, 2, 3 amino acid sequences are set forth in SEQ ID NOs: 29-31, respectively, and the selected VH2 CDRs 1, 2, 3 amino acid sequences are set forth in SEQ ID NOs: 36-38, respectively, and the selected VL2 CDRs 1, 2, 3 amino acid sequences are set forth in SEQ ID NOs: 29-31, respectively;
(18) the selected VH1 CDRs 1, 2, 3 amino acid sequences are set forth in SEQ ID NOs: 7-9, respectively, and the selected VL1 CDRs 1, 2, 3 amino acid sequences are set forth in SEQ ID NOs: 29-31, respectively, and the selected VH2 CDRs 1, 2, 3 amino acid sequences are set forth in SEQ ID NOs: 39-41, respectively, and the selected VL2 CDRs 1, 2, 3 amino acid sequences are set forth in SEQ ID NOs: 29-31, respectively;
(19) the selected VH1 CDRs 1, 2, 3 amino acid sequences are set forth in SEQ ID NOs: 7-9, respectively, and the selected VL1 CDRs 1, 2, 3 amino acid sequences are set forth in SEQ ID NOs: 29-31, respectively, and the selected VH2 CDRs 1, 2, 3 amino acid sequences are set forth in SEQ ID NOs: 42-44, respectively, and the selected VL2 CDRs 1, 2, 3 amino acid sequences are set forth in SEQ ID NOs: 29-31, respectively;
(20) the selected VH1 CDRs 1, 2, 3 amino acid sequences are set forth in SEQ ID NOs: 7-9, respectively, and the selected VL1 CDRs 1, 2, 3 amino acid sequences are set forth in SEQ ID NOs: 29-31, respectively, and the selected VH2 CDRs 1, 2, 3 amino acid sequences are set forth in SEQ ID NOs: 45-47, respectively, and the selected VL2 CDRs 1, 2, 3 amino acid sequences are set forth in SEQ ID NOs: 29-31, respectively;
(21) the selected VH1 CDRs 1, 2, 3 amino acid sequences are set forth in SEQ ID NOs: 7-9, respectively, and the selected VL1 CDRs 1, 2, 3 amino acid sequences are set forth in SEQ ID NOs: 29-31, respectively, and the selected VH2 CDRs 1, 2, 3 amino acid sequences are set forth in SEQ ID NOs: 48-50, respectively, and the selected VL2 CDRs 1, 2, 3 amino acid sequences are set forth in SEQ ID NOs: 29-31, respectively;
(22) the selected VH1 CDRs 1, 2, 3 amino acid sequences are set forth in SEQ ID NOs: 7-9, respectively, and the selected VL1 CDRs 1, 2, 3 amino acid sequences are set forth in SEQ ID NOs: 29-31, respectively, and the selected VH2 CDRs 1, 2, 3 amino acid sequences are set forth in SEQ ID NOs: 51-53, respectively, and the selected VL2 CDRs 1, 2, 3 amino acid sequences are set forth in SEQ ID NOs: 29-31, respectively;
(23) the selected VH1 CDRs 1, 2, 3 amino acid sequences are set forth in SEQ ID NOs: 7-9, respectively, and the selected VL1 CDRs 1, 2, 3 amino acid sequences are set forth in SEQ ID NOs: 29-31, respectively, and the selected VH2 CDRs 1, 2, 3 amino acid sequences are set forth in SEQ ID NOs: 54-56, respectively, and the selected VL2 CDRs 1, 2, 3 amino acid sequences are set forth in SEQ ID NOs: 29-31, respectively;
(24) the selected VH1 CDRs 1, 2, 3 amino acid sequences are set forth in SEQ ID NOs: 7-9, respectively, and the selected VL1 CDRs 1, 2, 3 amino acid sequences are set forth in SEQ ID NOs: 29-31, respectively, and the selected VH2 CDRs 1, 2, 3 amino acid sequences are set forth in SEQ ID NOs: 57-59, respectively, and the selected VL2 CDRs 1, 2, 3 amino acid sequences are set forth in SEQ ID NOs: 29-31, respectively;
(25) the selected VH1 CDRs 1, 2, 3 amino acid sequences are set forth in SEQ ID NOs: 10-12, respectively, and the selected VL1 CDRs 1, 2, 3 amino acid sequences are set forth in SEQ ID NOs: 29-31, respectively, and the selected VH2 CDRs 1, 2, 3 amino acid sequences are set forth in SEQ ID NOs: 36-38, respectively, and the selected VL2 CDRs 1, 2, 3 amino acid sequences are set forth in SEQ ID NOs: 29-31, respectively;
(26) the selected VH1 CDRs 1, 2, 3 amino acid sequences are set forth in SEQ ID NOs: 10-12, respectively, and the selected VL1 CDRs 1, 2, 3 amino acid sequences are set forth in SEQ ID NOs: 29-31, respectively, and the selected VH2 CDRs 1, 2, 3 amino acid sequences are set forth in SEQ ID NOs: 39-41, respectively, and the selected VL2 CDRs 1, 2, 3 amino acid sequences are set forth in SEQ ID NOs: 29-31, respectively;
(27) the selected VH1 CDRs 1, 2, 3 amino acid sequences are set forth in SEQ ID NOs: 10-12, respectively, and the selected VL1 CDRs 1, 2, 3 amino acid sequences are set forth in SEQ ID NOs: 29-31, respectively, and the selected VH2 CDRs 1, 2, 3 amino acid sequences are set forth in SEQ ID NOs: 42-44, respectively, and the selected VL2 CDRs 1, 2, 3 amino acid sequences are set forth in SEQ ID NOs: 29-31, respectively;
(28) the selected VH1 CDRs 1, 2, 3 amino acid sequences are set forth in SEQ ID NOs: 10-12, respectively, and the selected VL1 CDRs 1, 2, 3 amino acid sequences are set forth in SEQ ID NOs: 29-31, respectively, and the selected VH2 CDRs 1, 2, 3 amino acid sequences are set forth in SEQ ID NOs: 45-47, respectively, and the selected VL2 CDRs 1, 2, 3 amino acid sequences are set forth in SEQ ID NOs: 29-31, respectively;
(29) the selected VH1 CDRs 1, 2, 3 amino acid sequences are set forth in SEQ ID NOs: 10-12, respectively, and the selected VL1 CDRs 1, 2, 3 amino acid sequences are set forth in SEQ ID NOs: 29-31, respectively, and the selected VH2 CDRs 1, 2, 3 amino acid sequences are set forth in SEQ ID NOs: 48-50, respectively, and the selected VL2 CDRs 1, 2, 3 amino acid sequences are set forth in SEQ ID NOs: 29-31, respectively;
(30) the selected VH1 CDRs 1, 2, 3 amino acid sequences are set forth in SEQ ID NOs: 10-12, respectively, and the selected VL1 CDRs 1, 2, 3 amino acid sequences are set forth in SEQ ID NOs: 29-31, respectively, and the selected VH2 CDRs 1, 2, 3 amino acid sequences are set forth in SEQ ID NOs: 51-53, respectively, and the selected VL2 CDRs 1, 2, 3 amino acid sequences are set forth in SEQ ID NOs: 29-31, respectively;
(31) the selected VH1 CDRs 1, 2, 3 amino acid sequences are set forth in SEQ ID NOs: 10-12, respectively, and the selected VL1 CDRs 1, 2, 3 amino acid sequences are set forth in SEQ ID NOs: 29-31, respectively, and the selected VH2 CDRs 1, 2, 3 amino acid sequences are set forth in SEQ ID NOs: 54-56, respectively, and the selected VL2 CDRs 1, 2, 3 amino acid sequences are set forth in SEQ ID NOs: 29-31, respectively;
(32) the selected VH1 CDRs 1, 2, 3 amino acid sequences are set forth in SEQ ID NOs: 10-12, respectively, and the selected VL1 CDRs 1, 2, 3 amino acid sequences are set forth in SEQ ID NOs: 29-31, respectively, and the selected VH2 CDRs 1, 2, 3 amino acid sequences are set forth in SEQ ID NOs: 57-59, respectively, and the selected VL2 CDRs 1, 2, 3 amino acid sequences are set forth in SEQ ID NOs: 29-31, respectively;
(33) the selected VH1 CDRs 1, 2, 3 amino acid sequences are set forth in SEQ ID NOs: 13-15, respectively, and the selected VL1 CDRs 1, 2, 3 amino acid sequences are set forth in SEQ ID NOs: 32-34, respectively, and the selected VH2 CDRs 1, 2, 3 amino acid sequences are set forth in SEQ ID NOs: 60-62, respectively, and the selected VL2 CDRs 1, 2, 3 amino acid sequences are set forth in SEQ ID NOs: 32-34, respectively;
(34) the selected VH1 CDRs 1, 2, 3 amino acid sequences are set forth in SEQ ID NOs: 13-15, respectively, and the selected VL1 CDRs 1, 2, 3 amino acid sequences are set forth in SEQ ID NOs: 32-34, respectively, and the selected VH2 CDRs 1, 2, 3 amino acid sequences are set forth in SEQ ID NOs: 63-65, respectively, and the selected VL2 CDRs 1, 2, 3 amino acid sequences are set forth in SEQ ID NOs: 32-34, respectively;
(35) the selected VH1 CDRs 1, 2, 3 amino acid sequences are set forth in SEQ ID NOs: 13-15, respectively, and the selected VL1 CDRs 1, 2, 3 amino acid sequences are set forth in SEQ ID NOs: 32-34, respectively, and the selected VH2 CDRs 1, 2, 3 amino acid sequences are set forth in SEQ ID NOs: 66-68, respectively, and the selected VL2 CDRs 1, 2, 3 amino acid sequences are set forth in SEQ ID NOs: 32-34, respectively;
(36) the selected VH1 CDRs 1, 2, 3 amino acid sequences are set forth in SEQ ID NOs: 13-15, respectively, and the selected VL1 CDRs 1, 2, 3 amino acid sequences are set forth in SEQ ID NOs: 32-34, respectively, and the selected VH2 CDRs 1, 2, 3 amino acid sequences are set forth in SEQ ID NOs: 69-71, respectively, and the selected VL2 CDRs 1, 2, 3 amino acid sequences are set forth in SEQ ID NOs: 32-34, respectively;
(37) the selected VH1 CDRs 1, 2, 3 amino acid sequences are set forth in SEQ ID NOs: 13-15, respectively, and the selected VL1 CDRs 1, 2, 3 amino acid sequences are set forth in SEQ ID NOs: 32-34, respectively, and the selected VH2 CDRs 1, 2, 3 amino acid sequences are set forth in SEQ ID NOs: 72-74, respectively, and the selected VL2 CDRs 1, 2, 3 amino acid sequences are set forth in SEQ ID NOs: 32-34, respectively;
(38) the selected VH1 CDRs 1, 2, 3 amino acid sequences are set forth in SEQ ID NOs: 13-15, respectively, and the selected VL1 CDRs 1, 2, 3 amino acid sequences are set forth in SEQ ID NOs: 32-34, respectively, and the selected VH2 CDRs 1, 2, 3 amino acid sequences are set forth in SEQ ID NOs: 75-77, respectively, and the selected VL2 CDRs 1, 2, 3 amino acid sequences are set forth in SEQ ID NOs: 32-34, respectively;
(39) the selected VH1 CDRs 1, 2, 3 amino acid sequences are set forth in SEQ ID NOs: 13-15, respectively, and the selected VL1 CDRs 1, 2, 3 amino acid sequences are set forth in SEQ ID NOs: 32-34, respectively, and the selected VH2 CDRs 1, 2, 3 amino acid sequences are set forth in SEQ ID NOs: 78-80, respectively, and the selected VL2 CDRs 1, 2, 3 amino acid sequences are set forth in SEQ ID NOs: 32-34, respectively;
(40) the selected VH1 CDRs 1, 2, 3 amino acid sequences are set forth in SEQ ID NOs: 13-15, respectively, and the selected VL1 CDRs 1, 2, 3 amino acid sequences are set forth in SEQ ID NOs: 32-34, respectively, and the selected VH2 CDRs 1, 2, 3 amino acid sequences are set forth in SEQ ID NOs: 81-83, respectively, and the selected VL2 CDRs 1, 2, 3 amino acid sequences are set forth in SEQ ID NOs: 32-34, respectively;
(41) the selected VH1 CDRs 1, 2, 3 amino acid sequences are set forth in SEQ ID NOs: 16-18, respectively, and the selected VL1 CDRs 1, 2, 3 amino acid sequences are set forth in SEQ ID NOs: 32-34, respectively, and the selected VH2 CDRs 1, 2, 3 amino acid sequences are set forth in SEQ ID NOs: 60-62, respectively, and the selected VL2 CDRs 1, 2, 3 amino acid sequences are set forth in SEQ ID NOs: 32-34, respectively;
(42) the selected VH1 CDRs 1, 2, 3 amino acid sequences are set forth in SEQ ID NOs: 16-18, respectively, and the selected VL1 CDRs 1, 2, 3 amino acid sequences are set forth in SEQ ID NOs: 32-34, respectively, and the selected VH2 CDRs 1, 2, 3 amino acid sequences are set forth in SEQ ID NOs: 63-65, respectively, and the selected VL2 CDRs 1, 2, 3 amino acid sequences are set forth in SEQ ID NOs: 32-34, respectively;
(43) the selected VH1 CDRs 1, 2, 3 amino acid sequences are set forth in SEQ ID NOs: 16-18, respectively, and the selected VL1 CDRs 1, 2, 3 amino acid sequences are set forth in SEQ ID NOs: 32-34, respectively, and the selected VH2 CDRs 1, 2, 3 amino acid sequences are set forth in SEQ ID NOs: 66-68, respectively, and the selected VL2 CDRs 1, 2, 3 amino acid sequences are set forth in SEQ ID NOs: 32-34, respectively;
(44) the selected VH1 CDRs 1, 2, 3 amino acid sequences are set forth in SEQ ID NOs: 16-18, respectively, and the selected VL1 CDRs 1, 2, 3 amino acid sequences are set forth in SEQ ID NOs: 32-34, respectively, and the selected VH2 CDRs 1, 2, 3 amino acid sequences are set forth in SEQ ID NOs: 69-71, respectively, and the selected VL2 CDRs 1, 2, 3 amino acid sequences are set forth in SEQ ID NOs: 32-34, respectively;
(45) the selected VH1 CDRs 1, 2, 3 amino acid sequences are set forth in SEQ ID NOs: 16-18, respectively, and the selected VL1 CDRs 1, 2, 3 amino acid sequences are set forth in SEQ ID NOs: 32-34, respectively, and the selected VH2 CDRs 1, 2, 3 amino acid sequences are set forth in SEQ ID NOs: 72-74, respectively, and the selected VL2 CDRs 1, 2, 3 amino acid sequences are set forth in SEQ ID NOs: 32-34, respectively;
(46) the selected VH1 CDRs 1, 2, 3 amino acid sequences are set forth in SEQ ID NOs: 16-18, respectively, and the selected VL1 CDRs 1, 2, 3 amino acid sequences are set forth in SEQ ID NOs: 32-34, respectively, and the selected VH2 CDRs 1, 2, 3 amino acid sequences are set forth in SEQ ID NOs: 75-77, respectively, and the selected VL2 CDRs 1, 2, 3 amino acid sequences are set forth in SEQ ID NOs: 32-34, respectively;
(47) the selected VH1 CDRs 1, 2, 3 amino acid sequences are set forth in SEQ ID NOs: 16-18, respectively, and the selected VL1 CDRs 1, 2, 3 amino acid sequences are set forth in SEQ ID NOs: 32-34, respectively, and the selected VH2 CDRs 1, 2, 3 amino acid sequences are set forth in SEQ ID NOs: 78-80, respectively, and the selected VL2 CDRs 1, 2, 3 amino acid sequences are set forth in SEQ ID NOs: 32-34, respectively;
(48) the selected VH1 CDRs 1, 2, 3 amino acid sequences are set forth in SEQ ID NOs: 16-18, respectively, and the selected VL1 CDRs 1, 2, 3 amino acid sequences are set forth in SEQ ID NOs: 32-34, respectively, and the selected VH2 CDRs 1, 2, 3 amino acid sequences are set forth in SEQ ID NOs: 81-83, respectively, and the selected VL2 CDRs 1, 2, 3 amino acid sequences are set forth in SEQ ID NOs: 32-34, respectively;
(49) the selected VH1 CDRs 1, 2, 3 amino acid sequences are set forth in SEQ ID NOs: 19-21, respectively, and the selected VL1 CDRs 1, 2, 3 amino acid sequences are set forth in SEQ ID NOs: 32-34, respectively, and the selected VH2 CDRs 1, 2, 3 amino acid sequences are set forth in SEQ ID NOs: 60-62, respectively, and the selected VL2 CDRs 1, 2, 3 amino acid sequences are set forth in SEQ ID NOs: 32-34, respectively;
(50) the selected VH1 CDRs 1, 2, 3 amino acid sequences are set forth in SEQ ID NOs: 19-21, respectively, and the selected VL1 CDRs 1, 2, 3 amino acid sequences are set forth in SEQ ID NOs: 32-34, respectively, and the selected VH2 CDRs 1, 2, 3 amino acid sequences are set forth in SEQ ID NOs: 63-65, respectively, and the selected VL2 CDRs 1, 2, 3 amino acid sequences are set forth in SEQ ID NOs: 32-34, respectively;
(51) the selected VH1 CDRs 1, 2, 3 amino acid sequences are set forth in SEQ ID NOs: 19-21, respectively, and the selected VL1 CDRs 1, 2, 3 amino acid sequences are set forth in SEQ ID NOs: 32-34, respectively, and the selected VH2 CDRs 1, 2, 3 amino acid sequences are set forth in SEQ ID NOs: 66-68, respectively, and the selected VL2 CDRs 1, 2, 3 amino acid sequences are set forth in SEQ ID NOs: 32-34, respectively;
(52) the selected VH1 CDRs 1, 2, 3 amino acid sequences are set forth in SEQ ID NOs: 19-21, respectively, and the selected VL1 CDRs 1, 2, 3 amino acid sequences are set forth in SEQ ID NOs: 32-34, respectively, and the selected VH2 CDRs 1, 2, 3 amino acid sequences are set forth in SEQ ID NOs: 69-71, respectively, and the selected VL2 CDRs 1, 2, 3 amino acid sequences are set forth in SEQ ID NOs: 32-34, respectively;
(53) the selected VH1 CDRs 1, 2, 3 amino acid sequences are set forth in SEQ ID NOs: 19-21, respectively, and the selected VL1 CDRs 1, 2, 3 amino acid sequences are set forth in SEQ ID NOs: 32-34, respectively, and the selected VH2 CDRs 1, 2, 3 amino acid sequences are set forth in SEQ ID NOs: 72-74, respectively, and the selected VL2 CDRs 1, 2, 3 amino acid sequences are set forth in SEQ ID NOs: 32-34, respectively;
(54) the selected VH1 CDRs 1, 2, 3 amino acid sequences are set forth in SEQ ID NOs: 19-21, respectively, and the selected VL1 CDRs 1, 2, 3 amino acid sequences are set forth in SEQ ID NOs: 32-34, respectively, and the selected VH2 CDRs 1, 2, 3 amino acid sequences are set forth in SEQ ID NOs: 75-77, respectively, and the selected VL2 CDRs 1, 2, 3 amino acid sequences are set forth in SEQ ID NOs: 32-34, respectively;
(55) the selected VH1 CDRs 1, 2, 3 amino acid sequences are set forth in SEQ ID NOs: 19-21, respectively, and the selected VL1 CDRs 1, 2, 3 amino acid sequences are set forth in SEQ ID NOs: 32-34, respectively, and the selected VH2 CDRs 1, 2, 3 amino acid sequences are set forth in SEQ ID NOs: 78-80, respectively, and the selected VL2 CDRs 1, 2, 3 amino acid sequences are set forth in SEQ ID NOs: 32-34, respectively;
(56) the selected VH1 CDRs 1, 2, 3 amino acid sequences are set forth in SEQ ID NOs: 19-21, respectively, and the selected VL1 CDRs 1, 2, 3 amino acid sequences are set forth in SEQ ID NOs: 32-34, respectively, and the selected VH2 CDRs 1, 2, 3 amino acid sequences are set forth in SEQ ID NOs: 81-83, respectively, and the selected VL2 CDRs 1, 2, 3 amino acid sequences are set forth in SEQ ID NOs: 32-34, respectively;
(57) the selected VH1 CDRs 1, 2, 3 amino acid sequences are set forth in SEQ ID NOs: 22-24, respectively, and the selected VL1 CDRs 1, 2, 3 amino acid sequences are set forth in SEQ ID NOs: 32-34, respectively, and the selected VH2 CDRs 1, 2, 3 amino acid sequences are set forth in SEQ ID NOs: 60-62, respectively, and the selected VL2 CDRs 1, 2, 3 amino acid sequences are set forth in SEQ ID NOs: 32-34, respectively;
(58) the selected VH1 CDRs 1, 2, 3 amino acid sequences are set forth in SEQ ID NOs: 22-24, respectively, and the selected VL1 CDRs 1, 2, 3 amino acid sequences are set forth in SEQ ID NOs: 32-34, respectively, and the selected VH2 CDRs 1, 2, 3 amino acid sequences are set forth in SEQ ID NOs: 63-65, respectively, and the selected VL2 CDRs 1, 2, 3 amino acid sequences are set forth in SEQ ID NOs: 32-34, respectively;
(59) the selected VH1 CDRs 1, 2, 3 amino acid sequences are set forth in SEQ ID NOs: 22-24, respectively, and the selected VL1 CDRs 1, 2, 3 amino acid sequences are set forth in SEQ ID NOs: 32-34, respectively, and the selected VH2 CDRs 1, 2, 3 amino acid sequences are set forth in SEQ ID NOs: 66-68, respectively, and the selected VL2 CDRs 1, 2, 3 amino acid sequences are set forth in SEQ ID NOs: 32-34, respectively;
(60) the selected VH1 CDRs 1, 2, 3 amino acid sequences are set forth in SEQ ID NOs: 22-24, respectively, and the selected VL1 CDRs 1, 2, 3 amino acid sequences are set forth in SEQ ID NOs: 32-34, respectively, and the selected VH2 CDRs 1, 2, 3 amino acid sequences are set forth in SEQ ID NOs: 69-71, respectively, and the selected VL2 CDRs 1, 2, 3 amino acid sequences are set forth in SEQ ID NOs: 32-34, respectively;
(61) the selected VH1 CDRs 1, 2, 3 amino acid sequences are set forth in SEQ ID NOs: 22-24, respectively, and the selected VL1 CDRs 1, 2, 3 amino acid sequences are set forth in SEQ ID NOs: 32-34, respectively, and the selected VH2 CDRs 1, 2, 3 amino acid sequences are set forth in SEQ ID NOs: 72-74, respectively, and the selected VL2 CDRs 1, 2, 3 amino acid sequences are set forth in SEQ ID NOs: 32-34, respectively;
(62) the selected VH1 CDRs 1, 2, 3 amino acid sequences are set forth in SEQ ID NOs: 22-24, respectively, and the selected VL1 CDRs 1, 2, 3 amino acid sequences are set forth in SEQ ID NOs: 32-34, respectively, and the selected VH2 CDRs 1, 2, 3 amino acid sequences are set forth in SEQ ID NOs: 75-77, respectively, and the selected VL2 CDRs 1, 2, 3 amino acid sequences are set forth in SEQ ID NOs: 32-34, respectively;
(63) the selected VH1 CDRs 1, 2, 3 amino acid sequences are set forth in SEQ ID NOs: 22-24, respectively, and the selected VL1 CDRs 1, 2, 3 amino acid sequences are set forth in SEQ ID NOs: 32-34, respectively, and the selected VH2 CDRs 1, 2, 3 amino acid sequences are set forth in SEQ ID NOs: 78-80, respectively, and the selected VL2 CDRs 1, 2, 3 amino acid sequences are set forth in SEQ ID NOs: 32-34, respectively; and
(64) the selected VH1 CDRs 1, 2, 3 amino acid sequences are set forth in SEQ ID NOs: 22-24, respectively, and the selected VL1 CDRs 1, 2, 3 amino acid sequences are set forth in SEQ ID NOs: 32-34, respectively, and the selected VH2 CDRs 1, 2, 3 amino acid sequences are set forth in SEQ ID NOs: 81-83, respectively, and the selected VL2 CDRs 1, 2, 3 amino acid sequences are set forth in SEQ ID NOs: 32-34, respectively.

70. The antigen-binding protein construct of any one of claims 66-69, wherein the first heavy chain variable region comprises a sequence that is at least 80%, 85%, 90%, or 95% identical to SEQ ID NO: 25, the first light chain variable region comprises a sequence that is at least 80%, 85%, 90%, or 95% identical to SEQ ID NO: 35, the second heavy chain variable region comprises a sequence that is at least 80%, 85%, 90%, or 95% identical to SEQ ID NO: 84, and the second light chain variable region comprises a sequence that is at least 80%, 85%, 90%, or 95% identical to SEQ ID NO: 35.

71. The antigen-binding protein construct of any one of claims 66-69, wherein the first heavy chain variable region comprises a sequence that is at least 80%, 85%, 90%, or 95% identical to SEQ ID NO: 25, the first light chain variable region comprises a sequence that is at least 80%, 85%, 90%, or 95% identical to SEQ ID NO: 35, the second heavy chain variable region comprises a sequence that is at least 80%, 85%, 90%, or 95% identical to SEQ ID NO: 85, and the second light chain variable region comprises a sequence that is at least 80%, 85%, 90%, or 95% identical to SEQ ID NO: 35.

72. The antigen-binding protein construct of any one of claims 66-69, wherein the first heavy chain variable region comprises a sequence that is at least 80%, 85%, 90%, or 95% identical to SEQ ID NO: 25, the first light chain variable region comprises a sequence that is at least 80%, 85%, 90%, or 95% identical to SEQ ID NO: 35, the second heavy chain variable region comprises a sequence that is at least 80%, 85%, 90%, or 95% identical to SEQ ID NO: 86, and the second light chain variable region comprises a sequence that is at least 80%, 85%, 90%, or 95% identical to SEQ ID NO: 35.

73. The antigen-binding protein construct of any one of claims 66-69, wherein the first heavy chain variable region comprises a sequence that is at least 80%, 85%, 90%, or 95% identical to SEQ ID NO: 25, the first light chain variable region comprises a sequence that is at least 80%, 85%, 90%, or 95% identical to SEQ ID NO: 35, the second heavy chain variable region comprises a sequence that is at least 80%, 85%, 90%, or 95% identical to SEQ ID NO: 87, and the second light chain variable region comprises a sequence that is at least 80%, 85%, 90%, or 95% identical to SEQ ID NO: 35.

74. The antigen-binding protein construct of any one of claims 66-69, wherein the first heavy chain variable region comprises a sequence that is at least 80%, 85%, 90%, or 95% identical to SEQ ID NO: 25, the first light chain variable region comprises a sequence that is at least 80%, 85%, 90%, or 95% identical to SEQ ID NO: 35, the second heavy chain variable region comprises a sequence that is at least 80%, 85%, 90%, or 95% identical to SEQ ID NO: 88, and the second light chain variable region comprises a sequence that is at least 80%, 85%, 90%, or 95% identical to SEQ ID NO: 35.

75. The antigen-binding protein construct of any one of claims 66-69, wherein the first heavy chain variable region comprises a sequence that is at least 80%, 85%, 90%, or 95% identical to SEQ ID NO: 25, the first light chain variable region comprises a sequence that is at least 80%, 85%, 90%, or 95% identical to SEQ ID NO: 35, the second heavy chain variable region comprises a sequence that is at least 80%, 85%, 90%, or 95% identical to SEQ ID NO: 89, and the second light chain variable region comprises a sequence that is at least 80%, 85%, 90%, or 95% identical to SEQ ID NO: 35.

76. The antigen-binding protein construct of any one of claims 66-69, wherein the first heavy chain variable region comprises a sequence that is at least 80%, 85%, 90%, or 95% identical to SEQ ID NO: 25, the first light chain variable region comprises a sequence that is at least 80%, 85%, 90%, or 95% identical to SEQ ID NO: 35, the second heavy chain variable region comprises a sequence that is at least 80%, 85%, 90%, or 95% identical to SEQ ID NO: 90, and the second light chain variable region comprises a sequence that is at least 80%, 85%, 90%, or 95% identical to SEQ ID NO: 35.

77. The antigen-binding protein construct of any one of claims 66-69, wherein the first heavy chain variable region comprises a sequence that is at least 80%, 85%, 90%, or 95% identical to SEQ ID NO: 25, the first light chain variable region comprises a sequence that is at least 80%, 85%, 90%, or 95% identical to SEQ ID NO: 35, the second heavy chain variable region comprises a sequence that is at least 80%, 85%, 90%, or 95% identical to SEQ ID NO: 91, and the second light chain variable region comprises a sequence that is at least 80%, 85%, 90%, or 95% identical to SEQ ID NO: 35.

78. The antigen-binding protein construct of any one of claims 66-69, wherein the first heavy chain variable region comprises a sequence that is at least 80%, 85%, 90%, or 95% identical to SEQ ID NO: 26, the first light chain variable region comprises a sequence that is at least 80%, 85%, 90%, or 95% identical to SEQ ID NO: 35, the second heavy chain variable region comprises a sequence that is at least 80%, 85%, 90%, or 95% identical to SEQ ID NO: 84, and the second light chain variable region comprises a sequence that is at least 80%, 85%, 90%, or 95% identical to SEQ ID NO: 35.

79. The antigen-binding protein construct of any one of claims 66-69, wherein the first heavy chain variable region comprises a sequence that is at least 80%, 85%, 90%, or 95% identical to SEQ ID NO: 26, the first light chain variable region comprises a sequence that is at least 80%, 85%, 90%, or 95% identical to SEQ ID NO: 35, the second heavy chain variable region comprises a sequence that is at least 80%, 85%, 90%, or 95% identical to SEQ ID NO: 85, and the second light chain variable region comprises a sequence that is at least 80%, 85%, 90%, or 95% identical to SEQ ID NO: 35.

80. The antigen-binding protein construct of any one of claims 66-69, wherein the first heavy chain variable region comprises a sequence that is at least 80%, 85%, 90%, or 95% identical to SEQ ID NO: 26, the first light chain variable region comprises a sequence that is at least 80%, 85%, 90%, or 95% identical to SEQ ID NO: 35, the second heavy chain variable region comprises a sequence that is at least 80%, 85%, 90%, or 95% identical to SEQ ID NO: 86, and the second light chain variable region comprises a sequence that is at least 80%, 85%, 90%, or 95% identical to SEQ ID NO: 35.

81. The antigen-binding protein construct of any one of claims 66-69, wherein the first heavy chain variable region comprises a sequence that is at least 80%, 85%, 90%, or 95% identical to SEQ ID NO: 26, the first light chain variable region comprises a sequence that is at least 80%, 85%, 90%, or 95% identical to SEQ ID NO: 35, the second heavy chain variable region comprises a sequence that is at least 80%, 85%, 90%, or 95% identical to SEQ ID NO: 87, and the second light chain variable region comprises a sequence that is at least 80%, 85%, 90%, or 95% identical to SEQ ID NO: 35.

82. The antigen-binding protein construct of any one of claims 66-69, wherein the first heavy chain variable region comprises a sequence that is at least 80%, 85%, 90%, or 95% identical to SEQ ID NO: 26, the first light chain variable region comprises a sequence that is at least 80%, 85%, 90%, or 95% identical to SEQ ID NO: 35, the second heavy chain variable region comprises a sequence that is at least 80%, 85%, 90%, or 95% identical to SEQ ID NO: 88, and the second light chain variable region comprises a sequence that is at least 80%, 85%, 90%, or 95% identical to SEQ ID NO: 35.

83. The antigen-binding protein construct of any one of claims 66-69, wherein the first heavy chain variable region comprises a sequence that is at least 80%, 85%, 90%, or 95% identical to SEQ ID NO: 26, the first light chain variable region comprises a sequence that is at least 80%, 85%, 90%, or 95% identical to SEQ ID NO: 35, the second heavy chain variable region comprises a sequence that is at least 80%, 85%, 90%, or 95% identical to SEQ ID NO: 89, and the second light chain variable region comprises a sequence that is at least 80%, 85%, 90%, or 95% identical to SEQ ID NO: 35.

84. The antigen-binding protein construct of any one of claims 66-69, wherein the first heavy chain variable region comprises a sequence that is at least 80%, 85%, 90%, or 95% identical to SEQ ID NO: 26, the first light chain variable region comprises a sequence that is at least 80%, 85%, 90%, or 95% identical to SEQ ID NO: 35, the second heavy chain variable region comprises a sequence that is at least 80%, 85%, 90%, or 95% identical to SEQ ID NO: 90, and the second light chain variable region comprises a sequence that is at least 80%, 85%, 90%, or 95% identical to SEQ ID NO: 35.

85. The antigen-binding protein construct of any one of claims 66-69, wherein the first heavy chain variable region comprises a sequence that is at least 80%, 85%, 90%, or 95% identical to SEQ ID NO: 26, the first light chain variable region comprises a sequence that is at least 80%, 85%, 90%, or 95% identical to SEQ ID NO: 35, the second heavy chain variable region comprises a sequence that is at least 80%, 85%, 90%, or 95% identical to SEQ ID NO: 91, and the second light chain variable region comprises a sequence that is at least 80%, 85%, 90%, or 95% identical to SEQ ID NO: 35.

86. The antigen-binding protein construct of any one of claims 66-69, wherein the first heavy chain variable region comprises a sequence that is at least 80%, 85%, 90%, or 95% identical to SEQ ID NO: 27, the first light chain variable region comprises a sequence that is at least 80%, 85%, 90%, or 95% identical to SEQ ID NO: 35, the second heavy chain variable region comprises a sequence that is at least 80%, 85%, 90%, or 95% identical to SEQ ID NO: 84, and the second light chain variable region comprises a sequence that is at least 80%, 85%, 90%, or 95% identical to SEQ ID NO: 35.

87. The antigen-binding protein construct of any one of claims 66-69, wherein the first heavy chain variable region comprises a sequence that is at least 80%, 85%, 90%, or 95% identical to SEQ ID NO: 27, the first light chain variable region comprises a sequence that is at least 80%, 85%, 90%, or 95% identical to SEQ ID NO: 35, the second heavy chain variable region comprises a sequence that is at least 80%, 85%, 90%, or 95% identical to SEQ ID NO: 85, and the second light chain variable region comprises a sequence that is at least 80%, 85%, 90%, or 95% identical to SEQ ID NO: 35.

88. The antigen-binding protein construct of any one of claims 66-69, wherein the first heavy chain variable region comprises a sequence that is at least 80%, 85%, 90%, or 95% identical to SEQ ID NO: 27, the first light chain variable region comprises a sequence that is at least 80%, 85%, 90%, or 95% identical to SEQ ID NO: 35, the second heavy chain variable region comprises a sequence that is at least 80%, 85%, 90%, or 95% identical to SEQ ID NO: 86, and the second light chain variable region comprises a sequence that is at least 80%, 85%, 90%, or 95% identical to SEQ ID NO: 35.

89. The antigen-binding protein construct of any one of claims 66-69, wherein the first heavy chain variable region comprises a sequence that is at least 80%, 85%, 90%, or 95% identical to SEQ ID NO: 27, the first light chain variable region comprises a sequence that is at least 80%, 85%, 90%, or 95% identical to SEQ ID NO: 35, the second heavy chain variable region comprises a sequence that is at least 80%, 85%, 90%, or 95% identical to SEQ ID NO: 87, and the second light chain variable region comprises a sequence that is at least 80%, 85%, 90%, or 95% identical to SEQ ID NO: 35.

90. The antigen-binding protein construct of any one of claims 66-69, wherein the first heavy chain variable region comprises a sequence that is at least 80%, 85%, 90%, or 95% identical to SEQ ID NO: 27, the first light chain variable region comprises a sequence that is at least 80%, 85%, 90%, or 95% identical to SEQ ID NO: 35, the second heavy chain variable region comprises a sequence that is at least 80%, 85%, 90%, or 95% identical to SEQ ID NO: 88, and the second light chain variable region comprises a sequence that is at least 80%, 85%, 90%, or 95% identical to SEQ ID NO: 35.

91. The antigen-binding protein construct of any one of claims 66-69, wherein the first heavy chain variable region comprises a sequence that is at least 80%, 85%, 90%, or 95% identical to SEQ ID NO: 27, the first light chain variable region comprises a sequence that is at least 80%, 85%, 90%, or 95% identical to SEQ ID NO: 35, the second heavy chain variable region comprises a sequence that is at least 80%, 85%, 90%, or 95% identical to SEQ ID NO: 89, and the second light chain variable region comprises a sequence that is at least 80%, 85%, 90%, or 95% identical to SEQ ID NO: 35.

92. The antigen-binding protein construct of any one of claims 66-69, wherein the first heavy chain variable region comprises a sequence that is at least 80%, 85%, 90%, or 95% identical to SEQ ID NO: 27, the first light chain variable region comprises a sequence that is at least 80%, 85%, 90%, or 95% identical to SEQ ID NO: 35, the second heavy chain variable region comprises a sequence that is at least 80%, 85%, 90%, or 95% identical to SEQ ID NO: 90, and the second light chain variable region comprises a sequence that is at least 80%, 85%, 90%, or 95% identical to SEQ ID NO: 35.

93. The antigen-binding protein construct of any one of claims 66-69, wherein the first heavy chain variable region comprises a sequence that is at least 80%, 85%, 90%, or 95% identical to SEQ ID NO: 27, the first light chain variable region comprises a sequence that is at least 80%, 85%, 90%, or 95% identical to SEQ ID NO: 35, the second heavy chain variable region comprises a sequence that is at least 80%, 85%, 90%, or 95% identical to SEQ ID NO: 91, and the second light chain variable region comprises a sequence that is at least 80%, 85%, 90%, or 95% identical to SEQ ID NO: 35.

94. The antigen-binding protein construct of any one of claims 66-69, wherein the first heavy chain variable region comprises a sequence that is at least 80%, 85%, 90%, or 95% identical to SEQ ID NO: 28, the first light chain variable region comprises a sequence that is at least 80%, 85%, 90%, or 95% identical to SEQ ID NO: 35, the second heavy chain variable region comprises a sequence that is at least 80%, 85%, 90%, or 95% identical to SEQ ID NO: 84, and the second light chain variable region comprises a sequence that is at least 80%, 85%, 90%, or 95% identical to SEQ ID NO: 35.

95. The antigen-binding protein construct of any one of claims 66-69, wherein the first heavy chain variable region comprises a sequence that is at least 80%, 85%, 90%, or 95% identical to SEQ ID NO: 28, the first light chain variable region comprises a sequence that is at least 80%, 85%, 90%, or 95% identical to SEQ ID NO: 35, the second heavy chain variable region comprises a sequence that is at least 80%, 85%, 90%, or 95% identical to SEQ ID NO: 85, and the second light chain variable region comprises a sequence that is at least 80%, 85%, 90%, or 95% identical to SEQ ID NO: 35.

96. The antigen-binding protein construct of any one of claims 66-69, wherein the first heavy chain variable region comprises a sequence that is at least 80%, 85%, 90%, or 95% identical to SEQ ID NO: 28, the first light chain variable region comprises a sequence that is at least 80%, 85%, 90%, or 95% identical to SEQ ID NO: 35, the second heavy chain variable region comprises a sequence that is at least 80%, 85%, 90%, or 95% identical to SEQ ID NO: 86, and the second light chain variable region comprises a sequence that is at least 80%, 85%, 90%, or 95% identical to SEQ ID NO: 35.

97. The antigen-binding protein construct of any one of claims 66-69, wherein the first heavy chain variable region comprises a sequence that is at least 80%, 85%, 90%, or 95% identical to SEQ ID NO: 28, the first light chain variable region comprises a sequence that is at least 80%, 85%, 90%, or 95% identical to SEQ ID NO: 35, the second heavy chain variable region comprises a sequence that is at least 80%, 85%, 90%, or 95% identical to SEQ ID NO: 87, and the second light chain variable region comprises a sequence that is at least 80%, 85%, 90%, or 95% identical to SEQ ID NO: 35.

98. The antigen-binding protein construct of any one of claims 66-69, wherein the first heavy chain variable region comprises a sequence that is at least 80%, 85%, 90%, or 95% identical to SEQ ID NO: 28, the first light chain variable region comprises a sequence that is at least 80%, 85%, 90%, or 95% identical to SEQ ID NO: 35, the second heavy chain variable region comprises a sequence that is at least 80%, 85%, 90%, or 95% identical to SEQ ID NO: 88, and the second light chain variable region comprises a sequence that is at least 80%, 85%, 90%, or 95% identical to SEQ ID NO: 35.

99. The antigen-binding protein construct of any one of claims 66-69, wherein the first heavy chain variable region comprises a sequence that is at least 80%, 85%, 90%, or 95% identical to SEQ ID NO: 28, the first light chain variable region comprises a sequence that is at least 80%, 85%, 90%, or 95% identical to SEQ ID NO: 35, the second heavy chain variable region comprises a sequence that is at least 80%, 85%, 90%, or 95% identical to SEQ ID NO: 89, and the second light chain variable region comprises a sequence that is at least 80%, 85%, 90%, or 95% identical to SEQ ID NO: 35.

100. The antigen-binding protein construct of any one of claims 66-69, wherein the first heavy chain variable region comprises a sequence that is at least 80%, 85%, 90%, or 95% identical to SEQ ID NO: 28, the first light chain variable region comprises a sequence that is at least 80%, 85%, 90%, or 95% identical to SEQ ID NO: 35, the second heavy chain variable region comprises a sequence that is at least 80%, 85%, 90%, or 95% identical to SEQ ID NO: 90, and the second light chain variable region comprises a sequence that is at least 80%, 85%, 90%, or 95% identical to SEQ ID NO: 35.

101. The antigen-binding protein construct of any one of claims 66-69, wherein the first heavy chain variable region comprises a sequence that is at least 80%, 85%, 90%, or 95% identical to SEQ ID NO: 28, the first light chain variable region comprises a sequence that is at least 80%, 85%, 90%, or 95% identical to SEQ ID NO: 35, the second heavy chain variable region comprises a sequence that is at least 80%, 85%, 90%, or 95% identical to SEQ ID NO: 91, and the second light chain variable region comprises a sequence that is at least 80%, 85%, 90%, or 95% identical to SEQ ID NO: 35.

102. The antigen-binding protein construct of any one of claims 65-101, wherein the antigen-binding protein construct is a multi-specific antibody (e.g., a bispecific antibody).

103. The antigen-binding protein construct of any one of claims 65-101, wherein the first antigen-binding domain is a single-chain variable fragment (scFV); and/or the second antigen-binding domain is a scFv.

104. The antigen-binding protein construct of any one of claims 66-103, wherein the first light chain variable region and the second light chain variable region are identical.

105. A vector comprising one or more of the nucleic acids of any one of claims 22-26 and 60-64, a nucleic acid encoding the antibody or antigen-binding fragment thereof of any one of claims 1-21 and 27-59, or a nucleic acid encoding the antigen-binding protein construct of any one of claims 65-104.

106. A cell comprising the vector of claim 105.

107. The cell of claim 106, wherein the cell is a CHO cell.

108. A cell comprising one or more of the nucleic acids of any one of claims 22-26 and 60-64, a nucleic acid encoding the antibody or antigen-binding fragment thereof of any one of claims 1-21 and 27-59, or a nucleic acid encoding the antigen-binding protein construct of any one of claims 65-104.

109. A method of producing an antibody or an antigen-binding fragment thereof, or an antigen-binding protein construct, the method comprising
(a) culturing the cell of any one of claims 106-108 under conditions sufficient for the cell to produce the antibody or the antigen-binding fragment thereof, or the antigen-binding protein construct; and
(b) collecting the antibody or the antigen-binding fragment thereof, or the antigen-binding protein construct produced by the cell.

110. An antibody-drug conjugate comprising a therapeutic agent covalently bound to:
(a) the antibody or antigen-binding fragment thereof of any one of claims 1-21 and 27-59; or
(b) the antigen-binding protein construct of any one of claims 65-104.

111. The antibody drug conjugate of claim 110, wherein the therapeutic agent is a cytotoxic or cytostatic agent.

112. A method of treating a subject having cancer, the method comprising administering a therapeutically effective amount of a composition comprising the antibody or antigen-binding fragment thereof of any one of claims 1-21 and 27-59, the antigen-binding protein construct of any one of claims 65-104, or the antibody-drug conjugate of claim 110 or 111, to the subject.

113. The method of claim 112, wherein the subject has a solid tumor.

114. The method of claim 112, wherein the cancer is breast cancer, oropharyngeal cancer, ovarian cancer, bladder cancer, colon cancer, pancreas cancer, B cell lymphoma, hepatocellular carcinoma, or Non-Hodgkin's lymphoma.

115. The method of any one of claims 112-114, wherein the subject is a human.

116. The method of any one of claims 112-115, wherein the method further comprises administering an anti-PD1 antibody to the subject.

117. The method of any one of claims 112-116, wherein the method further comprises administering a chemotherapy to the subject.

118. A method of decreasing the rate of tumor growth, the method comprising contacting a tumor cell with an effective amount of a composition comprising the antibody or antigen-binding fragment thereof of any one of claims 1-21 and 27-59, the antigen-binding protein construct of any one of claims 65-104, or the antibody-drug conjugate of claim 110 or 111.

119. A method of killing a tumor cell, the method comprising contacting a tumor cell with an effective amount of a composition comprising the antibody or antigen-binding fragment thereof of any one of claims 1-21 and 27-59, the antigen-binding protein construct of any one of claims 65-104, or the antibody-drug conjugate of claim 110 or 111.

120. A pharmaceutical composition comprising a pharmaceutically acceptable carrier and
(a) the antibody or antigen-binding fragment thereof of any one of claims 1-21 and 27-59,
(b) the antigen-binding protein construct of any one of claims 65-104, or
(c) the antibody-drug conjugate of any one of claims 110 or 111.
